(19) United States
(12) Patent Application Publication
(10) Pub. No.: US 2024/0141308 A1
(43) Pub. Date: May 2, 2024

(54) ORGANOPHOSPHORUS NERVE AGENT HYDROLYZING ENZYMES

(71) Applicant: Ginkgo Bioworks, Inc., Boston, MA (US)

(72) Inventors: David Borhani, Boston, MA (US); Dylan Alexander Carlin, Jamaica Plain, MA (US); Alex Tucker, Boston, MA (US)

(73) Assignee: Ginkgo Bioworks, Inc., Boston, MA (US)

(21) Appl. No.: 18/283,688

(22) PCT Filed: Apr. 15, 2022
(86) PCT No.: PCT/US22/25011

§ 371 Date: Sep. 22, 2023

Related U.S. Application Data
(60) Provisional application No. 63/176,183, filed on Apr. 16, 2021.

(51) Int. Cl. C12N 9/16; A61K 38/46

(52) U.S. Cl. CPC C12N 9/16; A61K 38/465; C12Y 301/08001

(57) ABSTRACT

Aspects of the disclosure relate to phosphotriesterase (PTE) enzymes and PTE-related (PTER) enzymes and their use in hydrolyzing OPNAs.

### CROSS-REFERENCE TO RELATED APPLICATIONS

This application claims the benefit under 35 U.S.C. § 119(e) of U.S. Provisional Application No. 63/176,183, filed Apr. 16, 2021, entitled “ORGANOPHOSPHORUS NERVE AGENT HYDROLYZING ENZYMES,” the entire disclosure of which is hereby incorporated by reference in its entirety.

### STATEMENT OF GOVERNMENT SUPPORT

This invention was made with Government support under Contract No. 2014-14031000011 awarded by the Central Intelligence Agency. The Government has certain rights in the invention.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. The ASCII file, created on Apr. 14, 2022, is named G091970073W000-SEQ-OMJ.txt and is 2,764,019 bytes in size.

### FIELD OF THE INVENTION

The present disclosure relates to the use of organophosphorus nerve agent hydrolyzing enzymes in the inactivation or elimination of nerve agents such as VX, VR, GB and/or GD by reducing the activity of the nerve agents.

### BACKGROUND

Chemical Warfare Agents (CWAs) are among the deadliest known weapons of mass destruction (WMD). In particular, organophosphorus nerve agents (OPNAs) are a class of CWAs that act rapidly to cause respiratory arrest and death within minutes of cutaneous absorption or inhalation. Multiple hurdles exist to obtaining prophylactic, post-exposure prophylactic, and therapeutic medical countermeasures to OPNAs, including inadequate efficacy, inadequate kinetic parameters, lack of pan-OPNA activity (against the different stereoisomers of, e.g., V-agents and/or G-agents), high cost, and/or immunogenicity.

### SUMMARY

Aspects of the disclosure relate to host cells that comprise a heterologous polynucleotide encoding an OPNA hydrolyzing enzyme, wherein the OPNA hydrolyzing enzyme is a phosphotriesterase (PTE), and wherein the PTE comprises a sequence that is at least 90% identical to any one of SEQ ID NOs: 1-4. In some embodiments, the PTE comprises the sequence of any one of SEQ ID NOs: 1-4.

In some embodiments, the OPNA is a V-agent (such as VX or VR), a G-agent, a VG-agent, an A-agent, an organophosphorus pesticide, or any combination thereof.

In some embodiments, the host cell is a bacterial cell, an archaebacterial cell, a fungal cell, a yeast cell, an animal cell, a mammalian cell, or a human cell. In some embodiments, the host cell is a bacterial cell. In some embodiments, the bacterial cell is an Escherichia coli (E. coli) cell. In some embodiments, the bacterial cell is a Bacillus cell. In some embodiments, the host cell is a filamentous fungi cell or a yeast cell. In some embodiments, the E. coli cell is an E. coli BL21(DE3) cell.

In some embodiments, the PTE comprises one or more amino acid substitutions relative to the sequence of any one of SEQ ID NOs: 1-4. In some embodiments, one or more of the amino acid substitutions relative to the sequence of any one of SEQ ID NOs: 1-4 is within the active site of the PTE. In some embodiments, the PTE comprises a sequence that is at least 90% identical to any one of SEQ ID NOs: 36-795. In some embodiments, the PTE comprises the sequence of any one of SEQ ID NOs: 36-795. In some embodiments, the PTE has a K_{cat}/K_{M} value greater than 10⁷ M⁻¹ min⁻¹.

In some embodiments, the PTE has activity against VX and VR and the PTE comprises one or more of the following amino acid substitutions relative to SEQ ID NO: 1: V25M, I27F, I27M, I27T, V66I, L68N, L68M, L68C, T69S, V70P, V70C, V70T, L144I, A147G, A147F, T148V, T148C, T148M, V164I, S176T, S176M, S176V, S176C, S176Y, S176I, T177C, T179C, T179S, A181P, A181S, A181C, S208T, S208A, G228S, H263M, H263S, H263C, H263T, H263N, A265C, A265T, A265F, A265W, A265M, N266S, N266M, N266T, N266G, N266A, C267A, C267T, C267W, W284D, W284H, W284C, W284N, W284Y, Y286M, and/or Y286W.

In some embodiments, the PTE has activity against VX and VR and the PTE comprises one or more of the following amino acid substitutions relative to SEQ ID NO: 2: S2K, S2T, S2A, E3K, E3T, E3Q, L4I, L4V, N5R, N5Q, N5M, R8L, R8T, R8C, S10P, D12E, D12P, T13P, T13A, A14S, A14E, A14D, A15D, A15Q, A15E, L16M, V18M, V18I, M28D, T29S, T30S, T30W, T30P, E31G, I32V, I32M, I32W, I32F, A33W, E34Q, N35D, Y36F, Y36W, Y36H, E38D, A39P, W40F, D42N, E43D, D44E, D44N, V47I, V47M, A48E, A48W, D49H, D49W, D49C, D49M, V51I, V51L, K52R, R53Q, R53E, N55K, E56D, E56R, E56Q, L57F, A59E, A59Q, R60A, R60H, D63N, T64S, G72D, Y76N, Y76D, I77V, P78D, P78E, I80L, I80M, I80V, A81R, R82K, R82E, V83L, V83I, A84S, A85E, A85R, E86R, T87S, E88G, L89V, L89M, N90H, I91V, V92I, V93C, T99C, V103W, V103Y, V103H, M105W, Y106F, Y106H, Y106W, F107Y, F107W, F107M, Y109W, Y109F, L110M, L110W, E115D, G118T, G118S, E120D, I121Q, I121E, M122L, M122I, T123A, D124E, V127I, R128H, R128N, Q132D, Q132E, I134V, A135E, A135G, D136G, I139V, K140H, K140R, T150Y, T150S, T150H, P151W, P151H, P151N, P151D, V153I, P155E, P155D, G156W, E158H, A165C, Q166R, H168Q, G182D, L183T, L187H, L187M, L187F, E188W, Q190I, Q190L, K191R, F193L, E194D, E195D, L200P, S201N, R202H, R202K, V203C, V2031, 1214M, G215E, G215D, E219D, L220I, L220M, L220V, I221M, I221C, I221A, A222H, A223R, S225C, Y226W, L227V, L227I, D235C, A236H, A236W, A236K, L238M, L238H, P239S, F240W, F240D, F240Y, E241D, D242E, V244C, N245D, N245E, N245R, T246M, T246L, V247I, V247L, Q249E, Q249W, Q249R, Q249H, M250L, C251I, C251V, E252H, R253N, H255Y, H255W, K258H, K258R, M259I, A271G, L272H, L272W, L272M, D274G, D274W, E275K, V277W, S278R, Q279K, Q279R, Q279H, M281Y, M281F, P282G, N283D, N283G, H285G, L287T, H288F, H288Y, I289L, I289V, H290F, H290L, N291R, N291D, N291T, N291E, D292N, D292R, V293I, I294L, I294V, A296M, K298M, K298R, E299Q, E299K, E299R, R300A, T303S, T303D, D304E, D304Q, E305D, E305A, Q306D, Q306E, L307I, L307V, H308R, H308E, H308N, T309Q, T309K, T309R, L311M, L311F, L311T, V312I, D313E, R316A, R316K, R316Q, R317K, R317N, I318M, I318F, I318L, E320S, E320Q, E320D, Q322R, Q322E, Q322K, A324P, A324S, Y325W, Y325F, Y325H, E326Q, E326R, and/or E326K.

In some embodiments, the PTE has activity against VX and VR and the PTE comprises one or more of the following amino acid substitutions relative to SEQ ID NO: 3: V25A, V25T, V25I, I27F, I27T, I27M, I27L, L68N, L68P, L68Q, L68M, T69S, V70P, V70C, Y100E, Y100H, Y100D, Y100Q, L144I, C146V, C146I, A147C, T148I, T148A, T148V, V164I, S176L, S176H, S176V, S176C, S176I, S176Y, S176F, T177C, H178D, T179C, A181S, Q189M, Q189V, Q189A, Q189I, Q189C, G206S, S208A, S208T, S208C, G209N, G228S, V260I, S262G, H263M, H263G, H263T, H263Q, A265T, A265S, A265M, A265W, N266L, N266I, N266G, N266T, N266A, N266C, N266Q, C267T, C267W, W284E, and/or W284T.

In some embodiments, the PTE has activity against VX and VR and the PTE comprises one or more of the following amino acid substitutions relative to SEQ ID NO: 4: L20M, V25L, V25M, F26W, F26H, I27M, I27F, I27V, I27T, V66I, L68N, L68M, L68C, Y98W, F126M, L144I, C146V, A147I, A147G, A147S, A147M, T148M, T148C, T148I, T148V, V164T, V164I, H168S, V173C, S176T, S176M, S176H, H178D, A181S, A181G, Q189M, Q189V, Q189C, I204V, G206S, S208C, S208T, G209N, V260I, S262G, H263S, H263C, H263T, H263N, A265L, A265Q, A265T, A265S, A265Y, A265W, N266C, N266G, N266S, N266T, N266A, C267W, C267A, C267T, C267G, W284H, W284Y, W284M, W284F, W284N, Y286F, Y286M, and/or Y286W.

In some embodiments, the PTE has activity against VX and the PTE comprises one or more of the following amino acid substitutions relative to SEQ ID NO: 1: 127C, I27Q, I27Y, L68A, S176F, T177L, T179E, G209N, and/or N266W. In some embodiments, the PTE has activity against VX and the PTE comprises one or more of the following amino acid substitutions relative to SEQ ID NO: 2: E38H, I77P, V153W, E188C, L238Y, L276W, and/or A296R. In some embodiments, the PTE has activity against VX and the PTE comprises one or more of the following amino acid substitutions relative to SEQ ID NO: 3: V25C, I27Y, I27C, I27W, I27V, L68A, L73V, N266W, and/or N266H. In some embodiments, the PTE has activity against VX and the PTE comprises one or more of the following amino acid substitutions relative to SEQ ID NO: 4: I27C, T69S, F126C, S176Y, S176I, S176F, T177C, H178Y, Q189L, Q189I, Q189A, and/or S208A. In some embodiments, the PTE has activity against VR and the PTE comprises one or more of the following amino acid substitutions relative to SEQ ID NO: 1: V25I, I27W, K145R, S176H, S208C, G228A, H263F, A265L, W284Q, W284K, W284I, and/or W284R. In some embodiments, the PTE has activity against VR and the PTE comprises one or more of the following amino acid substitutions relative to SEQ ID NO: 3: Y98F, Q189L, G206N, H263F, W284K, and/or W284R. In some embodiments, the PTE has activity against VR and the PTE comprises one or more of the following amino acid substitutions relative to SEQ ID NO: 4: E23D, F26M, H178V, D210C, G228S, A265M, A265C, A265I, A265V, W284C, W284Q, and/or W284R.

Further aspects of the disclosure relate to methods of treating or protecting against OPNA toxicity, comprising administering to a subject in need thereof a therapeutically effective amount of an OPNA hydrolyzing enzyme, or a polynucleotide encoding an OPNA hydrolyzing enzyme, wherein the OPNA hydrolyzing enzyme is a phosphotriesterase (PTE), and wherein the PTE comprises a sequence that is at least 90% identical to any one of SEQ ID NOs: 1-4.

Further aspects of the disclosure relate to methods of treating or protecting against OPNA toxicity, comprising administering to a subject in need thereof a cell comprising a heterologous polynucleotide encoding a therapeutically effective amount of an OPNA hydrolyzing enzyme, wherein the OPNA hydrolyzing enzyme is a phosphotriesterase (PTE), and wherein the PTE comprises a sequence that is at least 90% identical to any one of SEQ ID NOs: 1-4. In some embodiments, the cell is a human cell, an animal cell, a yeast cell, or a bacterial cell.

Further aspects of the disclosure relate to methods of hydrolyzing or degrading an OPNA, comprising contacting an OPNA with an OPNA hydrolyzing enzyme, wherein the OPNA hydrolyzing enzyme is a phosphotriesterase (PTE), and wherein the PTE comprises a sequence that is at least 90% identical to any one of SEQ ID NOs: 1-4. Further aspects of the disclosure relate to methods of hydrolyzing or degrading an OPNA, comprising contacting an OPNA with a cell comprising a heterologous polynucleotide encoding an OPNA hydrolyzing enzyme, wherein the OPNA hydrolyzing enzyme is a phosphotriesterase (PTE), wherein the PTE comprises a sequence that is at least 90% identical to any one of SEQ ID NOs: 1-4, and wherein the cell is in a solution, in a sprayable form, in dried form, or in immobilized form.

In some embodiments, the cell is an archaebacterium cell or a soil bacterium cell, such as a Bacillus cell. In some embodiments, the PTE comprises the sequence of any one of SEQ ID NOs: 1-4. In some embodiments, the OPNA is a V-agent (such as VX or VR), a G-agent, a VG-agent, an A-agent, an organophosphorus pesticide, or any combination thereof.

In some embodiments, the PTE is recombinantly produced. In some embodiments, the PTE is recombinantly produced in a bacterial cell or archaebacterial cell. In some embodiments, the bacterial cell is an E. coli cell. In some embodiments, the bacterial cell is a Bacillus cell. In some embodiments, the PTE is recombinantly produced in a filamentous fungi cell or a yeast cell. In some embodiments, the E. cell is an E. coli BL21(DE3) cell. In some embodiments, the PTE comprises one or more amino acid substitutions relative to the sequence of any one of SEQ ID NOs: 1-4. In some embodiments, one or more of the amino acid substitutions relative to the sequence of any one of SEQ ID NOs: 1-4 is within the active site of the PTE. In some embodiments, the PTE comprises a sequence that is at least 90% identical to any one of SEQ ID NOs: 36-795. In some embodiments, the PTE comprises the sequence of any one of SEQ ID NOs: 36-795. In some embodiments, the PTE has a K_{cat}/K_{M} value greater than 10⁷ M⁻¹ min⁻¹.

In some embodiments, the PTE is applied to an article of clothing. In some embodiments, the method is a method of protecting a subject against exposure to an OPNA. In some embodiments, the method is a method of treating a subject that has been exposed to an OPNA.

Further aspects of the disclosure relate to OPNA hydrolyzing enzymes, wherein the OPNA hydrolyzing enzyme is a PTE, wherein the PTE comprises a sequence that is at least 90% identical to any one of SEQ ID NOs: 1-4, and wherein the sequence comprises one or more amino acid substitutions relative to the sequence of any one of SEQ ID NOs: 1-4.

In some embodiments, one or more of the amino acid substitutions relative to the sequence of any one of SEQ ID NOs: 1-4 is within the active site of the PTE. In some embodiments, the OPNA is a V-agent (such as VX or VR), a G-agent, a VG-agent, an A-agent, an organophosphorus pesticide, or any combination thereof. In some embodiments, the PTE is recombinantly produced.

In some embodiments, the PTE is recombinantly produced in a bacterial cell or an archaebacterial cell. In some embodiments, the bacterial cell is an E. coli cell. In some embodiments, the bacterial cell is a Bacillus cell. In some embodiments, the PTE is recombinantly produced in a filamentous fungi cell or a yeast cell. In some embodiments, the E. cell is an E. coli BL21(DE3) cell.

In some embodiments, the PTE comprises a sequence that is at least 90% identical to any one of SEQ ID NOs: 36-795. In some embodiments, the PTE comprises the sequence of any one of SEQ ID NOs: 36-795. In some embodiments, the PTE has a k_{cat}/K_{M} value greater than 107 M⁻¹ min⁻¹.

In some embodiments, the OPNA hydrolyzing enzyme has activity against VX and VR and the OPNA hydrolyzing enzyme comprises one or more of the following amino acid substitutions relative to SEQ ID NO: 1: V25M, I27F, I27M, I27T, V66I, L68N, L68M, L68C, T69S, V70P, V70C, V70T, L144I, A147G, A147F, T148V, T148C, T148M, V164I, S176T, S176M, S176V, S176C, S176Y, S176I, T177C, T179C, T179S, A181P, A181S, A181C, S208T, S208A, G228S, H263M, H263S, H263C, H263T, H263N, A265C, A265T, A265F, A265W, A265M, N266S, N266M, N266T, N266G, N266A, C267A, C267T, C267W, W284D, W284H, W284C, W284N, W284Y, Y286M, and/or Y286W.

In some embodiments, the OPNA hydrolyzing enzyme has activity against VX and VR and the OPNA hydrolyzing enzyme comprises one or more of the following amino acid substitutions relative to SEQ ID NO: 2: S2K, S2T, S2A, E3K, E3T, E3Q, L4I, L4V, N5R, N5Q, N5M, R8L, R8T, R8C, S10P, D12E, D12P, T13P, T13A, A14S, A14E, A14D, A15D, A15Q, A15E, L16M, V18M, V18I, M28D, T29S, T30S, T30W, T30P, E31G, I32V, I32M, I32W, I32F, A33W, E34Q, N35D, Y36F, Y36W, Y36H, E38D, A39P, W40F, D42N, E43D, D44E, D44N, V47I, V47M, A48E, A48W, D49H, D49W, D49C, D49M, V51I, V51L, K52R, R53Q, R53E, N55K, E56D, E56R, E56Q, L57F, A59E, A59Q, R60A, R60H, D63N, T64S, G72D, Y76N, Y76D, I77V, P78D, P78E, I80L, I80M, I80V, A81R, R82K, R82E, V83L, V83I, A84S, A85E, A85R, E86R, T87S, E88G, L89V, L89M, N90H, I91V, V92I, V93C, T99C, V103W, V103Y, V103H, M105W, Y106F, Y106H, Y106W, F107Y, F107W, F107M, Y109W, Y109F, L110M, L110W, E115D, G118T, G118S, E120D, I121Q, I121E, M122L, M122I, T123A, D124E, V127I, R128H, R128N, Q132D, Q132E, I134V, A135E, A135G, D136G, I139V, K140H, K140R, T150Y, T150S, T150H, P151W, P151H, P151N, P151D, V153I, P155E, P155D, G156W, E158H, A165C, Q166R, H168Q, G182D, L183T, L187H, L187M, L187F, E188W, Q190I, Q190L, K191R, F193L, E194D, E195D, L200P, S201N, R202H, R202K, V203C, V2031, 1214M, G215E, G215D, E219D, L220I, L220M, L220V, I221M, I221C, I221A, A222H, A223R, S225C, Y226W, L227V, L227I, D235C, A236H, A236W, A236K, L238M, L238H, P239S, F240W, F240D, F240Y, E241D, D242E, V244C, N245D, N245E, N245R, T246M, T246L, V247I, V247L, Q249E, Q249W, Q249R, Q249H, M250L, C251I, C251V, E252H, R253N, H255Y, H255W, K258H, K258R, M259I, A271G, L272H, L272W, L272M, D274G, D274W, E275K, V277W, S278R, Q279K, Q279R, Q279H, M281Y, M281F, P282G, N283D, N283G, H285G, L287T, H288F, H288Y, I289L, I289V, H290F, H290L, N291R, N291D, N291T, N291E, D292N, D292R, V293I, I294L, I294V, A296M, K298M, K298R, E299Q, E299K, E299R, R300A, T303S, T303D, D304E, D304Q, E305D, E305A, Q306D, Q306E, L307I, L307V, H308R, H308E, H308N, T309Q, T309K, T309R, L311M, L311F, L311T, V312I, D313E, R316A, R316K. R316Q, R317K, R317N, I318M, I318F, I318L, E320S, E320Q, E320D, Q322R, Q322E, Q322K, A324P, A324S, Y325W, Y325F, Y325H, E326Q, E326R, and/or E326K.

In some embodiments, the OPNA hydrolyzing enzyme has activity against VX and VR and the OPNA hydrolyzing enzyme comprises one or more of the following amino acid substitutions relative to SEQ ID NO: 3: V25A, V25T, V25I, I27F, I27T, I27M, I27L, L68N, L68P, L68Q, L68M, T69S, V70P, V70C, Y100E, Y100H, Y100D, Y100Q, L144I, C146V, C146I, A147C, T148I, T148A, T148V, V164I, S176L, S176H, S176V, S176C, S176I, S176Y, S176F, T177C, H178D, T179C, A181S, Q189M, Q189V, Q189A, Q189I, Q189C, G206S, S208A, S208T, S208C, G209N, G228S, V260I, S262G, H263M, H263G, H263T, H263Q, A265T, A265S, A265M, A265W, N266L, N266I, N266G, N266T, N266A, N266C, N266Q, C267T, C267W, W284E, and/or W284T.

In some embodiments, the OPNA hydrolyzing enzyme has activity against VX and VR and the OPNA hydrolyzing enzyme comprises one or more of the following amino acid substitutions relative to SEQ ID NO: 4: L20M, V25L, V25M, F26W, F26H, I27M, I27F, I27V, I27T, V66I, L68N, L68M, L68C, Y98W, F126M, L144I, C146V, A147I, A147G, A147S, A147M, T148M, T148C, T148I, T148V, V164T, V164I, H168S, V173C, S176T, S176M, S176H, H178D, A181S, A181G, Q189M, Q189V, Q189C, I204V, G206S, S208C, S208T, G209N, V260I, S262G, H263S, H263C, H263T, H263N, A265L, A265Q, A265T, A265S, A265Y, A265W, N266C, N266G, N266S, N266T, N266A, C267W, C267A, C267T, C267G, W284H, W284Y, W284M, W284F, W284N, Y286F, Y286M, and/or Y286W.

In some embodiments, the OPNA hydrolyzing enzyme has activity against VX and the OPNA hydrolyzing enzyme comprises one or more of the following amino acid substitutions relative to SEQ ID NO: 1: I27C, I27Q, I27Y, L68A, S176F, T177L, T179E, G209N, and/or N266W. In some embodiments, the OPNA hydrolyzing enzyme has activity against VX and the OPNA hydrolyzing enzyme comprises one or more of the following amino acid substitutions relative to SEQ ID NO: 2: E38H, I77P, V153W, E188C, L238Y, L276W, and/or A296R. In some embodiments, the OPNA hydrolyzing enzyme has activity against VX and the OPNA hydrolyzing enzyme comprises one or more of the following amino acid substitutions relative to SEQ ID NO: 3: V25C, I27Y, I27C, I27W, I27V, L68A, L73V, N266W, and/or N266H. In some embodiments, the OPNA hydrolyzing enzyme has activity against VX and the OPNA hydrolyzing enzyme comprises one or more of the following amino acid substitutions relative to SEQ ID NO: 4: I27C, T69S, F126C, S176Y, S176I, S176F, T177C, H178Y, Q189L, Q189I, Q189A, and/or S208A. In some embodiments, the OPNA hydrolyzing enzyme has activity against VR and the OPNA hydrolyzing enzyme comprises one or more of the following amino acid substitutions relative to SEQ ID NO: 1: V25I, I27W, K145R, S176H, S208C, G228A, H263F, A265L, W284Q, W284K, W284I, and/or W284R. In some embodiments, the OPNA hydrolyzing enzyme has activity against VR and the OPNA hydrolyzing enzyme comprises one or more of the following amino acid substitutions relative to SEQ ID NO: 3: Y98F, Q189L, G206N, H263F, W284K, and/or W284R. In some embodiments, the OPNA hydrolyzing enzyme has activity against VR and the OPNA hydrolyzing enzyme comprises one or more of the following amino acid substitutions relative to SEQ ID NO: 4: E23D, F26M, H178V, D210C, G228S, A265M, A265C, A265I, A265V, W284C, W284Q, and/or W284R.

Further aspects of the disclosure relate to host cells that comprise a heterologous polynucleotide encoding an OPNA hydrolyzing enzyme, wherein the OPNA hydrolyzing enzyme comprises a sequence that is at least 90% identical to any one of SEQ ID NOs: 6 and 796-956. In some embodiments, the OPNA hydrolyzing enzyme comprises the sequence of any one of SEQ ID NOs: 6 and 796-956.

In some embodiments, the OPNA is a V-agent (such as VX or VR), a G-agent, a VG-agent, an A-agent, an organophosphorus pesticide, or any combination thereof. In some embodiments, the host cell is a bacterial cell, an archaebacterial cell, a fungal cell, a yeast cell, an animal cell, a mammalian cell, or a human cell. In some embodiments, the host cell is a bacterial cell. In some embodiments, the bacterial cell is an Escherichia coli (E. coli) cell. In some embodiments, the bacterial cell is a Bacillus cell. In some embodiments, the host cell is a filamentous fungi cell or a yeast cell. In some embodiments, the E. coli cell is an E. coli BL21(DE3) cell. In some embodiments, the PTE has a K_{cat}/K_{M} value greater than 10⁷ M⁻¹ min⁻¹.

Further aspects of the disclosure relate to methods of treating or protecting against OPNA toxicity, comprising administering to a subject in need thereof a therapeutically effective amount of an OPNA hydrolyzing enzyme, or a polynucleotide encoding an OPNA hydrolyzing enzyme, wherein the OPNA hydrolyzing enzyme comprises a sequence that is at least 90% identical to any one of SEQ ID NOs: 6 and 796-956.

Further aspects of the disclosure relate to methods of hydrolyzing or degrading an OPNA, comprising administering to a subject in need thereof a cell comprising a heterologous polynucleotide encoding a therapeutically effective amount of an OPNA hydrolyzing enzyme, wherein the OPNA hydrolyzing enzyme comprises a sequence that is at least 90% identical to any one of SEQ ID NOs: 6 and 796-956. In some embodiments, the cell is a human cell, an animal cell, a yeast cell, or a bacterial cell.

Further aspects of the disclosure relate to methods of hydrolyzing or degrading an OPNA, comprising contacting an OPNA with an OPNA hydrolyzing enzyme, wherein the OPNA hydrolyzing enzyme comprises a sequence that is at least 90% identical to any one of SEQ ID NOs: 6 and 796-956.

Further aspects of the disclosure relate to methods of hydrolyzing or degrading an OPNA, comprising contacting an OPNA with a cell comprising a heterologous polynucleotide encoding an OPNA hydrolyzing enzyme, wherein the OPNA hydrolyzing enzyme comprises a sequence that is at least 90% identical to any one of SEQ ID NOs: 6 and 796-956 and wherein the cell is in a solution, in a sprayable form, in dried form, or in immobilized form. In some embodiments, the cell is an archaebacterium cell or a soil bacterium cell, such as a Bacillus cell.

Further aspects of the disclosure relate to an OPNA hydrolyzing enzyme, wherein the OPNA hydrolyzing enzyme comprises a sequence that is at least 90% identical to any one of SEQ ID NOs: 6 and 796-956. In some embodiments, the OPNA hydrolyzing enzyme comprises one or more amino acid substitutions relative to the sequence of SEQ ID NO: 6.

In some embodiments, the OPNA hydrolyzing enzyme has activity against VX and VR and the OPNA hydrolyzing enzyme comprises the following amino acid substitution relative to SEQ ID NO: 6: 1258V. In some embodiments, the OPNA hydrolyzing enzyme has activity against VR and the OPNA hydrolyzing enzyme comprises the following amino acid substitution relative to SEQ ID NO: 6: G229N. In some embodiments, the PTE has activity against GB and GD and the PTE comprises one or more of the following amino acid substitutions relative to SEQ ID NO: 1: A265Y, N266G, N266M, C267W, and/or W284H. In some embodiments, the PTE has activity against GB and GD and the PTE comprises one or more of the following amino acid substitutions relative to SEQ ID NO: 2: T29S, T99C, V103H, P151W, A236K, L272C, L272W, M281Y, and/or H285G. In some embodiments, the PTE has activity against GB and GD and the PTE comprises one or more of the following amino acid substitutions relative to SEQ ID NO: 3: Y100D, A265Y, N266I, N266L, and/or W284H.

In some embodiments, the PTE has activity against GB and GD and the PTE comprises one or more of the following amino acid substitutions relative to SEQ ID NO: 4: T148V, A265M, A265Y, N266T, C267W, and/or W284H. In some embodiments, the PTE has activity against VX, VR, GB, and GD and the PTE comprises one or more of the following amino acid substitutions relative to SEQ ID NO: 1: A265Y, N266M, and/or C267W. In some embodiments, the PTE has activity against VX, VR, GB, and GD and the PTE comprises one or more of the following amino acid substitutions relative to SEQ ID NO: 2: T99C, V103H, P151W, L272C, and/or L272W. In some embodiments, the PTE has activity against VX, VR, GB, and GD and the PTE comprises one or more of the following amino acid substitutions relative to SEQ ID NO: 3: A265Y, N266I, and/or N266L. In some embodiments, the PTE has activity against VR, GB, and GD and the PTE comprises the following amino acid substitution relative to SEQ ID NO: 1: W284H. In some embodiments, the PTE has activity against VR and GD and the PTE comprises the following amino acid substitution relative to SEQ ID NO: 1: A265Y. In some embodiments, the PTE has activity against VX, VR and GD and the PTE comprises the following amino acid substitution relative to SEQ ID NO: 1: A265Y. In some embodiments, the PTE has activity against VX, VR and GB and the PTE comprises one or more of the following amino acid substitutions relative to SEQ ID NO: 1: C267W and/or N266G. In some embodiments, the PTE has activity against VX, VR and GB and the PTE comprises one or more of the following amino acid substitutions relative to SEQ ID NO: 2: T99C, L272C, and/or L272W. In some embodiments, the PTE has activity against VX, VR and GB and the PTE comprises one of the following amino acid substitutions relative to SEQ ID NO: 3: N266I or N266L.

In some embodiments, the PTE has activity against VX and/or VR. In some embodiments, the PTE has activity against GB and/or GD. In some embodiments, the PTE has activity against VX, VR, GB, and GD.

In some embodiments, the OPNA hydrolyzing enzyme has activity against GB and GD and the OPNA hydrolyzing enzyme comprises one or more of the following amino acid substitutions relative to SEQ ID NO: 1: A265Y, N266G, N266M, C267W, and/or W284H. In some embodiments, the OPNA hydrolyzing enzyme has activity against GB and GD and the OPNA hydrolyzing enzyme comprises one or more of the following amino acid substitutions relative to SEQ ID NO: 2: T29S, T99C, V103H, P151W, A236K, L272C, L272W, M281Y, and/or H285G.

In some embodiments, the OPNA hydrolyzing enzyme has activity against GB and GD and the OPNA hydrolyzing enzyme comprises one or more of the following amino acid substitutions relative to SEQ ID NO: 3: Y100D, A265Y, N266I, N266L, and/or W284H. In some embodiments, the OPNA hydrolyzing enzyme has activity against GB and GD and the OPNA hydrolyzing enzyme comprises one or more of the following amino acid substitutions relative to SEQ ID NO: 4: T148V, A265M, A265Y, N266T, C267W, and/or W284H.

In some embodiments, the OPNA hydrolyzing enzyme has activity against VX, VR, GB, and GD and the OPNA hydrolyzing enzyme comprises one or more of the following amino acid substitutions relative to SEQ ID NO: 1: A265Y, N266M, and/or C267W.

In some embodiments, the OPNA hydrolyzing enzyme has activity against VX, VR, GB, and GD and the OPNA hydrolyzing enzyme comprises one or more of the following amino acid substitutions relative to SEQ ID NO: 2: T99C, V103H, P151W, L272C, and/or L272W. In some embodiments, the OPNA hydrolyzing enzyme has activity against VX, VR, GB, and GD and the OPNA hydrolyzing enzyme comprises one or more of the following amino acid substitutions relative to SEQ ID NO: 3: A265Y, N266I, and/or N266L. In some embodiments, the OPNA hydrolyzing enzyme has activity against VR, GB, and GD and the OPNA hydrolyzing enzyme comprises the following amino acid substitution relative to SEQ ID NO: 1: W284H. In some embodiments, the OPNA hydrolyzing enzyme has activity against VR and GD and the OPNA hydrolyzing enzyme comprises the following amino acid substitution relative to SEQ ID NO: 1: A265Y. In some embodiments, the OPNA hydrolyzing enzyme has activity against VX, VR and GD and the OPNA hydrolyzing enzyme comprises the following amino acid substitution relative to SEQ ID NO: 1: A265Y. In some embodiments, the OPNA hydrolyzing enzyme has activity against VX, VR and GB and the OPNA hydrolyzing enzyme comprises one or more of the following amino acid substitutions relative to SEQ ID NO: 1: C267W and/or N266G. In some embodiments, the OPNA hydrolyzing enzyme has activity against VX, VR and GB and the OPNA hydrolyzing enzyme comprises one or more of the following amino acid substitutions relative to SEQ ID NO: 2: T99C, L272C, and/or L272W. In some embodiments, the OPNA hydrolyzing enzyme has activity against VX, VR and GB and the OPNA hydrolyzing enzyme comprises one of the following amino acid substitutions relative to SEQ ID NO: 3: N266I or N266L.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used in this disclosure is for the purpose of description and should not be regarded as limiting. The use of “including,” “comprising,” or “having,” “containing,” “involving,” and variations of thereof in this disclosure, is meant to encompass the items listed thereafter and equivalents thereof as well as additional items. As used in this specification and the appended claims, the singular forms “a,” “an” and “the” include plural referents unless the content clearly dictates otherwise.

### BRIEF DESCRIPTION OF THE DRAWINGS

The following drawings form part of the present specification and are included to further demonstrate certain aspects of the present disclosure, which may be better understood by reference to one or more of these drawings in combination with the detailed description of specific embodiments presented in this disclosure. The accompanying drawings are not intended to be drawn to scale. The drawings are illustrative only and are not required for enablement of the disclosure. For purposes of clarity, not every component may be labeled in every drawing. In the drawings:

FIG. 1 is a schematic depicting a representative expression construct for bacterial expression of StrepII protein purification-tagged PTEs. The expression construct was 4,348 bp.

FIG. 2 depicts graphs showing purification and cryopreservation conditions on a representative set of PTEs. The least square means plots show the following freezing conditions: flash freeze, flash freeze+50 mM trehalose, and no treatment; and the presence or absence (false or true) of metals in purification.

FIGS. 3A-3B depict graphs showing screening activity data of PTE hydrolytic activity on VR and VX substrates based on an activity assay using purified PTEs. FIG. 3A depicts data from a primary screen. Library members are represented as open shapes: open squares (strain t402076), open triangles (strain t402353), open inverted triangles (strain t401393), open diamonds (strain t402181), and open circles (strains t402287, t402121, t401421, t401451, t402024, t402094, and t402397). Strain t401992 (filled circles) was used as a positive control and for determining hit ranking of the library members. Strain t402006 (filled triangles), expressing an engineered PTE, B. diminuta variant G1-C74 described in Cherney et al. (2013) ACS Chemical Biology, 8(11), 2394-2403, was also used as a positive control. Strain t339870 (filled square), expressing GFP, was used as a negative control. FIG. 3A shows the plotting of two bioreplicates in all cases except t339870, where the filled square represents the average of 34 bioreplicates. FIG. 3B depicts data from a secondary screen. Library members are represented as open shapes: open squares (strain t402076), open triangles (strain t402353), open inverted triangles (strain t401393), and open diamonds (strain t402181). Strain t339870 (filled square), expressing GFP, was used as a negative control. The data show the average of three bioreplicates and error bars representing standard deviation.

FIGS. 4A-4B depict graphs showing screening activity data of PTE hydrolytic activity on GB and GD substrates based on an activity assay using purified PTEs. FIG. 4A depicts data from a screen including positive controls. GB percent activity and GD percent activity were measured by the residual acetylcholinesterase activity. Library members are represented as open triangles. An uninhibited sample comprising no G-agent (filled inverted triangle) was used as a positive control and for normalizing data derived from each library member and control strain. Strain t402006 (filled triangle) was used as a positive G-agent hydrolase control. A sample comprising no G-agent degrading protein (filled square) was used as a negative control. FIG. 4B depicts a higher resolution plot of the data depicted in FIG. 4A. Library members are represented as open triangles. Strain t339870 (filled circle) comprising GFP but no G-agent degrading protein and was used as a negative control. A sample comprising no G-agent degrading protein (filled square) was also used as a negative control.

FIG. 5 depicts a graph showing the V-agent hydrolyzing activity of strains tested in Example 5. Strain t339870 comprising GFP but no V-agent degrading protein and was used as a negative control. VR and VX activity values were measured in mOD412/min/μg.

FIG. 6 depicts a graph showing the G-agent hydrolyzing activity of strains tested in Example 6. Strain t339870 comprising GFP but no G-agent degrading protein and was used as a negative control. Strain t402006 was used as a positive G-agent hydrolase control. GD activity values were measured as residual acetylcholinesterase activity.

FIGS. 7A-7B depict graphs showing activity data of PTE hydrolytic activity on GB and GD substrates based on an activity assay using purified PTEs. FIGS. 7A-7B specifically show the G-agent hydrolyzing activity of top strains that also exhibit V-agent hydrolyzing activity. FIG. 7A depicts data from a screen including negative controls. GB percent activity and GD percent activity are measured by the residual acetylcholinesterase activity. Library members are represented as open triangles. Strain t339870 (filled circle) comprises GFP but no G-agent degrading protein and was used as a negative control. A sample comprising no G-agent degrading protein (filled square) was also used as a negative control. FIG. 7B depicts the same data as FIG. 7A but on a log-log plot.

FIG. 8 depicts a graph showing activity data of PTE hydrolytic activity on VX and GD substrates based on an activity assay using purified PTEs. Library members are represented as open triangles. Strain t339870 (filled circle) comprises GFP but no G-agent degrading protein and was used as a negative control. A sample comprising no G-agent degrading protein (filled square) was also used as a negative control. An uninhibited sample comprising no G-agent (filled inverted triangle) was used as a positive control and for normalizing G-agent hydrolysis data derived from each library member and control strain. Strain t402006 (filled triangle) was used as a positive G-agent hydrolase control. VX activity values were measured in mOD412/min/μg. GD percent activity was measured by the residual acetylcholinesterase activity.

FIG. 9 depicts a graph showing activity data of PTE hydrolytic activity on VR and GD substrates based on an activity assay using purified PTEs. An uninhibited sample comprising no G-agent (filled inverted triangle) was used as a positive control and for normalizing G-agent hydrolysis data derived from each library member and control strain. Strain t402006 (filled triangle) was used as a positive G-agent hydrolase control. VR activity values were measured in mOD412/min/μg. GD percent activity was measured by the residual acetylcholinesterase activity.

FIG. 10 depicts a graph showing activity data of PTE hydrolytic activity on VX and GB substrates based on an activity assay using purified PTEs. An uninhibited sample comprising no G-agent (filled inverted triangle) was used as a positive control and for normalizing G-agent hydrolysis data derived from each library member and control strain. Strain t402006 (filled triangle) was used as a positive G-agent hydrolase control. VX activity values were measured in mOD412/min/μg. GB percent activity was measured by the residual acetylcholinesterase activity.

FIG. 11 depicts a graph showing activity data of PTE hydrolytic activity on VR and GB substrates based on an activity assay using purified PTEs. An uninhibited sample comprising no G-agent (filled inverted triangle) was used as a positive control and for normalizing G-agent hydrolysis data derived from each library member and control strain. Strain t402006 (filled triangle) was used as a positive G-agent hydrolase control. VR activity values were measured in mOD412/min/μg. GB percent activity was measured by the residual acetylcholinesterase activity.

### DETAILED DESCRIPTION OF THE INVENTION

This disclosure provides identification and production of organophosphorus nerve agent (OPNA) hydrolyzing enzymes using genetically modified host cells. For example, the OPNA hydrolyzing enzymes described in this disclosure can be used for degrading OPNAs, reducing the harmful effects of OPNAs, and/or hydrolyzing OPNAs. This disclosure describes recombinant production of OPNA hydrolyzing enzymes in host cells and the use of recombinantly produced OPNA hydrolyzing enzymes, polynucleotides encoding OPNA hydrolyzing enzymes, and/or cells comprising a heterologous polynucleotide encoding OPNA hydrolyzing enzymes for, e.g., administration to human subjects, or incorporation into or coating of materials, such as clothing or textile, or incorporation into or spraying onto other materials, such as dirt or water.

### Organophosphorus Nerve Agents (OPNA)

Organophosphorus compounds are organic chemicals derived from phosphoric, phosphonic, or phosphinic acids and their derivatives. Organophosphorus compounds include nerve agents, i.e., organophosphorus nerve agents (OPNAs), which are a class of chemical compounds that act rapidly to cause respiratory arrest and death within minutes of cutaneous absorption or inhalation or ingestion. Certain OPNAs are used as chemical warfare agents (CWAs). OPNAs are also extensively used worldwide as pesticides, which can cause great hazards to human health. Terrorist attacks employing OPNAs, as well as accidental exposure to or intentional poisonings employing prevalent OPNA pesticides such as chlorpyriphos, malathion, fenitrothion, and monocrotophos present significant risks to human health worldwide.

OPNAs can be generally classified into five types: (1) G-agents; (2) V-agents, where “V” stands for venomous; (3) GV-agents, which have the combined properties of both G-agents and V-agents; (4) A-agents, also known as Novichok agents; and (5) organophosphorus pesticides. The first four types have been developed primarily for use as and/or used as CWAs. G-agents include, but are not limited to, tabun (GA), sarin (GB), soman (GD), and cyclosarin (GF). V-agents include, but are not limited to, CVX (also known as Chinese VX), VE, VG, VM, VR (also known as Russian VX), and VX. GV-agents include, but are not limited to GV itself, namely 2-dimethylaminoethyl-(dimethylamido)-fluorophosphate. A-agents include but are not limited to, substance-33, A-230, A-232, and A-234. Novichok-5 and Novichok-7 each comprise so-called “binary munitions,” that is two chemical compounds that, when mixed, form A-232 and A-234, respectively. Organophosphorus pesticides include, but are not limited to, parathion, chlorpyriphos, malathion, fenitrothion, and monocrotophos. The types and toxicity of OPNAs are further described, for example, in Mukherjee and Gupta (Organophosphorus Nerve Agents: Types, Toxicity, and Treatments; Journal of Toxicology, (2020) Article ID 3007984), which is incorporated by reference in its entirety.

The V-agents VX, O-ethyl S-(2-diisopropylaminoethyl) methylphosphonothiolate, and VR, O-isobutyl S-[2-(diethylamino)ethyl] methylphosphonothiolate, are among the most potent and dangerous OPNAs. The chemical structures of VX and VR are shown below:

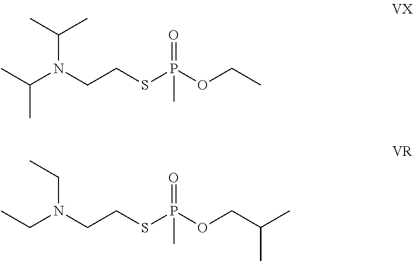

The V-agent VE is also known as O-ethyl S-[2-(diethylamino)ethyl]ethylphosphonothiolate. The V-agent VG is also known as 0,0-iethyl S-[2-(diethylamino)ethyl] phosphorothiolate. The V-agent VM is also known as O-ethyl S-[2-(diethylamino)ethyl] methylphosphonothiolate.

The G-agents GB, Propan-2-yl-methylphosphonofluoridate (also known as “Sarin”), and GD, 0-pinacolyl methylphosphonofluoridate (also known as “Soman”), are extremely toxic and, like VX and VR, are among the most potent and dangerous OPNAs. The chemical structures of GB and GD are shown below:

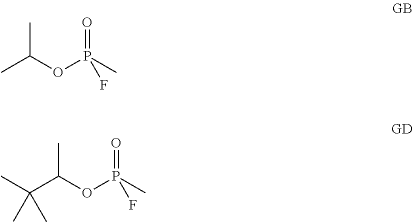

The extreme toxicity of OPNAs largely derives from their ability to irreversibly inhibit acetylcholinesterase (AChE), a vital enzyme that catalyzes the breakdown of the excitatory neurotransmitter acetylcholine (ACh). AChE inhibition by OPNAs leads to ACh buildup at neuronal synapses, at neuromuscular junctions, and in the bloodstream. This ACh excess causes debilitating symptoms including the continuous transmission of excitatory nerve impulses and the resulting involuntary muscle contractions, constriction of pupils, profuse salivation, lacrimation, urination, defecation, gastrointestinal distress, emesis, convulsions, and possibly death. ACh binding at nicotinic receptors results in muscle fasciculations, cramps, weakness, paralysis, and areflexia. ACh can also stimulate the brain where it can induce seizures and coma. OPNA exposure often results in long-term neuropsychiatric sequelae, including disturbances in memory, sleep, and vigilance; depression; anxiety and irritability; and intellectual deficits. Nerve agent toxicity affects all organ systems leading to a multitude of signs and symptoms quickly after exposure.

Currently, no prophylactic effective medical countermeasures (MCMs) are FDA-approved for the prevention of the effects of OPNA intoxication. Approved pretreatments (pyridostigmine bromide) and post-exposure countermeasures (atropine, 2-PAM, and diazepam) do not effectively prevent or mitigate all symptoms of intoxication, especially long-term neuropsychiatric sequelae. Prophylactic and therapeutic MCMs that are currently in development, such as human butyrylcholinesterase (BChE), a stoichiometric pan-OPNA neutralizer, and bacterial OPNA-degrading enzymes, all exhibit one or more deficiencies. Such deficiencies include but are not limited to: 1) kinetic parameters inadequate to neutralize OPNAs prior to AChE inhibition; 2) inadequate activity against multiple OPNA classes (e.g., G-agents, V-agents, A-agents, and others); 3) low bioavailability and inadequate pharmacokinetics (PK); 4) immunogenicity, which results in the elicitation of neutralizing antibody and/or anaphylaxis-provoking responses upon repeated dosing; 5) inconvenient routes of administration; 6) scale-up and manufacturing issues; and 7) high cost.

### OPNA Hydrolyzing Enzymes

Methods and compositions described in this disclosure include OPNA hydrolyzing enzymes. As used in the present disclosure, “an OPNA hydrolyzing enzyme” (which is used interchangeably in this disclosure with “OPNA degrading enzyme”) refers to an enzyme that is capable of, directly or indirectly, hydrolyzing, impacting and/or decreasing the level and/or activity of one or more OPNAs.

In some embodiments, an OPNA hydrolyzing enzyme is a V-agent hydrolyzing enzyme and/or a G-agent hydrolyzing enzyme. As used in the present disclosure, a “V-agent hydrolyzing enzyme” (which is used interchangeably in this disclosure with “V-agent degrading enzyme”) refers to an enzyme that is capable of, directly or indirectly, hydrolyzing, impacting and/or decreasing the level and/or activity of one or more V-agents. In some embodiments, the V-agent is VX. In some embodiments, the V-agent is VR. As used in the present disclosure, a “G-agent hydrolyzing enzyme” (which is used interchangeably in this disclosure with “G-agent degrading enzyme”) refers to an enzyme that is capable of, directly or indirectly, hydrolyzing, impacting and/or decreasing the level and/or activity of one or more G-agents. In some embodiments, the G-agent is GB. In some embodiments, the G-agent is GD.

An OPNA hydrolyzing enzyme, V-agent hydrolyzing enzyme, and/or G-agent hydrolyzing enzyme associated with the disclosure may be a phosphotriesterase (PTE) (EC. 3.1.8.1). As used in this disclosure, a “phosphotriesterase” or “PTE” refers to a metalloenzyme that is capable of hydrolyzing an ester linkage in an organophosphate, an organophosphonate, and/or an organophosphinate. PTEs cleave a labile ester bond of the organophosphate, the organophosphonate, and/or the organophosphinate, and this reaction neutralizes the organophosphate, organophosphonate, and/or organophosphinate molecule. PTEs contain a distorted (β/α)s or triose phosphate isomerase (TIM)-barrel, which includes a core barrel of eight parallel β-strands surrounded by eight α-helices with the two ends of the barrel being formed by the loops connecting each β-strand to the subsequent α-helix, and by the loops connecting each α-helix to the subsequent β-strand. The active site of a PTE is located at the C-terminal end (as defined by the orientation of the [parallel] core β-strands) of the TIM-barrel. The active site contains a binuclear metal center ligated to residues from the C-terminal ends of the core β-strands, with the substrate binding site being formed by the loops that make up the C-terminal end of the barrel (i.e., those loops connecting each β-strand to the subsequent α-helix). Thus, PTEs are generally associated with one or two metal cations, including divalent cations such as, for example and without limitation, Zn²⁺, Co²⁺, Cd²⁺, Mn²⁺, Ni²⁺, Fe²⁺, Mg²⁺, Ca²⁺, Cu²⁺, Ag⁺, and Hg²⁺.

The biological functions and catalytic mechanisms of PTEs are further described, for example, in Bigley and Raushel (Catalytic mechanisms for phosphotriesterases; Biochem Biophys Acta, (2013) 1834 (1): 443-453), which is incorporated by reference in its entirety. See also Shapir et al., J Bacteriol. (2002) 184(19): 5376-84; Chae et al., Arch Biochem Biophys. (1995) 316(2): 765-72; Porzio et al., Chem Biol Interact. (2013) 203(1):251-6; Hiblot et al. PLoS One (2013) 8(9): e75272; Suzumoto et al., Int J Mol Sci. (2020) 21(5): 1683; and Merone et al., Extremophiles (2005) 9(4):297-305; the contents of which are incorporated by reference in their entireties. Serdar et al., describes the PTE derived from B. diminuta in Applied and Environmental Microbiology, 1982, 44(1) 246-249, which is incorporated by reference in its entirety. The B. diminuta PTE has been shown to hydrolyze and inactivate the nerve agents VX and VR.

A schematic of VX hydrolysis is shown below:

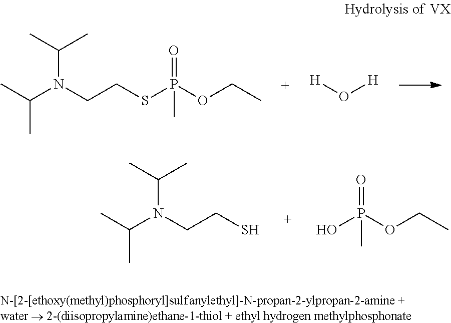

As illustrated in the above schematic of VX hydrolysis, N-[2-[ethoxy(methyl)phosphoryl]sulfanylethyl]-N-propan-2-ylpropan-2-amine in the presence of water is hydrolyzed to form 2-(diisopropylamino)ethane-1-thiol and ethyl hydrogen methylphosphonate.

A schematic of VR hydrolysis is shown below:

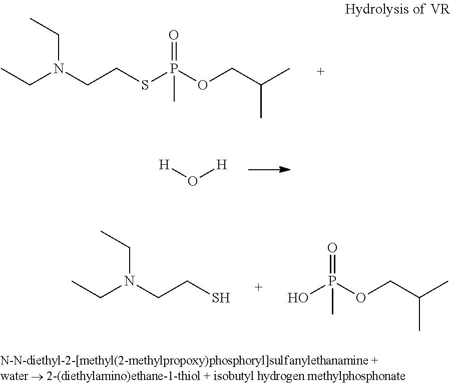

As illustrated in the above schematic of VR hydrolysis, N, N-diethyl-2-[methyl(2-methylpropoxy)phosphoryl]sulfanylethanamine in the presence of water is hydrolyzed to form 2-(diethylamino)ethane-1-thiol and isobutyl hydrogen methylphosphonate.

A schematic of GB (Sarin) hydrolysis is shown below in which Sarin is converted to isopropyl methylphosphate and then to methylphosphonic acid:

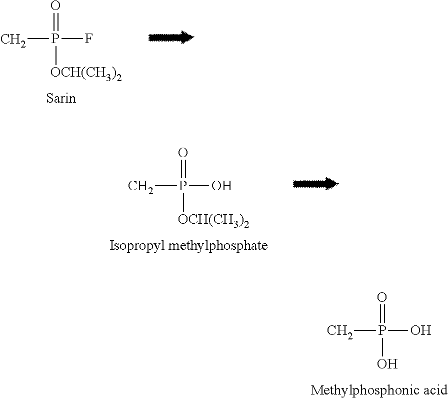

A schematic of GD (Soman) hydrolysis is shown below, in which Soman is converted to pinacolyl methylphosphonic acid and then to methylphosphonic acid:

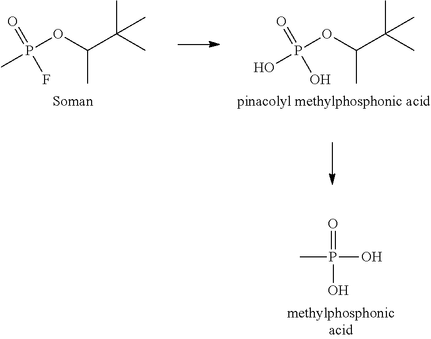

In some embodiments, the V-agent is VX. In some embodiments, the V-agent is VR. In some embodiments, the G-agent is GB. In some embodiments, the G-agent is GD. In some embodiments, the OPNA hydrolyzing enzyme, the V-agent hydrolyzing enzyme, or the G-agent hydrolyzing enzyme is a PTE. In some embodiments, the PTE has hydrolase activity on VR, VX, GB, and/or GD. In some embodiments, the PTE has hydrolase activity at least on both VR and VX. In some embodiments, the PTE has hydrolase activity at least on both GB and GD. In some embodiments, the PTE has hydrolase activity at least on both GB and VR. In some embodiments, the PTE has hydrolase activity at least on both GB and VX. In some embodiments, the PTE has hydrolase activity at least on both GD and VR. In some embodiments, the PTE has hydrolase activity at least on both GD and VX. In some embodiments, the PTE has hydrolase activity at least on VX, VR, and GD. In some embodiments, the PTE has hydrolase activity at least on VX, VR, and GB. In some embodiments, the PTE has hydrolase activity at least on VX, GB, and GD. In some embodiments, the PTE has hydrolase activity at least on VR, GB, and GD. In some embodiments, the PTE has hydrolase activity at least on VX, VR, GB, and GD.

In some embodiments, the OPNA hydrolyzing enzyme, the V-agent hydrolyzing enzyme, or the G-agent hydrolyzing enzyme is a B. diminuta PTE or variant thereof.

In some embodiments, the OPNA hydrolyzing enzyme or the V-agent hydrolyzing enzyme is the B. diminuta PTE variant G1-C74 (Cherny et al. (2013) ACS Chemical Biology, 8(11), 2394-2403). The B. diminuta PTE variant G1-C74 is provided by SEQ ID NO: 5 (expressed in strain t402006 described in the Examples):

(SEQ ID NO: 5)
MIGTGDRINTVRGPITISEAGFTLTHEHICGSSAGFLRAWPEFFGSRAAL
VEKAVRGLRRARAAGVRTIVDVSTFDAGRDVSLLAEVSRAADVHIVAATG
LWFDPPLSMRLRSVEELTQFFLREIQYGIEDTGIRAGIIKVATTGKATPF
QELVLKAAARASLATGVPVTTHTAASQRDGEQQAAIFESEGLSPSRVCIG
HSDDTDDLSYLTALAARGYLIGLDNIPHSAIGLEDNASASALLGSRSWQT
RALLIKALIDQGYMKQILVSNDWLFGFSSYVTNIMDVMDRVNPDGMAFIP
LRVIPFLREKGVPQETLAGITVTNPARFLSPTLRAS.

A non-limiting example of a nucleotide sequence encoding SEQ ID NO: 5 is provided by SEQ ID NO: 14:

(SEQ ID NO: 14)
atgattggcacgggtgatcgaatcaatactgtacgtggccctatcaccat
aagcgaggcgggtttcacactgactcatgaacacatctgtggatcctctg
ctggttttttacgcgcgtggccggaatttttcggctcgagggcagctctg
gtggaaaaagcagttcggggtctgcgtcgcgctcgtgccgcaggcgttag
aaccattgtggacgtatcaaccttcgatgctggtcgtgacgtcagccttc
tggcagaggtttctcgtgctgccgacgtacacattgtggctgcaactggt
ctgtggttcgatccacccctgtccatgcgtctgcgctcagttgaagagct
gactcagtttttcctccgtgaaatccagtatggtatcgaagataccggca
tccgcgctggaatcattaaagttgcgaccacggggaaagccaccccgttc
caggaattggtactgaaagctgcggcacgtgcgtccctcgcaactggcgt
cccggttactacgcacacggctgcttctcagcgcgacggcgaacagcagg
cagccatttttgaaagcgagggtctgtccccgtctagggtctgcateggc
catagcgacgacaccgatgatttaagctacttaacagcactggccgcccg
tgggtacctgataggcctggataacatcccgcacagcgctatcggactgg
aagacaacgcgagtgcttctgctctgctgggttcccgttcctggcaaact
agagcactgctgatcaaggctctgattgatcaggggtacatgaaacaaat
ccttgtgagcaacgactggctgttcggtttcagttcgtatgttaccaaca
tcatggacgttatggatcgcgtgaaccctgacggtatggcgttcattccg
ttgagagtgatcccattcctgcgtgagaaaggcgtaccccaggaaaccct
ggccggtatcactgtaacaaatccggctcgctttctgtctccgactctgc
gcgcatct.

In some embodiments, the OPNA hydrolyzing enzyme, the V-agent hydrolyzing enzyme, or the G-agent hydrolyzing enzyme is a Mycobacterium sp. 852014-52450_SCH5900713 PTE. The Mycobacterium sp. 852014-52450_SCH5900713 PTE provided by SEQ ID NO: 1 was identified in the screen described in Example 1 (expressed in strain t402181):

(SEQ ID NO: 1)
MSELNTARGAIDTTDLGVTLMHEHVFIMTTEIALNYPEAWGDEDKRVA
DAVSRLNELKARGVDTIVDLTVIGLGRYIPRIARVAAATELNIVVATG
LYTYNDVPFRFHYEGPGGMLDGPEIMTEMFVRDIEQGIADTGVKAGIL
KCATDEPGITPGVERVLRAVAQAHKRTGVPISTHTHAGLRRGLEQQRI
FEEEGVDLTRVVIGHSGDSTDIGYLEELIAAGSYLGMDRFGLDVISPF
EERVKIVAQMCERGHADKMVLSHDANCYFDALPEELVPQMAPNWHYLH
IHNDVIPALKERGVTDEQLKTMLVDNPRRIFERQGAYE.

A non-limiting example of a nucleotide sequence encoding SEQ ID NO: 1 is provided by SEQ ID NO: 10;

(SEQ ID NO: 10)
atgtccgagctgaataccgcccgtggggctattgatacaacggaccta
ggcgtgactcttatgcacgaacatgtattcatcatgaccactgaaatc
gcactgaactatccggaagcttggggtgacgaagataaacgcgtcgcg
gacgcagtttctcggctcaacgagctgaaggctaggggcgttgatact
atagttgatctgaccgtaatcggtctgggtcgttacattccacgtatc
gcgcgagtggccgctgctaccgaattaaacatcgtcgtggcgaccggc
ctgtacacttataacgacgttccttttcgtttccactacgaaggcccc
gggggtatgttggacggtccggagattatgacggaaatgttcgttcgc
gatatcgagcagggaatcgcagacactggcgtaaaagcaggcattctg
aaatgtgccacagatgaaccaggtatcaccccgggtgttgaacgtgta
ctgcgcgctgtagcgcaagcgcataagagaacgggtgttccgatctca
acccacacccacgctggcctgcgtcgtggcctggagcagcagcgtatt
tttgaagaagaaggcgtggacctgactcgcgtcgtgattggtcattcg
ggtgacagcactgatatcggctacttggaggaattgatcgctgcgggt
tcttatctgggcatggatcgcttcggactcgacgttatcagcccgttt
gaagaacgtgtgaaaattgttgcccagatgtgcgagcgcggtcacgca
gataaaatggttctgtctcacgacgccaattgctacttcgacgcactc
ccggaagaactggtcccgcaaatggctcctaactggcactacctgcat
atacataatgatgtgatcccagcactgaaagagcgaggtgtgaccgat
gaacagctgaagactatgctggtagacaacccgcgtaggatcttcgag
cgccagggcgcttacgaa.

In some embodiments, the OPNA hydrolyzing enzyme, the V-agent hydrolyzing enzyme, or the G-agent hydrolyzing enzyme is a Mycobacterium colombiense PTE. The Mycobacterium colombiense PTE provided by SEQ ID NO: 2 was identified in the screen described in Example 1 (expressed in strain t401393):

(SEQ ID NO: 2)
MSELNTARGSIDTAALGVTLMHEHVFIMTTEIAENYPEAWGDEDRRVA
DAVKRLNELKARGVDTIVDLTVIGLGRYIPRIARVAAETELNIVVATG
LYTYNDVPMYFHYLGPGGELGGPEIMTDMFVRDIEQGIADTGIKAGIL
KCATDTPGVTPGVERVLRAVAQAHKRTGVPISTHTHAGLRRGLEQQKI
FEEEGVDLSRVIIGHSGDSTDIGYLEELIAAGSYLGMDRFGIDAFLPF
EDRVNTVAQMCERGHADKMVLSHDANCYFDALPDELVSQVMPNWHYLH
IHNDVIPALKERGVTDEQLHTMLVDNPRRIFERQGAYE.

A non-limiting example of a nucleotide sequence encoding SEQ ID NO: 2 is provided by SEQ ID NO: 11:

(SEQ ID NO: 11)
atgagtgagcttaacacggcaaggggttcgatcgacactgccgcgctc
ggcgtaaccctgatgcatgaacacgtgtttattatgactaccgaaatc
gctgaaaattatcccgaggcttggggagatgaagaccgtagagttgct
gatgcagtcaagcgtttaaacgaactgaaagcgcggggcgttgacaca
atagttgatttgactgtaattggtctggggcgttacatcccgcgcatc
gcccgagtggctgcggaaaccgagctgaacattgtagttgctactggt
ctgtacacttacaacgacgttccgatgtatttccactacctgggccca
ggcggtgaactgggtggcccggaaatcatgaccgatatgttcgtgcgt
gacatcgagcaaggtatcgctgataccggaattaaagcaggtatcctc
aaatgcgcaactgacacacctggcgtcaccccgggcgtggaacgtgtt
ctgcgtgctgtagcccaggcacataaacgcaccggtgtcccgatctcc
acgcacacccatgcaggcctgcgccgtggcctggaacagcagaagatt
tttgaagaagagggggttgatctgagccgcgtgattatcggtcactct
ggcgactctactgacatcggttatctggaggaattgattgcgggggta
gctacctaggcatggatcgtttcggtatcgacgctttcctgccgttcg
aggaccgagtaaacacggttgcccagatgtgtgaacgcggccacgccg
ataaaatggttctgtcccacgatgctaattgctactttgacgcgctgc
ctgatgaactggtatctcaagtgatgccaaactggcattatttacaca
tccataacgacgtaatcccggctttaaaagaacgcggcgttaccgatg
agcagctgcacactatgctggttgacaacccgagaagaatcttcgaac
gtcagggtgcttacgaa.

In some embodiments, the OPNA hydrolyzing enzyme, the V-agent hydrolyzing enzyme, or the G-agent hydrolyzing enzyme is a Mycobacterium asiaticum PTE. The Mycobacterium asiaticum PTE provided by SEQ ID NO: 3 was identified in the screen as described in Example 1 (expressed in strain t402076):

(SEQ ID NO: 3)
MSELNTARGPIDTADLGVTLMHEHVFIMTTEIALNYPDAWGDEEQRVA
DAITRLNELKSRGVDTIIDLTVIGLGRYIPRIARVAAETELNIVVATG
LYTYNDIPFRFHYEGPGGLLGGPEIMTDMFVRDIEEGIADTGIKAGIL
KCATDEPGITPGVERVLRAVAQAHKRTGVPISTHTHAGLRRGLEQQRI
FEEEGVDLTRVIIGHSGDSTDVGYLEELIAAGSYLGMDRFGLDVISPF
EQRVDIVAQMCERGHADKMVLSHDANCYFDALPEELVPQMAPNWHYLH
IHNDVLPALKERGVTDEQIHTMLVENPRKIFDRQGAYQ.

A non-limiting example of a nucleotide sequence encoding SEQ ID NO: 3 is provided by SEQ ID NO: 12:

(SEQ ID NO: 12)
atgagcgaactgaatacggcccgcggtcccattgatactgctgactta
ggagtaaccctgatgcacgagcatgtgtttatcatgacaactgaaatc
gcacttaactatcctgacgcgtggggcgatgaagagcaacgtgtcgct
gacgcaataacccgtttgaacgaactgaaatcgagaggggttgatacc
attatcgacctgactgttatcggtctgggtcgctacattccacgtatc
gctcgtgtggcggccgaaaccgagctgaacatcgttgtagctactggc
ctctacacttataacgatatcccgttccgattccactacgaaggcccg
ggcggtctgcttggtggcccggaaataatgaccgacatgtttgttcgg
gatatcgaagaaggcatcgcggatacgggtattaaggcaggtatttta
aaatgtgctacggacgaaccgggtatcaccccgggcgtcgagcgcgtt
ctgcgtgctgtggcgcaggctcataaacgcactggcgtacctatctct
acccacactcacgccggtctgcgtcgcgggctggaacagcagcgtatt
ttcgaggaagagggcgtggacctgacacgtgttatcatcgggcattcc
ggtgactcaaccgatgtaggctacctggaagaactgattgcggcaggt
agttatctgggtatggatcgcttcggcttggacgtgatcagcccgttt
gagcaacgtgttgatattgtagcgcagatgtgcgaacgcggtcacgct
gataagatggtcctgtctcacgacgcgaactgctacttcgacgctcta
ccagaagaactggttccccagatggcaccgaattggcattatttgcac
atccacaacgacgttctgccagcactgaaagagcgtggagtcaccgac
gaacagattcatacgatgttagttgaaaacccgcgtaaaatttttgat
cgtcagggtgcttaccag.

In some embodiments, the OPNA hydrolyzing enzyme, the V-agent hydrolyzing enzyme, or the G-agent hydrolyzing enzyme is a Mycobacterium gordonae PTE. The Mycobacterium gordonae PTE provided by SEQ ID NO: 4 was identified in the screen described in Example 1 (expressed in strain t402353):

(SEQ ID NO: 4)
MSELNTARGTIDTADLGVTLMHEHVFIMTTEIALNYPEAWGDEEKRVA
DAVARLNELKSRGVDTIVDLTVIGLGRYIPRIARVAAQTELNIVVATG
LYTYNDIPFRFHYEGPGGMLDGPEIMTDMFVRDIEQGIADTGIKAGIL
KCATDEPGITPGVERVLRAVAQAHKRTGVPISTHTHAGLRRGLEQQRI
FEEEGVDLTRVIIGHSGDSTDVGYLEELISAGSYLGMDRFGLDVLLPF
EERVQIVATMCERGHADKMVLSHDANCYFDALPEQLVPQMAPNWHYLH
IHNDVIPALKHRGVTDEQIHTMLVENPRKIFDRQGAYQ.

A non-limiting example of a nucleotide sequence encoding SEQ ID NO: 4 is provided by SEQ ID NO: 13:

(SEQ ID NO: 13)
atgtctgaactgaatacagctcgcggcacgatagacactgccgatctt
ggtgtcaccctcatgcatgagcacgtttttatcatgaccactgaaatc
gcgctaaactatccagaagcatggggtgatgaagagaaacgtgtagct
gacgctgttgcccgtctgaacgaactgaagtcacgaggggtggatacc
attgttgacctgaccgtgattggcctgggtcgctacatccctcgtatc
gcacgtgtggcggctcagacggaattaaacattgtcgttgcaactggc
ctgtacacatacaatgatattccgttccgcttccactatgagggcccg
ggaggtatgctggacggtccggaaatcatgactgatatgtttgtgcgt
gacatcgaacaaggcatcgcggacaccgggatcaaagctggtatcctt
aaatgcgccaccgatgaaccgggcattactccaggtgtagaaagggtt
ttgcgtgctgtagcgcaggctcacaaacgtacgggcgtcccgatttcc
actcatacccacgcaggtctgcgccgcggcctggagcagcagcgcatc
ttcgaagaggaaggtgttgacctcactcgtgttatcataggtcacagc
ggcgattcgaccgatgtcgggtacctggaagaactgattagcgcaggt
tcttatttaggcatggaccgtttcggcctcgatgtgctgctgcccttt
gaggagcgggtacaaatcgttgcaacaatgtgtgaaagaggccatgcc
gacaagatggtactgtcccacgatgcgaactgctacttcgacgctctg
ccggaacagctggtaccgcagatggctccaaactggcattacctgcac
atccataacgacgtgatccctgcgctgaaacaccgcggtgttactgac
gaacaaatccacacgatgctggttgaaaacccgcgtaaaatcttcgac
cgtcagggtgcctatcag.

In some embodiments, the OPNA hydrolyzing enzyme, the V-agent hydrolyzing enzyme, or the G-agent hydrolyzing enzyme is a Brevundimonas diminuta PTE. A Brevundimonas diminuta PTE is provided by SEQ ID NO: 7. This sequence corresponds to the amino acid sequence of UniprotKB Accession No. P0A434, except that the signal sequence is removed and a methionine residue is added at the N-terminus:

(SEQ ID NO: 7)
MIGTGDRINTVRGPITISEAGFTLTHEHICGSSAGFLRAWPEFFGSRK
ALAEKAVRGLRRARAAGVRTIVDVSTFDIGRDVSLLAEVSRAADVHIV
AATGLWFDPPLSMRLRSVEELTQFFLREIQYGIEDTGIRAGIIKVATT
GKATPFQELVLKAAARASLATGVPVTTHTAASQRDGEQQAAIFESEGL
SPSRVCIGHSDDTDDLSYLTALAARGYLIGLDHIPHSAIGLEDNASAS
ALLGIRSWQTRALLIKALIDQGYMKQILVSNDWLFGFSSYVTNIMDVM
DRVNPDGMAFIPLRVIPFLREKGVPQETLAGITVTNPARFLSPTLRAS.

A non-limiting example of a nucleotide sequence encoding SEQ ID NO: 7 is provided by SEQ ID NO: 16:

(SEQ ID NO: 16)
atgattggcacgggtgatcgaatcaatactgtacgtggccctatcacc
ataagcgaggcgggtttcacactgactcatgaacacatctgtggatcc
tctgctggttttttacgcgcgtggccggaatttttcggctcgaggaaa
gctctggcggaaaaagcagttcggggtctgcgtcgcgctcgtgccgca
ggcgttagaaccattgtggacgtatcaaccttcgatatcggtcgtgac
gtcagccttctggcagaggtttctcgtgctgccgacgtacacattgtg
gctgcaactggtctgtggttcgatccacccctgtccatgcgtctgcgc
tcagttgaagagctgactcagtttttcctccgtgaaatccagtatggt
atcgaagataccggcatccgcgctggaatcattaaagttgcgaccacg
gggaaagccaccccgttccaggaattggtactgaaagctgcggcacgt
gcgtccctcgcaactggcgtcccggttactacgcacacggctgcttct
cagcgcgacggcgaacagcaggcagccatttttgaaagcgagggtctg
tccccgtctagggtctgcatcggccatagcgacgacaccgatgattta
agctacttaacagcactggccgcccgtgggtacctgataggcctggat
catatcccgcacagcgctatcggactggaagacaacgcgagtgcttct
gctctgctgggtattcgttcctggcaaactagagcactgctgatcaag
gctctgattgatcaggggtacatgaaacaaatccttgtgagcaacgac
tggctgttcggtttcagttcgtatgttaccaacatcatggacgttatg
gatcgcgtgaaccctgacggtatggcgttcattccgttgagagtgatc
ccattcctgcgtgagaaaggcgtaccccaggaaaccctggccggtatc
actgtaacaaatccggctcgctttctgtctccgactctgcgcgcatct

In some embodiments, the OPNA hydrolyzing enzyme, the V-agent hydrolyzing enzyme, or the G-agent hydrolyzing enzyme is a Brevundimonas diminuta PTE comprising the sequence of SEQ ID NO: 8:

(SEQ ID NO: 8)
GDRINTVRGPITISEAGFTLTHEHICGSSAGFLRAWPEFFGSRAALVE
KAVRGLRRARAAGVRTIVDVSTFDAGRDVSLLAEVSRAADVHIVAATG
LWEDPPLSMRLRSVEELTQFFLREIQYGIEDTGIRAGIIKVATNGKAT
PFQELVLRAAARASLATGVPVTTHTAASQRDGEQQAAIFESEGLSPSR
VCIGHSDDTDDLSYLTALAARGYLIGLDGIPHSAIGLEDNASASELLG
IRSWQTRALLIKALIDQGYMKQILVSNDWLFGFSSYVTNIMDVMDSVN
PDGMAFIPLRVIPFLREKGVPQETLAGITVTNPARFLSPTLRAS.

SEQ ID NO: 8 corresponds to a PTE variant, referred to as IVH3, which contains an N-terminal truncation and multiple amino acid substitutions relative to the parent sequence corresponding to SEQ ID NO: 7. The catalytic mechanisms and efficiencies of PTE variants, including IVH3, are further described, for example, in Goldsmith et al. (Catalytic efficiencies of directly evolved phosphotriesterase variants with structurally different organophosphorus compounds in vitro, Arch Toxicol, (2016) 90 (11): 2711-2724), which is incorporated by reference in its entirety.

In some embodiments, the OPNA hydrolyzing enzyme, the V-agent hydrolyzing enzyme, or the G-agent hydrolyzing enzyme is a phosphotriesterase-related protein (PTER). PTERs (also referred to as PTE-homology proteins (PHPs)) are members of the amidohydrolase superfamily and are a group of proteins evolutionarily related to PTEs. PTERs share both sequence homology and structural similarity to PTEs, including a binuclear (Zn²⁺ or other metal ion) metal center. Several differences have been noted in the active site of PTERs relative to PTEs. For example, both human and E. coli PTER have Tyr128/Tyr84 (human/E. coli) in the active site, instead of the Trp131, which is present in the B. diminuta PTE. Both human and E. coli PTER also have Glu169/Glu125 (Human/E. coli), rather than carboxylated Lys169, which is present in PTEs. Both human and E. coli PTER also have a 1-residue insertion, adjacent to the Glu169/Glu125 residue. PTERs are described further in Roodveldt et al. (2005) Biochemistry, 44(38), 12728-12736; Buchbinder et al. (1998) Biochemistry, 37, 5096-5106; Hou et al. (1996) Gene, 168(2), 157-163; and Wang et al. (2011) Agricultural Sciences, 02(04), 406-412, each of which is incorporated by reference in its entirety in this disclosure.

In some embodiments, the PTER has hydrolase activity at least on both GB and GD. In some embodiments, the PTER has hydrolase activity at least on both GB and VR. In some embodiments, the PTER has hydrolase activity on VR or VX. In some embodiments, the PTER has hydrolase activity at least on both VR and VX. In some embodiments, the PTER has hydrolase activity at least on both GB and VX. In some embodiments, the PTER has hydrolase activity at least on both GD and VR. In some embodiments, the PTER has hydrolase activity at least on both GD and VX. In some embodiments, the PTER has hydrolase activity at least on VX, VR, and GD. In some embodiments, the PTER has hydrolase activity at least on VX, VR, and GB. In some embodiments, the PTER has hydrolase activity at least on VX, GB, and GD. In some embodiments, the PTER has hydrolase activity at least on VR, GB, and GD. In some embodiments, the PTER has hydrolase activity at least on VX, VR, GB, and GD. In some embodiments, the OPNA hydrolyzing enzyme, the V-agent hydrolyzing enzyme, or the G-agent hydrolyzing enzyme is a Prosthecomicrobium hirschii PTER. The Prosthecomicrobium hirschii PTER provided by SEQ ID NO: 6 is expressed in strain t401609 described in the Examples:

(SEQ ID NO: 6)
MAAAETGTGHARIETVLGPIAPAALGATLMHEHLLCDLTPPARRGQGL
PEPEITLETIFDMGYRPGRYHGNHRLQDVALATREAAAFKADGGGAIV
ELTTGGIVPDPEGLAAIARGAGIHVVLGAGFYTENFLDAETLALSTEA
LTEITLGQLTEGAWGTKVRCGLIGEIGCSWPLTPFERRSLKSGARAQI
ATGAAITIHPGRDRAAPDEILDVLEAEGADLSRVVIGHMDRTLLDDAD
VVALARRGSIVEYDFFGIEHSNYWLGVVDIPNDWMRIRALRKLFDAGL
GDRVVISHDICTRTRLQSLGGHGYGHILRNVVPLMRDRGFSQSEIDLL
LLETPRRILTIGG

A non-limiting example of a nucleotide sequence encoding SEQ ID NO: 6 is provided by SEQ ID NO: 15:

(SEQ ID NO: 15)
atggcagctgccgagacagggacgggccacgcgcgtatcgaaactgta
ttaggtcccattgctccagcagccctgggtgctaccctgatgcatgaa
caccttctgtgcgacctcactccgcctgcgcggcgcggccaaggtctg
ccggaaccggagatcaccctggaaactatatttgatatgggctatagg
ccgggccgttaccatggtaatcaccgactgcaagacgtcgctctggcg
acccgtgaagccgcagctttcaaggcggatggaggtggcgctatcgtt
gagctaaccacgggcggtattgttccggatccagaaggcctggctgca
atcgcccgcggtgccgggattcatgtggtattgggtgcgggcttctac
accgaaaactttctggacgctgaaactcttgctctgtccacagaggca
ttgactgaaatcaccctgggccagttaaccgaaggtgcatggggaact
aaagtgcgttgtggtctgatcggcgagatcggatgcagctggccgctg
acgcctttcgaacgccgttcactgaaatctggtgctcgtgcacagatt
gctactggcgcggctatcaccattcacccgggtcgcgaccgcgcggca
ccggatgagatcctggacgttctggaagcggaaggcgctgatctatcg
cgtgtcgttataggtcacatggaccgtacactgctggacgatgcagat
gttgttgcactcgctcgccgtggtagcatcgtagagtatgatttcttc
ggcatcgaacactctaactactggctgggggtggtagacattcctaac
gattggatgcgtatccgggcgctgagaaaattgtttgacgccggtctt
ggagacagggtcgtaatcagccatgatatttgcactcgtacccgtctg
caatccctgggcggccatggttacgggcacatcctgcgcaacgtggtt
ccgcttatgcgcgatcgtggtttcagtcagtctgaaatcgacctcctg
ctgctggaaaccccacgacgtattctgaccattggtggg.

In some embodiments, the PTER is a Homo sapiens PTER. The amino acid sequence of Homo sapiens PTER can be found at UniProt Accession No. Q96BW5 and is provided as SEQ ID NO: 9 below:

(SEQ ID NO: 9)
MSSLSGKVQTVLGLVEPSKLGRTLTHEHLAMTFDCCYCPPPPCQEAIS
KEPIVMKNLYWIQKNAYSHKENLQLNQETEAIKEELLYFKANGGGALV
ENTTTGISRDTQTLKRLAEETGVHIISGAGFYVDATHSSETRAMSVEQ
LTDVLMNEILHGADGTSIKCGIIGEIGCSWPLTESERKVLQATAHAQA
QLGCPVIIHPGRSSRAPFQIIRILQEAGADISKTVMSHLDRTILDKKE
LLEFAQLGCYLEYDLFGTELLHYQLGPDIDMPDDNKRIRRVRLLVEEG
CEDRILVAHDIHTKTRLMKYGGHGYSHILTNVVPKMLLRGITENVLDK
ILIENPKQWLTFK

A non-limiting example of a nucleotide sequence encoding SEQ ID NO: 9 is provided by SEQ ID NO: 17:

(SEQ ID NO: 17)
atgtcttccttaagtggaaaagtccaaaccgttttgggccttgtagag
ccaagcaaactgggccgtaccctgacccatgaacacctggccatgacc
tttgactgctgttactgtccacctcccccgtgccaggaagctatttcc
aaagaacctatcgtgatgaaaaatttatattggattcagaaaaacgcc
tattcccataaagaaaaccttcaattaaatcaggagacagaagccata
aaggaagaactgttgtattttaaagctaatggtggaggggctttggtg
gaaaacacaaccactgggattagccgagacacacagacgttgaagagg
cttgcagaagagactggcgtccatatcatatctggagccgggttttat
gtggatgcaactcactcctcagagaccagggccatgtcagtggagcag
cttaccgatgtccttatgaatgaaattctccatggagctgatggaacc
agtatcaagtgtggcattattggagaaattggttgctcctggcctttg
actgagagtgaaagaaaggttctccaggccacagctcatgcccaggct
cagcttggttgtcctgttattatccatcctggacggagctccagggca
ccatttcagattatccgaatattgcaagaagcaggcgcagacatctcc
aaaacagtcatgtcacacctggataggactattcttgataagaaagag
ctcttggagtttgctcaacttggctgctacttggaatatgatctcttt
ggtactgaactacttcattaccaactcggcccagatattgacatgcct
gatgataacaaaagaattagaagggtgcgtctcctggtggaagagggc
tgtgaagatcgaattctggtagcacatgacatacatacgaaaacccgg
ctgatgaaatatggaggtcacggctattctcatatactcaccaatgtt
gttcctaaaatgttgctgagaggcataactgagaatgtgcttgataag
attctaatagagaaccctaagcaatggctaactttcaaatag

In some embodiments, the present disclosure provides an OPNA hydrolyzing enzyme that is a PTE or PTER. In some embodiments, the OPNA hydrolyzing enzyme is a V-agent hydrolyzing enzyme. In some embodiments, the V-agent hydrolyzing enzyme is active against VX. In some embodiments, the V-agent hydrolyzing enzyme is active against VR. In some embodiments, the V-agent hydrolyzing enzyme is active against both VX and VR. In some embodiments, the OPNA hydrolyzing enzyme is a G-agent hydrolyzing enzyme. In some embodiments, the G-agent hydrolyzing enzyme is active against GB. In some embodiments, the G-agent hydrolyzing enzyme is active against GD. In some embodiments, the G-agent hydrolyzing enzyme is active against both GB and GD. In some embodiments, the OPNA hydrolyzing enzyme has activity as both a V-agent hydrolyzing enzyme and a G-agent hydrolyzing enzyme.

In some embodiments, PTEs or PTERs associated with the disclosure are active against one or more of: (1) G-agents, including but not limited to, tabun (GA), sarin (GB), soman (GD), and cyclosarin (GF); (2) V-agents, including but not limited to, CVX (also known as Chinese VX), VE, VG, VM, VR (also known as Russian VX), and VX; (3) GV-agents, including but not limited to, GV itself, namely 2-dimethylaminoethyl-(dimethylamido)-fluorophosphate; (4) A-agents, including but not limited to, substance-33, A-230, A-232, and A-234; and/or (5) other OPNAs, including but not limited to, organophosphorus pesticides such as parathion, chlorpyriphos, malathion, fenitrothion, and monocrotophos.

It should be appreciated that sequences disclosed in this application may or may not contain signal peptides and/or secretion signals. The sequences disclosed in this application encompass versions with or without signal peptides and/or secretion signals. It should also be understood that amino acid sequences disclosed in this application may be depicted with or without a start codon (M). The sequences disclosed in this application encompass versions with or without start codons. Accordingly, in some instances amino acid numbering may correspond to amino acid sequences containing a signal peptide and/or secretion signal and/or a start codon, while in other instances, amino acid numbering may correspond to amino acid sequences that do not contain a signal peptide and/or a secretion signal and/or a start codon. It should also be understood that sequences disclosed in this application may be depicted with or without a stop codon. The sequences disclosed in this application encompass versions with or without stop codons. Aspects of the disclosure encompass OPNA hydrolyzing enzymes or V-agent hydrolyzing enzymes comprising any of the sequences described in this application and fragments thereof.

In some embodiments, a PTE provided in this disclosure comprises an amino acid sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 1-5, 7 or 8, including all values in between. In some embodiments, the PTE comprises an amino acid sequence that is at least 90% identical to any one of SEQ ID NOs: 1-5, 7 or 8. In some embodiments, the PTE comprises or consists of an amino acid sequence corresponding to any one of SEQ ID NOs: 1-5, 7 or 8.

In some embodiments, a PTE provided in this disclosure is encoded by a nucleotide sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 10-14, including all values in between. In some embodiments, the PTE is encoded by a nucleotide sequence that is at least 90% identical to any one of SEQ ID NOs: 10-14. In some embodiments, the PTE is encoded by a nucleotide sequence corresponding to any one of SEQ ID NOs: 10-14.

In some embodiments, a PTER provided in this disclosure comprises an amino acid sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 6 or 9, including all values in between. In some embodiments, the PTER comprises an amino acid sequence that is at least 90% identical to SEQ ID NO: 6 or 9. In some embodiments, the PTER comprises or consists of an amino acid sequence corresponding to SEQ ID NO: 6 or 9.

In some embodiments, a PTER provided in this disclosure is encoded by a nucleotide sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NOs: 15 or 17, including all values in between. In some embodiments, the PTER is encoded by a nucleotide sequence corresponding to SEQ ID NO: 15 or 17.

Unless otherwise noted, the term “sequence identity,” which is used interchangeably in this disclosure with the term “percent identity,” as known in the art, refers to a relationship between the sequences of two polypeptides or polynucleotides, as determined by sequence comparison (alignment). In some embodiments, sequence identity is determined across the entire length of a sequence. In some embodiments, sequence identity is determined over a region (e.g., a stretch of amino acids or nucleic acids, e.g., the sequence spanning an active site) of a sequence. For example, in some embodiments, sequence identity is determined over a region corresponding to at least 30%, at least 40%, at least 50%, at least 60%, at least 70%, at least 80%, at least 90%, at least 95%, or over 100% of the length of the reference sequence.

Identity of related amino acid or nucleotide sequences can be readily calculated by any of the methods known to one of ordinary skill in the art. The percent identity of two sequences (e.g., nucleic acid or amino acid sequences) may, for example, be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST® and XBLAST® programs (version 2.0) of Altschul et al., J. Mol. Biol. 215:403-10, 1990. BLAST® protein searches can be performed, for example, with the XBLAST program, score=50, wordlength=3 to obtain amino acid sequences homologous to the proteins described in this application. Where gaps exist between two sequences, Gapped BLAST® can be utilized, for example, as described in Altschul et al., Nucleic Acids Res. 25(17):3389-3402, 1997. When utilizing BLAST® and Gapped BLAST® programs, the default parameters of the respective programs (e.g., XBLAST® and NBLAST®) can be used, or the parameters can be adjusted appropriately as would be understood by one of ordinary skill in the art.

Another local alignment technique which may be used, for example, is based on the Smith-Waterman algorithm (Smith, T. F. & Waterman, M. S. (1981) “Identification of common molecular subsequences.” J. Mol. Biol. 147:195-197). A general global alignment technique which may be used, for example, is the Needleman-Wunsch algorithm (Needleman, S. B. & Wunsch, C. D. (1970) “A general method applicable to the search for similarities in the amino acid sequences of two proteins.” J. Mol. Biol. 48:443-453), which is based on dynamic programming.

More recently, a Fast Optimal Global Sequence Alignment Algorithm (FOGSAA) was developed that purportedly produces global alignment of nucleic acid and amino acid sequences faster than other optimal global alignment methods, including the Needleman-Wunsch algorithm. In some embodiments, the identity of two polypeptides is determined by aligning the two amino acid sequences, calculating the number of identical amino acids, and dividing by the length of one of the amino acid sequences. In some embodiments, the identity of two nucleic acids is determined by aligning the two nucleotide sequences and calculating the number of identical nucleotide and dividing by the length of one of the nucleic acids.

For multiple sequence alignments, computer programs including Clustal Omega (Sievers et al., Mol Syst Biol. 2011 Oct. 11; 7:539) may be used.

In preferred embodiments, a nucleotide or amino acid sequence is found to have a specified percent identity to a reference sequence, such as a sequence disclosed in this application and/or recited in the claims when sequence identity is determined using the algorithm of Karlin and Altschul Proc. NatL. Acad. Sci. USA 87:2264-68, 1990, modified as in Karlin and Altschul Proc. Natl. Acad. Sci. USA 90:5873-77, 1993 (e.g., BLAST®, NBLAST®, XBLAST® or Gapped BLAST® programs, using default parameters of the respective programs).

In some embodiments, a nucleotide or amino acid sequence is found to have a specified percent identity to a reference sequence, such as a sequence disclosed in this application and/or recited in the claims when sequence identity is determined using the Smith-Waterman algorithm (Smith, T. F. & Waterman, M. S. (1981) “Identification of common molecular subsequences.” J. Mol. Biol. 147:195-197) or the Needleman-Wunsch algorithm (Needleman, S. B. & Wunsch, C. D. (1970) “A general method applicable to the search for similarities in the amino acid sequences of two proteins.” J. Mol. Biol. 48:443-453) using default parameters.

In some embodiments, a nucleotide or amino acid sequence, is found to have a specified percent identity to a reference sequence, such as a sequence disclosed in this application and/or recited in the claims when sequence identity is determined using a Fast Optimal Global Sequence Alignment Algorithm (FOGSAA) using default parameters.

In some embodiments, a nucleotide or amino acid sequence, is found to have a specified percent identity to a reference sequence, such as a sequence disclosed in this application and/or recited in the claims when sequence identity is determined using Clustal Omega (Sievers et al., Mol Syst Biol. 2011 Oct. 11; 7:539) using default parameters.

PTEs or PTERs associated with the disclosure may comprise wildtype sequences or may be engineered. PTEs or PTERs associated with the disclosure may comprise one or more amino acid substitutions, additions, deletions, insertions, or truncations relative to a reference sequence (e.g., relative to any one of SEQ ID NOs: 1-4 or 6). PTEs or PTERs associated with the disclosure may be naturally occurring or may be synthetic. PTEs or PTERs associated with the disclosure can include fragments or peptides of PTEs or PTERs, such as fragments or peptides that preserve the activity of a full-length PTE or PTER. PTEs or PTERs associated with the disclosure include truncated forms of PTEs or PTERs, such as truncated forms that preserve the activity of a full-length PTE or PTER.

In some embodiments, the coding sequence of a PTE or a PTER comprises a mutation at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more than 100 positions relative to a reference coding sequence. In some embodiments, the coding sequence of a PTE or a PTER comprises a mutation in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more codons of the coding sequence relative to a reference coding sequence. As will be understood by one of ordinary skill in the art, a mutation within a codon may or may not change the amino acid that is encoded by the codon due to degeneracy of the genetic code. In some embodiments, the one or more mutations in the coding sequence do not alter the amino acid sequence of the coding sequence (e.g., a PTE or a PTER) relative to the amino acid sequence of a reference polypeptide (e.g a PTE or a PTER). In other embodiments, the one or more mutations in the coding sequence do alter the amino acid sequence of the coding sequence (e.g., a PTE or a PTER) relative to the amino acid sequence of a reference polypeptide (e.g a PTE or a PTER).

In some embodiments, a PTE or PTER comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 60, at least 70, at least 80, at least 90, or at least 100 amino acid substitutions, deletions, or additions relative to any one of SEQ ID NO: 1-4 or 6, a PTE or PTER in Table 5 or 7, or a PTE or PTER otherwise described in this disclosure.

In some embodiments, a PTE or PTER provided in this disclosure comprises an amino acid sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 36-956.

In some embodiments, a PTE comprises: T at a residue corresponding to residue 22 in SEQ ID NO: 1; S at a residue corresponding to residue 22 in SEQ ID NO: 1; D at a residue corresponding to residue 23 in SEQ ID NO: 1; N at a residue corresponding to residue 24 in SEQ ID NO: 1; I at a residue corresponding to residue 25 in SEQ ID NO: 1; M at a residue corresponding to residue 25 in SEQ ID NO: 1; M at a residue corresponding to residue 27 in SEQ ID NO: 1; K at a residue corresponding to residue 27 in SEQ ID NO: 1; H at a residue corresponding to residue 27 in SEQ ID NO: 1; T at a residue corresponding to residue 27 in SEQ ID NO: 1; C at a residue corresponding to residue 27 in SEQ ID NO: 1; W at a residue corresponding to residue 27 in SEQ ID NO: 1; R at a residue corresponding to residue 27 in SEQ ID NO: 1; F at a residue corresponding to residue 27 in SEQ ID NO: 1; E at a residue corresponding to residue 27 in SEQ ID NO: 1; N at a residue corresponding to residue 27 in SEQ ID NO: 1; Y at a residue corresponding to residue 27 in SEQ ID NO: 1; Q at a residue corresponding to residue 27 in SEQ ID NO: 1; P at a residue corresponding to residue 27 in SEQ ID NO: 1; D at a residue corresponding to residue 27 in SEQ ID NO: 1; I at a residue corresponding to residue 66 in SEQ ID NO: 1; N at a residue corresponding to residue 68 in SEQ ID NO: 1; M at a residue corresponding to residue 68 in SEQ ID NO: 1; Q at a residue corresponding to residue 68 in SEQ ID NO: 1; A at a residue corresponding to residue 68 in SEQ ID NO: 1; C at a residue corresponding to residue 68 in SEQ ID NO: 1; S at a residue corresponding to residue 69 in SEQ ID NO: 1; Q at a residue corresponding to residue 69 in SEQ ID NO: 1; C at a residue corresponding to residue 70 in SEQ ID NO: 1; P at a residue corresponding to residue 70 in SEQ ID NO: 1; T at a residue corresponding to residue 70 in SEQ ID NO: 1; H at a residue corresponding to residue 73 in SEQ ID NO: 1; M at a residue corresponding to residue 73 in SEQ ID NO: 1; C at a residue corresponding to residue 73 in SEQ ID NO: 1; W at a residue corresponding to residue 98 in SEQ ID NO: 1; F at a residue corresponding to residue 98 in SEQ ID NO: 1; H at a residue corresponding to residue 98 in SEQ ID NO: 1; I at a residue corresponding to residue 144 in SEQ ID NO: 1; S at a residue corresponding to residue 145 in SEQ ID NO: 1; R at a residue corresponding to residue 145 in SEQ ID NO: 1; E at a residue corresponding to residue 145 in SEQ ID NO: 1; C at a residue corresponding to residue 145 in SEQ ID NO: 1; F at a residue corresponding to residue 146 in SEQ ID NO: 1; G at a residue corresponding to residue 147 in SEQ ID NO: 1; F at a residue corresponding to residue 147 in SEQ ID NO: 1; M at a residue corresponding to residue 148 in SEQ ID NO: 1; V at a residue corresponding to residue 148 in SEQ ID NO: 1; C at a residue corresponding to residue 148 in SEQ ID NO: 1; I at a residue corresponding to residue 164 in SEQ ID NO: 1; Y at a residue corresponding to residue 176 in SEQ ID NO: 1; I at a residue corresponding to residue 176 in SEQ ID NO: 1; C at a residue corresponding to residue 176 in SEQ ID NO: 1; H at a residue corresponding to residue 176 in SEQ ID NO: 1; F at a residue corresponding to residue 176 in SEQ ID NO: 1; W at a residue corresponding to residue 176 in SEQ ID NO: 1; M at a residue corresponding to residue 176 in SEQ ID NO: 1; T at a residue corresponding to residue 176 in SEQ ID NO: 1; V at a residue corresponding to residue 176 in SEQ ID NO: 1; V at a residue corresponding to residue 177 in SEQ ID NO: 1; L at a residue corresponding to residue 177 in SEQ ID NO: 1; C at a residue corresponding to residue 177 in SEQ ID NO: 1; S at a residue corresponding to residue 178 in SEQ ID NO: 1; A at a residue corresponding to residue 178 in SEQ ID NO: 1; T at a residue corresponding to residue 178 in SEQ ID NO: 1; C at a residue corresponding to residue 179 in SEQ ID NO: 1; V at a residue corresponding to residue 179 in SEQ ID NO: 1; S at a residue corresponding to residue 179 in SEQ ID NO: 1; E at a residue corresponding to residue 179 in SEQ ID NO: 1; P at a residue corresponding to residue 181 in SEQ ID NO: 1; H at a residue corresponding to residue 181 in SEQ ID NO: 1; S at a residue corresponding to residue 181 in SEQ ID NO: 1; C at a residue corresponding to residue 181 in SEQ ID NO: 1; A at a residue corresponding to residue 206 in SEQ ID NO: 1; M at a residue corresponding to residue 206 in SEQ ID NO: 1; S at a residue corresponding to residue 206 in SEQ ID NO: 1; C at a residue corresponding to residue 208 in SEQ ID NO: 1; T at a residue corresponding to residue 208 in SEQ ID NO: 1; Q at a residue corresponding to residue 208 in SEQ ID NO: 1; A at a residue corresponding to residue 208 in SEQ ID NO: 1; N at a residue corresponding to residue 209 in SEQ ID NO: 1; T at a residue corresponding to residue 210 in SEQ ID NO: 1; E at a residue corresponding to residue 210 in SEQ ID NO: 1; S at a residue corresponding to residue 210 in SEQ ID NO: 1; A at a residue corresponding to residue 228 in SEQ ID NO: 1; E at a residue corresponding to residue 228 in SEQ ID NO: 1; S at a residue corresponding to residue 228 in SEQ ID NO: 1; H at a residue corresponding to residue 228 in SEQ ID NO: 1; P at a residue corresponding to residue 230 in SEQ ID NO: 1; L at a residue corresponding to residue 231 in SEQ ID NO: 1; G at a residue corresponding to residue 231 in SEQ ID NO: 1; H at a residue corresponding to residue 231 in SEQ ID NO: 1; M at a residue corresponding to residue 231 in SEQ ID NO: 1; W at a residue corresponding to residue 231 in SEQ ID NO: 1; T at a residue corresponding to residue 231 in SEQ ID NO: 1; Q at a residue corresponding to residue 231 in SEQ ID NO: 1; C at a residue corresponding to residue 262 in SEQ ID NO: 1; S at a residue corresponding to residue 263 in SEQ ID NO: 1; N at a residue corresponding to residue 263 in SEQ ID NO: 1; T at a residue corresponding to residue 263 in SEQ ID NO: 1; C at a residue corresponding to residue 263 in SEQ ID NO: 1; F at a residue corresponding to residue 263 in SEQ ID NO: 1; M at a residue corresponding to residue 263 in SEQ ID NO: 1; N at a residue corresponding to residue 264 in SEQ ID NO: 1; S at a residue corresponding to residue 264 in SEQ ID NO: 1; P at a residue corresponding to residue 264 in SEQ ID NO: 1; T at a residue corresponding to residue 265 in SEQ ID NO: 1; F at a residue corresponding to residue 265 in SEQ ID NO: 1; W at a residue corresponding to residue 265 in SEQ ID NO: 1; C at a residue corresponding to residue 265 in SEQ ID NO: 1; L at a residue corresponding to residue 265 in SEQ ID NO: 1; M at a residue corresponding to residue 265 in SEQ ID NO: 1; Y at a residue corresponding to residue 265 in SEQ ID NO: 1; A at a residue corresponding to residue 266 in SEQ ID NO: 1; M at a residue corresponding to residue 266 in SEQ ID NO: 1; G at a residue corresponding to residue 266 in SEQ ID NO: 1; S at a residue corresponding to residue 266 in SEQ ID NO: 1; W at a residue corresponding to residue 266 in SEQ ID NO: 1; T at a residue corresponding to residue 266 in SEQ ID NO: 1; A at a residue corresponding to residue 267 in SEQ ID NO: 1; T at a residue corresponding to residue 267 in SEQ ID NO: 1; W at a residue corresponding to residue 267 in SEQ ID NO: 1; D at a residue corresponding to residue 284 in SEQ ID NO: 1; Y at a residue corresponding to residue 284 in SEQ ID NO: 1; Q at a residue corresponding to residue 284 in SEQ ID NO: 1; I at a residue corresponding to residue 284 in SEQ ID NO: 1; N at a residue corresponding to residue 284 in SEQ ID NO: 1; C at a residue corresponding to residue 284 in SEQ ID NO: 1; P at a residue corresponding to residue 284 in SEQ ID NO: 1; H at a residue corresponding to residue 284 in SEQ ID NO: 1; R at a residue corresponding to residue 284 in SEQ ID NO: 1; K at a residue corresponding to residue 284 in SEQ ID NO: 1; F at a residue corresponding to residue 284 in SEQ ID NO: 1; W at a residue corresponding to residue 286 in SEQ ID NO: 1; and/or M at a residue corresponding to residue 286 in SEQ ID NO: 1.

In some embodiments, a PTE comprises: K at a residue corresponding to residue 2 in SEQ ID NO: 2; T at a residue corresponding to residue 2 in SEQ ID NO: 2; A at a residue corresponding to residue 2 in SEQ ID NO: 2; T at a residue corresponding to residue 3 in SEQ ID NO: 2; K at a residue corresponding to residue 3 in SEQ ID NO: 2; Q at a residue corresponding to residue 3 in SEQ ID NO: 2; V at a residue corresponding to residue 4 in SEQ ID NO: 2; I at a residue corresponding to residue 4 in SEQ ID NO: 2; Q at a residue corresponding to residue 5 in SEQ ID NO: 2; M at a residue corresponding to residue 5 in SEQ ID NO: 2; R at a residue corresponding to residue 5 in SEQ ID NO: 2; T at a residue corresponding to residue 8 in SEQ ID NO: 2; L at a residue corresponding to residue 8 in SEQ ID NO: 2; C at a residue corresponding to residue 8 in SEQ ID NO: 2; P at a residue corresponding to residue 10 in SEQ ID NO: 2; P at a residue corresponding to residue 12 in SEQ ID NO: 2; E at a residue corresponding to residue 12 in SEQ ID NO: 2; P at a residue corresponding to residue 13 in SEQ ID NO: 2; A at a residue corresponding to residue 13 in SEQ ID NO: 2; S at a residue corresponding to residue 14 in SEQ ID NO: 2; E at a residue corresponding to residue 14 in SEQ ID NO: 2; D at a residue corresponding to residue 14 in SEQ ID NO: 2; Q at a residue corresponding to residue 15 in SEQ ID NO: 2; E at a residue corresponding to residue 15 in SEQ ID NO: 2; D at a residue corresponding to residue 15 in SEQ ID NO: 2; M at a residue corresponding to residue 16 in SEQ ID NO: 2; I at a residue corresponding to residue 18 in SEQ ID NO: 2; M at a residue corresponding to residue 18 in SEQ ID NO: 2; D at a residue corresponding to residue 28 in SEQ ID NO: 2; S at a residue corresponding to residue 29 in SEQ ID NO: 2; W at a residue corresponding to residue 30 in SEQ ID NO: 2; S at a residue corresponding to residue 30 in SEQ ID NO: 2; P at a residue corresponding to residue 30 in SEQ ID NO: 2; G at a residue corresponding to residue 31 in SEQ ID NO: 2; V at a residue corresponding to residue 32 in SEQ ID NO: 2; M at a residue corresponding to residue 32 in SEQ ID NO: 2; F at a residue corresponding to residue 32 in SEQ ID NO: 2; W at a residue corresponding to residue 32 in SEQ ID NO: 2; W at a residue corresponding to residue 33 in SEQ ID NO: 2; Q at a residue corresponding to residue 34 in SEQ ID NO: 2; D at a residue corresponding to residue 35 in SEQ ID NO: 2; H at a residue corresponding to residue 36 in SEQ ID NO: 2; F at a residue corresponding to residue 36 in SEQ ID NO: 2; W at a residue corresponding to residue 36 in SEQ ID NO: 2; D at a residue corresponding to residue 38 in SEQ ID NO: 2; H at a residue corresponding to residue 38 in SEQ ID NO: 2; P at a residue corresponding to residue 39 in SEQ ID NO: 2; F at a residue corresponding to residue 40 in SEQ ID NO: 2; N at a residue corresponding to residue 42 in SEQ ID NO: 2; D at a residue corresponding to residue 43 in SEQ ID NO: 2; N at a residue corresponding to residue 44 in SEQ ID NO: 2; E at a residue corresponding to residue 44 in SEQ ID NO: 2; I at a residue corresponding to residue 47 in SEQ ID NO: 2; M at a residue corresponding to residue 47 in SEQ ID NO: 2; W at a residue corresponding to residue 48 in SEQ ID NO: 2; E at a residue corresponding to residue 48 in SEQ ID NO: 2; C at a residue corresponding to residue 49 in SEQ ID NO: 2; W at a residue corresponding to residue 49 in SEQ ID NO: 2; H at a residue corresponding to residue 49 in SEQ ID NO: 2; M at a residue corresponding to residue 49 in SEQ ID NO: 2; I at a residue corresponding to residue 51 in SEQ ID NO: 2; L at a residue corresponding to residue 51 in SEQ ID NO: 2; R at a residue corresponding to residue 52 in SEQ ID NO: 2; E at a residue corresponding to residue 53 in SEQ ID NO: 2; Q at a residue corresponding to residue 53 in SEQ ID NO: 2; K at a residue corresponding to residue 55 in SEQ ID NO: 2; Q at a residue corresponding to residue 56 in SEQ ID NO: 2; R at a residue corresponding to residue 56 in SEQ ID NO: 2; D at a residue corresponding to residue 56 in SEQ ID NO: 2; Y at a residue corresponding to residue 57 in SEQ ID NO: 2; F at a residue corresponding to residue 57 in SEQ ID NO: 2; E at a residue corresponding to residue 59 in SEQ ID NO: 2; Q at a residue corresponding to residue 59 in SEQ ID NO: 2; A at a residue corresponding to residue 60 in SEQ ID NO: 2; H at a residue corresponding to residue 60 in SEQ ID NO: 2; N at a residue corresponding to residue 63 in SEQ ID NO: 2; S at a residue corresponding to residue 64 in SEQ ID NO: 2; D at a residue corresponding to residue 72 in SEQ ID NO: 2; R at a residue corresponding to residue 74 in SEQ ID NO: 2; D at a residue corresponding to residue 76 in SEQ ID NO: 2; N at a residue corresponding to residue 76 in SEQ ID NO: 2; V at a residue corresponding to residue 77 in SEQ ID NO: 2; P at a residue corresponding to residue 77 in SEQ ID NO: 2; E at a residue corresponding to residue 78 in SEQ ID NO: 2; D at a residue corresponding to residue 78 in SEQ ID NO: 2; L at a residue corresponding to residue 80 in SEQ ID NO: 2; M at a residue corresponding to residue 80 in SEQ ID NO: 2; V at a residue corresponding to residue 80 in SEQ ID NO: 2; R at a residue corresponding to residue 81 in SEQ ID NO: 2; K at a residue corresponding to residue 82 in SEQ ID NO: 2; E at a residue corresponding to residue 82 in SEQ ID NO: 2; I at a residue corresponding to residue 83 in SEQ ID NO: 2; L at a residue corresponding to residue 83 in SEQ ID NO: 2; S at a residue corresponding to residue 84 in SEQ ID NO: 2; R at a residue corresponding to residue 85 in SEQ ID NO: 2; E at a residue corresponding to residue 85 in SEQ ID NO: 2; R at a residue corresponding to residue 86 in SEQ ID NO: 2; S at a residue corresponding to residue 87 in SEQ ID NO: 2; G at a residue corresponding to residue 88 in SEQ ID NO: 2; V at a residue corresponding to residue 89 in SEQ ID NO: 2; M at a residue corresponding to residue 89 in SEQ ID NO: 2; H at a residue corresponding to residue 90 in SEQ ID NO: 2; V at a residue corresponding to residue 91 in SEQ ID NO: 2; I at a residue corresponding to residue 92 in SEQ ID NO: 2; C at a residue corresponding to residue 93 in SEQ ID NO: 2; C at a residue corresponding to residue 99 in SEQ ID NO: 2; Y at a residue corresponding to residue 103 in SEQ ID NO: 2; W at a residue corresponding to residue 103 in SEQ ID NO: 2; H at a residue corresponding to residue 103 in SEQ ID NO: 2; W at a residue corresponding to residue 105 in SEQ ID NO: 2; H at a residue corresponding to residue 106 in SEQ ID NO: 2; W at a residue corresponding to residue 106 in SEQ ID NO: 2; F at a residue corresponding to residue 106 in SEQ ID NO: 2; M at a residue corresponding to residue 107 in SEQ ID NO: 2; Y at a residue corresponding to residue 107 in SEQ ID NO: 2; W at a residue corresponding to residue 107 in SEQ ID NO: 2; R at a residue corresponding to residue 108 in SEQ ID NO: 2; W at a residue corresponding to residue 109 in SEQ ID NO: 2; F at a residue corresponding to residue 109 in SEQ ID NO: 2; W at a residue corresponding to residue 110 in SEQ ID NO: 2; M at a residue corresponding to residue 110 in SEQ ID NO: 2; D at a residue corresponding to residue 115 in SEQ ID NO: 2; T at a residue corresponding to residue 118 in SEQ ID NO: 2; S at a residue corresponding to residue 118 in SEQ ID NO: 2; D at a residue corresponding to residue 120 in SEQ ID NO: 2; E at a residue corresponding to residue 121 in SEQ ID NO: 2; Q at a residue corresponding to residue 121 in SEQ ID NO: 2; L at a residue corresponding to residue 122 in SEQ ID NO: 2; I at a residue corresponding to residue 122 in SEQ ID NO: 2; A at a residue corresponding to residue 123 in SEQ ID NO: 2; E at a residue corresponding to residue 124 in SEQ ID NO: 2; I at a residue corresponding to residue 127 in SEQ ID NO: 2; N at a residue corresponding to residue 128 in SEQ ID NO: 2; H at a residue corresponding to residue 128 in SEQ ID NO: 2; D at a residue corresponding to residue 132 in SEQ ID NO: 2; E at a residue corresponding to residue 132 in SEQ ID NO: 2; V at a residue corresponding to residue 134 in SEQ ID NO: 2; G at a residue corresponding to residue 135 in SEQ ID NO: 2; E at a residue corresponding to residue 135 in SEQ ID NO: 2; G at a residue corresponding to residue 136 in SEQ ID NO: 2; V at a residue corresponding to residue 139 in SEQ ID NO: 2; H at a residue corresponding to residue 140 in SEQ ID NO: 2; R at a residue corresponding to residue 140 in SEQ ID NO: 2; S at a residue corresponding to residue 150 in SEQ ID NO: 2; Y at a residue corresponding to residue 150 in SEQ ID NO: 2; H at a residue corresponding to residue 150 in SEQ ID NO: 2; W at a residue corresponding to residue 151 in SEQ ID NO: 2; N at a residue corresponding to residue 151 in SEQ ID NO: 2; D at a residue corresponding to residue 151 in SEQ ID NO: 2; H at a residue corresponding to residue 151 in SEQ ID NO: 2; I at a residue corresponding to residue 153 in SEQ ID NO: 2; W at a residue corresponding to residue 153 in SEQ ID NO: 2; D at a residue corresponding to residue 155 in SEQ ID NO: 2; E at a residue corresponding to residue 155 in SEQ ID NO: 2; W at a residue corresponding to residue 156 in SEQ ID NO: 2; E at a residue corresponding to residue 157 in SEQ ID NO: 2; H at a residue corresponding to residue 158 in SEQ ID NO: 2; C at a residue corresponding to residue 165 in SEQ ID NO: 2; R at a residue corresponding to residue 166 in SEQ ID NO: 2; Q at a residue corresponding to residue 168 in SEQ ID NO: 2; D at a residue corresponding to residue 182 in SEQ ID NO: 2; T at a residue corresponding to residue 183 in SEQ ID NO: 2; F at a residue corresponding to residue 187 in SEQ ID NO: 2; M at a residue corresponding to residue 187 in SEQ ID NO: 2; H at a residue corresponding to residue 187 in SEQ ID NO: 2; C at a residue corresponding to residue 188 in SEQ ID NO: 2; W at a residue corresponding to residue 188 in SEQ ID NO: 2; I at a residue corresponding to residue 190 in SEQ ID NO: 2; L at a residue corresponding to residue 190 in SEQ ID NO: 2; R at a residue corresponding to residue 191 in SEQ ID NO: 2; L at a residue corresponding to residue 193 in SEQ ID NO: 2; D at a residue corresponding to residue 194 in SEQ ID NO: 2; D at a residue corresponding to residue 195 in SEQ ID NO: 2; P at a residue corresponding to residue 200 in SEQ ID NO: 2; N at a residue corresponding to residue 201 in SEQ ID NO: 2; H at a residue corresponding to residue 202 in SEQ ID NO: 2; K at a residue corresponding to residue 202 in SEQ ID NO: 2; C at a residue corresponding to residue 203 in SEQ ID NO: 2; I at a residue corresponding to residue 203 in SEQ ID NO: 2; M at a residue corresponding to residue 214 in SEQ ID NO: 2; H at a residue corresponding to residue 214 in SEQ ID NO: 2; D at a residue corresponding to residue 215 in SEQ ID NO: 2; E at a residue corresponding to residue 215 in SEQ ID NO: 2; D at a residue corresponding to residue 219 in SEQ ID NO: 2; M at a residue corresponding to residue 220 in SEQ ID NO: 2; V at a residue corresponding to residue 220 in SEQ ID NO: 2; I at a residue corresponding to residue 220 in SEQ ID NO: 2; M at a residue corresponding to residue 221 in SEQ ID NO: 2; A at a residue corresponding to residue 221 in SEQ ID NO: 2; C at a residue corresponding to residue 221 in SEQ ID NO: 2; H at a residue corresponding to residue 222 in SEQ ID NO: 2; R at a residue corresponding to residue 223 in SEQ ID NO: 2; C at a residue corresponding to residue 225 in SEQ ID NO: 2; W at a residue corresponding to residue 226 in SEQ ID NO: 2; V at a residue corresponding to residue 227 in SEQ ID NO: 2; I at a residue corresponding to residue 227 in SEQ ID NO: 2; C at a residue corresponding to residue 235 in SEQ ID NO: 2; K at a residue corresponding to residue 236 in SEQ ID NO: 2; H at a residue corresponding to residue 236 in SEQ ID NO: 2; W at a residue corresponding to residue 236 in SEQ ID NO: 2; Y at a residue corresponding to residue 238 in SEQ ID NO: 2; H at a residue corresponding to residue 238 in SEQ ID NO: 2; M at a residue corresponding to residue 238 in SEQ ID NO: 2; S at a residue corresponding to residue 239 in SEQ ID NO: 2; D at a residue corresponding to residue 240 in SEQ ID NO: 2; W at a residue corresponding to residue 240 in SEQ ID NO: 2; Y at a residue corresponding to residue 240 in SEQ ID NO: 2; D at a residue corresponding to residue 241 in SEQ ID NO: 2; E at a residue corresponding to residue 242 in SEQ ID NO: 2; C at a residue corresponding to residue 244 in SEQ ID NO: 2; D at a residue corresponding to residue 245 in SEQ ID NO: 2; R at a residue corresponding to residue 245 in SEQ ID NO: 2; E at a residue corresponding to residue 245 in SEQ ID NO: 2; M at a residue corresponding to residue 246 in SEQ ID NO: 2; L at a residue corresponding to residue 246 in SEQ ID NO: 2; L at a residue corresponding to residue 247 in SEQ ID NO: 2; I at a residue corresponding to residue 247 in SEQ ID NO: 2; E at a residue corresponding to residue 249 in SEQ ID NO: 2; W at a residue corresponding to residue 249 in SEQ ID NO: 2; R at a residue corresponding to residue 249 in SEQ ID NO: 2; H at a residue corresponding to residue 249 in SEQ ID NO: 2; L at a residue corresponding to residue 250 in SEQ ID NO: 2; V at a residue corresponding to residue 251 in SEQ ID NO: 2; I at a residue corresponding to residue 251 in SEQ ID NO: 2; H at a residue corresponding to residue 252 in SEQ ID NO: 2; N at a residue corresponding to residue 253 in SEQ ID NO: 2; Y at a residue corresponding to residue 255 in SEQ ID NO: 2; W at a residue corresponding to residue 255 in SEQ ID NO: 2; R at a residue corresponding to residue 258 in SEQ ID NO: 2; H at a residue corresponding to residue 258 in SEQ ID NO: 2; I at a residue corresponding to residue 259 in SEQ ID NO: 2; G at a residue corresponding to residue 271 in SEQ ID NO: 2; H at a residue corresponding to residue 272 in SEQ ID NO: 2; W at a residue corresponding to residue 272 in SEQ ID NO: 2; M at a residue corresponding to residue 272 in SEQ ID NO: 2; C at a residue corresponding to residue 272 in SEQ ID NO: 2; G at a residue corresponding to residue 274 in SEQ ID NO: 2; W at a residue corresponding to residue 274 in SEQ ID NO: 2; K at a residue corresponding to residue 275 in SEQ ID NO: 2; W at a residue corresponding to residue 276 in SEQ ID NO: 2; W at a residue corresponding to residue 277 in SEQ ID NO: 2; R at a residue corresponding to residue 278 in SEQ ID NO: 2; K at a residue corresponding to residue 279 in SEQ ID NO: 2; R at a residue corresponding to residue 279 in SEQ ID NO: 2; H at a residue corresponding to residue 279 in SEQ ID NO: 2; F at a residue corresponding to residue 281 in SEQ ID NO: 2; Y at a residue corresponding to residue 281 in SEQ ID NO: 2; G at a residue corresponding to residue 282 in SEQ ID NO: 2; D at a residue corresponding to residue 283 in SEQ ID NO: 2; G at a residue corresponding to residue 283 in SEQ ID NO: 2; G at a residue corresponding to residue 285 in SEQ ID NO: 2; T at a residue corresponding to residue 287 in SEQ ID NO: 2; F at a residue corresponding to residue 288 in SEQ ID NO: 2; Y at a residue corresponding to residue 288 in SEQ ID NO: 2; L at a residue corresponding to residue 289 in SEQ ID NO: 2; V at a residue corresponding to residue 289 in SEQ ID NO: 2; L at a residue corresponding to residue 290 in SEQ ID NO: 2; F at a residue corresponding to residue 290 in SEQ ID NO: 2; D at a residue corresponding to residue 291 in SEQ ID NO: 2; E at a residue corresponding to residue 291 in SEQ ID NO: 2; T at a residue corresponding to residue 291 in SEQ ID NO: 2; R at a residue corresponding to residue 291 in SEQ ID NO: 2; N at a residue corresponding to residue 292 in SEQ ID NO: 2; R at a residue corresponding to residue 292 in SEQ ID NO: 2; I at a residue corresponding to residue 293 in SEQ ID NO: 2; F at a residue corresponding to residue 293 in SEQ ID NO: 2; L at a residue corresponding to residue 294 in SEQ ID NO: 2; V at a residue corresponding to residue 294 in SEQ ID NO: 2; R at a residue corresponding to residue 296 in SEQ ID NO: 2; M at a residue corresponding to residue 296 in SEQ ID NO: 2; M at a residue corresponding to residue 298 in SEQ ID NO: 2; R at a residue corresponding to residue 298 in SEQ ID NO: 2; K at a residue corresponding to residue 299 in SEQ ID NO: 2; R at a residue corresponding to residue 299 in SEQ ID NO: 2; Q at a residue corresponding to residue 299 in SEQ ID NO: 2; A at a residue corresponding to residue 300 in SEQ ID NO: 2; D at a residue corresponding to residue 303 in SEQ ID NO: 2; S at a residue corresponding to residue 303 in SEQ ID NO: 2; Q at a residue corresponding to residue 304 in SEQ ID NO: 2; E at a residue corresponding to residue 304 in SEQ ID NO: 2; D at a residue corresponding to residue 305 in SEQ ID NO: 2; A at a residue corresponding to residue 305 in SEQ ID NO: 2; D at a residue corresponding to residue 306 in SEQ ID NO: 2; E at a residue corresponding to residue 306 in SEQ ID NO: 2; V at a residue corresponding to residue 307 in SEQ ID NO: 2; I at a residue corresponding to residue 307 in SEQ ID NO: 2; E at a residue corresponding to residue 308 in SEQ ID NO: 2; N at a residue corresponding to residue 308 in SEQ ID NO: 2; R at a residue corresponding to residue 308 in SEQ ID NO: 2; K at a residue corresponding to residue 309 in SEQ ID NO: 2; Q at a residue corresponding to residue 309 in SEQ ID NO: 2; R at a residue corresponding to residue 309 in SEQ ID NO: 2; M at a residue corresponding to residue 311 in SEQ ID NO: 2; T at a residue corresponding to residue 311 in SEQ ID NO: 2; F at a residue corresponding to residue 311 in SEQ ID NO: 2; I at a residue corresponding to residue 312 in SEQ ID NO: 2; E at a residue corresponding to residue 313 in SEQ ID NO: 2; A at a residue corresponding to residue 316 in SEQ ID NO: 2; K at a residue corresponding to residue 316 in SEQ ID NO: 2; Q at a residue corresponding to residue 316 in SEQ ID NO: 2; N at a residue corresponding to residue 317 in SEQ ID NO: 2; K at a residue corresponding to residue 317 in SEQ ID NO: 2; L at a residue corresponding to residue 318 in SEQ ID NO: 2; F at a residue corresponding to residue 318 in SEQ ID NO: 2; M at a residue corresponding to residue 318 in SEQ ID NO: 2; S at a residue corresponding to residue 320 in SEQ ID NO: 2; Q at a residue corresponding to residue 320 in SEQ ID NO: 2; D at a residue corresponding to residue 320 in SEQ ID NO: 2; R at a residue corresponding to residue 322 in SEQ ID NO: 2; K at a residue corresponding to residue 322 in SEQ ID NO: 2; E at a residue corresponding to residue 322 in SEQ ID NO: 2; P at a residue corresponding to residue 324 in SEQ ID NO: 2; S at a residue corresponding to residue 324 in SEQ ID NO: 2; H at a residue corresponding to residue 325 in SEQ ID NO: 2; W at a residue corresponding to residue 325 in SEQ ID NO: 2; F at a residue corresponding to residue 325 in SEQ ID NO: 2; Q at a residue corresponding to residue 326 in SEQ ID NO: 2; K at a residue corresponding to residue 326 in SEQ ID NO: 2; and/or R at a residue corresponding to residue 326 in SEQ ID NO: 2.

In some embodiments, a PTE comprises: T at a residue corresponding to residue 22 in SEQ ID NO: 3; N at a residue corresponding to residue 22 in SEQ ID NO: 3; S at a residue corresponding to residue 22 in SEQ ID NO: 3; D at a residue corresponding to residue 23 in SEQ ID NO: 3; N at a residue corresponding to residue 24 in SEQ ID NO: 3; T at a residue corresponding to residue 25 in SEQ ID NO: 3; C at a residue corresponding to residue 25 in SEQ ID NO: 3; I at a residue corresponding to residue 25 in SEQ ID NO: 3; A at a residue corresponding to residue 25 in SEQ ID NO: 3; F at a residue corresponding to residue 27 in SEQ ID NO: 3; W at a residue corresponding to residue 27 in SEQ ID NO: 3; C at a residue corresponding to residue 27 in SEQ ID NO: 3; L at a residue corresponding to residue 27 in SEQ ID NO: 3; V at a residue corresponding to residue 27 in SEQ ID NO: 3; T at a residue corresponding to residue 27 in SEQ ID NO: 3; Y at a residue corresponding to residue 27 in SEQ ID NO: 3; M at a residue corresponding to residue 27 in SEQ ID NO: 3; P at a residue corresponding to residue 68 in SEQ ID NO: 3; N at a residue corresponding to residue 68 in SEQ ID NO: 3; M at a residue corresponding to residue 68 in SEQ ID NO: 3; A at a residue corresponding to residue 68 in SEQ ID NO: 3; Q at a residue corresponding to residue 68 in SEQ ID NO: 3; S at a residue corresponding to residue 69 in SEQ ID NO: 3; P at a residue corresponding to residue 70 in SEQ ID NO: 3; C at a residue corresponding to residue 70 in SEQ ID NO: 3; M at a residue corresponding to residue 73 in SEQ ID NO: 3; C at a residue corresponding to residue 73 in SEQ ID NO: 3; V at a residue corresponding to residue 73 in SEQ ID NO: 3; H at a residue corresponding to residue 73 in SEQ ID NO: 3; H at a residue corresponding to residue 98 in SEQ ID NO: 3; C at a residue corresponding to residue 98 in SEQ ID NO: 3; F at a residue corresponding to residue 98 in SEQ ID NO: 3; Q at a residue corresponding to residue 100 in SEQ ID NO: 3; H at a residue corresponding to residue 100 in SEQ ID NO: 3; E at a residue corresponding to residue 100 in SEQ ID NO: 3; D at a residue corresponding to residue 100 in SEQ ID NO: 3; I at a residue corresponding to residue 144 in SEQ ID NO: 3; T at a residue corresponding to residue 145 in SEQ ID NO: 3; C at a residue corresponding to residue 145 in SEQ ID NO: 3; S at a residue corresponding to residue 145 in SEQ ID NO: 3; E at a residue corresponding to residue 145 in SEQ ID NO: 3; Q at a residue corresponding to residue 145 in SEQ ID NO: 3; V at a residue corresponding to residue 146 in SEQ ID NO: 3; Y at a residue corresponding to residue 146 in SEQ ID NO: 3; L at a residue corresponding to residue 146 in SEQ ID NO: 3; I at a residue corresponding to residue 146 in SEQ ID NO: 3; W at a residue corresponding to residue 147 in SEQ ID NO: 3; H at a residue corresponding to residue 147 in SEQ ID NO: 3; C at a residue corresponding to residue 147 in SEQ ID NO: 3; W at a residue corresponding to residue 148 in SEQ ID NO: 3; I at a residue corresponding to residue 148 in SEQ ID NO: 3; A at a residue corresponding to residue 148 in SEQ ID NO: 3; V at a residue corresponding to residue 148 in SEQ ID NO: 3; Y at a residue corresponding to residue 148 in SEQ ID NO: 3; I at a residue corresponding to residue 164 in SEQ ID NO: 3; L at a residue corresponding to residue 176 in SEQ ID NO: 3; C at a residue corresponding to residue 176 in SEQ ID NO: 3; H at a residue corresponding to residue 176 in SEQ ID NO: 3; I at a residue corresponding to residue 176 in SEQ ID NO: 3; F at a residue corresponding to residue 176 in SEQ ID NO: 3; W at a residue corresponding to residue 176 in SEQ ID NO: 3; V at a residue corresponding to residue 176 in SEQ ID NO: 3; Y at a residue corresponding to residue 176 in SEQ ID NO: 3; C at a residue corresponding to residue 177 in SEQ ID NO: 3; D at a residue corresponding to residue 178 in SEQ ID NO: 3; T at a residue corresponding to residue 178 in SEQ ID NO: 3; R at a residue corresponding to residue 178 in SEQ ID NO: 3; A at a residue corresponding to residue 178 in SEQ ID NO: 3; S at a residue corresponding to residue 178 in SEQ ID NO: 3; C at a residue corresponding to residue 179 in SEQ ID NO: 3; M at a residue corresponding to residue 179 in SEQ ID NO: 3; F at a residue corresponding to residue 179 in SEQ ID NO: 3; R at a residue corresponding to residue 181 in SEQ ID NO: 3; S at a residue corresponding to residue 181 in SEQ ID NO: 3; C at a residue corresponding to residue 181 in SEQ ID NO: 3; A at a residue corresponding to residue 189 in SEQ ID NO: 3; V at a residue corresponding to residue 189 in SEQ ID NO: 3; E at a residue corresponding to residue 189 in SEQ ID NO: 3; M at a residue corresponding to residue 189 in SEQ ID NO: 3; I at a residue corresponding to residue 189 in SEQ ID NO: 3; C at a residue corresponding to residue 189 in SEQ ID NO: 3; L at a residue corresponding to residue 189 in SEQ ID NO: 3; N at a residue corresponding to residue 206 in SEQ ID NO: 3; S at a residue corresponding to residue 206 in SEQ ID NO: 3; M at a residue corresponding to residue 207 in SEQ ID NO: 3; N at a residue corresponding to residue 207 in SEQ ID NO: 3; A at a residue corresponding to residue 208 in SEQ ID NO: 3; C at a residue corresponding to residue 208 in SEQ ID NO: 3; T at a residue corresponding to residue 208 in SEQ ID NO: 3; N at a residue corresponding to residue 209 in SEQ ID NO: 3; E at a residue corresponding to residue 210 in SEQ ID NO: 3; W at a residue corresponding to residue 210 in SEQ ID NO: 3; C at a residue corresponding to residue 210 in SEQ ID NO: 3; H at a residue corresponding to residue 210 in SEQ ID NO: 3; P at a residue corresponding to residue 210 in SEQ ID NO: 3; Q at a residue corresponding to residue 228 in SEQ ID NO: 3; D at a residue corresponding to residue 228 in SEQ ID NO: 3; C at a residue corresponding to residue 228 in SEQ ID NO: 3; S at a residue corresponding to residue 228 in SEQ ID NO: 3; N at a residue corresponding to residue 230 in SEQ ID NO: 3; H at a residue corresponding to residue 231 in SEQ ID NO: 3; Q at a residue corresponding to residue 231 in SEQ ID NO: 3; M at a residue corresponding to residue 231 in SEQ ID NO: 3; W at a residue corresponding to residue 231 in SEQ ID NO: 3; T at a residue corresponding to residue 231 in SEQ ID NO: 3; N at a residue corresponding to residue 231 in SEQ ID NO: 3; I at a residue corresponding to residue 260 in SEQ ID NO: 3; G at a residue corresponding to residue 262 in SEQ ID NO: 3; Q at a residue corresponding to residue 263 in SEQ ID NO: 3; F at a residue corresponding to residue 263 in SEQ ID NO: 3; M at a residue corresponding to residue 263 in SEQ ID NO: 3; G at a residue corresponding to residue 263 in SEQ ID NO: 3; T at a residue corresponding to residue 263 in SEQ ID NO: 3; T at a residue corresponding to residue 264 in SEQ ID NO: 3; P at a residue corresponding to residue 264 in SEQ ID NO: 3; S at a residue corresponding to residue 264 in SEQ ID NO: 3; Y at a residue corresponding to residue 265 in SEQ ID NO: 3; I at a residue corresponding to residue 265 in SEQ ID NO: 3; W at a residue corresponding to residue 265 in SEQ ID NO: 3; M at a residue corresponding to residue 265 in SEQ ID NO: 3; S at a residue corresponding to residue 265 in SEQ ID NO: 3; T at a residue corresponding to residue 265 in SEQ ID NO: 3; L at a residue corresponding to residue 266 in SEQ ID NO: 3; Q at a residue corresponding to residue 266 in SEQ ID NO: 3; G at a residue corresponding to residue 266 in SEQ ID NO: 3; H at a residue corresponding to residue 266 in SEQ ID NO: 3; C at a residue corresponding to residue 266 in SEQ ID NO: 3; A at a residue corresponding to residue 266 in SEQ ID NO: 3; T at a residue corresponding to residue 266 in SEQ ID NO: 3; W at a residue corresponding to residue 266 in SEQ ID NO: 3; I at a residue corresponding to residue 266 in SEQ ID NO: 3; T at a residue corresponding to residue 267 in SEQ ID NO: 3; W at a residue corresponding to residue 267 in SEQ ID NO: 3; K at a residue corresponding to residue 284 in SEQ ID NO: 3; E at a residue corresponding to residue 284 in SEQ ID NO: 3; I at a residue corresponding to residue 284 in SEQ ID NO: 3; P at a residue corresponding to residue 284 in SEQ ID NO: 3; T at a residue corresponding to residue 284 in SEQ ID NO: 3; H at a residue corresponding to residue 284 in SEQ ID NO: 3; and/or R at a residue corresponding to residue 284 in SEQ ID NO: 3.

In some embodiments, a PTE comprises: M at a residue corresponding to residue 20 in SEQ ID NO: 4; D at a residue corresponding to residue 23 in SEQ ID NO: 4; M at a residue corresponding to residue 25 in SEQ ID NO: 4; L at a residue corresponding to residue in SEQ ID NO: 4; L at a residue corresponding to residue 26 in SEQ ID NO: 4; H at a residue corresponding to residue 26 in SEQ ID NO: 4; M at a residue corresponding to residue 26 in SEQ ID NO: 4; W at a residue corresponding to residue 26 in SEQ ID NO: 4; W at a residue corresponding to residue 27 in SEQ ID NO: 4; C at a residue corresponding to residue 27 in SEQ ID NO: 4; Y at a residue corresponding to residue 27 in SEQ ID NO: 4; T at a residue corresponding to residue 27 in SEQ ID NO: 4; V at a residue corresponding to residue 27 in SEQ ID NO: 4; F at a residue corresponding to residue 27 in SEQ ID NO: 4; M at a residue corresponding to residue 27 in SEQ ID NO: 4; I at a residue corresponding to residue 66 in SEQ ID NO: 4; M at a residue corresponding to residue 68 in SEQ ID NO: 4; A at a residue corresponding to residue 68 in SEQ ID NO: 4; C at a residue corresponding to residue 68 in SEQ ID NO: 4; N at a residue corresponding to residue 68 in SEQ ID NO: 4; S at a residue corresponding to residue 69 in SEQ ID NO: 4; Q at a residue corresponding to residue 69 in SEQ ID NO: 4; at a residue corresponding to residue 73 in SEQ ID NO: 4; C at a residue corresponding to residue 73 in SEQ ID NO: 4; C at a residue corresponding to residue 98 in SEQ ID NO: 4; W at a residue corresponding to residue 98 in SEQ ID NO: 4; C at a residue corresponding to residue 126 in SEQ ID NO: 4; L at a residue corresponding to residue 126 in SEQ ID NO: 4; M at a residue corresponding to residue 126 in SEQ ID NO: 4; I at a residue corresponding to residue 144 in SEQ ID NO: 4; Q at a residue corresponding to residue 145 in SEQ ID NO: 4; C at a residue corresponding to residue 145 in SEQ ID NO: 4; T at a residue corresponding to residue 145 in SEQ ID NO: 4; R at a residue corresponding to residue 145 in SEQ ID NO: 4; S at a residue corresponding to residue 145 in SEQ ID NO: 4; E at a residue corresponding to residue 145 in SEQ ID NO: 4; F at a residue corresponding to residue 146 in SEQ ID NO: 4; I at a residue corresponding to residue 146 in SEQ ID NO: 4; V at a residue corresponding to residue 146 in SEQ ID NO: 4; G at a residue corresponding to residue 147 in SEQ ID NO: 4; S at a residue corresponding to residue 147 in SEQ ID NO: 4; M at a residue corresponding to residue 147 in SEQ ID NO: 4; I at a residue corresponding to residue 147 in SEQ ID NO: 4; Y at a residue corresponding to residue 148 in SEQ ID NO: 4; M at a residue corresponding to residue 148 in SEQ ID NO: 4; C at a residue corresponding to residue 148 in SEQ ID NO: 4; V at a residue corresponding to residue 148 in SEQ ID NO: 4; W at a residue corresponding to residue 148 in SEQ ID NO: 4; I at a residue corresponding to residue 148 in SEQ ID NO: 4; I at a residue corresponding to residue 164 in SEQ ID NO: 4; T at a residue corresponding to residue 164 in SEQ ID NO: 4; S at a residue corresponding to residue 168 in SEQ ID NO: 4; C at a residue corresponding to residue 173 in SEQ ID NO: 4; Y at a residue corresponding to residue 176 in SEQ ID NO: 4; W at a residue corresponding to residue 176 in SEQ ID NO: 4; L at a residue corresponding to residue 176 in SEQ ID NO: 4; M at a residue corresponding to residue 176 in SEQ ID NO: 4; H at a residue corresponding to residue 176 in SEQ ID NO: 4; I at a residue corresponding to residue 176 in SEQ ID NO: 4; F at a residue corresponding to residue 176 in SEQ ID NO: 4; T at a residue corresponding to residue 176 in SEQ ID NO: 4; V at a residue corresponding to residue 177 in SEQ ID NO: 4; C at a residue corresponding to residue 177 in SEQ ID NO: 4; I at a residue corresponding to residue 177 in SEQ ID NO: 4; T at a residue corresponding to residue 178 in SEQ ID NO: 4; S at a residue corresponding to residue 178 in SEQ ID NO: 4; Q at a residue corresponding to residue 178 in SEQ ID NO: 4; Y at a residue corresponding to residue 178 in SEQ ID NO: 4; R at a residue corresponding to residue 178 in SEQ ID NO: 4; V at a residue corresponding to residue 178 in SEQ ID NO: 4; D at a residue corresponding to residue 178 in SEQ ID NO: 4; I at a residue corresponding to residue 179 in SEQ ID NO: 4; M at a residue corresponding to residue 179 in SEQ ID NO: 4; N at a residue corresponding to residue 179 in SEQ ID NO: 4; F at a residue corresponding to residue 179 in SEQ ID NO: 4; W at a residue corresponding to residue 179 in SEQ ID NO: 4; D at a residue corresponding to residue 179 in SEQ ID NO: 4; H at a residue corresponding to residue 181 in SEQ ID NO: 4; S at a residue corresponding to residue 181 in SEQ ID NO: 4; M at a residue corresponding to residue 181 in SEQ ID NO: 4; C at a residue corresponding to residue 181 in SEQ ID NO: 4; W at a residue corresponding to residue 181 in SEQ ID NO: 4; G at a residue corresponding to residue 181 in SEQ ID NO: 4; M at a residue corresponding to residue 189 in SEQ ID NO: 4; V at a residue corresponding to residue 189 in SEQ ID NO: 4; I at a residue corresponding to residue 189 in SEQ ID NO: 4; L at a residue corresponding to residue 189 in SEQ ID NO: 4; C at a residue corresponding to residue 189 in SEQ ID NO: 4; E at a residue corresponding to residue 189 in SEQ ID NO: 4; A at a residue corresponding to residue 189 in SEQ ID NO: 4; V at a residue corresponding to residue 204 in SEQ ID NO: 4; S at a residue corresponding to residue 206 in SEQ ID NO: 4; N at a residue corresponding to residue 206 in SEQ ID NO: 4; N at a residue corresponding to residue 207 in SEQ ID NO: 4; A at a residue corresponding to residue 208 in SEQ ID NO: 4; C at a residue corresponding to residue 208 in SEQ ID NO: 4; M at a residue corresponding to residue 208 in SEQ ID NO: 4; T at a residue corresponding to residue 208 in SEQ ID NO: 4; N at a residue corresponding to residue 209 in SEQ ID NO: 4; C at a residue corresponding to residue 209 in SEQ ID NO: 4; M at a residue corresponding to residue 210 in SEQ ID NO: 4; E at a residue corresponding to residue 210 in SEQ ID NO: 4; W at a residue corresponding to residue 210 in SEQ ID NO: 4; C at a residue corresponding to residue 210 in SEQ ID NO: 4; C at a residue corresponding to residue 228 in SEQ ID NO: 4; S at a residue corresponding to residue 228 in SEQ ID NO: 4; A at a residue corresponding to residue 228 in SEQ ID NO: 4; Q at a residue corresponding to residue 228 in SEQ ID NO: 4; S at a residue corresponding to residue 230 in SEQ ID NO: 4; F at a residue corresponding to residue 231 in SEQ ID NO: 4; N at a residue corresponding to residue 231 in SEQ ID NO: 4; G at a residue corresponding to residue 231 in SEQ ID NO: 4; Q at a residue corresponding to residue 231 in SEQ ID NO: 4; H at a residue corresponding to residue 231 in SEQ ID NO: 4; W at a residue corresponding to residue 231 in SEQ ID NO: 4; C at a residue corresponding to residue 231 in SEQ ID NO: 4; M at a residue corresponding to residue 231 in SEQ ID NO: 4; T at a residue corresponding to residue 231 in SEQ ID NO: 4; I at a residue corresponding to residue 260 in SEQ ID NO: 4; G at a residue corresponding to residue 262 in SEQ ID NO: 4; C at a residue corresponding to residue 262 in SEQ ID NO: 4; T at a residue corresponding to residue 263 in SEQ ID NO: 4; C at a residue corresponding to residue 263 in SEQ ID NO: 4; N at a residue corresponding to residue 263 in SEQ ID NO: 4; S at a residue corresponding to residue 263 in SEQ ID NO: 4; F at a residue corresponding to residue 263 in SEQ ID NO: 4; Y at a residue corresponding to residue 263 in SEQ ID NO: 4; F at a residue corresponding to residue 264 in SEQ ID NO: 4; N at a residue corresponding to residue 264 in SEQ ID NO: 4; M at a residue corresponding to residue 265 in SEQ ID NO: 4; V at a residue corresponding to residue 265 in SEQ ID NO: 4; W at a residue corresponding to residue 265 in SEQ ID NO: 4; S at a residue corresponding to residue 265 in SEQ ID NO: 4; T at a residue corresponding to residue 265 in SEQ ID NO: 4; C at a residue corresponding to residue 265 in SEQ ID NO: 4; Y at a residue corresponding to residue 265 in SEQ ID NO: 4; Q at a residue corresponding to residue 265 in SEQ ID NO: 4; I at a residue corresponding to residue 265 in SEQ ID NO: 4; L at a residue corresponding to residue 265 in SEQ ID NO: 4; C at a residue corresponding to residue 266 in SEQ ID NO: 4; A at a residue corresponding to residue 266 in SEQ ID NO: 4; G at a residue corresponding to residue 266 in SEQ ID NO: 4; T at a residue corresponding to residue 266 in SEQ ID NO: 4; R at a residue corresponding to residue 266 in SEQ ID NO: 4; H at a residue corresponding to residue 266 in SEQ ID NO: 4; S at a residue corresponding to residue 266 in SEQ ID NO: 4; G at a residue corresponding to residue 267 in SEQ ID NO: 4; T at a residue corresponding to residue 267 in SEQ ID NO: 4; A at a residue corresponding to residue 267 in SEQ ID NO: 4; W at a residue corresponding to residue 267 in SEQ ID NO: 4; R at a residue corresponding to residue 284 in SEQ ID NO: 4; D at a residue corresponding to residue 284 in SEQ ID NO: 4; F at a residue corresponding to residue 284 in SEQ ID NO: 4; T at a residue corresponding to residue 284 in SEQ ID NO: 4; K at a residue corresponding to residue 284 in SEQ ID NO: 4; C at a residue corresponding to residue 284 in SEQ ID NO: 4; P at a residue corresponding to residue 284 in SEQ ID NO: 4; N at a residue corresponding to residue 284 in SEQ ID NO: 4; Q at a residue corresponding to residue 284 in SEQ ID NO: 4; E at a residue corresponding to residue 284 in SEQ ID NO: 4; M at a residue corresponding to residue 284 in SEQ ID NO: 4; H at a residue corresponding to residue 284 in SEQ ID NO: 4; Y at a residue corresponding to residue 284 in SEQ ID NO: 4; F at a residue corresponding to residue 286 in SEQ ID NO: 4; M at a residue corresponding to residue 286 in SEQ ID NO: 4; and/or W at a residue corresponding to residue 286 in SEQ ID NO: 4. In some embodiments, a PTER comprises: I at a residue corresponding to residue 29 in SEQ ID NO: 6; R at a residue corresponding to residue 31 in SEQ ID NO: 6; S at a residue corresponding to residue 31 in SEQ ID NO: 6; M at a residue corresponding to residue 34 in SEQ ID NO: 6; C at a residue corresponding to residue 34 in SEQ ID NO: 6; I at a residue corresponding to residue 34 in SEQ ID NO: 6; V at a residue corresponding to residue 34 in SEQ ID NO: 6; I at a residue corresponding to residue 35 in SEQ ID NO: 6; M at a residue corresponding to residue 35 in SEQ ID NO: 6; E at a residue corresponding to residue 72 in SEQ ID NO: 6; W at a residue corresponding to residue 72 in SEQ ID NO: 6; C at a residue corresponding to residue 72 in SEQ ID NO: 6; R at a residue corresponding to residue 72 in SEQ ID NO: 6; N at a residue corresponding to residue 72 in SEQ ID NO: 6; Q at a residue corresponding to residue 72 in SEQ ID NO: 6; D at a residue corresponding to residue 72 in SEQ ID NO: 6; M at a residue corresponding to residue 72 in SEQ ID NO: 6; F at a residue corresponding to residue 72 in SEQ ID NO: 6; T at a residue corresponding to residue 73 in SEQ ID NO: 6; M at a residue corresponding to residue 73 in SEQ ID NO: 6; N at a residue corresponding to residue 73 in SEQ ID NO: 6; I at a residue corresponding to residue 73 in SEQ ID NO: 6; K at a residue corresponding to residue 73 in SEQ ID NO: 6; C at a residue corresponding to residue 73 in SEQ ID NO: 6; H at a residue corresponding to residue 73 in SEQ ID NO: 6; F at a residue corresponding to residue 74 in SEQ ID NO: 6; I at a residue corresponding to residue 74 in SEQ ID NO: 6; H at a residue corresponding to residue 74 in SEQ ID NO: 6; M at a residue corresponding to residue 74 in SEQ ID NO: 6; W at a residue corresponding to residue 74 in SEQ ID NO: 6; T at a residue corresponding to residue 75 in SEQ ID NO: 6; C at a residue corresponding to residue 75 in SEQ ID NO: 6; E at a residue corresponding to residue 75 in SEQ ID NO: 6; H at a residue corresponding to residue 75 in SEQ ID NO: 6; K at a residue corresponding to residue 75 in SEQ ID NO: 6; F at a residue corresponding to residue 75 in SEQ ID NO: 6; R at a residue corresponding to residue 75 in SEQ ID NO: 6; V at a residue corresponding to residue 95 in SEQ ID NO: 6; L at a residue corresponding to residue 95 in SEQ ID NO: 6; M at a residue corresponding to residue 95 in SEQ ID NO: 6; I at a residue corresponding to residue 96 in SEQ ID NO: 6; T at a residue corresponding to residue 96 in SEQ ID NO: 6; C at a residue corresponding to residue 96 in SEQ ID NO: 6; T at a residue corresponding to residue 98 in SEQ ID NO: 6; I at a residue corresponding to residue 98 in SEQ ID NO: 6; V at a residue corresponding to residue 98 in SEQ ID NO: 6; C at a residue corresponding to residue 98 in SEQ ID NO: 6; M at a residue corresponding to residue 98 in SEQ ID NO: 6; A at a residue corresponding to residue 98 in SEQ ID NO: 6; N at a residue corresponding to residue 99 in SEQ ID NO: 6; S at a residue corresponding to residue 99 in SEQ ID NO: 6; V at a residue corresponding to residue 100 in SEQ ID NO: 6; C at a residue corresponding to residue 100 in SEQ ID NO: 6; N at a residue corresponding to residue 100 in SEQ ID NO: 6; S at a residue corresponding to residue 100 in SEQ ID NO: 6; M at a residue corresponding to residue 103 in SEQ ID NO: 6; C at a residue corresponding to residue 103 in SEQ ID NO: 6; R at a residue corresponding to residue 104 in SEQ ID NO: 6; G at a residue corresponding to residue 104 in SEQ ID NO: 6; N at a residue corresponding to residue 106 in SEQ ID NO: 6; I at a residue corresponding to residue 110 in SEQ ID NO: 6; L at a residue corresponding to residue 127 in SEQ ID NO: 6; Y at a residue corresponding to residue 127 in SEQ ID NO: 6; H at a residue corresponding to residue 127 in SEQ ID NO: 6; H at a residue corresponding to residue 128 in SEQ ID NO: 6; V at a residue corresponding to residue 129 in SEQ ID NO: 6; Y at a residue corresponding to residue 129 in SEQ ID NO: 6; K at a residue corresponding to residue 129 in SEQ ID NO: 6; G at a residue corresponding to residue 129 in SEQ ID NO: 6; M at a residue corresponding to residue 129 in SEQ ID NO: 6; A at a residue corresponding to residue 169 in SEQ ID NO: 6; T at a residue corresponding to residue 169 in SEQ ID NO: 6; K at a residue corresponding to residue 169 in SEQ ID NO: 6; C at a residue corresponding to residue 169 in SEQ ID NO: 6; I at a residue corresponding to residue 199 in SEQ ID NO: 6; V at a residue corresponding to residue 199 in SEQ ID NO: 6; S at a residue corresponding to residue 199 in SEQ ID NO: 6; H at a residue corresponding to residue 199 in SEQ ID NO: 6; W at a residue corresponding to residue 199 in SEQ ID NO: 6; M at a residue corresponding to residue 199 in SEQ ID NO: 6; N at a residue corresponding to residue 199 in SEQ ID NO: 6; Y at a residue corresponding to residue 199 in SEQ ID NO: 6; A at a residue corresponding to residue 199 in SEQ ID NO: 6; Q at a residue corresponding to residue 199 in SEQ ID NO: 6; C at a residue corresponding to residue 200 in SEQ ID NO: 6; V at a residue corresponding to residue 200 in SEQ ID NO: 6; R at a residue corresponding to residue 201 in SEQ ID NO: 6; C at a residue corresponding to residue 201 in SEQ ID NO: 6; P at a residue corresponding to residue 204 in SEQ ID NO: 6; H at a residue corresponding to residue 204 in SEQ ID NO: 6; Y at a residue corresponding to residue 204 in SEQ ID NO: 6; M at a residue corresponding to residue 204 in SEQ ID NO: 6; G at a residue corresponding to residue 204 in SEQ ID NO: 6; N at a residue corresponding to residue 204 in SEQ ID NO: 6; C at a residue corresponding to residue 204 in SEQ ID NO: 6; E at a residue corresponding to residue 204 in SEQ ID NO: 6; A at a residue corresponding to residue 204 in SEQ ID NO: 6; V at a residue corresponding to residue 228 in SEQ ID NO: 6; L at a residue corresponding to residue 228 in SEQ ID NO: 6; M at a residue corresponding to residue 228 in SEQ ID NO: 6; C at a residue corresponding to residue 229 in SEQ ID NO: 6; N at a residue corresponding to residue 229 in SEQ ID NO: 6; T at a residue corresponding to residue 229 in SEQ ID NO: 6; S at a residue corresponding to residue 229 in SEQ ID NO: 6; L at a residue corresponding to residue 231 in SEQ ID NO: 6; I at a residue corresponding to residue 231 in SEQ ID NO: 6; V at a residue corresponding to residue 231 in SEQ ID NO: 6; C at a residue corresponding to residue 231 in SEQ ID NO: 6; E at a residue corresponding to residue 232 in SEQ ID NO: 6; N at a residue corresponding to residue 232 in SEQ ID NO: 6; G at a residue corresponding to residue 232 in SEQ ID NO: 6; I at a residue corresponding to residue 236 in SEQ ID NO: 6; F at a residue corresponding to residue 236 in SEQ ID NO: 6; M at a residue corresponding to residue 236 in SEQ ID NO: 6; W at a residue corresponding to residue 236 in SEQ ID NO: 6; L at a residue corresponding to residue 251 in SEQ ID NO: 6; M at a residue corresponding to residue 251 in SEQ ID NO: 6; N at a residue corresponding to residue 252 in SEQ ID NO: 6; A at a residue corresponding to residue 252 in SEQ ID NO: 6; G at a residue corresponding to residue 252 in SEQ ID NO: 6; Q at a residue corresponding to residue 252 in SEQ ID NO: 6; S at a residue corresponding to residue 252 in SEQ ID NO: 6; N at a residue corresponding to residue 254 in SEQ ID NO: 6; Y at a residue corresponding to residue 255 in SEQ ID NO: 6; W at a residue corresponding to residue 255 in SEQ ID NO: 6; M at a residue corresponding to residue 255 in SEQ ID NO: 6; W at a residue corresponding to residue 258 in SEQ ID NO: 6; F at a residue corresponding to residue 258 in SEQ ID NO: 6; N at a residue corresponding to residue 258 in SEQ ID NO: 6; L at a residue corresponding to residue 258 in SEQ ID NO: 6; V at a residue corresponding to residue 258 in SEQ ID NO: 6; H at a residue corresponding to residue 258 in SEQ ID NO: 6; P at a residue corresponding to residue 258 in SEQ ID NO: 6; K at a residue corresponding to residue 258 in SEQ ID NO: 6; C at a residue corresponding to residue 270 in SEQ ID NO: 6; V at a residue corresponding to residue 270 in SEQ ID NO: 6; H at a residue corresponding to residue 270 in SEQ ID NO: 6; L at a residue corresponding to residue 270 in SEQ ID NO: 6; P at a residue corresponding to residue 270 in SEQ ID NO: 6; A at a residue corresponding to residue 295 in SEQ ID NO: 6; N at a residue corresponding to residue 296 in SEQ ID NO: 6; C at a residue corresponding to residue 296 in SEQ ID NO: 6; T at a residue corresponding to residue 296 in SEQ ID NO: 6; Q at a residue corresponding to residue 296 in SEQ ID NO: 6; M at a residue corresponding to residue 296 in SEQ ID NO: 6; G at a residue corresponding to residue 297 in SEQ ID NO: 6; N at a residue corresponding to residue 297 in SEQ ID NO: 6; S at a residue corresponding to residue 297 in SEQ ID NO: 6; V at a residue corresponding to residue 298 in SEQ ID NO: 6; M at a residue corresponding to residue 298 in SEQ ID NO: 6; M at a residue corresponding to residue 299 in SEQ ID NO: 6; H at a residue corresponding to residue 299 in SEQ ID NO: 6; F at a residue corresponding to residue 299 in SEQ ID NO: 6; G at a residue corresponding to residue 299 in SEQ ID NO: 6; W at a residue corresponding to residue 299 in SEQ ID NO: 6; S at a residue corresponding to residue 299 in SEQ ID NO: 6; T at a residue corresponding to residue 299 in SEQ ID NO: 6; A at a residue corresponding to residue 299 in SEQ ID NO: 6; Q at a residue corresponding to residue 303 in SEQ ID NO: 6; and/or F at a residue corresponding to residue 312 in SEQ ID NO: 6.

In some embodiments, a PTER comprises: G at a residue corresponding to residue 31 in SEQ ID NO: 9; V at a residue corresponding to residue 98 in SEQ ID NO: 9; W at a residue corresponding to residue 128 in SEQ ID NO: 9; E at a residue corresponding to residue 129 in SEQ ID NO: 9; H at a residue corresponding to residue 200 in SEQ ID NO: 9; T at a residue corresponding to residue 201 in SEQ ID NO: 9; G at a residue corresponding to residue 229 in SEQ ID NO: 9; S at a residue corresponding to residue 231 in SEQ ID NO: 9; D at a residue corresponding to residue 233 in SEQ ID NO: 9; G at a residue corresponding to residue 255 in SEQ ID NO: 9; A at a residue corresponding to residue 171 in SEQ ID NO: 9; N at a residue corresponding to residue 173 in SEQ ID NO: 9; I at a residue corresponding to residue 228 in SEQ ID NO: 9; and/or L at a residue corresponding to residue 244 in SEQ ID NO: 9.

In some embodiments, a PTE or PTER has high hydrolase activity on VX and high hydrolase activity on VR. In some embodiments, the PTE or PTER comprises: D at a residue corresponding to residue 100 in SEQ ID NO: 3; L at a residue corresponding to residue 266 in SEQ ID NO: 3; I at a residue corresponding to residue 266 in SEQ ID NO: 3; Q at a residue corresponding to residue 100 in SEQ ID NO: 3; M at a residue corresponding to residue 266 in SEQ ID NO: 1; K at a residue corresponding to residue 236 in SEQ ID NO: 2; W at a residue corresponding to residue 151 in SEQ ID NO: 2; G at a residue corresponding to residue 266 in SEQ ID NO: 3; P at a residue corresponding to residue 70 in SEQ ID NO: 3; T at a residue corresponding to residue 266 in SEQ ID NO: 3; H at a residue corresponding to residue 103 in SEQ ID NO: 2; W at a residue corresponding to residue 272 in SEQ ID NO: 2; A at a residue corresponding to residue 266 in SEQ ID NO: 3; T at a residue corresponding to residue 266 in SEQ ID NO: 1; C at a residue corresponding to residue 266 in SEQ ID NO: 3; G at a residue corresponding to residue 266 in SEQ ID NO: 1; T at a residue corresponding to residue 266 in SEQ ID NO: 4; M at a residue corresponding to residue 263 in SEQ ID NO: 3; and/or M at a residue corresponding to residue 27 in SEQ ID NO: 3.

In some embodiments, a PTE or PTER has high hydrolase activity on VX and hydrolase activity on VR. In some embodiments, the PTE or PTER comprises: S at a residue corresponding to residue 29 in SEQ ID NO: 2; H at a residue corresponding to residue 151 in SEQ ID NO: 2; Y at a residue corresponding to residue 281 in SEQ ID NO: 2; M at a residue corresponding to residue 272 in SEQ ID NO: 2; C at a residue corresponding to residue 99 in SEQ ID NO: 2; H at a residue corresponding to residue 36 in SEQ ID NO: 2; F at a residue corresponding to residue 281 in SEQ ID NO: 2; I at a residue corresponding to residue 259 in SEQ ID NO: 2; W at a residue corresponding to residue 32 in SEQ ID NO: 2; V at a residue corresponding to residue 148 in SEQ ID NO: 4; D at a residue corresponding to residue 283 in SEQ ID NO: 2; H at a residue corresponding to residue 238 in SEQ ID NO: 2; F at a residue corresponding to residue 288 in SEQ ID NO: 2; C at a residue corresponding to residue 49 in SEQ ID NO: 2; N at a residue corresponding to residue 151 in SEQ ID NO: 2; W at a residue corresponding to residue 267 in SEQ ID NO: 3; Q at a residue corresponding to residue 266 in SEQ ID NO: 3; I at a residue corresponding to residue 83 in SEQ ID NO: 2; E at a residue corresponding to residue 291 in SEQ ID NO: 2; E at a residue corresponding to residue 121 in SEQ ID NO: 2; G at a residue corresponding to residue 31 in SEQ ID NO: 2; M at a residue corresponding to residue 147 in SEQ ID NO: 4; L at a residue corresponding to residue 290 in SEQ ID NO: 2; D at a residue corresponding to residue 151 in SEQ ID NO: 2; P at a residue corresponding to residue 30 in SEQ ID NO: 2; R at a residue corresponding to residue 191 in SEQ ID NO: 2; M at a residue corresponding to residue 89 in SEQ ID NO: 2; Y at a residue corresponding to residue 288 in SEQ ID NO: 2; L at a residue corresponding to residue 250 in SEQ ID NO: 2; I at a residue corresponding to residue 260 in SEQ ID NO: 3; F at a residue corresponding to residue 32 in SEQ ID NO: 2; I at a residue corresponding to residue 122 in SEQ ID NO: 2; Q at a residue corresponding to residue 320 in SEQ ID NO: 2; D at a residue corresponding to residue 28 in SEQ ID NO: 2; V at a residue corresponding to residue 251 in SEQ ID NO: 2; N at a residue corresponding to residue 63 in SEQ ID NO: 2; E at a residue corresponding to residue 15 in SEQ ID NO: 2; R at a residue corresponding to residue 245 in SEQ ID NO: 2; N at a residue corresponding to residue 308 in SEQ ID NO: 2; G at a residue corresponding to residue 263 in SEQ ID NO: 3; A at a residue corresponding to residue 13 in SEQ ID NO: 2; D at a residue corresponding to residue 303 in SEQ ID NO: 2; V at a residue corresponding to residue 294 in SEQ ID NO: 2; F at a residue corresponding to residue 40 in SEQ ID NO: 2; D at a residue corresponding to residue 194 in SEQ ID NO: 2; I at a residue corresponding to residue 18 in SEQ ID NO: 2; M at a residue corresponding to residue 47 in SEQ ID NO: 2; D at a residue corresponding to residue 78 in SEQ ID NO: 2; D at a residue corresponding to residue 320 in SEQ ID NO: 2; T at a residue corresponding to residue 263 in SEQ ID NO: 3; Q at a residue corresponding to residue 263 in SEQ ID NO: 3; T at a residue corresponding to residue 311 in SEQ ID NO: 2; H at a residue corresponding to residue 325 in SEQ ID NO: 2; W at a residue corresponding to residue 106 in SEQ ID NO: 2; R at a residue corresponding to residue 309 in SEQ ID NO: 2; M at a residue corresponding to residue 5 in SEQ ID NO: 2; M at a residue corresponding to residue 107 in SEQ ID NO: 2; M at a residue corresponding to residue 49 in SEQ ID NO: 2; S at a residue corresponding to residue 118 in SEQ ID NO: 2; E at a residue corresponding to residue 53 in SEQ ID NO: 2; L at a residue corresponding to residue 318 in SEQ ID NO: 2; H at a residue corresponding to residue 90 in SEQ ID NO: 2; D at a residue corresponding to residue 155 in SEQ ID NO: 2; I at a residue corresponding to residue 127 in SEQ ID NO: 2; L at a residue corresponding to residue 246 in SEQ ID NO: 2; Q at a residue corresponding to residue 304 in SEQ ID NO: 2; M at a residue corresponding to residue 16 in SEQ ID NO: 2; R at a residue corresponding to residue 292 in SEQ ID NO: 2; E at a residue corresponding to residue 78 in SEQ ID NO: 2; V at a residue corresponding to residue 148 in SEQ ID NO: 3; R at a residue corresponding to residue 85 in SEQ ID NO: 2; and/or N at a residue corresponding to residue 317 in SEQ ID NO: 2.

In some embodiments, a PTE or PTER has hydrolase activity on VX and high hydrolase activity on VR. In some embodiments, the PTE or PTER comprises: H at a residue corresponding to residue 284 in SEQ ID NO: 1; F at a residue corresponding to residue 265 in SEQ ID NO: 1; M at a residue corresponding to residue 265 in SEQ ID NO: 3; H at a residue corresponding to residue 284 in SEQ ID NO: 4; Y at a residue corresponding to residue 265 in SEQ ID NO: 4; W at a residue corresponding to residue 265 in SEQ ID NO: 3; Y at a residue corresponding to residue 284 in SEQ ID NO: 4; C at a residue corresponding to residue 284 in SEQ ID NO: 1; N at a residue corresponding to residue 284 in SEQ ID NO: 1; Y at a residue corresponding to residue 284 in SEQ ID NO: 1; W at a residue corresponding to residue 265 in SEQ ID NO: 1; A at a residue corresponding to residue 266 in SEQ ID NO: 1; M at a residue corresponding to residue 284 in SEQ ID NO: 4; W at a residue corresponding to residue 265 in SEQ ID NO: 4; M at a residue corresponding to residue 265 in SEQ ID NO: 1; F at a residue corresponding to residue 284 in SEQ ID NO: 4; G at a residue corresponding to residue 285 in SEQ ID NO: 2; A at a residue corresponding to residue 266 in SEQ ID NO: 4; C at a residue corresponding to residue 70 in SEQ ID NO: 3; W at a residue corresponding to residue 267 in SEQ ID NO: 1; L at a residue corresponding to residue 27 in SEQ ID NO: 3; G at a residue corresponding to residue 283 in SEQ ID NO: 2; N at a residue corresponding to residue 284 in SEQ ID NO: 4; and/or T at a residue corresponding to residue 284 in SEQ ID NO: 3.

In some embodiments, a PTE or PTER has hydrolase activity on VX and VR. In some embodiments, the PTE or PTER comprises: F at a residue corresponding to residue 27 in SEQ ID NO: 3; Q at a residue corresponding to residue 309 in SEQ ID NO: 2; I at a residue corresponding to residue 164 in SEQ ID NO: 3; V at a residue corresponding to residue 139 in SEQ ID NO: 2; R at a residue corresponding to residue 291 in SEQ ID NO: 2; H at a residue corresponding to residue 187 in SEQ ID NO: 2; W at a residue corresponding to residue 325 in SEQ ID NO: 2; E at a residue corresponding to residue 59 in SEQ ID NO: 2; W at a residue corresponding to residue 103 in SEQ ID NO: 2; D at a residue corresponding to residue 284 in SEQ ID NO: 1; I at a residue corresponding to residue 190 in SEQ ID NO: 2; H at a residue corresponding to residue 252 in SEQ ID NO: 2; N at a residue corresponding to residue 292 in SEQ ID NO: 2; W at a residue corresponding to residue 240 in SEQ ID NO: 2; R at a residue corresponding to residue 322 in SEQ ID NO: 2; C at a residue corresponding to residue 266 in SEQ ID NO: 4; Q at a residue corresponding to residue 299 in SEQ ID NO: 2; T at a residue corresponding to residue 265 in SEQ ID NO: 3; H at a residue corresponding to residue 49 in SEQ ID NO: 2; I at a residue corresponding to residue 220 in SEQ ID NO: 2; M at a residue corresponding to residue 296 in SEQ ID NO: 2; M at a residue corresponding to residue 311 in SEQ ID NO: 2; M at a residue corresponding to residue 187 in SEQ ID NO: 2; L at a residue corresponding to residue 190 in SEQ ID NO: 2; D at a residue corresponding to residue 182 in SEQ ID NO: 2; H at a residue corresponding to residue 272 in SEQ ID NO: 2; P at a residue corresponding to residue 70 in SEQ ID NO: 1; I at a residue corresponding to residue 144 in SEQ ID NO: 3; E at a residue corresponding to residue 322 in SEQ ID NO: 2; I at a residue corresponding to residue 247 in SEQ ID NO: 2; V at a residue corresponding to residue 258 in SEQ ID NO: 6; D at a residue corresponding to residue 35 in SEQ ID NO: 2; K at a residue corresponding to residue 279 in SEQ ID NO: 2; L at a residue corresponding to residue 176 in SEQ ID NO: 3; D at a residue corresponding to residue 15 in SEQ ID NO: 2; Q at a residue corresponding to residue 15 in SEQ ID NO: 2; M at a residue corresponding to residue 238 in SEQ ID NO: 2; A at a residue corresponding to residue 25 in SEQ ID NO: 3; W at a residue corresponding to residue 156 in SEQ ID NO: 2; A at a residue corresponding to residue 316 in SEQ ID NO: 2; H at a residue corresponding to residue 202 in SEQ ID NO: 2; L at a residue corresponding to residue 247 in SEQ ID NO: 2; W at a residue corresponding to residue 226 in SEQ ID NO: 2; P at a residue corresponding to residue 39 in SEQ ID NO: 2; A at a residue corresponding to residue 60 in SEQ ID NO: 2; D at a residue corresponding to residue 219 in SEQ ID NO: 2; D at a residue corresponding to residue 245 in SEQ ID NO: 2; H at a residue corresponding to residue 258 in SEQ ID NO: 2; S at a residue corresponding to residue 14 in SEQ ID NO: 2; V at a residue corresponding to residue 32 in SEQ ID NO: 2; K at a residue corresponding to residue 275 in SEQ ID NO: 2; S at a residue corresponding to residue 30 in SEQ ID NO: 2; E at a residue corresponding to residue 44 in SEQ ID NO: 2; N at a residue corresponding to residue 76 in SEQ ID NO: 2; L at a residue corresponding to residue 8 in SEQ ID NO: 2; W at a residue corresponding to residue 267 in SEQ ID NO: 4; K at a residue corresponding to residue 202 in SEQ ID NO: 2; H at a residue corresponding to residue 176 in SEQ ID NO: 3; L at a residue corresponding to residue 265 in SEQ ID NO: 4; K at a residue corresponding to residue 309 in SEQ ID NO: 2; L at a residue corresponding to residue 294 in SEQ ID NO: 2; W at a residue corresponding to residue 26 in SEQ ID NO: 4; E at a residue corresponding to residue 14 in SEQ ID NO: 2; G at a residue corresponding to residue 271 in SEQ ID NO: 2; R at a residue corresponding to residue 86 in SEQ ID NO: 2; D at a residue corresponding to residue 305 in SEQ ID NO: 2; A at a residue corresponding to residue 267 in SEQ ID NO: 4; D at a residue corresponding to residue 43 in SEQ ID NO: 2; K at a residue corresponding to residue 316 in SEQ ID NO: 2; Y at a residue corresponding to residue 103 in SEQ ID NO: 2; F at a residue corresponding to residue 290 in SEQ ID NO: 2; C at a residue corresponding to residue 93 in SEQ ID NO: 2; I at a residue corresponding to residue 147 in SEQ ID NO: 4; G at a residue corresponding to residue 266 in SEQ ID NO: 4; C at a residue corresponding to residue 225 in SEQ ID NO: 2; P at a residue corresponding to residue 324 in SEQ ID NO: 2; E at a residue corresponding to residue 85 in SEQ ID NO: 2; P at a residue corresponding to residue 181 in SEQ ID NO: 1; N at a residue corresponding to residue 201 in SEQ ID NO: 2; M at a residue corresponding to residue 221 in SEQ ID NO: 2; T at a residue corresponding to residue 164 in SEQ ID NO: 4; M at a residue corresponding to residue 263 in SEQ ID NO: 1; P at a residue corresponding to residue 200 in SEQ ID NO: 2; K at a residue corresponding to residue 3 in SEQ ID NO: 2; W at a residue corresponding to residue 30 in SEQ ID NO: 2; L at a residue corresponding to residue 80 in SEQ ID NO: 2; Q at a residue corresponding to residue 121 in SEQ ID NO: 2; M at a residue corresponding to residue 80 in SEQ ID NO: 2; S at a residue corresponding to residue 181 in SEQ ID NO: 3; R at a residue corresponding to residue 308 in SEQ ID NO: 2; F at a residue corresponding to residue 27 in SEQ ID NO: 1; D at a residue corresponding to residue 56 in SEQ ID NO: 2; I at a residue corresponding to residue 51 in SEQ ID NO: 2; S at a residue corresponding to residue 263 in SEQ ID NO: 1; S at a residue corresponding to residue 69 in SEQ ID NO: 3; C at a residue corresponding to residue 70 in SEQ ID NO: 1; S at a residue corresponding to residue 64 in SEQ ID NO: 2; Y at a residue corresponding to residue 255 in SEQ ID NO: 2; F at a residue corresponding to residue 36 in SEQ ID NO: 2; D at a residue corresponding to residue 132 in SEQ ID NO: 2; A at a residue corresponding to residue 300 in SEQ ID NO: 2; M at a residue corresponding to residue 25 in SEQ ID NO: 1; G at a residue corresponding to residue 262 in SEQ ID NO: 3; L at a residue corresponding to residue 193 in SEQ ID NO: 2; M at a residue corresponding to residue 220 in SEQ ID NO: 2; K at a residue corresponding to residue 2 in SEQ ID NO: 2; I at a residue corresponding to residue 4 in SEQ ID NO: 2; V at a residue corresponding to residue 89 in SEQ ID NO: 2; A at a residue corresponding to residue 267 in SEQ ID NO: 1; R at a residue corresponding to residue 81 in SEQ ID NO: 2; D at a residue corresponding to residue 115 in SEQ ID NO: 2; K at a residue corresponding to residue 82 in SEQ ID NO: 2; V at a residue corresponding to residue 80 in SEQ ID NO: 2; Y at a residue corresponding to residue 150 in SEQ ID NO: 2; C at a residue corresponding to residue 203 in SEQ ID NO: 2; Q at a residue corresponding to residue 59 in SEQ ID NO: 2; F at a residue corresponding to residue 325 in SEQ ID NO: 2; G at a residue corresponding to residue 274 in SEQ ID NO: 2; G at a residue corresponding to residue 282 in SEQ ID NO: 2; D at a residue corresponding to residue 291 in SEQ ID NO: 2; K at a residue corresponding to residue 299 in SEQ ID NO: 2; P at a residue corresponding to residue 13 in SEQ ID NO: 2; E at a residue corresponding to residue 135 in SEQ ID NO: 2; F at a residue corresponding to residue 187 in SEQ ID NO: 2; R at a residue corresponding to residue 299 in SEQ ID NO: 2; M at a residue corresponding to residue 18 in SEQ ID NO: 2; V at a residue corresponding to residue 134 in SEQ ID NO: 2; N at a residue corresponding to residue 42 in SEQ ID NO: 2; S at a residue corresponding to residue 263 in SEQ ID NO: 4; M at a residue corresponding to residue 27 in SEQ ID NO: 1; D at a residue corresponding to residue 14 in SEQ ID NO: 2; E at a residue corresponding to residue 124 in SEQ ID NO: 2; P at a residue corresponding to residue 10 in SEQ ID NO: 2; I at a residue corresponding to residue 307 in SEQ ID NO: 2; K at a residue corresponding to residue 317 in SEQ ID NO: 2; Y at a residue corresponding to residue 107 in SEQ ID NO: 2; S at a residue corresponding to residue 150 in SEQ ID NO: 2; T at a residue corresponding to residue 208 in SEQ ID NO: 1; R at a residue corresponding to residue 56 in SEQ ID NO: 2; I at a residue corresponding to residue 148 in SEQ ID NO: 3; T at a residue corresponding to residue 267 in SEQ ID NO: 4; M at a residue corresponding to residue 32 in SEQ ID NO: 2; L at a residue corresponding to residue 289 in SEQ ID NO: 2; I at a residue corresponding to residue 164 in SEQ ID NO: 1; T at a residue corresponding to residue 8 in SEQ ID NO: 2; H at a residue corresponding to residue 60 in SEQ ID NO: 2; D at a residue corresponding to residue 306 in SEQ ID NO: 2; E at a residue corresponding to residue 313 in SEQ ID NO: 2; Q at a residue corresponding to residue 316 in SEQ ID NO: 2; R at a residue corresponding to residue 52 in SEQ ID NO: 2; V at a residue corresponding to residue 4 in SEQ ID NO: 2; S at a residue corresponding to residue 320 in SEQ ID NO: 2; S at a residue corresponding to residue 324 in SEQ ID NO: 2; G at a residue corresponding to residue 136 in SEQ ID NO: 2; D at a residue corresponding to residue 195 in SEQ ID NO: 2; Q at a residue corresponding to residue 34 in SEQ ID NO: 2; S at a residue corresponding to residue 228 in SEQ ID NO: 3; V at a residue corresponding to residue 148 in SEQ ID NO: 1; Q at a residue corresponding to residue 326 in SEQ ID NO: 2; E at a residue corresponding to residue 48 in SEQ ID NO: 2; F at a residue corresponding to residue 311 in SEQ ID NO: 2; L at a residue corresponding to residue 25 in SEQ ID NO: 4; M at a residue corresponding to residue 214 in SEQ ID NO: 2; M at a residue corresponding to residue 286 in SEQ ID NO: 1; E at a residue corresponding to residue 306 in SEQ ID NO: 2; D at a residue corresponding to residue 178 in SEQ ID NO: 4; N at a residue corresponding to residue 253 in SEQ ID NO: 2; I at a residue corresponding to residue 312 in SEQ ID NO: 2; T at a residue corresponding to residue 2 in SEQ ID NO: 2; T at a residue corresponding to residue 3 in SEQ ID NO: 2; E at a residue corresponding to residue 132 in SEQ ID NO: 2; D at a residue corresponding to residue 38 in SEQ ID NO: 2; I at a residue corresponding to residue 92 in SEQ ID NO: 2; T at a residue corresponding to residue 25 in SEQ ID NO: 3; R at a residue corresponding to residue 279 in SEQ ID NO: 2; M at a residue corresponding to residue 20 in SEQ ID NO: 4; Q at a residue corresponding to residue 53 in SEQ ID NO: 2; V at a residue corresponding to residue 220 in SEQ ID NO: 2; D at a residue corresponding to residue 240 in SEQ ID NO: 2; E at a residue corresponding to residue 308 in SEQ ID NO: 2; G at a residue corresponding to residue 88 in SEQ ID NO: 2; W at a residue corresponding to residue 107 in SEQ ID NO: 2; Q at a residue corresponding to residue 3 in SEQ ID NO: 2; S at a residue corresponding to residue 87 in SEQ ID NO: 2; I at a residue corresponding to residue 153 in SEQ ID NO: 2; Q at a residue corresponding to residue 56 in SEQ ID NO: 2; F at a residue corresponding to residue 106 in SEQ ID NO: 2; T at a residue corresponding to residue 118 in SEQ ID NO: 2; Q at a residue corresponding to residue 168 in SEQ ID NO: 2; W at a residue corresponding to residue 49 in SEQ ID NO: 2; E at a residue corresponding to residue 304 in SEQ ID NO: 2; R at a residue corresponding to residue 326 in SEQ ID NO: 2; E at a residue corresponding to residue 249 in SEQ ID NO: 2; V at a residue corresponding to residue 176 in SEQ ID NO: 3; C at a residue corresponding to residue 244 in SEQ ID NO: 2; H at a residue corresponding to residue 106 in SEQ ID NO: 2; V at a residue corresponding to residue 227 in SEQ ID NO: 2; E at a residue corresponding to residue 242 in SEQ ID NO: 2; E at a residue corresponding to residue 245 in SEQ ID NO: 2; L at a residue corresponding to residue 122 in SEQ ID NO: 2; W at a residue corresponding to residue 36 in SEQ ID NO: 2; I at a residue corresponding to residue 293 in SEQ ID NO: 2; M at a residue corresponding to residue 126 in SEQ ID NO: 4; T at a residue corresponding to residue 183 in SEQ ID NO: 2; K at a residue corresponding to residue 322 in SEQ ID NO: 2; V at a residue corresponding to residue 146 in SEQ ID NO: 3; H at a residue corresponding to residue 150 in SEQ ID NO: 2; E at a residue corresponding to residue 12 in SEQ ID NO: 2; I at a residue corresponding to residue 47 in SEQ ID NO: 2; L at a residue corresponding to residue 51 in SEQ ID NO: 2; W at a residue corresponding to residue 249 in SEQ ID NO: 2; T at a residue corresponding to residue 267 in SEQ ID NO: 1; A at a residue corresponding to residue 2 in SEQ ID NO: 2; R at a residue corresponding to residue 258 in SEQ ID NO: 2; V at a residue corresponding to residue 307 in SEQ ID NO: 2; C at a residue corresponding to residue 176 in SEQ ID NO: 3; I at a residue corresponding to residue 144 in SEQ ID NO: 1; I at a residue corresponding to residue 251 in SEQ ID NO: 2; R at a residue corresponding to residue 5 in SEQ ID NO: 2; H at a residue corresponding to residue 279 in SEQ ID NO: 2; M at a residue corresponding to residue 298 in SEQ ID NO: 2; V at a residue corresponding to residue 77 in SEQ ID NO: 2; D at a residue corresponding to residue 120 in SEQ ID NO: 2; R at a residue corresponding to residue 223 in SEQ ID NO: 2; S at a residue corresponding to residue 266 in SEQ ID NO: 4; R at a residue corresponding to residue 166 in SEQ ID NO: 2; S at a residue corresponding to residue 181 in SEQ ID NO: 1; E at a residue corresponding to residue 82 in SEQ ID NO: 2; L at a residue corresponding to residue 83 in SEQ ID NO: 2; A at a residue corresponding to residue 123 in SEQ ID NO: 2; H at a residue corresponding to residue 128 in SEQ ID NO: 2; H at a residue corresponding to residue 236 in SEQ ID NO: 2; M at a residue corresponding to residue 25 in SEQ ID NO: 4; D at a residue corresponding to residue 76 in SEQ ID NO: 2; V at a residue corresponding to residue 91 in SEQ ID NO: 2; K at a residue corresponding to residue 326 in SEQ ID NO: 2; A at a residue corresponding to residue 208 in SEQ ID NO: 3; C at a residue corresponding to residue 265 in SEQ ID NO: 1; T at a residue corresponding to residue 291 in SEQ ID NO: 2; T at a residue corresponding to residue 267 in SEQ ID NO: 3; N at a residue corresponding to residue 128 in SEQ ID NO: 2; N at a residue corresponding to residue 209 in SEQ ID NO: 4; R at a residue corresponding to residue 249 in SEQ ID NO: 2; T at a residue corresponding to residue 70 in SEQ ID NO: 1; E at a residue corresponding to residue 215 in SEQ ID NO: 2; C at a residue corresponding to residue 235 in SEQ ID NO: 2; I at a residue corresponding to residue 203 in SEQ ID NO: 2; C at a residue corresponding to residue 263 in SEQ ID NO: 4; S at a residue corresponding to residue 239 in SEQ ID NO: 2; Y at a residue corresponding to residue 240 in SEQ ID NO: 2; G at a residue corresponding to residue 147 in SEQ ID NO: 4; C at a residue corresponding to residue 8 in SEQ ID NO: 2; D at a residue corresponding to residue 215 in SEQ ID NO: 2; W at a residue corresponding to residue 48 in SEQ ID NO: 2; C at a residue corresponding to residue 165 in SEQ ID NO: 2; W at a residue corresponding to residue 274 in SEQ ID NO: 2; W at a residue corresponding to residue 277 in SEQ ID NO: 2; C at a residue corresponding to residue 263 in SEQ ID NO: 1; I at a residue corresponding to residue 176 in SEQ ID NO: 3; T at a residue corresponding to residue 208 in SEQ ID NO: 3; T at a residue corresponding to residue 263 in SEQ ID NO: 1; F at a residue corresponding to residue 286 in SEQ ID NO: 4; N at a residue corresponding to residue 68 in SEQ ID NO: 3; A at a residue corresponding to residue 305 in SEQ ID NO: 2; E at a residue corresponding to residue 100 in SEQ ID NO: 3; C at a residue corresponding to residue 179 in SEQ ID NO: 1; M at a residue corresponding to residue 246 in SEQ ID NO: 2; P at a residue corresponding to residue 12 in SEQ ID NO: 2; H at a residue corresponding to residue 222 in SEQ ID NO: 2; S at a residue corresponding to residue 147 in SEQ ID NO: 4; S at a residue corresponding to residue 265 in SEQ ID NO: 3; W at a residue corresponding to residue 109 in SEQ ID NO: 2; H at a residue corresponding to residue 140 in SEQ ID NO: 2; M at a residue corresponding to residue 318 in SEQ ID NO: 2; S at a residue corresponding to residue 181 in SEQ ID NO: 4; Q at a residue corresponding to residue 265 in SEQ ID NO: 4; E at a residue corresponding to residue 155 in SEQ ID NO: 2; W at a residue corresponding to residue 236 in SEQ ID NO: 2; T at a residue corresponding to residue 263 in SEQ ID NO: 4; Q at a residue corresponding to residue 5 in SEQ ID NO: 2; N at a residue corresponding to residue 44 in SEQ ID NO: 2; D at a residue corresponding to residue 241 in SEQ ID NO: 2; M at a residue corresponding to residue 110 in SEQ ID NO: 2; W at a residue corresponding to residue 255 in SEQ ID NO: 2; V at a residue corresponding to residue 289 in SEQ ID NO: 2; F at a residue corresponding to residue 318 in SEQ ID NO: 2; R at a residue corresponding to residue 140 in SEQ ID NO: 2; T at a residue corresponding to residue 287 in SEQ ID NO: 2; S at a residue corresponding to residue 168 in SEQ ID NO: 4; K at a residue corresponding to residue 55 in SEQ ID NO: 2; D at a residue corresponding to residue 178 in SEQ ID NO: 3; M at a residue corresponding to residue 27 in SEQ ID NO: 4; C at a residue corresponding to residue 173 in SEQ ID NO: 4; S at a residue corresponding to residue 84 in SEQ ID NO: 2; T at a residue corresponding to residue 265 in SEQ ID NO: 1; S at a residue corresponding to residue 303 in SEQ ID NO: 2; T at a residue corresponding to residue 176 in SEQ ID NO: 4; C at a residue corresponding to residue 221 in SEQ ID NO: 2; C at a residue corresponding to residue 177 in SEQ ID NO: 3; T at a residue corresponding to residue 265 in SEQ ID NO: 4; A at a residue corresponding to residue 208 in SEQ ID NO: 1; I at a residue corresponding to residue 227 in SEQ ID NO: 2; N at a residue corresponding to residue 209 in SEQ ID NO: 3; G at a residue corresponding to residue 147 in SEQ ID NO: 1; T at a residue corresponding to residue 176 in SEQ ID NO: 1; C at a residue corresponding to residue 208 in SEQ ID NO: 4; I at a residue corresponding to residue 260 in SEQ ID NO: 4; W at a residue corresponding to residue 110 in SEQ ID NO: 2; G at a residue corresponding to residue 135 in SEQ ID NO: 2; A at a residue corresponding to residue 221 in SEQ ID NO: 2; R at a residue corresponding to residue 278 in SEQ ID NO: 2; C at a residue corresponding to residue 179 in SEQ ID NO: 3; M at a residue corresponding to residue 176 in SEQ ID NO: 1; S at a residue corresponding to residue 179 in SEQ ID NO: 1; W at a residue corresponding to residue 286 in SEQ ID NO: 1; W at a residue corresponding to residue 188 in SEQ ID NO: 2; F at a residue corresponding to residue 147 in SEQ ID NO: 1; V at a residue corresponding to residue 176 in SEQ ID NO: 1; T at a residue corresponding to residue 208 in SEQ ID NO: 4; S at a residue corresponding to residue 265 in SEQ ID NO: 4; C at a residue corresponding to residue 181 in SEQ ID NO: 1; F at a residue corresponding to residue 109 in SEQ ID NO: 2; M at a residue corresponding to residue 189 in SEQ ID NO: 3; W at a residue corresponding to residue 33 in SEQ ID NO: 2; N at a residue corresponding to residue 68 in SEQ ID NO: 1; C at a residue corresponding to residue 147 in SEQ ID NO: 3; C at a residue corresponding to residue 148 in SEQ ID NO: 1; R at a residue corresponding to residue 298 in SEQ ID NO: 2; H at a residue corresponding to residue 158 in SEQ ID NO: 2; M at a residue corresponding to residue 148 in SEQ ID NO: 4; S at a residue corresponding to residue 266 in SEQ ID NO: 1; H at a residue corresponding to residue 249 in SEQ ID NO: 2; C at a residue corresponding to residue 177 in SEQ ID NO: 1; M at a residue corresponding to residue 286 in SEQ ID NO: 4; D at a residue corresponding to residue 72 in SEQ ID NO: 2; I at a residue corresponding to residue 66 in SEQ ID NO: 4; I at a residue corresponding to residue 164 in SEQ ID NO: 4; M at a residue corresponding to residue 148 in SEQ ID NO: 1; C at a residue corresponding to residue 176 in SEQ ID NO: 1; F at a residue corresponding to residue 27 in SEQ ID NO: 4; P at a residue corresponding to residue 68 in SEQ ID NO: 3; Y at a residue corresponding to residue 176 in SEQ ID NO: 3; S at a residue corresponding to residue 206 in SEQ ID NO: 4; I at a residue corresponding to residue 66 in SEQ ID NO: 1; T at a residue corresponding to residue 27 in SEQ ID NO: 3; V at a residue corresponding to residue 189 in SEQ ID NO: 3; C at a residue corresponding to residue 148 in SEQ ID NO: 4; I at a residue corresponding to residue 148 in SEQ ID NO: 4; S at a residue corresponding to residue 69 in SEQ ID NO: 1; Y at a residue corresponding to residue 176 in SEQ ID NO: 1; Q at a residue corresponding to residue 68 in SEQ ID NO: 3; F at a residue corresponding to residue 176 in SEQ ID NO: 3; E at a residue corresponding to residue 284 in SEQ ID NO: 3; H at a residue corresponding to residue 100 in SEQ ID NO: 3; G at a residue corresponding to residue 267 in SEQ ID NO: 4; N at a residue corresponding to residue 68 in SEQ ID NO: 4; M at a residue corresponding to residue 68 in SEQ ID NO: 4; M at a residue corresponding to residue 68 in SEQ ID NO: 1; W at a residue corresponding to residue 286 in SEQ ID NO: 4; C at a residue corresponding to residue 68 in SEQ ID NO: 4; W at a residue corresponding to residue 105 in SEQ ID NO: 2; H at a residue corresponding to residue 26 in SEQ ID NO: 4; I at a residue corresponding to residue 144 in SEQ ID NO: 4; V at a residue corresponding to residue 146 in SEQ ID NO: 4; M at a residue corresponding to residue 176 in SEQ ID NO: 4; M at a residue corresponding to residue 189 in SEQ ID NO: 4; N at a residue corresponding to residue 263 in SEQ ID NO: 1; C at a residue corresponding to residue 208 in SEQ ID NO: 3; V at a residue corresponding to residue 27 in SEQ ID NO: 4; I at a residue corresponding to residue 176 in SEQ ID NO: 1; G at a residue corresponding to residue 262 in SEQ ID NO: 4; A at a residue corresponding to residue 189 in SEQ ID NO: 3; H at a residue corresponding to residue 176 in SEQ ID NO: 4; A at a residue corresponding to residue 148 in SEQ ID NO: 3; I at a residue corresponding to residue 189 in SEQ ID NO: 3; C at a residue corresponding to residue 68 in SEQ ID NO: 1; S at a residue corresponding to residue 206 in SEQ ID NO: 3; T at a residue corresponding to residue 27 in SEQ ID NO: 4; T at a residue corresponding to residue 27 in SEQ ID NO: 1; N at a residue corresponding to residue 263 in SEQ ID NO: 4; C at a residue corresponding to residue 189 in SEQ ID NO: 3; V at a residue corresponding to residue 204 in SEQ ID NO: 4; I at a residue corresponding to residue 146 in SEQ ID NO: 3; S at a residue corresponding to residue 228 in SEQ ID NO: 1; F at a residue corresponding to residue 57 in SEQ ID NO: 2; W at a residue corresponding to residue 98 in SEQ ID NO: 4; I at a residue corresponding to residue 25 in SEQ ID NO: 3; M at a residue corresponding to residue 68 in SEQ ID NO: 3; V at a residue corresponding to residue 189 in SEQ ID NO: 4; G at a residue corresponding to residue 181 in SEQ ID NO: 4; C at a residue corresponding to residue 189 in SEQ ID NO: 4; D at a residue corresponding to residue 100 in SEQ ID NO: 3; L at a residue corresponding to residue 266 in SEQ ID NO: 3; I at a residue corresponding to residue 266 in SEQ ID NO: 3; Q at a residue corresponding to residue 100 in SEQ ID NO: 3; M at a residue corresponding to residue 266 in SEQ ID NO: 1; K at a residue corresponding to residue 236 in SEQ ID NO: 2; W at a residue corresponding to residue 151 in SEQ ID NO: 2; G at a residue corresponding to residue 266 in SEQ ID NO: 3; P at a residue corresponding to residue 70 in SEQ ID NO: 3; T at a residue corresponding to residue 266 in SEQ ID NO: 3; H at a residue corresponding to residue 103 in SEQ ID NO: 2; W at a residue corresponding to residue 272 in SEQ ID NO: 2; A at a residue corresponding to residue 266 in SEQ ID NO: 3; T at a residue corresponding to residue 266 in SEQ ID NO: 1; C at a residue corresponding to residue 266 in SEQ ID NO: 3; G at a residue corresponding to residue 266 in SEQ ID NO: 1; T at a residue corresponding to residue 266 in SEQ ID NO: 4; M at a residue corresponding to residue 263 in SEQ ID NO: 3; M at a residue corresponding to residue 27 in SEQ ID NO: 3; S at a residue corresponding to residue 29 in SEQ ID NO: 2; H at a residue corresponding to residue 151 in SEQ ID NO: 2; Y at a residue corresponding to residue 281 in SEQ ID NO: 2; M at a residue corresponding to residue 272 in SEQ ID NO: 2; C at a residue corresponding to residue 99 in SEQ ID NO: 2; H at a residue corresponding to residue 36 in SEQ ID NO: 2; F at a residue corresponding to residue 281 in SEQ ID NO: 2; I at a residue corresponding to residue 259 in SEQ ID NO: 2; W at a residue corresponding to residue 32 in SEQ ID NO: 2; V at a residue corresponding to residue 148 in SEQ ID NO: 4; D at a residue corresponding to residue 283 in SEQ ID NO: 2; H at a residue corresponding to residue 238 in SEQ ID NO: 2; F at a residue corresponding to residue 288 in SEQ ID NO: 2; C at a residue corresponding to residue 49 in SEQ ID NO: 2; N at a residue corresponding to residue 151 in SEQ ID NO: 2; W at a residue corresponding to residue 267 in SEQ ID NO: 3; Q at a residue corresponding to residue 266 in SEQ ID NO: 3; I at a residue corresponding to residue 83 in SEQ ID NO: 2; E at a residue corresponding to residue 291 in SEQ ID NO: 2; E at a residue corresponding to residue 121 in SEQ ID NO: 2; G at a residue corresponding to residue 31 in SEQ ID NO: 2; M at a residue corresponding to residue 147 in SEQ ID NO: 4; L at a residue corresponding to residue 290 in SEQ ID NO: 2; D at a residue corresponding to residue 151 in SEQ ID NO: 2; P at a residue corresponding to residue 30 in SEQ ID NO: 2; R at a residue corresponding to residue 191 in SEQ ID NO: 2; M at a residue corresponding to residue 89 in SEQ ID NO: 2; Y at a residue corresponding to residue 288 in SEQ ID NO: 2; L at a residue corresponding to residue 250 in SEQ ID NO: 2; I at a residue corresponding to residue 260 in SEQ ID NO: 3; F at a residue corresponding to residue 32 in SEQ ID NO: 2; I at a residue corresponding to residue 122 in SEQ ID NO: 2; Q at a residue corresponding to residue 320 in SEQ ID NO: 2; D at a residue corresponding to residue 28 in SEQ ID NO: 2; V at a residue corresponding to residue 251 in SEQ ID NO: 2; N at a residue corresponding to residue 63 in SEQ ID NO: 2; E at a residue corresponding to residue 15 in SEQ ID NO: 2; R at a residue corresponding to residue 245 in SEQ ID NO: 2; N at a residue corresponding to residue 308 in SEQ ID NO: 2; G at a residue corresponding to residue 263 in SEQ ID NO: 3; A at a residue corresponding to residue 13 in SEQ ID NO: 2; D at a residue corresponding to residue 303 in SEQ ID NO: 2; V at a residue corresponding to residue 294 in SEQ ID NO: 2; F at a residue corresponding to residue 40 in SEQ ID NO: 2; D at a residue corresponding to residue 194 in SEQ ID NO: 2; I at a residue corresponding to residue 18 in SEQ ID NO: 2; M at a residue corresponding to residue 47 in SEQ ID NO: 2; D at a residue corresponding to residue 78 in SEQ ID NO: 2; D at a residue corresponding to residue 320 in SEQ ID NO: 2; T at a residue corresponding to residue 263 in SEQ ID NO: 3; Q at a residue corresponding to residue 263 in SEQ ID NO: 3; T at a residue corresponding to residue 311 in SEQ ID NO: 2; H at a residue corresponding to residue 325 in SEQ ID NO: 2; W at a residue corresponding to residue 106 in SEQ ID NO: 2; R at a residue corresponding to residue 309 in SEQ ID NO: 2; M at a residue corresponding to residue 5 in SEQ ID NO: 2; M at a residue corresponding to residue 107 in SEQ ID NO: 2; M at a residue corresponding to residue 49 in SEQ ID NO: 2; S at a residue corresponding to residue 118 in SEQ ID NO: 2; E at a residue corresponding to residue 53 in SEQ ID NO: 2; L at a residue corresponding to residue 318 in SEQ ID NO: 2; H at a residue corresponding to residue 90 in SEQ ID NO: 2; D at a residue corresponding to residue 155 in SEQ ID NO: 2; I at a residue corresponding to residue 127 in SEQ ID NO: 2; L at a residue corresponding to residue 246 in SEQ ID NO: 2; Q at a residue corresponding to residue 304 in SEQ ID NO: 2; M at a residue corresponding to residue 16 in SEQ ID NO: 2; R at a residue corresponding to residue 292 in SEQ ID NO: 2; E at a residue corresponding to residue 78 in SEQ ID NO: 2; V at a residue corresponding to residue 148 in SEQ ID NO: 3; R at a residue corresponding to residue 85 in SEQ ID NO: 2; N at a residue corresponding to residue 317 in SEQ ID NO: 2; H at a residue corresponding to residue 284 in SEQ ID NO: 1; F at a residue corresponding to residue 265 in SEQ ID NO: 1; M at a residue corresponding to residue 265 in SEQ ID NO: 3; H at a residue corresponding to residue 284 in SEQ ID NO: 4; Y at a residue corresponding to residue 265 in SEQ ID NO: 4; W at a residue corresponding to residue 265 in SEQ ID NO: 3; Y at a residue corresponding to residue 284 in SEQ ID NO: 4; C at a residue corresponding to residue 284 in SEQ ID NO: 1; N at a residue corresponding to residue 284 in SEQ ID NO: 1; Y at a residue corresponding to residue 284 in SEQ ID NO: 1; W at a residue corresponding to residue 265 in SEQ ID NO: 1; A at a residue corresponding to residue 266 in SEQ ID NO: 1; M at a residue corresponding to residue 284 in SEQ ID NO: 4; W at a residue corresponding to residue 265 in SEQ ID NO: 4; M at a residue corresponding to residue 265 in SEQ ID NO: 1; F at a residue corresponding to residue 284 in SEQ ID NO: 4; G at a residue corresponding to residue 285 in SEQ ID NO: 2; A at a residue corresponding to residue 266 in SEQ ID NO: 4; C at a residue corresponding to residue 70 in SEQ ID NO: 3; W at a residue corresponding to residue 267 in SEQ ID NO: 1; L at a residue corresponding to residue 27 in SEQ ID NO: 3; G at a residue corresponding to residue 283 in SEQ ID NO: 2; N at a residue corresponding to residue 284 in SEQ ID NO: 4; and/or T at a residue corresponding to residue 284 in SEQ ID NO: 3.

In some embodiments, a PTE or PTER has hydrolase activity on VX. In some embodiments, the PTE or PTER comprises: W at a residue corresponding to residue 266 in SEQ ID NO: 3; Y at a residue corresponding to residue 238 in SEQ ID NO: 2; W at a residue corresponding to residue 266 in SEQ ID NO: 1; N at a residue corresponding to residue 209 in SEQ ID NO: 1; P at a residue corresponding to residue 77 in SEQ ID NO: 2; H at a residue corresponding to residue 38 in SEQ ID NO: 2; C at a residue corresponding to residue 188 in SEQ ID NO: 2; C at a residue corresponding to residue 27 in SEQ ID NO: 4; C at a residue corresponding to residue 27 in SEQ ID NO: 1; C at a residue corresponding to residue 126 in SEQ ID NO: 4; Y at a residue corresponding to residue 27 in SEQ ID NO: 3; V at a residue corresponding to residue 73 in SEQ ID NO: 3; C at a residue corresponding to residue 27 in SEQ ID NO: 3; Y at a residue corresponding to residue 176 in SEQ ID NO: 4; F at a residue corresponding to residue 176 in SEQ ID NO: 1; E at a residue corresponding to residue 179 in SEQ ID NO: 1; W at a residue corresponding to residue 153 in SEQ ID NO: 2; W at a residue corresponding to residue 276 in SEQ ID NO: 2; C at a residue corresponding to residue 25 in SEQ ID NO: 3; W at a residue corresponding to residue 27 in SEQ ID NO: 3; V at a residue corresponding to residue 27 in SEQ ID NO: 3; L at a residue corresponding to residue 189 in SEQ ID NO: 4; C at a residue corresponding to residue 231 in SEQ ID NO: 6; S at a residue corresponding to residue 69 in SEQ ID NO: 4; R at a residue corresponding to residue 296 in SEQ ID NO: 2; H at a residue corresponding to residue 266 in SEQ ID NO: 3; C at a residue corresponding to residue 177 in SEQ ID NO: 4; Q at a residue corresponding to residue 27 in SEQ ID NO: 1; I at a residue corresponding to residue 176 in SEQ ID NO: 4; I at a residue corresponding to residue 189 in SEQ ID NO: 4; A at a residue corresponding to residue 189 in SEQ ID NO: 4; A at a residue corresponding to residue 208 in SEQ ID NO: 4; F at a residue corresponding to residue 176 in SEQ ID NO: 4; A at a residue corresponding to residue 68 in SEQ ID NO: 3; Y at a residue corresponding to residue 27 in SEQ ID NO: 1; A at a residue corresponding to residue 68 in SEQ ID NO: 1; Y at a residue corresponding to residue 178 in SEQ ID NO: 4; H at a residue corresponding to residue 128 in SEQ ID NO: 6; and/or L at a residue corresponding to residue 177 in SEQ ID NO: 1.

In some embodiments, a PTE or PTER has hydrolase activity on VR. In some embodiments, the PTE or PTER comprises: M at a residue corresponding to residue 265 in SEQ ID NO: 4; Q at a residue corresponding to residue 284 in SEQ ID NO: 1; C at a residue corresponding to residue 284 in SEQ ID NO: 4; Q at a residue corresponding to residue 284 in SEQ ID NO: 4; L at a residue corresponding to residue 265 in SEQ ID NO: 1; K at a residue corresponding to residue 284 in SEQ ID NO: 1; K at a residue corresponding to residue 284 in SEQ ID NO: 3; C at a residue corresponding to residue 265 in SEQ ID NO: 4; N at a residue corresponding to residue 206 in SEQ ID NO: 3; F at a residue corresponding to residue 263 in SEQ ID NO: 3; I at a residue corresponding to residue 25 in SEQ ID NO: 1; S at a residue corresponding to residue 228 in SEQ ID NO: 4; N at a residue corresponding to residue 229 in SEQ ID NO: 6; R at a residue corresponding to residue 284 in SEQ ID NO: 4; W at a residue corresponding to residue 27 in SEQ ID NO: 1; Q at a residue corresponding to residue 303 in SEQ ID NO: 6; I at a residue corresponding to residue 265 in SEQ ID NO: 4; C at a residue corresponding to residue 208 in SEQ ID NO: 1; A at a residue corresponding to residue 228 in SEQ ID NO: 1; L at a residue corresponding to residue 189 in SEQ ID NO: 3; R at a residue corresponding to residue 284 in SEQ ID NO: 3; I at a residue corresponding to residue 284 in SEQ ID NO: 1; R at a residue corresponding to residue 284 in SEQ ID NO: 1; C at a residue corresponding to residue 210 in SEQ ID NO: 4; V at a residue corresponding to residue 265 in SEQ ID NO: 4; M at a residue corresponding to residue 204 in SEQ ID NO: 6; R at a residue corresponding to residue 145 in SEQ ID NO: 1; H at a residue corresponding to residue 176 in SEQ ID NO: 1; F at a residue corresponding to residue 263 in SEQ ID NO: 1; D at a residue corresponding to residue 23 in SEQ ID NO: 4; F at a residue corresponding to residue 98 in SEQ ID NO: 3; M at a residue corresponding to residue 26 in SEQ ID NO: 4; and/or V at a residue corresponding to residue 178 in SEQ ID NO: 4.

In some embodiments, a PTE or PTER has hydrolase activity on GB and GD. In some embodiments, the PTE or PTER comprises: Y at a residue corresponding to residue 265 in SEQ ID NO: 1; M at a residue corresponding to residue 266 in SEQ ID NO: 1; G at a residue corresponding to residue 266 in SEQ ID NO: 1; W at a residue corresponding to residue 267 in SEQ ID NO: 1; H at a residue corresponding to residue 284 in SEQ ID NO: 1; S at a residue corresponding to residue 29 in SEQ ID NO: 2; C at a residue corresponding to residue 99 in SEQ ID NO: 2; H at a residue corresponding to residue 103 in SEQ ID NO: 2; W at a residue corresponding to residue 151 in SEQ ID NO: 2; K at a residue corresponding to residue 236 in SEQ ID NO: 2; C at a residue corresponding to residue 272 in SEQ ID NO: 2; W at a residue corresponding to residue 272 in SEQ ID NO: 2; Y at a residue corresponding to residue 281 in SEQ ID NO: 2; G at a residue corresponding to residue 285 in SEQ ID NO: 2; D at a residue corresponding to residue 100 in SEQ ID NO: 3; Y at a residue corresponding to residue 265 in SEQ ID NO: 3; I at a residue corresponding to residue 266 in SEQ ID NO: 3; L at a residue corresponding to residue 266 in SEQ ID NO: 3; H at a residue corresponding to residue 284 in SEQ ID NO: 3; V at a residue corresponding to residue 148 in SEQ ID NO: 4; M at a residue corresponding to residue 265 in SEQ ID NO: 4; Y at a residue corresponding to residue 265 in SEQ ID NO: 4; T at a residue corresponding to residue 266 in SEQ ID NO: 4; W at a residue corresponding to residue 267 in SEQ ID NO: 4; H at a residue corresponding to residue 284 in SEQ ID NO: 4; K at a residue corresponding to residue 75 in SEQ ID NO: 6; H at a residue corresponding to residue 128 in SEQ ID NO: 6; N at a residue corresponding to residue 229 in SEQ ID NO: 6; C at a residue corresponding to residue 231 in SEQ ID NO: 6; and/or Q at a residue corresponding to residue 303 in SEQ ID NO: 6.

In some embodiments, a PTE or PTER has hydrolase activity on VR, VX, GB, and GD. In some embodiments, the PTE or PTER comprises: L at a residue corresponding to residue 266 in SEQ ID NO: 3; and/or M at a residue corresponding to residue 266 in SEQ ID NO: 1.

In some embodiments, a PTE or PTER has hydrolase activity on VR and GD. In some embodiments, the PTE or PTER comprises: Y at a residue corresponding to residue 265 in SEQ ID NO: 1.

In some embodiments, a PTE or PTER has hydrolase activity on VR, GB, and GD. In some embodiments, the PTE or PTER comprises: H at a residue corresponding to residue 284 in SEQ ID NO: 1.

In some embodiments, a PTE or PTER comprises one or more of the amino acid substitutions discussed in Examples 2 and 5-6 and provided in Tables 7 and 9-10. In some embodiments, a PTE or PTER contains one or more amino acid substitutions in one or more residues within the active site. The active site of a PTE or a PTER may be identified by generating the three-dimensional structure of the PTE or PTER and identifying the residues within a particular distance of the catalytic center and/or within a particular distance of a docked substrate within the PTE or PTER. As used herein, a residue is within the active site of a PTE or PTER if it is within about 12 angstroms of the catalytic center of the PTE or PTER and/or within about 12 angstroms of a docked substrate within the PTE or PTER.

In some embodiments, a PTE or PTER contains one or more amino acid substitutions in one or more residues within the first shell. As used herein, a residue is within the first shell if it has at least one non-hydrogen atom within about 6 angstroms of the catalytic center of the PTE or PTER and/or has at least one non-hydrogen atom within about 6 angstroms of a docked substrate within the PTE or PTER.

In some embodiments, a PTE or PTER contains one or more amino acid substitutions in one or more residues within the second shell. As used herein, a residue is within the second shell if it has at least one non-hydrogen atom within about 8 angstroms of the catalytic center of the PTE or PTER and/or has at least one non-hydrogen atom within about 8 angstroms of a docked substrate within the PTE or PTER, but is not within the first shell.

In some embodiments, a PTE or PTER contains one or more amino acid substitutions in one or more residues within the third shell. As used herein, a residue is within the third shell if it has at least one non-hydrogen atom within about 10 angstroms of the catalytic center of the PTE or PTER and/or has at least one non-hydrogen atom within about 10 angstroms of a docked substrate within the PTE or PTER, but is not within the first or second shells.

In some embodiments, a PTE or PTER contains one or more amino acid substitutions in one or more residues within the fourth shell. As used herein, a residue is within the fourth shell if it has at least one non-hydrogen atom within about 12 angstroms of the catalytic center of the PTE or PTER and/or has at least one non-hydrogen atom within about 12 angstroms of a docked substrate within the PTE or PTER, but is not within the first, second, or third shells.

Without wishing to be bound by any theory, the first four shells of residues (shells 1-4) may be considered “around the active site.” In some embodiments, shells around the active site may be mutated to increase protein stability and/or protein activity.

In some embodiments, a PTE or PTER contains one or more mutations, for example, one or more substitutions, insertions and/or deletions in one or more distal residues. As used herein, a residue is a distal residue if it is not within shells 1-4. Without wishing to be bound by any theory, distal residues may be considered “outside of the active site.” In some embodiments, mutation of distal residues can increase protein stability.

In some embodiments, a PTE or a PTER comprises one or more mutations in one or more residues within the active site of the PTE or PTER. In some embodiments, the PTE or PTER comprises one or more mutations in one or more residues within the active site relative to a PTE provided by SEQ ID NO: 1. In some embodiments, the PTE comprises one or more mutations in one or more residues within the active site relative to a PTE provided by SEQ ID NO: 2. In some embodiments, the PTE comprises one or more mutations in one or more residues within the active site relative to a PTE provided by SEQ ID NO: 3. In some embodiments, the PTE comprises one or more mutations in one or more residues within the active site relative to a PTE provided by SEQ ID NO: 4. In some embodiments, the PTE comprises no more than 2 amino acid substitutions, insertions, additions or deletions relative to the sequence of any one of SEQ ID NOs: 1-4. In some embodiments, the PTER comprises one or more mutations in one or more residues within the active site relative to the PTER provided by SEQ ID NO: 6. In some embodiments, the PTER comprises no more than 2 amino acid substitutions, insertions, additions or deletions relative to the sequence of SEQ ID NO: 6.

In some embodiments, a PTE comprises an amino acid substitution relative to the sequence of SEQ ID NO: 1 at position Y98 within SEQ ID NO: 1. In some embodiments, the PTE comprises a Y98W substitution relative to the sequence of SEQ ID NO: 1. In some embodiments, a PTE comprises an amino acid substitution relative to the sequence of SEQ ID NO: 2 at position V244 within SEQ ID NO: 2. In some embodiments, the PTE comprises a V244C substitution relative to the sequence of SEQ ID NO: 2. In some embodiments, a PTE comprises an amino acid substitution relative to the sequence of SEQ ID NO: 4 at position N266 within SEQ ID NO: 4. In some embodiments, the PTE comprises a N266C substitution relative to the sequence of SEQ ID NO: 4.

In some embodiments, a PTE or PTER is tagged. For example, in some embodiments, a PTE or PTER is tagged with a StrepII tag at the C-terminus. In other embodiments, a PTE or PTER is tagged with a StrepII tag at the N-terminus. The Strep-tag® system is a method which allows the purification and detection of proteins by affinity chromatography. The Strep-tag II is a synthetic peptide consisting of eight amino acids (Trp-Ser-His-Pro-Gln-Phe-Glu-Lys). In some embodiments, a PTE or PTER can be tagged by using any system that is suitable for the purification and/or production processes of PTEs and/or PTERs.

In some embodiments, a PTE or PTER has hydrolase activity on an OPNA (e.g., on a V-agent and/or a G-agent) that is at least 5%, at least 10%, at least 15%, least 20%, at least 25%, at least 30%, least 35%, at least 40%, at least 45%, least 50%, at least 55%, at least 60%, at least 65%, at least 70%, least 75%, at least 80%, at least 85%, at least 90%, at least 95%, at least 100%, at least 200%, at least 300%, at least 400%, or at least 500% of the hydrolase activity of a reference PTE or PTER. In some embodiments, a PTE has hydrolase activity on VR that is at least 10% of the hydrolase activity of a reference PTE or PTER. In some embodiments, a PTE has hydrolase activity on VX that is at least 35% of the hydrolase activity of a reference PTE or PTER. In some embodiments, a reference PTE or PTER is any one of SEQ ID NOs: 1-8.

In some embodiments, a PTE or PTER that has at least one amino acid substitution relative to a reference PTE or PTER has hydrolase activity on an OPNA (e.g., on a V-agent and/or a G-agent) that is at least 5%, at least 10%, at least 15%, least 20%, at least 25%, at least 30%, least 35%, at least 40%, at least 45%, least 50%, at least 55%, at least 60%, at least 65%, at least 70%, least 75%, at least 80%, at least 85%, at least 90%, at least 95%, at least 100%, at least 200%, at least 300%, at least 400%, or at least 500% of the hydrolase activity of the reference PTE or PTER. In some embodiments, a reference PTE or PTER is any one of SEQ ID NOs: 1-8.

In some embodiments, a PTE or PTER is capable of eliciting lower immunogenicity when administered to a subject compared to a reference PTE or PTER. In some embodiments, the PTE or PTER is capable of eliciting lower immunogenicity by at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, compared with a reference PTE or PTER. In some embodiments, a reference PTE or PTER is any one of SEQ ID NOs: 1-8.

Without wishing to be bound by any theory, lower immunogenicity may indicate that a PTE or PTER elicits a lower or weaker neutralizing antibody response and/or a lower or weaker anaphylaxis-provoking response than a reference PTE or PTER. In some embodiments, the lower immunogenicity occurs upon repeated dosing of a PTE or PTER. In some embodiments, a reference PTE or PTER is any one of SEQ ID NOs: 1-8.

In some embodiments, immunogenicity can be determined by prediction of MHC-II binding using a publicly available algorithm. In some embodiments, an orthogonal sequence-based approach can be used for predicting immunogenicity based on comparison to human proteins.

Catalytic efficiency of enzymes associated with the disclosure for the hydrolysis of V-agents and/or G-agents can be shown as a K_{cat}/K_{M} value or ratio, or as a specificity constant. One of ordinary skill in the art would understand that a higher K_{cat}/K_{M} value generally corresponds to higher and better catalytic efficiency of an enzyme. A Kcaₜ/K_{M} value or ratio can be calculated by determining the ratio of K_{obs}, the first-order degradation constant, and [E], the enzyme concentration. Calculation of K_{cat}/K_{M} values is further discussed in Dawson et al. (Degradation of nerve agents by an organophosphate-degrading agent (OpdA); J Hazard Mater, (2008), 157(2-3):308-314), which is incorporated by reference in its entirety.

In some embodiments, a PTE or PTER has a K_{cat}/K_{M} value greater than 10 M⁻¹ min⁻¹, greater than 10² M⁻¹ min⁻¹, greater than 10³ M⁻¹ min⁻¹, greater than 10⁴ M⁻¹ min⁻¹, greater than 10⁵ M⁻¹ min⁻¹, greater than 10⁶ M⁻¹ min⁻¹, greater than 10⁷ M⁻¹ min⁻¹, greater than 10⁸ M⁻¹ min⁻¹, greater than 10⁹ M⁻¹ min⁻¹, or greater than 10¹⁰ M⁻¹ min⁻¹, including all values in between. In some embodiments, a PTE or PTER has a K_{cat}/K_{M} value greater than 10⁷ M⁻¹ min⁻¹. In some embodiments, a PTE or PTER having a Kcaₜ/K_{M} value greater than 10⁷ M⁻¹ min⁻¹ may efficiently catalyze nerve agents such as VX, VR, GB, and/or GD.

### Methods of Use of OPNA Hydrolyzing Enzymes

Aspects of the present disclosure relate to administering a PTE or PTER to hydrolyze or degrade an OPNA such as a V-agent or a G-agent. For example, enzymes that regulate hydrolysis of nerve agents described in this disclosure can be used for degrading OPNAs, such as V-agents and/or G-agents, and therefore relieving or reducing the toxicity to a subject caused by such OPNAs. In some embodiments, enzymes that hydrolyze nerve agents described in this disclosure can relieve or reduce debilitating symptoms caused by nerve agents as described in this disclosure.

Enzymes that regulate hydrolysis of nerve agents described in this disclosure include but are not limited to PTEs and PTERs. In some embodiments, enzymes that regulate hydrolysis of the nerve agents described in this disclosure are PTEs. In some embodiments, enzymes that regulate hydrolysis of nerve agents described in this disclosure are PTERs. In some embodiments, enzymes that regulate hydrolysis of nerve agents described in this disclosure can comprise any combination of OPNA hydrolyzing enzymes that is able to relieve or reduce the toxicity to a subject caused by an OPNA, such as a V-agent and/or a G-agent.

The present disclosure provides that a PTE or PTER can be used to hydrolyze or degrade an OPNA, such as a V-agent and/or a G-agent, or to treat, protect against, prevent, relieve, or reduce the toxicity to a subject caused by such OPNAs, such as V-agents and/or a G-agent. In some embodiments, treating protecting against, preventing, relieving, or reducing the toxicity involves hydrolyzing and/or neutralizing the nerve agents and/or stopping the spread of the nerve agents in a subject.

In some embodiments, the present disclosure provides a method of hydrolyzing or degrading VX. In some embodiments, the present disclosure provides a method of hydrolyzing or degrading VR. In some embodiments, the present disclosure provides a method of hydrolyzing or degrading GB. In some embodiments, the present disclosure provides a method of hydrolyzing or degrading GD. In some embodiments, methods include administering a PTE or PTER that comprises the sequence of any one of SEQ ID NOs: 1-4 or 6. In some embodiments, the PTE or PTER comprises a sequence that is at least 90% identical to any one of SEQ ID NOs: 1-4 and 6. In some embodiments, the present disclosure provides a method of hydrolyzing or degrading an OPNA, such as a V-agent and/or a G-agent, by administering a PTE or PTER that is capable of eliciting lower immunogenicity when administered to a subject compared to the immunogenicity elicited by an enzyme comprising the sequence of SEQ ID NO: 5.

Subjects associated with the disclosure include human and non-human subjects. In some embodiments, a non-human subject is a non-human primate. In some embodiments, a non-human subject is a companion animal or a farm animal. In some embodiments, the subject is a human subject. In some embodiments, the subject is a human subject having, suspected of having, or at risk of developing toxicity caused by a nerve agent such as VR, VX, GB, and/or GD. In some embodiments, the subject is affected by, suspected of being affected by, or at risk of being affected by toxicity caused by a nerve agent such as VR, VX, GB, and/or GD. In some embodiments, the subject is developing, will be developing, or has developed toxicity caused by a nerve agent such as VR, VX, GB, and/or GD.

Such a subject can be identified by routine examination, e.g., visual inspection, routine laboratory tests, physical exams, as would be understood by one of ordinary skill in the art. Cholinesterase levels can help establish a diagnosis and be an accurate predictor of prognosis. Without wishing to be bound by any theory, assays of red blood cell (RBC) AChE may provide information about the degree of toxicity and may inform whether subsequent dosing of oximes may be required. Follow up measurements of RBC AChE may demonstrate the reactivation of the enzyme over time and the effectiveness of treatment. If clinically suspected, a trial of atropine 1 mg in adults (0.01 mg/kg in children) can be used to assess for clinical improvement.

Such a subject may exhibit one or more symptoms or signs of toxicity such as weak muscle and urination. In some embodiments, such a subject may have one or more of risk factors associated with the development of the toxicity reactions including, in some embodiments, mutations or polymorphisms in one or more OPNA hydrolyzing enzymes in the subject.

In some embodiments, a PTE or PTER associated with the disclosure may be administered to a subject before the subject comes into contact with a V-agent and/or a G-agent. In such embodiments, the PTE or PTER may protect against or prevent harmful effects of the V-agent and/or G-agent on the subject.

In some embodiments, the PTE or PTER is expressed by bacteria to hydrolyze or degrade a V-agent and/or a G-agent, or to protect against, prevent, relieve, or reduce the toxicity to a subject caused by such V-agents and/or G-agents. In some embodiments, the bacteria (e.g., mutualistic or commensal flora) that express PTE or PTER are located on the skin of a subject. In some embodiments, the bacteria that express PTE or PTER are located in the gastrointestinal tract of a subject. In some embodiments, the bacteria that express PTE or PTER are located in the lungs of a subject. In some embodiments, the bacteria that express PTE or PTER are located in or on the eyes of a subject. In some embodiments, bacteria that express a PTE or PTER are administered to a subject.

Any of the OPNA hydrolyzing enzymes associated with the disclosure, including OPNA hydrolyzing enzymes expressed in or on a suitable cell type, or polynucleotides encoding OPNA hydrolyzing enzymes, can be administered to a human or an animal in need of a therapeutically effective amount of said enzyme for the purpose of treating, or providing pre- or post-exposure prophylaxis for, exposure of said human or animal to an OPNA, such as a V-agent and/or a G-agent.

In some embodiments, a cell that expresses an OPNA hydrolyzing enzyme is administered to a subject. The enzyme-expressing cell may be administered by any of a number of routes including, but not limited to, intraarterial, intravenous, intrathecal, intraperitoneal, intramuscular, intradermal, subdermal, cutaneous, transdermal, inhalation, intraocular, sublingual, buccal, otic, vaginal, rectal, nasal, or oral administration. The cell type used may depend on the route of administration; by way of non-limiting examples, human cells may be preferred for intravenous administration, whereas bacterial or yeast or fungal cells may be preferred for oral administration.

The administered enzyme-expressing cell may be contained, if necessary, in a suitable dosing formulation, well-known in the art to vary depending upon the chosen route of administration.

Any of the OPNA hydrolyzing enzymes associated with the disclosure, which optionally may be incorporated into carriers such as liposomes, or optionally may be modified by polymers such as polyethylene glycol or other suitable polymers, may be administered to a human or an animal in need of a therapeutically effective amount of said enzyme for the purpose of treating, or providing pre- or post-exposure prophylaxis for, exposure of said human or animal to an OPNA such as a V-agent and/or a G-agent.

In other embodiments, an OPNA hydrolyzing enzyme is administered to a subject. The enzyme may be administered by any of a number of routes including, but not limited to, intraarterial, intravenous, intrathecal, intraperitoneal, intramuscular, intradermal, subdermal, cutaneous, transdermal, inhalation, intraocular, sublingual, buccal, otic, vaginal, rectal, nasal, or oral administration.

The administered enzyme may be contained, if necessary, in a suitable dosing formulation, well-known in the art to vary depending upon the chosen route of administration.

In other embodiments, a polynucleotide that encodes an OPNA hydrolyzing enzyme is administered to a subject. The enzyme-encoding polynucleotide may be administered by any of a number of routes including, but not limited to, intraarterial, intravenous, intrathecal, intraperitoneal, intramuscular, intradermal, subdermal, cutaneous, transdermal, inhalation, intraocular, sublingual, buccal, otic, vaginal, rectal, nasal, or oral administration. In some embodiments, the polynucleotide is an RNA. In other embodiments, the polynucleotide is a DNA. The polynucleotide type used may depend on the route of administration; by way of non-limiting examples, RNA may be preferred for intramuscular administration, whereas DNA may be preferred for nasal administration.

The administered enzyme-encoding polynucleotide may be contained, if necessary, in a suitable dosing formulation, well-known in the art to vary depending upon the chosen route of administration; by way of non-limiting example, the formulation may comprise liposomes encapsulating the polynucleotide.

In some embodiments, the PTE or PTER is applied to or embedded or incorporated in a material such as a synthetic textile-like film that can be used to construct a protective suit. In some embodiments, the PTE or PTER is incorporated into materials such as textile-like films formed by nanoporous polymer membranes such as poly(isoprene-b-styrene-b-4-vinylpyridine) membranes and macroporous nylon supports. The PTE or PTER applied to or embedded or incorporated in the protective suit can hydrolyze or degrade a V-agent and/or a G-agent to protect against, prevent, relieve, or reduce the toxicity to a subject caused by such V-agents and/or G-agents. In some embodiments, the protective suit can be worn by a subject in need of protection against such V-agents and/or G-agents. In some embodiments, the protective suit is a uniform (e.g., an army uniform). In some embodiments, the PTE or PTER is in the form of a dried enzyme. In some embodiments, the PTE or PTER is dissolved in a liquid, such as a layer of liquid.

Any of the OPNA hydrolyzing enzymes associated with the disclosure, which optionally may be incorporated into carriers such as liposomes or living or dead cells, or optionally may be modified by polymers such as polyethylene glycol or other suitable polymers, may be impregnated in, or attached to using a suitable linking method, the fibers or fabric or surface of clothing or a wearable suit, for the purpose of degrading or destroying some or all of any OPNA, such as a V-agent and/or a G-agent, that contacts said clothing or suit.

An impregnated enzyme, optionally incorporated into carriers, may fill voids in the fibers, fabric, or surface, and may be retained by non-covalent molecular (van der Waals) forces and/or electrostatic forces. An attached enzyme, optionally incorporated into carriers, may be covalently linked to the fibers, fabric, or surface, for example by using ultraviolet (UV) light crosslinking, other similar non-specific crosslinking methods, or by using any of a wide variety of functional chemistry-specific or -general crosslinking agents, for example as described in ThermoFisher “Chemistry of Crosslinking” (https://www.thermofisher.com/us/en/home/life-science/protein-biology/protein-biology-leaming-center/protein-biology-resource-library/pierce-protein-methods/chemistry-crosslinking.html; accessed Mar. 22, 2021).

Examples of crosslinking agents include, but are not limited to, carboxyl-to-amine reactive groups such as carbodiimides; amine-reactive groups such as N-hydroxy-succinimide esters, imidoesters, pentafluorophenyl esters, and hydroxymethyl phosphines; sulfhydryl-reactive groups such as maleimides, haloacetyls (typically bromo- or iodo-), pyridyldisulfides, thiosulfonate, and vinylsulfones; and photoreactive groups, such as diazirines and aryl azides, and hydroxyl-reactive groups such as isocyanates.

Any of the OPNA hydrolyzing enzymes associated with the disclosure, which optionally may be incorporated into carriers such as liposomes or living or dead cells, or optionally may be modified by polymers such as polyethylene glycol or other suitable polymers, may be used to decontaminate solid or liquid or gaseous items contaminated by OPNAs, such as V-agents and/or G-agents, by contacting the contaminated item with the enzyme.

Contacting may be done in various ways including, but not limited to, for example, by passing gaseous or liquid items through or over a solution of enzyme (optionally in or on a carrier), or packed or fluidized beds of enzyme (optionally in or on a carrier) adsorbed or attached to or immobilized on a suitable packing medium (e.g. [suitably functionalized] agarose beads, polymeric beads, or silica particles) by spraying or otherwise depositing (e.g. mixing) dissolved, dried (freeze-dried or spray-dried), isolated, or immobilized enzyme (optionally in a carrier) onto or into solid, liquid, or gaseous items, including onto the surfaces of solid or liquid items.

Items to be decontaminated may include, but are not limited to, water and water supplies, air, soil, plants, permanent or temporary buildings, housing or other living spaces, offices or other work spaces, furniture, equipment, vehicles, clothing, foodstuffs, animals, humans, or animal or human skin.

In some embodiments, the PTE or PTER functions as a bioscavenger.

In some embodiments, methods described in this disclosure involve hydrolyzing a V-agent and/or a G-agent by at least 5%, 10%, 20%, 30%, 40%, 50%, 60%, 70%, 80%, 90%, or 100% more than that of a control, or by at least 2-fold, at least 5-fold, at least 10-fold, at least 20-fold, at least 50-fold, at least 100-fold, or at least 1000-fold compared to that of a control. In some embodiments, methods described in this disclosure comprise degrading a V-agent and/or a G-agent by at least 5%, 10%, 20%, 30%, 40%, 50%, 60%, 70%, 80%, 90%, 100%, more than that of a control, or by at least 2-fold, at least 5-fold, at least 10-fold, at least 20-fold, at least 50-fold, at least 100-fold, or at least 1000-fold compared to that of a control. In some embodiments, a control is a PTE or PTER comprising any one of SEQ ID NOs: 1-8.

In some embodiments, methods described in this disclosure comprise reducing the toxicity caused by a V-agent and/or a G-agent by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to the toxicity caused by the V-agent and/or G-agent in the absence of a PTE or PTER or in the presence of a control PTE or PTER. In some embodiments, methods described in this disclosure comprise relieving or reducing symptoms caused by a V-agent and/or a G-agent by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to the relief or reduction of symptoms in the absence of a PTE or PTER or in the presence of a control. In some embodiments, methods described in this disclosure comprise reducing immunogenicity by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to that of a control. In some embodiments, a control is a PTE or PTER comprising any one of SEQ ID NOs: 1-8.

The term “effective amount” or “amount effective” or “therapeutically effective amount” in the context of dose for administration to a subject refers to an amount of the dose that produces one or more desired responses in a subject. An effective amount can involve reducing the level of an undesired response. An effective amount can also involve delaying the occurrence or onset of an undesired response. An effective amount can also involve enhancing the level of a desired response such as a therapeutic endpoint or result. In some embodiments, administration of a PTE or PTER results in a preventative result or therapeutic result or endpoint with respect to the symptoms of toxicity caused by a V-agent such as VR or VX and/or a G-agent such as GB or GD. The achievement of any of the foregoing can be monitored by routine methods and/or any of the methods disclosed in the present application.

Effective amounts will depend on the particular subject being treated; the severity of a condition; the individual patient parameters including age, physical condition, size and weight; the duration of the treatment; the nature of concurrent therapy (if any); the specific route of administration and like factors.

### Human Microbiome Commensals

Commensals can be generated that express PTE or PTER. In some embodiments, species of Lactobacillus sp., which can have beneficial effects if applied on the skin or administered orally, are used to generate commensals. Non-limiting examples include L. delbrueckii, L. plantarum, L. rhamnosus, L. reuteri, L. paracasei, L. johnsonii, and L. lacti;

Probiotic bacteria selected for oral administration, include but are not limited to: Bifidobacteria sp., E. coli (for example, E. coli Nissle 1917), Bacteroides (for example, B. thetaiotaomicron), and Lactococcus sp.; or yeasts including Saccharomyces bulardii.

Bacteria selected from natural skin commensals for skin application include but are not limited to: Corynebacterium sp., Staphylococcus sp. (for example, S. epidermidis and S. hominis), Propionibacterium sp., Streptococcus sp., Micrococcus sp., Betaproteobacterium sp., Brevibacterium sp., and Dermabacter sp.; or fungi including Malassezia sp., Aspergillus sp., and Candida sp.

Bacteria selected from natural lung commensals for lung application include but are not limited to: Pseudomonas sp., Streptococcus sp., Prevotella sp., Fusobacterium sp., Haemophilus sp., Veillonella sp., and Porphyromonas sp.

Bacteria selected from natural eye commensals for eye application include but are not limited to: Staphylococcus sp., Propionibacterium sp., Corynebacterium sp., Staphylococci sp., and Pseudomonas sp.

Microbial strains can be engineered to express a PTE or PTER (e.g., a PTE or PTER having an amino acid sequence described herein, or having a sequence at least 80, 85, 90 or 95% identical to an amino acid sequence described herein) by introduction into the microbe a nucleic acid (e.g., a self-replicating plasmid, or a heterologous nucleic acid integrated into a chromosome, etc.) comprising a nucleotide sequence encoding a PTE or PTER. Any of the standard protocols well-known in the field for introducing nucleic acids into a microorganism can be used, e.g., those described in: Sambrook et al., 2012, Molecular Cloning: A Laboratory Manual, volumes 1-4, Cold Spring Harbor Press, NY; Nat Methods. (2008) 5(2): 135-146; Frenzel et al. Front Immunol. (2013) 4: 217; and Kunert and Reinhart, Appl Microbiol Biotechnol. (2016) 100: 3451-3461), and in other bacteriology, virology and molecular biology manuals. Viruses and phages, which are useful as vectors, can be used for introducing nucleic acids into mammalian and bacterial cells. Examples include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

Selected strains can be engineered for expression of the recombinant constructs using transformation methods including electroporation and conjugation techniques, such as those described in Sambrook et al., 2012. Electroporation can be executed as in Grosser and Richardson 2014 (DOI 10.1007/7651_2014_183, for S. epidermidis), or Welker et al., 2015 (doi: 10.1093/femsle/fnu033 for Lactobacillus). Conjugation techniques enable the transformation of the target strain by transferring a mobilized plasmid from a donor strain; the latter can be, as non-limiting examples: B. subtilis or E. coli, as in Samperio et al., 2021 (doi: 10.3389/fmicb.2021.606629, for Lactobacillus and Staphylococcus sp.).

Strain commensals engineered as described above can be cultured, isolated and formulated for application to human skin.

For example, a strain commensal can be constructed by introduction of a gene encoding a PTE or PTER (e.g., a PTE or PTER having an amino acid sequence described herein, or having a sequence at least 80, 85, 90 or 95% identical to an amino acid sequence described herein) on a suitable vector (e.g., a plasmid, suicide vector, heterologous nucleic acid designed for homologous introduction into a chromosome, etc.). The coding segment for the PTE or PTER can be operably linked to a promoter at the 5′-end and, optionally, a terminator at the 3′-end. The vector can be designed to comprise a selectable marker (e.g., an antibiotic marker, a gene necessary for growth of the bacterium, a fluorescence marker, etc.), such that the bacteria comprising the vector (and therefore the PTE or PTER gene) can be easily selected. Optionally, in the case of a self-replicating vector such as a plasmid, the vector can comprise an origin of replication and any additional factors (e.g., a replicase) required for replication at the original of replication within the microbe. In the case of a suicide vector or a heterologous nucleic acid designed for homologous recombination into a chromosome, no origin of replication is required.

The cells can be grown in fermentation media and conditions appropriate for each species, including rich media (for example Tryptic Soy Broth, TSB, or other media formulations with yeast extract and tryptone) and minimal media with nitrogen sources, carbohydrates, salts, and micronutrients. The cells are grown to sufficient densities, isolated from the fermentation broth, and then lyophilized for storage in a viable and non-proliferative stage.

For application to the skin, a microbe expressing a PTE or PTER can be formulated in an administration vehicle such as a liquid, lotion, ointment, cream or gel suitable for rubbing. spraying or other methods of administration to the skin. The administration vehicle can also optionally comprise various other components such as: a colorant, a nutrient nutritious to the microbe, a scent, a sunblock, a deodorant (e.g., an agent capable of reducing sweat malodor, which may be the microbe or another agent added to the administration vehicle), an antiperspirant, a moisturizer, etc. The formulation can then be applied to selected area of the skin at different concentrations ranging from 10⁶ CFU/mL to 10⁸ CFU/mL.

In the case of a microbe comprising a gene for a PTE or PTER and intended for human consumption, the microbe can be one suitable for growth within the human digestive tract including but not limited to a probiotic microbe described herein. Non-limiting examples of microorganisms suitable for human consumption include: Lactobacillus species such as L. bulgaricus, L. plantarum, L. paraplantarum, L. corynformis, L. brevis, L. acidophilus, L. rhamnosus, L. helveticus, L. kefiranofaciens, and L. lactis; Streptococcus species such as S. thermophilus; Leuconostoc species such as L. mesenteroides, L. citreum, and L. argentinum; and Pediococcus species such as P. pentosaceus. Additional non-limiting examples of microbes expressing a PTE or PTER for human consumption include Saccharomyces boulardii, Bifidobacterium bifidum, Bacillus coagulans, Gluconacetobacter xylinus, Acetobacter pasteurianus, Acetobacter aceti, Gluconobacter oxydans and Zygosaccharomyces species.

The microbe can be administered as a component in an administration vehicle which can be, for example, a food (e.g., a yogurt), a pill, a pellet, a gelcap, wherein the administration vehicle can further comprise any one or more of: a colorant, a flavor, a bulking agent, a sweetener. The administration vehicle can be, or can be a component in or combined with, a food (e.g., a meat, a vegetable, a starch) or a drink or other liquid or solid intended for human consumption. In various means of application, the administration vehicle can be administered orally (e.g., as a food, pill) or anally (e.g., as a suppository).

In the case of administration to either the skin or the digestive tract, the administration vehicle can further comprise a matrix optionally comprising additional components as part of the formulation, including cryo- and lyoprotectants, such as carbohydrates and peptides, and fatty alcohols.

Optionally, microbes which are administered to the digestive system can comprise lyophilized but viable cells. Additional steps of reactivation may be included, wherein the lyophilized cells undergo subsequent cycles of growth to achieve the desired seed inoculum.

### Compositions, Kits, and Administration

The present disclosure provides compositions, including pharmaceutical compositions, comprising one or more PTEs or PTERs, and optionally a pharmaceutically acceptable excipient. In certain embodiments, a PTE or PTER described in this application is provided in an effective amount in a composition such as a pharmaceutical composition. Compositions, such as pharmaceutical compositions, described in this application can be prepared by any method known in the art.

Pharmaceutical compositions can be prepared, packaged, and/or sold in bulk, as a single unit dose, and/or as a plurality of single unit doses. A “unit dose” is a discrete amount of a pharmaceutical composition comprising a predetermined amount of an active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described in this application will vary, depending upon the identity, size, and/or condition of the subject treated and further depending upon the route by which the composition is to be administered. The composition may comprise, e.g., between 0.1% and 100% (w/w) active ingredient.

The term “pharmaceutically acceptable excipient” or “pharmaceutically acceptable carrier” means a pharmacologically inactive material used together with a pharmacologically active material to formulate the compositions. Pharmaceutically acceptable excipients comprise a variety of materials known in the art, including but not limited to saccharides (such as glucose, lactose, and the like), preservatives such as antimicrobial agents, reconstitution aids, colorants, saline (such as phosphate buffered saline), and buffers. Any one of the compositions provided in the present application may include a pharmaceutically acceptable excipient or carrier.

Pharmaceutically acceptable excipients used in the manufacture of pharmaceutical compositions can include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition. Exemplary excipients include diluents, dispersing and/or granulating agents, surface active agents and/or emulsifiers, disintegrating agents, binding agents, preservatives, buffering agents, lubricating agents, and/or oils (e.g., synthetic oils, semi-synthetic oils).

The term “pharmaceutically acceptable salt” refers to those salts which are, within the scope of sound medical judgment, suitable for use in contact with the tissues of humans and lower animals without undue toxicity and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, which is incorporated by reference in its entirety. Pharmaceutically acceptable salts of the compounds disclosed in this application include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N+(C1-4 alkyl)4-salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Exemplary diluents can include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents can include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers can include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor*), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents can include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives can include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants can include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents can include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives can include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives can include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives can include ethanol, polyethylene glycol, phenol, phenolic compounds, bisphenol, chlorobutanol, hydroxybenzoate, and phenylethyl alcohol.

Exemplary acidic preservatives can include vitamin A, vitamin C, vitamin E, beta-carotene, citric acid, acetic acid, dehydroacetic acid, ascorbic acid, sorbic acid, and phytic acid.

Other preservatives can include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents can include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

In some embodiments, compositions comprising one or more PTE or PTERs are formulated for subcutaneous injection. In some embodiments, compositions comprising one or more PTE or PTERs are formulated for intramuscular injection. Compositions described in this disclosure can be administered via any route that is suitable for the composition and the subject in need thereof.

Injectable preparations, for example sterile injectable aqueous or oleaginous suspensions, can be formulated according to known methods using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose, any bland fixed oil can be employed, including synthetic mono- or di-glycerides. In addition, fatty acids such as oleic acid can be used in the preparation of injectables. The injectable formulations can be sterilized, for example, by filtration through a bacterial-retaining filter, or by incorporating sterilizing agents in the form of sterile solid compositions, which can be dissolved or dispersed in sterile water or other sterile injectable medium prior to use.

Although the descriptions of pharmaceutical compositions provided in this application are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

PTE or PTERs provided in this application are typically formulated in dosage unit form for ease of administration and uniformity of dosage. It will be understood, however, that the total daily usage of the compositions described in this application can be decided by a physician within the scope of sound medical judgment. The specific therapeutically effective dose level for any particular subject or organism will depend upon a variety of factors including the level of toxicity, the age, body weight, general health, and gender of the subject; the time of administration, route of administration, and rate of excretion of the specific active ingredient employed; the duration of the treatment; the PTE or PTER used in combination or coincidental with the specific active ingredient employed; and like factors well known in the medical arts.

In some embodiments, the PTE or PTER or compositions disclosed in this application are formulated and/or administered in nanoparticles. Nanoparticles are particles in the nanoscale. In some embodiments, nanoparticles are less than 1 μm in diameter. In some embodiments, nanoparticles are between about 1 and 100 nm in diameter. Nanoparticles include organic nanoparticles, such as dendrimers, liposomes, or polymeric nanoparticles. Nanoparticles also include inorganic nanoparticles, such as fullerenes, quantum dots, and gold nanoparticles. Compositions may comprise an aggregate of nanoparticles. In some embodiments, the aggregate of nanoparticles is homogeneous, while in other embodiments the aggregate of nanoparticles is heterogeneous.

The exact amount of a PTE or PTER, or composition comprising a PTE or PTER, required to achieve an effective amount will vary from subject to subject, depending, for example, on age, and general condition of a subject, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of an enzyme described in this application. Dosage forms may be administered at a variety of frequencies. In certain embodiments, when multiple doses are administered to a subject, the frequency of administering the multiple doses to the subject is three doses a day, two doses a day, one dose a day, one dose every other day, one dose every third day, one dose every week, one dose every two weeks, one dose every three weeks, or one dose every four weeks, or less frequent than every four weeks. In certain embodiments, the frequency of administering the multiple doses to the subject is one dose per day. In certain embodiments, the frequency of administering the multiple doses to the is two doses per day. In certain embodiments, the frequency of administering the multiple doses to the subject is three doses per day. In certain embodiments, when multiple doses are administered to a subject, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject. In certain embodiments, the duration between the first dose and last dose of the multiple doses is three months, six months, or one year. In certain embodiments, the duration between the first dose and last dose of the multiple doses is the lifetime of the subject. In some embodiments, dose ranging studies can be conducted to establish optimal therapeutic or effective amounts of the component(s) to be present in dosage forms. In embodiments, the component(s) are present in dosage forms in an amount effective to generate a preventative or therapeutic response to various symptoms of toxicity caused by an OPNA such as a V-agent and/or a G-agent.

Compositions as described in this application can be administered in combination with one or more additional pharmaceutical agents (e.g., therapeutically and/or prophylactically active agents). The compounds or compositions can be administered in combination with additional pharmaceutical agents that improve their activity, improve bioavailability, improve safety, reduce and/or modify metabolism, inhibit excretion, and/or modify distribution in a subject.

Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for hydrolyzing or degrading a V-agent, or alleviating the symptoms or toxicity caused by a V-agent and/or a G-agent. In some embodiments, compositions can be administered concurrently with, prior to, or subsequent to one or more additional pharmaceutical agents.

Also encompassed by the disclosure are kits (e.g., pharmaceutical packs) comprising a composition comprising one or more PTE or PTER for use in administering the composition for hydrolyzing or degrading an OPNA such as a V-agent and/or a G-agent. The kits provided may comprise a composition, such as a pharmaceutical composition comprising a PTE or PTER described in this application, and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or a PTE or PTER described in this application. Thus, in one aspect, provided are kits including a container comprising a composition, PTE, or PTER described in this application.

In certain embodiments, a kit described in this application further includes instructions for using the kit. A kit may also include information as required by a regulatory agency such as the U.S. Food and Drug Administration (FDA). In certain embodiments, the information included in the kits is prescribing information. A kit may also include one or more additional pharmaceutical agents described in this application as a separate composition.

### Host Cells

The term “host cell” refers to a cell that can be used to express a polynucleotide, such as a polynucleotide that encodes a OPNA hydrolyzing enzyme, such as a V-agent hydrolyzing enzyme and/or a G-agent hydrolyzing enzyme. The terms “genetically modified host cell,” “recombinant host cell,” and “recombinant strain” are used interchangeably and refer to a host cell that has been genetically modified by, e.g., cloning and transformation methods, or by other methods known in the art (e.g., selective editing methods). Thus, the terms include a host cell (e.g., bacterial cell, yeast cell, fungal cell, insect cell, plant cell, mammalian cell, human cell, etc.) that has been genetically altered, modified, or engineered, so that it exhibits an altered, modified, or different genotype and/or phenotype, as compared to the naturally-occurring cell from which it was derived. It is understood that the term “cell,” as used in this application, may refer to a single cell or a population of cells, such as a population of cells belonging to the same cell line or strain. Use of the singular term “cell” should not be construed to refer explicitly to a single cell rather than a population of cells.

The term “heterologous” with respect to a polynucleotide, such as a polynucleotide comprising a gene, is used interchangeably with the term “exogenous” and the term “recombinant” and refers to: a polynucleotide that has been artificially supplied to a biological system; a polynucleotide that has been modified within a biological system, or a polynucleotide whose expression or regulation has been manipulated within a biological system. A heterologous polynucleotide that is introduced into or expressed in a host cell may be a polynucleotide that comes from a different organism or species from the host cell, or may be a synthetic polynucleotide, or may be a polynucleotide that is also endogenously expressed in the same organism or species as the host cell. For example, a polynucleotide that is endogenously expressed in a host cell may be considered heterologous when it is situated non-naturally in the host cell; expressed recombinantly in the host cell, either stably or transiently; modified within the host cell; selectively edited within the host cell; expressed in a copy number that differs from the naturally occurring copy number within the host cell; or expressed in a non-natural way within the host cell, such as by manipulating regulatory regions that control expression of the polynucleotide. In some embodiments, a heterologous polynucleotide is a polynucleotide that is endogenously expressed in a host cell but whose expression is driven by a promoter that does not naturally regulate expression of the polynucleotide. In other embodiments, a heterologous polynucleotide is a polynucleotide that is endogenously expressed in a host cell and whose expression is driven by a promoter that does naturally regulate expression of the polynucleotide, but the promoter or another regulatory region is modified. In some embodiments, the promoter is recombinantly activated or repressed. For example, gene-editing based techniques may be used to regulate expression of a polynucleotide, including an endogenous polynucleotide, from a promoter, including an endogenous promoter. See, e.g., Chavez et al., Nat Methods. 2016 July; 13(7): 563-567. A heterologous polynucleotide may comprise a wild-type sequence or a mutant sequence as compared with a reference polynucleotide sequence.

Suitable host cells include, but are not limited to: yeast cells, bacterial cells (including Gram-positive and Gram-negative cells), archaebacteria, algal cells, plant cells, fungal cells, lichen, corals, insect cells, invertebrate cells, insect cells, fish cells, bird cells, reptile cells, amphibian cells, animal cells, including mammalian cells, and human cells.

Suitable yeast host cells include, but are not limited to: Candida, Hansenula, Saccharomyces, Schizosaccharomyces, Pichia, Kluyveromyces, and Yarrowia. In some embodiments, the yeast cell is Hansenula polymorpha, Saccharomyces cerevisiae, Saccaromyces carlsbergensis, Saccharomyces diastaticus, Saccharomyces norbensis, Saccharomyces kluyveri, Schizosaccharomyces pombe, Komagataella phaffii, formerly known as Pichia pastoris, Pichia finlandica, Pichia trehalophila, Pichia kodamae, Pichia membranaefaciens, Pichia opuntiae, Pichia thermotolerans, Pichia salictaria, Pichia quercuum, Pichia pijperi, Pichia stipitis, Pichia methanolica, Pichia angusta, Kluyveromyces lactis, Candida albicans, or Yarrowia lipolytica.

In some embodiments, the yeast strain is an industrial polyploid yeast strain. Other non-limiting examples of fungal cells include cells obtained from Aspergillus spp., Penicillium spp., Fusarium spp., Rhizopus spp., Acremonium spp., Neurospora spp., Sordaria spp., Magnaporthe spp., Allomyces spp., Ustilago spp., Botrytis spp., and Trichoderma spp.

In certain embodiments, the host cell is an algal cell such as, Chlamydomonas (e.g., C. Reinhardtii) and Phormidium (P. sp. ATCC29409).

In other embodiments, the host cell is a prokaryotic cell. Suitable prokaryotic cells include gram-positive, gram-negative, and gram-variable bacterial cells. The host cell may be a species of, but not limited to: Agrobacterium, Alicyclobacillus, Anabaena, Anacystis, Acinetobacter, Acidothermus, Arthrobacter, Azobacter, Bacillus, Bifidobacterium, Brevibacterium, Butyrivibrio, Buchnera, Campestris, Camplyobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomonospora, Saccharopolyspora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia, and Zymomonas.

In some embodiments, the bacterial host strain is an industrial strain. Numerous bacterial industrial strains are known and suitable for the methods and compositions described in this application. In some embodiments, the bacterial host cell is of the Agrobacterium species (e.g., A. radiobacter, A. rhizogenes, A. rubi), the Arthrobacterspecies (e.g., A. aurescens, A. citreus, A. globformis, A. hydrocarboglutamicus, A. mysorens, A. nicotianae, A. paraffineus, A. protophonniae, A. roseoparaffinus, A. sulfureus, A. ureafaciens), or the Bacillus species (e.g., B. thuringiensis, B. anthracis, B. megaterium, B. subtilis, B. lentus, B. circulars, B. pumilus, B. lautus, B. coagulans, B. brevis, B. firmus, B. alkaophius, B. licheniformis, B. clausii, B. stearothermophilus, B. halodurans and B. amyloliquefaciens). In particular embodiments, the host cell will be an industrial Bacillus strain including but not limited to B. subtilis, B. pumilus, B. licheniformis, B. megaterium, B. clausii, B. stearothermophilus and B. amyloliquefaciens. In some embodiments, the host cell will be an industrial Clostridium species (e.g., C. acetobutylicum, C. tetani E88, C. lituseburense, C. saccharobutylicum, C. perfringens, C. beijerinckii). In some embodiments, the host cell will be an industrial Corynebacterium species (e.g., C. glutamicum, C. acetoacidophilum). In some embodiments, the host cell will be an industrial Escherichia species (e.g., E. coli). In some embodiments, the host cell will be an industrial Erwinia species (e.g., E. uredovora, E. carotovora, E. ananas, E. herbicola, E. punctata, E. terreus). In some embodiments, the host cell will be an industrial Pantoea species (e.g., P. citrea, P. agglomerans). In some embodiments, the host cell will be an industrial Pseudomonas species, (e.g., P. putida, P. aeruginosa, P. mevalonii). In some embodiments, the host cell will be an industrial Streptococcus species (e.g., S. equisimiles, S. pyogenes, S. uberis). In some embodiments, the host cell will be an industrial Streptomyces species (e.g., S. ambofaciens, S. achromogenes, S. avermitilis, S. coelicolor, S. aureofaciens, S. aureus, S. fungicidicus, S. griseus, S. lividans). In some embodiments, the host cell will be an industrial Zymomonas species (e.g., Z. mobilis, Z. lipolytica), and the like.

The present disclosure is suitable for use with an E. coli cell. In some embodiments, the E. coli cell is an E. coli BL21(DE3) cell. The present disclosure is suitable for use with a Bacillus cell. The present disclosure is suitable for use with a filamentous fungi cell. The present disclosure is suitable for use with a yeast cell.

The present disclosure may also be suitable for use with a variety of animal cell types, including mammalian cells, for example, human (including HEK 293, HEK 293T, A549, HepG2, HeLa, WI38, PER.C6 and Bowes melanoma cells), non-human primate (including COS-1, COS-7) mouse (including 3T3, C2C12, ROS 17/2.8 (osteosarcoma cells), NS0, NS1, Sp2/0), hamster (CHO, BHK), monkey (COS, FRhL, Vero), insect cells, for example fall armyworm (including Sf9 and Sf21), silkmoth (including BmN), cabbage looper (including BTI-Tn-5B1-4) and common fruit fly (including Schneider 2), and hybridoma cell lines.

In various embodiments, strains that may be used in the practice of the disclosure including both prokaryotic and eukaryotic strains, and are readily accessible to the public from a number of culture collections such as American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL).

In some embodiments, a host cell produces an OPNA hydrolyzing enzyme, such as a V-agent hydrolyzing enzyme and/or a G-agent hydrolyzing enzyme, including but not limited to an OPNA hydrolyzing enzyme having the sequence of any one of SEQ ID NOs: 1-4 or 6. In some embodiments, a PTE or PTER comprises an amino acid sequence that is at least 90% identical to any one of SEQ ID NOs: 1-4 and 6. In some embodiments, a host cell comprises a polynucleotide encoding an OPNA hydrolyzing enzyme, wherein the polynucleotide comprises the sequence of any one of SEQ ID NOs: 10-13 and 15. In some embodiments, a host cell comprises a polynucleotide encoding a OPNA hydrolyzing enzyme, wherein the polynucleotide comprises a sequence that is at least 90% identical to any one of SEQ ID NOs: 10-13 and 15.

### Culturing of Host Cells

Any of the cells disclosed in this application can be cultured in media of any type (rich or minimal) and any composition prior to, during, and/or after contact and/or integration of a polynucleotide. The conditions of the culture or culturing process can be optimized through routine experimentation as would be understood by one of ordinary skill in the art. In some embodiments, the selected media is supplemented with various components. In some embodiments, the concentration and amount of a supplemental component is optimized. In some embodiments, other aspects of the media and growth conditions (e.g., pH, temperature, etc.) are optimized through routine experimentation. In some embodiments, the frequency that the media is supplemented with one or more supplemental components, and the amount of time that the cell is cultured, is optimized.

Culturing of the cells described in this application can be performed in culture vessels known and used in the art. In some embodiments, an aerated reaction vessel (e.g., a stirred tank reactor) is used to culture the cells. In some embodiments, a bioreactor or fermenter is used to culture the cell. Thus, in some embodiments, the cells are used in fermentation. As used in this application, the terms “bioreactor” and “fermenter” are interchangeably used and refer to an enclosure, or partial enclosure, in which a biological, biochemical and/or chemical reaction takes place that involves a living organism or part of a living organism. A “large-scale bioreactor” or “industrial-scale bioreactor” is a bioreactor that is used to generate a product on a commercial or quasi-commercial scale. Large scale bioreactors typically have volumes in the range of liters, hundreds of liters, thousands of liters, or more.

Non-limiting examples of bioreactors include: stirred tank fermenters, bioreactors agitated by rotating mixing devices, chemostats, bioreactors agitated by shaking devices, airlift fermenters, packed-bed reactors, fixed-bed reactors, fluidized bed bioreactors, bioreactors employing wave induced agitation, centrifugal bioreactors, roller bottles, and hollow fiber bioreactors, roller apparatuses (for example benchtop, cart-mounted, and/or automated varieties), vertically-stacked plates, spinner flasks, stirring or rocking flasks, shaken multi-well plates, MD bottles, T-flasks, Roux bottles, multiple-surface tissue culture propagators, modified fermenters, and coated beads (e.g., beads coated with serum proteins, nitrocellulose, or carboxymethyl cellulose to prevent cell attachment).

In some embodiments, the bioreactor includes a cell culture system where the cell (e.g., yeast cell) is in contact with moving liquids and/or gas bubbles. In some embodiments, the cell or cell culture is grown in suspension. In other embodiments, the cell or cell culture is attached to a solid phase carrier. Non-limiting examples of a carrier system includes microcarriers (e.g., polymer spheres, microbeads, and microdisks that can be porous or non-porous), cross-linked beads (e.g., dextran) charged with specific chemical groups (e.g., tertiary amine groups), 2D microcarriers including cells trapped in nonporous polymer fibers, 3D carriers (e.g., carrier fibers, hollow fibers, multicartridge reactors, and semi-permeable membranes that can comprising porous fibers), microcarriers having reduced ion exchange capacity, encapsulation cells, capillaries, and aggregates. In some embodiments, carriers are fabricated from materials such as dextran, gelatin, glass, or cellulose.

In some embodiments, industrial-scale processes are operated in continuous, semi-continuous or non-continuous modes. Non-limiting examples of operation modes are batch, fed batch, extended batch, repetitive batch, draw/fill, rotating-wall, spinning flask, and/or perfusion mode of operation. In some embodiments, a bioreactor allows continuous or semi-continuous replenishment of the substrate stock, for example a carbohydrate source and/or continuous or semi-continuous separation of the product, from the bioreactor.

In some embodiments, the bioreactor or fermenter includes a sensor and/or a control system to measure and/or adjust reaction parameters. Non-limiting examples of reaction parameters include biological parameters (e.g., growth rate, cell size, cell number, cell density, cell type, or cell state, etc.), chemical parameters (e.g., pH, redox-potential, concentration of reaction substrate and/or product, concentration of dissolved gases, such as oxygen concentration and CO₂ concentration, nutrient concentrations, metabolite concentrations, concentration of an oligopeptide, concentration of an amino acid, concentration of a vitamin, concentration of a hormone, concentration of an additive, serum concentration, ionic strength, concentration of an ion, relative humidity, molarity, osmolarity, concentration of other chemicals, for example buffering agents, adjuvants, or reaction by-products), physical/mechanical parameters (e.g., density, conductivity, degree of agitation, pressure, and flow rate, shear stress, shear rate, viscosity, color, turbidity, light absorption, mixing rate, conversion rate, as well as thermodynamic parameters, such as temperature, light intensity/quality, etc.). Sensors to measure the parameters described in this application are well known to one of ordinary skill in the relevant mechanical and electronic arts. Control systems to adjust the parameters in a bioreactor based on the inputs from a sensor described in this application are well known to one of ordinary skill in the art in bioreactor engineering.

In some embodiments, the method involves batch fermentation (e.g., shake flask fermentation). General considerations for batch fermentation (e.g., shake flask fermentation) include the level of oxygen and glucose. For example, batch fermentation (e.g., shake flask fermentation) may be oxygen and glucose limited, so in some embodiments, the capability of a strain to perform in a well-designed fed-batch fermentation is underestimated.

In some embodiments, the cells of the present disclosure are adapted to produce an OPNA hydrolyzing enzyme, such as a V-agent hydrolyzing enzyme and/or a G-agent hydrolyzing enzyme, in vivo. In some embodiments, the cells are adapted to secrete an OPNA hydrolyzing enzyme including a PTE or PTER. In some embodiments, the cells of the present disclosure are lysed, and the remaining lysates are recovered for subsequent use. In some embodiments, any of the methods described in this application may include isolation and/or purification of an OPNA hydrolyzing enzyme. For example, the isolation and/or purification can involve one or more of cell lysis, centrifugation, extraction, column chromatography, distillation, crystallization, and lyophilization.

### Expression of Polynucleotides in Host Cells

Aspects of the present disclosure relate to recombinant proteins, functional mutants and variants thereof, as well as their uses. For example, the methods described in this application may be used to produce an OPNA hydrolyzing enzyme, such as a V-agent hydrolyzing enzyme and/or a G-agent hydrolyzing enzyme. The methods may comprise using a host cell comprising a protein or peptide disclosed in this application, cell lysate, isolated protein or peptide, or any combination thereof. Methods comprising recombinant expression of genes encoding a protein or peptide disclosed in this application in a host cell are encompassed by the present disclosure.

Aspects of the disclosure relate to polynucleotides encoding an OPNA hydrolyzing enzyme, such as a V-agent hydrolyzing enzyme and/or a G-agent hydrolyzing enzyme, wherein the polynucleotide comprises the sequence of any one of SEQ ID NOs: 10-13 and 15. Further aspects of the disclosure relate to polynucleotides encoding an OPNA hydrolyzing enzyme, wherein the polynucleotide comprises a sequence that is at least 90% identical to any one of SEQ ID NOs: 10-13 and 15.

A polynucleotide encoding any of the recombinant polypeptides (e.g., PTE or PTER) described in this application may be incorporated into any appropriate vector through any method known in the art. For example, the vector may be an expression vector, including but not limited to a viral vector (e.g., a lentiviral, retroviral, adenoviral, or adeno-associated viral vector), any vector suitable for transient expression, any vector suitable for constitutive expression, or any vector suitable for inducible expression (e.g., a galactose-inducible or doxycycline-inducible vector).

A vector encoding any of the recombinant polypeptides (e.g., PTE or PTER) described in this application may be introduced into a suitable host cell using any method known in the art. Non-limiting examples of yeast transformation protocols are described in Gietz et al., Yeast transformation can be conducted by the LiAc/SS Carrier DNA/PEG method. Methods Mol Biol. 2006; 313:107-20, which is hereby incorporated by reference in its entirety. Host cells may be cultured under any conditions suitable as would be understood by one of ordinary skill in the art. For example, any media, temperature, and incubation conditions known in the art may be used. For host cells carrying an inducible vector, cells may be cultured with an appropriate inducible agent to promote expression.

In some embodiments, a vector replicates autonomously in the cell. In some embodiments, a vector integrates into a chromosome within a cell. A vector can contain one or more endonuclease restriction sites that are cut by a restriction endonuclease to insert and ligate a nucleic acid containing a gene described in this application to produce a recombinant vector that is able to replicate in a cell. Vectors can be composed of DNA or RNA. Cloning vectors include, but are not limited to: plasmids, fosmids, phagemids, virus genomes and artificial chromosomes. As used in this application, the terms “expression vector” or “expression construct” refer to a nucleic acid construct, generated recombinantly or synthetically, with a series of specified nucleic acid elements that permit transcription of a particular nucleic acid in a host cell. In some embodiments, the nucleotide sequence of a gene described in this application is inserted into a cloning vector so that it is operably joined to regulatory sequences and, in some embodiments, expressed as an RNA transcript. In some embodiments, the vector contains one or more markers, such as a selectable marker as described in this application, to identify cells transformed or transfected with the recombinant vector. In some embodiments, a host cell has already been transformed with one or more vectors. In some embodiments, a host cell that has been transformed with one or more vectors is subsequently transformed with one or more vectors. In some embodiments, a host cell is transformed simultaneously with more than one vector. In some embodiments, a cell that has been transformed with a vector or an expression cassette incorporates all or part of the vector or expression cassette into its genome. In some embodiments, the nucleotide sequence of a gene described in this application is codon-optimized. Codon optimization may increase production of the gene product by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%, including all values in between) relative to a reference sequence that is not codon-optimized.

In some embodiments, the polynucleotide encoding any of the proteins described in this application is under the control of regulatory sequences (e.g., enhancer sequences). In some embodiments, a polynucleotide is expressed under the control of a promoter. The promoter can be a native promoter, e.g., the promoter of the gene in its endogenous context, which provides normal regulation of expression of the gene. Alternatively, a promoter can be a promoter that is different from the native promoter of the gene, e.g., the promoter is different from the promoter of the gene in its endogenous context.

In some embodiments, the promoter is a eukaryotic promoter. Non-limiting examples of eukaryotic promoters include TDH3, PGK1, PKC1, PDC1, TEF1, TEF2, RPL18B, SSA1, TDH2, PYK1, TPI1, GAL1, GAL10, GAL7, GAL3, GAL2, MET3, MET25, HXT3, HXT7, ACT1, ADH1, ADH2, CUP1-1, ENO2, and SOD1, as would be known to one of ordinary skill in the art (see, e.g., Addgene website: blog.addgene.org/plasmids-101-the-promoter-region). In some embodiments, the promoter is a prokaryotic promoter (e.g., bacteriophage or bacterial promoter). Non-limiting examples of bacteriophage promoters include Plsicon, T3, T7, SP6, and PL. Non-limiting examples of bacterial promoters include Pbad, PmgrB, Ptrc2, Plac/ara, Ptac, and Pm.

In some embodiments, the promoter is an inducible promoter. As used in this application, an “inducible promoter” is a promoter controlled by the presence or absence of a molecule. Non-limiting examples of inducible promoters include chemically regulated promoters and physically regulated promoters. For chemically regulated promoters, the transcriptional activity can be regulated by one or more compounds, such as methanol, alcohol, tetracycline, galactose, a steroid, a metal, an amino acid, or other compounds. For physically regulated promoters, transcriptional activity can be regulated by a phenomenon such as light or temperature. Non-limiting examples of tetracycline-regulated promoters include anhydrotetracycline (aTc)-responsive promoters and other tetracycline-responsive promoter systems (e.g., a tetracycline repressor protein (tetR), a tetracycline operator sequence (tetO) and a tetracycline transactivator fusion protein (tTA)). Non-limiting examples of steroid-regulated promoters include promoters based on the rat glucocorticoid receptor, human estrogen receptor, moth ecdysone receptors, and promoters from the steroid/retinoid/thyroid receptor superfamily. Non-limiting examples of metal-regulated promoters include promoters derived from metallothionein (proteins that bind and sequester metal ions) genes. Non-limiting examples of pathogenesis-regulated promoters include promoters induced by salicylic acid, ethylene or benzothiadiazole (BTH). Non-limiting examples of temperature/heat-inducible promoters include heat shock promoters. Non-limiting examples of light-regulated promoters include light responsive promoters from plant cells. In certain embodiments, the inducible promoter is a galactose-inducible promoter. In some embodiments, the inducible promoter is induced by one or more physiological conditions (e.g., pH, temperature, radiation, osmotic pressure, saline gradients, cell surface binding, or concentration of one or more extrinsic or intrinsic inducing agents). Non-limiting examples of an extrinsic inducer or inducing agent include amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or any combination.

In some embodiments, the promoter is a constitutive promoter. As used in this application, a “constitutive promoter” refers to an unregulated promoter that allows continuous transcription of a gene. Non-limiting examples of a constitutive promoter include TDH3, PGK1, PKC1, PDC1, TEF1, TEF2, RPL18B, SSA1, TDH2, PYK1, TPI1, HXT3, HXT7, ACT1, ADH1, ADH2, ENO2, and SOD1. Other inducible promoters or constitutive promoters, including synthetic promoters, that may be known to one of ordinary skill in the art are also contemplated.

Regulatory sequences needed for gene expression may vary between species or cell types, but generally include, as necessary, 5′-non-transcribed and 5′-non-translated sequences involved with the initiation of transcription and translation respectively, such as a TATA box, capping sequence, CAAT sequence, and the like. In particular, such 5′-non-transcribed regulatory sequences will include a promoter region which includes a promoter sequence for transcriptional control of the operably joined gene. Regulatory sequences may also include enhancer sequences or upstream activator sequences. Vectors may include 5′-leader or signal sequences. The regulatory sequence may also include a terminator sequence. In some embodiments, a terminator sequence marks the end of a gene in DNA during transcription. The choice and design of one or more appropriate vectors suitable for inducing expression of one or more genes described in this application in a heterologous organism is within the ability and discretion of one of ordinary skill in the art. Expression vectors containing the necessary elements for expression are commercially available and known to one of ordinary skill in the art (see, e.g., Sambrook et al., Molecular Cloning: A Laboratory Manual, Fourth Edition, Cold Spring Harbor Laboratory Press, 2012).

The skilled artisan will also realize that mutations in a recombinant polypeptide (e.g., a PTE or PTER) coding sequence may result in conservative amino acid substitutions to provide functionally equivalent variants of the foregoing polypeptides, e.g., variants that retain the activities of the polypeptides. As used in this application, a “conservative amino acid substitution” refers to an amino acid substitution that does not alter the relative charge or size characteristics or functional activity of the protein in which the amino acid substitution is made.

In some instances, an amino acid is characterized by its R group (see, e.g., Table 1). For example, an amino acid may comprise a nonpolar aliphatic R group, a positively charged R group, a negatively charged R group, a nonpolar aromatic R group, or a polar uncharged R group. Non-limiting examples of an amino acid comprising a nonpolar aliphatic R group include alanine, glycine, valine, leucine, methionine, and isoleucine. Non-limiting examples of an amino acid comprising a positively charged R group includes lysine, arginine, and histidine. Non-limiting examples of an amino acid comprising a negatively charged R group include aspartate and glutamate. Non-limiting examples of an amino acid comprising a nonpolar, aromatic R group include phenylalanine, tyrosine, and tryptophan. Non-limiting examples of an amino acid comprising a polar uncharged R group include serine, threonine, cysteine, proline, asparagine, and glutamine.

Non-limiting examples of functionally equivalent variants of polypeptides may include conservative amino acid substitutions in the amino acid sequences of proteins disclosed in this application. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D. Additional non-limiting examples of conservative amino acid substitutions are provided in Table 1.

In some embodiments, 1, 2, 3, 4, 5, 6, 7, 8, 9, 10, 11, 12, 13, 14, 15, 16, 17, 18, 19, 20 or more than 20 residues can be changed when preparing variant polypeptides. In some embodiments, amino acids are replaced by conservative amino acid substitutions. In some embodiments, amino acids are replaced by non-conservative amino acid substitutions.

TABLE 1
Non-limiting Examples of conservative amino acid substitutions

| Original | | Conservative Amino |
|---|---|---|
| Residue | R Group Type | Acid Substitutions |
| Ala (A) | nonpolar aliphatic | Cys, Gly, Ser |
| Arg (R) | positively charged | His, Lys |
| Asn (N) | polar uncharged | Asp, Gln, Glu |
| Asp (D) | negatively charged | Asn, Gln, Glu |
| Cys (C) | polar uncharged | Ala, Ser |
| Gln (Q) | polar uncharged | Asn, Asp, Glu |
| Glu (E) | negatively charged | Asn, Asp, Gln |
| Gly (G) | nonpolar aliphatic | Ala, Ser |
| His (H) | positively charged | Arg, Tyr, Trp |
| Ile (I) | nonpolar aliphatic | Leu, Met, Val |
| Leu (L) | nonpolar aliphatic | Ile, Met, Val |
| Lys (K) | positively charged | Arg, His |
| Met (M) | nonpolar aliphatic | Ile, Leu, Phe, Val |
| Pro (P) | polar uncharged | |
| Phe (F) | nonpolar aromatic | Met, Trp, Tyr |
| Ser (S) | polar uncharged | Ala, Gly, Thr |
| Thr (T) | polar uncharged | Ala, Asn, Ser |
| Trp (W) | nonpolar aromatic | His, Phe, Tyr, Met |
| Tyr (Y) | nonpolar aromatic | His, Phe, Trp |
| Val (V) | nonpolar aliphatic | Ile, Leu, Met, Thr |

Mutations (e.g., substitutions, insertions, additions, deletions, or truncations) can be made in a nucleotide sequence by a variety of methods known to one of ordinary skill in the art. For example, mutations (e.g., substitutions, insertions, additions, deletions, or truncations) can be made by PCR-directed mutation, site-directed mutagenesis according to the method of Kunkel (Kunkel, Proc. Nat. Acad. Sci. U.S.A. 82: 488-492, 1985), by chemical synthesis of a gene encoding a polypeptide, by gene editing methods, or by insertions, such as insertion of a tag (e.g., a His tag or a GFP tag). Mutations can include, for example, substitutions, insertions, additions, deletions, truncations, and translocations, generated by any method known in the art. Methods for producing mutations may be found in in references such as Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York, 2010.

It is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited in the present application are incorporated by reference for the purposes or subject matter referenced in this disclosure.

### EXAMPLES

In order that the invention described in the present application may be more fully understood, the following examples are set forth. The examples described in this application are offered to illustrate the systems and methods provided in this disclosure and are not to be construed in any way as limiting their scope.

### Example 1: Identification of OPNA Hydrolyzing Enzymes that Hydrolyze VX and/or VR Nerve Agents

To identify OPNA hydrolyzing enzymes that can hydrolyze VX and/or VR, a library of candidate PTEs was designed from sequences in metagenomic databases with similarity to PTE from B. dininuta (SEQ ID NO: 18, which corresponds to the amino acid sequence of UniprotKB Accession No. POA434).

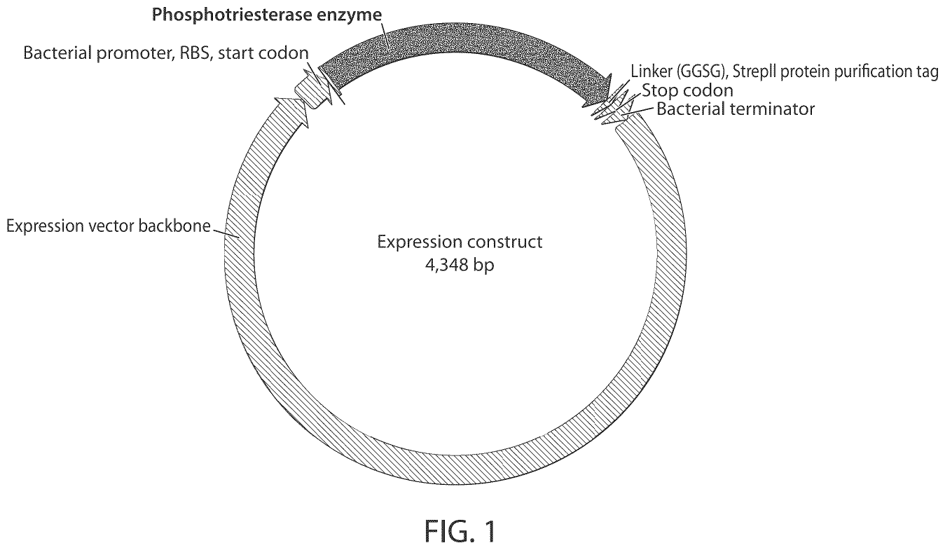

Each candidate enzyme sequence was tagged with a C-terminal StrepII affinity tag and flexible linker to enable purification. Nucleotide sequences were recoded for expression in E. coli and synthesized in the replicative expression vector shown in FIG. 1. Each candidate enzyme expression construct was transformed into an E. coli B3L21(DE3) strain. Strain t339870, expressing a StrepII-tagged fluorescent (GFP) protein (SEQ ID NO: 20), was included in the library screen as a negative control for enzyme activity. Strain t401992, expressing a StrepII-tagged PTE from B. diminuta (SEQ ID NO: 7, which corresponds to the amino acid sequence of UniprotKB Accession No. P0A434, except that the signal sequence was removed and a methionine residue was added at the N-terminus), was included in the library as a positive control and was used to establish hit ranking. Strain t402006, expressing the B. diminuta PTE variant G1-C74 (SEQ ID NO: 5) was also used as a positive control.

VX and YR hydrolysis activity of purified PTEs (prepared and stored in a PTE-specific buffer containing 100 mM HEPES, pH 8.0, 150 mMk NaCl, 50 mM biotin, 0.1 mM CoCl₂, 10% glycerol) was measured by monitoring thiol release using 5,5′-dithiobis(2-nitro-benzoic acid) (DTNB; aka Ellman's reagent). The assay was conducted in 96-well, clear-bottom microplates. Enzyme samples were prepared by mixing 25 μl of each purified enzyme (64-4900 ng/pi) with 45 μl of DTNB reagent solution (50 mM HEPES, pH 7.6, 137 mM NaCl, 2.7 mM KCl, 8 mM DTNB, 1 mM CoCl). The assay was initiated with 50 μl of VX or VR racemates (3 mM). The final volume for each reaction was 120 μl. Hydrolysis of VX and VR (corresponding to the increase in Ellman's chromophore concentration) was monitored at A412 (ε=13600 M−1 cm⁻¹) for 10 minutes at room temperature. Kinetic parameters were calculated as mOD412 min⁻¹ mg⁻¹.

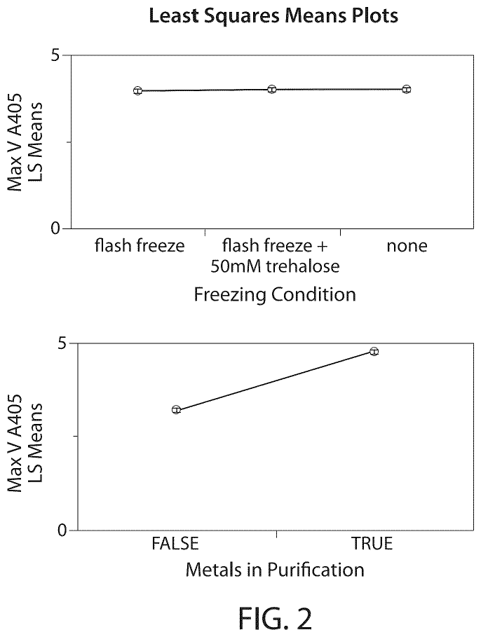

Representative PTEs were tested for their stability and showed no statistically significant loss of activity over 23 hours of sample time. Purification and cryopreservation conditions (flash freeze, flash freeze+50 mM trehalose, and no treatment) were determined on a representative set of PTEs and showed that all cryopreservation conditions resulted in similar enzyme activity. (FIG. 2). Metals and elution buffer were added in the PTE (“TRUE” group) for testing their activity. FIG. 2 shows that addition of metals in the purification and elution buffers helped increase activity of the enzymes.

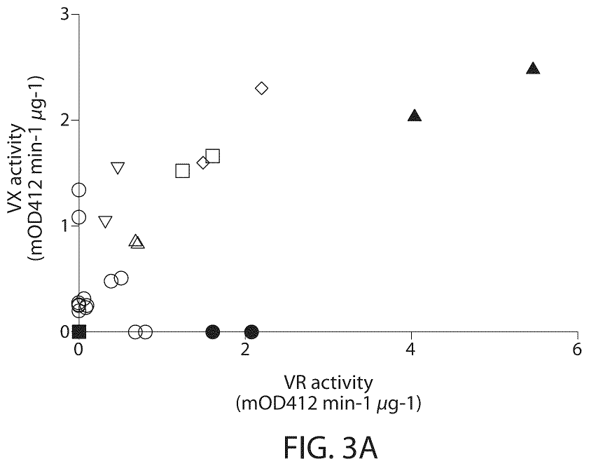

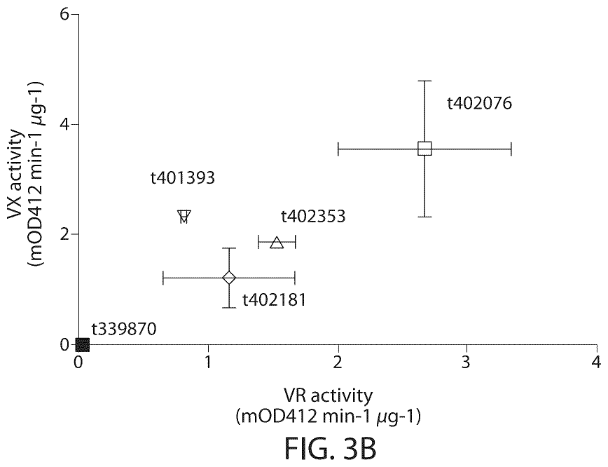

The activity of candidate PTEs on VX and VR was examined. As shown in FIG. 3A, several library PTEs (expressed by strains t402353, t402181, t401393, and t402076) were identified for which each replicate produced detectable amounts of VX and/or VR hydrolytic activity, including >10% of the average VR hydrolytic rate of positive control t401992 on VR (FIG. 3A; Table 2). While the positive control PTE from B. dininuia (SEQ ID NO: 7) expressed by strain t401992 was only active against VR, strains t402353, t402181, t401393, and t402076 were surprisingly found to be active against both VX and VR. Table 2 shows the quantity, purity, and the activity against VX and VR of these candidate PTEs.

To confirm the activity of the candidate PTEs identified in the primary screen, a secondary screen was performed. The experimental protocol for the secondary screen was the same as the primary screen, except that three replicates per strain were tested. The overall trends in VX and VR hydrolytic activity by strains t402353, t402181, t401393, and t402076 observed in the primary screen were also observed in the secondary screen (FIG. 3B; Table 3).

Sequences for PTEs described in Example 1, including candidate PTEs for which data are provided in FIGS. 3A and 3B are provided in Table 5.

TABLE 2
Results of Primary Screen

| | Primary Screen Data | | | | |
|---|---|---|---|---|---|
| | | | | VR activity | VX activity |
| | | Quantity | Purity | (mOD412 min⁻¹ | (mOD412 min⁻¹ |
| Strain | Replicate | (μg) | (%) | μg⁻¹) | μg⁻¹) |
| t402353 | 1 | 71.1 | 97.1 | 0.68 | 0.87 |
| | 2 | 74.0 | 94.9 | 0.71 | 0.85 |
| t402181 | 1 | 29.0 | 68.0 | 2.20 | 2.30 |
| | 2 | 23.3 | 100 | 1.50 | 1.60 |
| t401393 | 1 | 50.7 | 99.3 | 0.47 | 1.54 |
| | 2 | 43.1 | 96.4 | 0.32 | 1.03 |
| t402076 | 1 | 40.3 | 79.4 | 1.61 | 1.66 |
| | 2 | 41.6 | 79.6 | 1.25 | 1.52 |
| t402287 | 1 | 60.1 | 94.9 | 0.51 | 0.51 |
| | 2 | 72.5 | 97.1 | 0.39 | 0.48 |
| t402121 | 1 | 23.3 | 100 | 0.80 | 0 |
| | 2 | 21.3 | 100 | 0.68 | 0 |
| t401421 | 1 | 32.6 | 100 | 0 | 0.28 |
| | 2 | 67.9 | 95.8 | 0 | 0.26 |
| t401451 | 1 | 107 | 99.6 | 0.06 | 0.31 |
| | 2 | 79.7 | 99.5 | 0 | 0.25 |
| t402024 | 1 | 9.9 | 95.0 | 0 | 1.34 |
| | 2 | 11.5 | 94.0 | 0 | 1.08 |
| t402094 | 1 | 584 | 98.6 | 0.09 | 0.25 |
| | 2 | 449 | 98.9 | 0.08 | 0.23 |
| t402397 | 1 | 88.3 | 97.0 | 0 | 0.2 |
| | 2 | 83.8 | 96.7 | 0 | 0.2 |
| t401992 | 1 | 7.7 | 59.0 | 2.08 | 0 |
| (Positive | 2 | 13.0 | 54.9 | 1.61 | 0 |
| Control) | | | | | |
| t402006 | 1 | 41.0 | 92.3 | 5.47 | 2.50 |
| (Positive | 2 | 49.6 | 90.3 | 4.04 | 2.05 |
| Control) | | | | | |
| t339870 | Average of 34 | 104.5 (59.7) | 81.9 (38.4) | 0 | 0 |
| (Negative | reps | | | | |
| Control) | | | | | |

TABLE 3
Results of Secondary Screen

| | Secondary Screen Data | | | |
|---|---|---|---|---|
| | | | VR activity*^{‡} | VX activity*^{‡} |
| | | | (mOD412 | (mOD412 |
| | Quantity*^{†} | Purity*^{†} | min⁻¹ | min⁻¹ |
| Strain | (μg) | (%) | μg⁻¹) | μg⁻¹) |
| t402353 | 70.8 | 99.98 | 1.53 | 1.87 |
| | (12) | (0.08) | (0.14) | (0.11) |
| t402181 | 38.4 | 100.00 | 1.16 | 1.22 |
| | (7.2) | (0.00) | (0.51) | (0.54) |
| t401393 | 121.2 | 99.70 | 0.81 | 2.33 |
| | (21.6) | (0.24) | (0.02) | (0.03) |
| t402076 | 31.2 | 99.69 | 2.67 | 3.56 |
| | (8.4) | (0.52) | (0.67) | (1.24) |
| t402006 | 62.4 | 95.45 | 7.71 | 3.48 |
| (Positive | (9.6) | (0.83) | (0.46) | (0.13) |
| Control) | | | | |
| t339870 | 33.6 | 98.82 | 0.03 | 0 |
| (Negative | (6) | (0.30) | (0.03) | |
| Control) | | | | |

*Reported as AVG (S.D.); For “Quantity” and “Purity” measurements, samples with no detectable protein were omitted from calculations. For t401393, 20 of 24 replicate samples had detectable protein. For t402181, 23 of 24 replicate had detectable protein. For all other enzymes and controls, all samples yielded protein.
^{†}Average of 24 replicates
^{‡}Average of 3 replicates

Table 4 shows sequence identity of candidate PTEs identified in the primary screen relative to the previously-described IVI-13 PTE variant (Goldsmith et al. (2015) Arch Toxicol 90 (11): 2711-2724. All of the candidate PTEs identified had low (33-34%) identity to the IVH3 PTE variant.

TABLE 4
Sequence Identity Matrix for Enzymes Identified
in Primary Screen (% identity)

| % Identity | t402353 | t402076 | t401393 | t402181 | IVH3 |
|---|---|---|---|---|---|
| t402353 | | 94 | 87 | 92 | 33 |
| (SEQ ID | | | | | |
| NO: 4) | | | | | |
| t402076 | 94 | | 87 | 91 | 33 |
| (SEQ ID | | | | | |
| NO: 3) | | | | | |
| t401393 | 87 | 87 | | 90 | 34 |
| (SEQ ID | | | | | |
| NO: 2) | | | | | |
| t402181 | 92 | 91 | 90 | | 34 |
| (SEQ ID | | | | | |
| NO: 1) | | | | | |
| IVH3 (SEQ | 33 | 33 | 34 | 34 | |
| ID NO: 8) | | | | | |

TABLE 5
Sequences of Candidate PTEs and PTERs described in Example 1

| | | PTE or | PTE or |
|---|---|---|---|
| | | PTER Amino | PTER |
| | Source organism of PTE or | Acid SEQ | Nucleotide |
| Strain ID | PTER | ID NO | SEQ ID NO |
| t402181 | Mycobacterium sp. 852014- | 1 | 10 |
| | 52450_SCH5900713 | | |
| t401393 | Mycobacterium colombiense | 2 | 11 |
| t402076 | Mycobacterium asiaticum | 3 | 12 |
| t402353 | Mycobacterium gordonae | 4 | 13 |
| t402006 | B. diminuta (variant G1-C74) | 5 | 14 |
| t401609 | Prosthecomicrobium hirschii | 6 | 15 |
| t401992 | Brevundimonas diminuta | 7 | 16 |
| N/A | Brevundimonas diminuta | 8 | |
| | (IVH3) | | |
| N/A | Homo sapiens (PTER) | 9 | 17 |
| N/A | Brevundimonas diminuta | 18 | 19 |
| | (UniProt No. P0A434) | | |
| t339870 | N/A | N/A | N/A |
| (negative | | | |
| control) | | | |
| t402287 | Mycobacterium kansasii 824 | 22 | 23 |
| t402121 | Desulfatibacillum | 24 | 25 |
| | alkenivorans DSM 16219 | | |
| t401421 | Rhizobiaceae bacterium | 26 | 27 |
| t401451 | Providencia heimbachae | 28 | 29 |
| | ATCC 35613 | | |
| t402024 | Pricia antarctica | 30 | 31 |
| t402094 | Proteus mirabilis | 32 | 33 |
| t402397 | Salmonella enterica subsp. | 34 | 35 |
| | enterica serovar Give str. S5- | | |
| | 487 | | |

### Example 2: Enzyme Engineering

Four PTEs and one PIER identified in Example 1 (expressed in strains t402353 (SEQ ID NO: 4), t402181 (SEQ ID NO: 1), t401393 (SEQ ID NO: 2), t402076 (SEQ ID NO: 3) and t401609 (SEQ ID NO: 6)) were selected as templates for engineering to increase activity against V-agents. Approximately 2,053 rationally engineered enzymes were created. The screening included the generation of two libraries for enzyme engineering.

The first library (“PTE library”) was created using PTEs identified in Example 1, which were expressed in strains t402353 (SEQ ID NO: 4), t402181 (SEQ ID NO: 1), t401393 (SEQ ID NO: 2), and t402076 (SEQ ID NO: 3).

SEQ ID NOs: 1-4 were used as templates for enzyme engineering in which active site mutations, mutations at residues distal to the active site, and combinations thereof were created. More specifically, amino acid substitutions were generated at or near the active site, at residues throughout the protein structure (e.g., distal residues), and at a broader constellation of amino acid residues around the active site. The resulting PTE library was screened for increased activity against VX and VR.

The second library (“PTER library”) was created using a PTER identified in Example 1. The PTER expressed in strain t401609 (SEQ ID NO: 6) was used as a template to engineer and screen for increased activity against VX and VR.

The following table summarizes the PTE and PTER templates used and the number of engineered enzymes obtained and screened for VX and VR activities in the libraries generated.

TABLE 6
Enzymes Engineered & Screened for each Enzyme Template

| | | Number of Enzymes |
|---|---|---|
| Library | Enzyme Template | Engineered & Screened |
| PTE | t402181 (SEQ ID NO: 1) | 355 |
| PTE | t401393 (SEQ ID NO: 2) | 434 |
| PTE | t402076 (SEQ ID NO: 3) | 405 |
| PTE | t402353 (SEQ ID NO: 4) | 422 |
| PTER | t401609 (SEQ ID NO: 6) | 437 |

The following table provides the VX and VR activities of each engineered enzyme obtained by mutating one of the five PTE or PTER templates identified in Table 6, the point mutation in each engineered enzyme relative to the enzyme template, and the source organism of each enzyme template.

TABLE 7
Point Mutations of the PTEs and PTERs

| | | | | PTE or | | |
|---|---|---|---|---|---|---|
| | | Template | Mutation | PTER | | |
| Strain | Source organism of | SEQ ID | (relative to | SEQ ID | VX | VR |
| ID | template PTE or PTER | NO: | Template) | NO: | Activity | Activity |
| t815701 | Mycobacterium sp. 852014- | 1 | H22S | 36 | − | − |
| | 52450_SCH5900713 | | | | | |
| t815697 | Mycobacterium sp. 852014- | 1 | H22T | 37 | − | − |
| | 52450_SCH5900713 | | | | | |
| t808998 | Mycobacterium sp. 852014- | 1 | E23D | 38 | − | − |
| | 52450_SCH5900713 | | | | | |
| t810722 | Mycobacterium sp. 852014- | 1 | H24N | 39 | − | − |
| | 52450_SCH5900713 | | | | | |
| t809489 | Mycobacterium sp. 852014- | 1 | V25I | 40 | − | + |
| | 52450_SCH5900713 | | | | | |
| t815553 | Mycobacterium sp. 852014- | 1 | V25M | 41 | + | + |
| | 52450_SCH5900713 | | | | | |
| t809589 | Mycobacterium sp. 852014- | 1 | I27C | 42 | + | − |
| | 52450_SCH5900713 | | | | | |
| t815653 | Mycobacterium sp. 852014- | 1 | I27D | 43 | − | − |
| | 52450_SCH5900713 | | | | | |
| t809899 | Mycobacterium sp. 852014- | 1 | I27E | 44 | − | − |
| | 52450_SCH5900713 | | | | | |
| t809726 | Mycobacterium sp. 852014- | 1 | I27F | 45 | + | + |
| | 52450_SCH5900713 | | | | | |
| t809143 | Mycobacterium sp. 852014- | 1 | I27H | 46 | − | − |
| | 52450_SCH5900713 | | | | | |
| t809274 | Mycobacterium sp. 852014- | 1 | I27K | 47 | − | − |
| | 52450_SCH5900713 | | | | | |
| t810592 | Mycobacterium sp. 852014- | 1 | I27M | 48 | + | + |
| | 52450_SCH5900713 | | | | | |
| t809768 | Mycobacterium sp. 852014- | 1 | I27N | 49 | − | − |
| | 52450_SCH5900713 | | | | | |
| t809763 | Mycobacterium sp. 852014- | 1 | I27P | 50 | − | − |
| | 52450_SCH5900713 | | | | | |
| t810146 | Mycobacterium sp. 852014- | 1 | I27Q | 51 | + | − |
| | 52450_SCH5900713 | | | | | |
| t809437 | Mycobacterium sp. 852014- | 1 | I27R | 52 | − | − |
| | 52450_SCH5900713 | | | | | |
| t809236 | Mycobacterium sp. 852014- | 1 | I27T | 53 | + | + |
| | 52450_SCH5900713 | | | | | |
| t809422 | Mycobacterium sp. 852014- | 1 | I27W | 54 | − | + |
| | 52450_SCH5900713 | | | | | |
| t810109 | Mycobacterium sp. 852014- | 1 | I27Y | 55 | + | − |
| | 52450_SCH5900713 | | | | | |
| 1810457 | Mycobacterium sp. 852014- | 1 | V66I | 56 | + | + |
| | 52450_SCH5900713 | | | | | |
| t810104 | Mycobacterium sp. 852014- | 1 | L68A | 57 | + | − |
| | 52450_SCH5900713 | | | | | |
| t815549 | Mycobacterium sp. 852014- | 1 | L68C | 58 | + | + |
| | 52450_SCH5900713 | | | | | |
| t809678 | Mycobacterium sp. 852014- | 1 | L68M | 59 | + | + |
| | 52450_SCH5900713 | | | | | |
| t809453 | Mycobacterium sp. 852014- | 1 | L68N | 60 | + | + |
| | 52450_SCH5900713 | | | | | |
| t810140 | Mycobacterium sp. 852014- | 1 | L68Q | 61 | − | − |
| | 52450_SCH5900713 | | | | | |
| t809665 | Mycobacterium sp. 852014- | 1 | T69Q | 62 | − | − |
| | 52450_SCH5900713 | | | | | |
| t810417 | Mycobacterium sp. 852014- | 1 | T69S | 63 | + | + |
| | 52450_SCH5900713 | | | | | |
| t809186 | Mycobacterium sp. 852014- | 1 | V70C | 64 | + | + |
| | 52450_SCH5900713 | | | | | |
| t808987 | Mycobacterium sp. 852014- | 1 | V70P | 65 | + | + |
| | 52450_SCH5900713 | | | | | |
| t809003 | Mycobacterium sp. 852014- | 1 | V70T | 66 | + | + |
| | 52450_SCH5900713 | | | | | |
| t809611 | Mycobacterium sp. 852014- | 1 | L73C | 67 | − | − |
| | 52450_SCH5900713 | | | | | |
| t809144 | Mycobacterium sp. 852014- | 1 | L73H | 68 | − | − |
| | 52450_SCH5900713 | | | | | |
| t808376 | Mycobacterium sp. 852014- | 1 | L73M | 69 | − | − |
| | 52450_SCH5900713 | | | | | |
| t815714 | Mycobacterium sp. 852014- | 1 | Y98F | 70 | − | − |
| | 52450_SCH5900713 | | | | | |
| t815674 | Mycobacterium sp. 852014- | 1 | Y98H | 71 | − | − |
| | 52450_SCH5900713 | | | | | |
| t810561 | Mycobacterium sp. 852014- | 1 | Y98W | 72 | − | − |
| | 52450_SCH5900713 | | | | | |
| t808454 | Mycobacterium sp. 852014- | 1 | L144I | 73 | + | + |
| | 52450_SCH5900713 | | | | | |
| t815547 | Mycobacterium sp. 852014- | 1 | K145C | 74 | − | − |
| | 52450_SCH5900713 | | | | | |
| t810029 | Mycobacterium sp. 852014- | 1 | K145E | 75 | − | − |
| | 52450_SCH5900713 | | | | | |
| t809984 | Mycobacterium sp. 852014- | 1 | K145R | 76 | − | + |
| | 52450_SCH5900713 | | | | | |
| t810407 | Mycobacterium sp. 852014- | 1 | K145S | 77 | − | − |
| | 52450_SCH5900713 | | | | | |
| t810694 | Mycobacterium sp. 852014- | 1 | C146F | 78 | − | − |
| | 52450_SCH5900713 | | | | | |
| t809999 | Mycobacterium sp. 852014- | 1 | A147F | 79 | + | + |
| | 52450_SCH5900713 | | | | | |
| t809221 | Mycobacterium sp. 852014- | 1 | A147G | 80 | + | + |
| | 52450_SCH5900713 | | | | | |
| t810241 | Mycobacterium sp. 852014- | 1 | T148C | 81 | + | + |
| | 52450_SCH5900713 | | | | | |
| t809389 | Mycobacterium sp. 852014- | 1 | T148M | 82 | + | + |
| | 52450_SCH5900713 | | | | | |
| t809523 | Mycobacterium sp. 852014- | 1 | T148V | 83 | + | + |
| | 52450_SCH5900713 | | | | | |
| t810462 | Mycobacterium sp. 852014- | 1 | V164I | 84 | + | + |
| | 52450_SCH5900713 | | | | | |
| t810059 | Mycobacterium sp. 852014- | 1 | S176C | 85 | + | + |
| | 52450_SCH5900713 | | | | | |
| t810245 | Mycobacterium sp. 852014- | 1 | S176F | 86 | + | − |
| | 52450_SCH5900713 | | | | | |
| t808431 | Mycobacterium sp. 852014- | 1 | S176H | 87 | − | + |
| | 52450_SCH5900713 | | | | | |
| t809325 | Mycobacterium sp. 852014- | 1 | S176I | 88 | + | + |
| | 52450_SCH5900713 | | | | | |
| t810123 | Mycobacterium sp. 852014- | 1 | S176M | 89 | + | + |
| | 52450_SCH5900713 | | | | | |
| t808423 | Mycobacterium sp. 852014- | 1 | S176T | 90 | + | + |
| | 52450_SCH5900713 | | | | | |
| t810584 | Mycobacterium sp. 852014- | 1 | S176V | 91 | + | + |
| | 52450_SCH5900713 | | | | | |
| t809985 | Mycobacterium sp. 852014- | 1 | S176W | 92 | − | − |
| | 52450_SCH5900713 | | | | | |
| t808372 | Mycobacterium sp. 852014- | 1 | S176Y | 93 | + | + |
| | 52450_SCH5900713 | | | | | |
| t810576 | Mycobacterium sp. 852014- | 1 | T177C | 94 | + | + |
| | 52450_SCH5900713 | | | | | |
| t810329 | Mycobacterium sp. 852014- | 1 | T177L | 95 | + | − |
| | 52450_SCH5900713 | | | | | |
| t809702 | Mycobacterium sp. 852014- | 1 | T177V | 96 | − | − |
| | 52450_SCH5900713 | | | | | |
| t809917 | Mycobacterium sp. 852014- | 1 | H178A | 97 | − | − |
| | 52450_SCH5900713 | | | | | |
| t809622 | Mycobacterium sp. 852014- | 1 | H178S | 98 | − | − |
| | 52450_SCH5900713 | | | | | |
| t810350 | Mycobacterium sp. 852014- | 1 | H178T | 99 | − | − |
| | 52450_SCH5900713 | | | | | |
| t809244 | Mycobacterium sp. 852014- | 1 | T179C | 100 | + | + |
| | 52450_SCH5900713 | | | | | |
| t815702 | Mycobacterium sp. 852014- | 1 | T179E | 101 | + | − |
| | 52450_SCH5900713 | | | | | |
| t809750 | Mycobacterium sp. 852014- | 1 | T179S | 102 | + | + |
| | 52450_SCH5900713 | | | | | |
| t809908 | Mycobacterium sp. 852014- | 1 | T179V | 103 | − | − |
| | 52450_SCH5900713 | | | | | |
| t815687 | Mycobacterium sp. 852014- | 1 | A181C | 104 | + | + |
| | 52450_SCH5900713 | | | | | |
| t810201 | Mycobacterium sp. 852014- | 1 | A181H | 105 | − | − |
| | 52450_SCH5900713 | | | | | |
| t809507 | Mycobacterium sp. 852014- | 1 | A181P | 106 | + | + |
| | 52450_SCH5900713 | | | | | |
| t810151 | Mycobacterium sp. 852014- | 1 | A181S | 107 | + | − |
| | 52450_SCH5900713 | | | | | |
| t810498 | Mycobacterium sp. 852014- | 1 | G206A | 108 | − | − |
| | 52450_SCH5900713 | | | | | |
| t810248 | Mycobacterium sp. 852014- | 1 | G206M | 109 | − | − |
| | 52450_SCH5900713 | | | | | |
| t815564 | Mycobacterium sp. 852014- | 1 | G206S | 110 | − | − |
| | 52450_SCH5900713 | | | | | |
| t827637 | Mycobacterium sp. 852014- | 1 | S208A | 111 | + | + |
| | 52450_SCH5900713 | | | | | |
| t809994 | Mycobacterium sp. 852014- | 1 | S208C | 112 | − | − |
| | 52450_SCH5900713 | | | | | |
| t815617 | Mycobacterium sp. 852014- | 1 | S208Q | 113 | − | − |
| | 52450_SCH5900713 | | | | | |
| t815710 | Mycobacterium sp. 852014- | 1 | S208T | 114 | + | + |
| | 52450_SCH5900713 | | | | | |
| t809819 | Mycobacterium sp. 852014- | 1 | G209N | 115 | + | − |
| | 52450_SCH5900713 | | | | | |
| 809328 | Mycobacterium sp. 852014- | 1 | D210E | 116 | − | − |
| | 52450_SCH5900713 | | | | | |
| t809745 | Mycobacterium sp. 852014- | 1 | D210S | 117 | − | − |
| | 52450_SCH5900713 | | | | | |
| t809592 | Mycobacterium sp. 852014- | 1 | D210T | 118 | − | − |
| | 52450_SCH5900713 | | | | | |
| t808958 | Mycobacterium sp. 852014- | 1 | G228A | 119 | − | − |
| | 52450_SCH5900713 | | | | | |
| t809482 | Mycobacterium sp. 852014- | 1 | G228E | 120 | − | − |
| | 52450_SCH5900713 | | | | | |
| t809892 | Mycobacterium sp. 852014- | 1 | G228H | 121 | − | − |
| | 52450_SCH5900713 | | | | | |
| t809646 | Mycobacterium sp. 852014- | 1 | G228S | 122 | + | + |
| | 52450_SCH5900713 | | | | | |
| t810244 | Mycobacterium sp. 852014- | 1 | D230P | 123 | − | − |
| | 52450_SCH5900713 | | | | | |
| t809406 | Mycobacterium sp. 852014- | 1 | R231G | 124 | − | − |
| | 52450_SCH5900713 | | | | | |
| t809666 | Mycobacterium sp. 852014- | 1 | R231H | 125 | − | − |
| | 52450_SCH5900713 | | | | | |
| t809190 | Mycobacterium sp. 852014- | 1 | R231L | 126 | − | − |
| | 52450_SCH5900713 | | | | | |
| t809840 | Mycobacterium sp. 852014- | 1 | R231M | 127 | − | − |
| | 52450_SCH5900713 | | | | | |
| t810312 | Mycobacterium sp. 852014- | 1 | R231Q | 128 | − | − |
| | 52450_SCH5900713 | | | | | |
| t809715 | Mycobacterium sp. 852014- | 1 | R231T | 129 | − | − |
| | 52450_SCH5900713 | | | | | |
| t809725 | Mycobacterium sp. 852014- | 1 | R231W | 130 | − | − |
| | 52450_SCH5900713 | | | | | |
| t810370 | Mycobacterium sp. 852014- | 1 | S262C | 131 | − | − |
| | 52450_SCH5900713 | | | | | |
| t810172 | Mycobacterium sp. 852014- | 1 | H263C | 132 | + | + |
| | 52450_SCH5900713 | | | | | |
| t810546 | Mycobacterium sp. 852014- | 1 | H263F | 133 | − | − |
| | 52450_SCH5900713 | | | | | |
| t815676 | Mycobacterium sp. 852014- | 1 | H263M | 134 | + | + |
| | 52450_SCH5900713 | | | | | |
| t809447 | Mycobacterium sp. 852014- | 1 | H263N | 135 | + | + |
| | 52450_SCH5900713 | | | | | |
| t809354 | Mycobacterium sp. 852014- | 1 | H263S | 136 | + | + |
| | 52450_SCH5900713 | | | | | |
| t810220 | Mycobacterium sp. 852014- | 1 | H263T | 137 | + | + |
| | 52450_SCH5900713 | | | | | |
| t810126 | Mycobacterium sp. 852014- | 1 | D264N | 138 | − | − |
| | 52450_SCH5900713 | | | | | |
| t809771 | Mycobacterium sp. 852014- | 1 | D264P | 139 | − | − |
| | 52450_SCH5900713 | | | | | |
| t810280 | Mycobacterium sp. 852014- | 1 | D264S | 140 | − | − |
| | 52450_SCH5900713 | | | | | |
| t809652 | Mycobacterium sp. 852014- | 1 | A265C | 141 | + | + |
| | 52450_SCH5900713 | | | | | |
| t809524 | Mycobacterium sp. 852014- | 1 | A265F | 142 | + | ++ |
| | 52450_SCH5900713 | | | | | |
| t809968 | Mycobacterium sp. 852014- | 1 | A265L | 143 | − | − |
| | 52450_SCH5900713 | | | | | |
| t809930 | Mycobacterium sp. 852014- | 1 | A265M | 144 | + | ++ |
| | 52450_SCH5900713 | | | | | |
| t810397 | Mycobacterium sp. 852014- | 1 | A265T | 145 | + | + |
| | 52450_SCH5900713 | | | | | |
| t809365 | Mycobacterium sp. 852014- | 1 | A265W | 146 | + | ++ |
| | 52450_SCH5900713 | | | | | |
| t810027 | Mycobacterium sp. 852014- | 1 | A265Y | 147 | + | ++ |
| | 52450_SCH5900713 | | | | | |
| t810150 | Mycobacterium sp. 852014- | 1 | N266A | 148 | + | ++ |
| | 52450_SCH5900713 | | | | | |
| t810304 | Mycobacterium sp. 852014- | 1 | N266G | 149 | ++ | ++ |
| | 52450_SCH5900713 | | | | | |
| t810152 | Mycobacterium sp. 852014- | 1 | N266M | 150 | ++ | ++ |
| | 52450_SCH5900713 | | | | | |
| t810211 | Mycobacterium sp. 852014- | 1 | N266S | 151 | + | + |
| | 52450_SCH5900713 | | | | | |
| t810436 | Mycobacterium sp. 852014- | 1 | N266T | 152 | ++ | ++ |
| | 52450_SCH5900713 | | | | | |
| t810063 | Mycobacterium sp. 852014- | 1 | N266W | 153 | + | − |
| | 52450_SCH5900713 | | | | | |
| t809175 | Mycobacterium sp. 852014- | 1 | C267A | 154 | + | + |
| | 52450_SCH5900713 | | | | | |
| t809459 | Mycobacterium sp. 852014- | 1 | C267T | 155 | + | + |
| | 52450_SCH5900713 | | | | | |
| t810455 | Mycobacterium sp. 852014- | 1 | C267W | 156 | + | ++ |
| | 52450_SCH5900713 | | | | | |
| t809976 | Mycobacterium sp. 852014- | 1 | W284C | 157 | + | ++ |
| | 52450_SCH5900713 | | | | | |
| t810677 | Mycobacterium sp. 852014- | 1 | W284D | 158 | + | + |
| | 52450_SCH5900713 | | | | | |
| t809638 | Mycobacterium sp. 852014- | 1 | W284F | 159 | + | ++ |
| | 52450_SCH5900713 | | | | | |
| t810083 | Mycobacterium sp. 852014- | 1 | W284H | 160 | + | ++ |
| | 52450_SCH5900713 | | | | | |
| t809820 | Mycobacterium sp. 852014- | 1 | W284I | 161 | − | − |
| | 52450_SCH5900713 | | | | | |
| t810651 | Mycobacterium sp. 852014- | 1 | W284K | 162 | − | − |
| | 52450_SCH5900713 | | | | | |
| t809778 | Mycobacterium sp. 852014- | 1 | W284N | 163 | + | ++ |
| | 52450_SCH5900713 | | | | | |
| t810001 | Mycobacterium sp. 852014- | 1 | W284P | 164 | − | − |
| | 52450_SCH5900713 | | | | | |
| t809423 | Mycobacterium sp. 852014- | 1 | W284Q | 165 | − | − |
| | 52450_SCH5900713 | | | | | |
| t810168 | Mycobacterium sp. 852014- | 1 | W284R | 166 | − | − |
| | 52450_SCH5900713 | | | | | |
| t809282 | Mycobacterium sp. 852014- | 1 | W284Y | 167 | + | ++ |
| | 52450_SCH5900713 | | | | | |
| t810643 | Mycobacterium sp. 852014- | 1 | Y286M | 168 | + | + |
| | 52450_SCH5900713 | | | | | |
| t809673 | Mycobacterium sp. 852014- | 1 | Y286W | 169 | + | + |
| | 52450_SCH5900713 | | | | | |
| t810593 | Mycobacterium colombiense | 2 | S2A | 170 | + | + |
| t809215 | Mycobacterium colombiense | 2 | S2K | 171 | + | + |
| t809576 | Mycobacterium colombiense | 2 | S2T | 172 | + | + |
| t810372 | Mycobacterium colombiense | 2 | E3K | 173 | + | + |
| t809716 | Mycobacterium colombiense | 2 | E3Q | 174 | + | + |
| t810634 | Mycobacterium colombiense | 2 | E3T | 175 | + | + |
| t815609 | Mycobacterium colombiense | 2 | L4I | 176 | + | + |
| t810601 | Mycobacterium colombiense | 2 | L4V | 177 | + | + |
| t809997 | Mycobacterium colombiense | 2 | N5M | 178 | ++ | + |
| t808393 | Mycobacterium colombiense | 2 | N5Q | 179 | + | + |
| t810725 | Mycobacterium colombiense | 2 | N5R | 180 | + | + |
| t809630 | Mycobacterium colombiense | 2 | R8C | 181 | + | + |
| t809755 | Mycobacterium colombiense | 2 | R8L | 182 | + | + |
| t809803 | Mycobacterium colombiense | 2 | R8T | 183 | + | + |
| t810636 | Mycobacterium colombiense | 2 | S10P | 184 | + | + |
| t810494 | Mycobacterium colombiense | 2 | D12E | 185 | + | + |
| t815586 | Mycobacterium colombiense | 2 | D12P | 186 | + | + |
| t810625 | Mycobacterium colombiense | 2 | T13A | 187 | ++ | + |
| 1810518 | Mycobacterium colombiense | 2 | T13P | 188 | + | + |
| t810079 | Mycobacterium colombiense | 2 | A14D | 189 | + | + |
| t810330 | Mycobacterium colombiense | 2 | A14E | 190 | + | + |
| t809945 | Mycobacterium colombiense | 2 | A14S | 191 | + | + |
| t809883 | Mycobacterium colombiense | 2 | A15D | 192 | + | + |
| t809514 | Mycobacterium colombiense | 2 | A15E | 193 | ++ | + |
| t809260 | Mycobacterium colombiense | 2 | A15Q | 194 | + | + |
| t809657 | Mycobacterium colombiense | 2 | L16M | 195 | ++ | + |
| t809539 | Mycobacterium colombiense | 2 | V18I | 196 | ++ | + |
| t810317 | Mycobacterium colombiense | 2 | V18M | 197 | + | + |
| t809485 | Mycobacterium colombiense | 2 | M28D | 198 | ++ | + |
| t808375 | Mycobacterium colombiense | 2 | T29S | 199 | ++ | + |
| t815628 | Mycobacterium colombiense | 2 | T30P | 200 | ++ | + |
| t810452 | Mycobacterium colombiense | 2 | T30S | 201 | + | + |
| t810689 | Mycobacterium colombiense | 2 | T30W | 202 | + | + |
| t810671 | Mycobacterium colombiense | 2 | E31G | 203 | ++ | + |
| t810432 | Mycobacterium colombiense | 2 | I32F | 204 | ++ | + |
| t810080 | Mycobacterium colombiense | 2 | I32M | 205 | + | + |
| t809159 | Mycobacterium colombiense | 2 | I32V | 206 | + | + |
| t809612 | Mycobacterium colombiense | 2 | I32W | 207 | ++ | + |
| t810438 | Mycobacterium colombiense | 2 | A33W | 208 | + | + |
| t815614 | Mycobacterium colombiense | 2 | E34Q | 209 | + | + |
| t809358 | Mycobacterium colombiense | 2 | N35D | 210 | + | + |
| t810086 | Mycobacterium colombiense | 2 | Y36F | 211 | + | + |
| t809758 | Mycobacterium colombiense | 2 | Y36H | 212 | ++ | + |
| t810039 | Mycobacterium colombiense | 2 | Y36W | 213 | + | + |
| t810728 | Mycobacterium colombiense | 2 | E38D | 214 | + | + |
| t810276 | Mycobacterium colombiense | 2 | E38H | 215 | + | − |
| t809540 | Mycobacterium colombiense | 2 | A39P | 216 | + | + |
| t809728 | Mycobacterium colombiense | 2 | W40F | 217 | ++ | + |
| t810380 | Mycobacterium colombiense | 2 | D42N | 218 | + | + |
| t809916 | Mycobacterium colombiense | 2 | E43D | 219 | + | + |
| t810533 | Mycobacterium colombiense | 2 | D44E | 220 | + | + |
| t810037 | Mycobacterium colombiense | 2 | D44N | 221 | + | + |
| t810493 | Mycobacterium colombiense | 2 | V47I | 222 | + | + |
| t809120 | Mycobacterium colombiense | 2 | V47M | 223 | ++ | + |
| t809211 | Mycobacterium colombiense | 2 | A48E | 224 | + | + |
| t808983 | Mycobacterium colombiense | 2 | A48W | 225 | + | + |
| t809676 | Mycobacterium colombiense | 2 | D49C | 226 | ++ | + |
| t810605 | Mycobacterium colombiense | 2 | D49H | 227 | + | + |
| t815618 | Mycobacterium colombiense | 2 | D49M | 228 | ++ | + |
| t809911 | Mycobacterium colombiense | 2 | D49W | 229 | + | + |
| t809369 | Mycobacterium colombiense | 2 | V51I | 230 | + | + |
| t808456 | Mycobacterium colombiense | 2 | V51L | 231 | + | + |
| t810516 | Mycobacterium colombiense | 2 | K52R | 232 | + | + |
| t808988 | Mycobacterium colombiense | 2 | R53E | 233 | ++ | + |
| t815571 | Mycobacterium colombiense | 2 | R53Q | 234 | + | + |
| t810446 | Mycobacterium colombiense | 2 | N55K | 235 | + | + |
| t815638 | Mycobacterium colombiense | 2 | E56D | 236 | + | + |
| t809610 | Mycobacterium colombiense | 2 | E56Q | 237 | + | + |
| t815570 | Mycobacterium colombiense | 2 | E56R | 238 | + | + |
| t810313 | Mycobacterium colombiense | 2 | L57F | 239 | + | + |
| t809618 | Mycobacterium colombiense | 2 | L57Y | 240 | − | − |
| t809338 | Mycobacterium colombiense | 2 | A59E | 241 | + | + |
| t810119 | Mycobacterium colombiense | 2 | A59Q | 242 | + | + |
| t810002 | Mycobacterium colombiense | 2 | R60A | 243 | + | + |
| t809857 | Mycobacterium colombiense | 2 | R60H | 244 | + | + |
| t809484 | Mycobacterium colombiense | 2 | D63N | 245 | ++ | + |
| t810434 | Mycobacterium colombiense | 2 | T64S | 246 | + | + |
| t809563 | Mycobacterium colombiense | 2 | G72D | 247 | + | + |
| t809947 | Mycobacterium colombiense | 2 | G74R | 248 | − | − |
| t810719 | Mycobacterium colombiense | 2 | Y76D | 249 | + | + |
| t808455 | Mycobacterium colombiense | 2 | Y76N | 250 | + | + |
| t815585 | Mycobacterium colombiense | 2 | I77P | 251 | + | − |
| t810069 | Mycobacterium colombiense | 2 | I77V | 252 | + | + |
| t810302 | Mycobacterium colombiense | 2 | P78D | 253 | ++ | + |
| t809559 | Mycobacterium colombiense | 2 | P78E | 254 | ++ | + |
| t810212 | Mycobacterium colombiense | 2 | I80L | 255 | + | + |
| t810224 | Mycobacterium colombiense | 2 | I80M | 256 | + | + |
| t808449 | Mycobacterium colombiense | 2 | I80V | 257 | + | + |
| t810612 | Mycobacterium colombiense | 2 | A81R | 258 | + | + |
| t810603 | Mycobacterium colombiense | 2 | R82E | 259 | + | + |
| t809637 | Mycobacterium colombiense | 2 | R82K | 260 | + | + |
| t809025 | Mycobacterium colombiense | 2 | V83I | 261 | ++ | + |
| t809654 | Mycobacterium colombiense | 2 | V83L | 262 | + | + |
| t810480 | Mycobacterium colombiense | 2 | A84S | 263 | + | + |
| t810050 | Mycobacterium colombiense | 2 | A85E | 264 | + | + |
| t809428 | Mycobacterium colombiense | 2 | A85R | 265 | ++ | + |
| t810120 | Mycobacterium colombiense | 2 | E86R | 266 | + | + |
| t810377 | Mycobacterium colombiense | 2 | T87S | 267 | + | + |
| t810466 | Mycobacterium colombiense | 2 | E88G | 268 | + | + |
| t810529 | Mycobacterium colombiense | 2 | L89M | 269 | ++ | + |
| t810401 | Mycobacterium colombiense | 2 | L89V | 270 | + | + |
| t809084 | Mycobacterium colombiense | 2 | N90H | 271 | ++ | + |
| t810414 | Mycobacterium colombiense | 2 | I91V | 272 | + | + |
| t809671 | Mycobacterium colombiense | 2 | V92I | 273 | + | + |
| t809816 | Mycobacterium colombiense | 2 | V93C | 274 | + | + |
| t810450 | Mycobacterium colombiense | 2 | T99C | 275 | ++ | + |
| t810588 | Mycobacterium colombiense | 2 | V103H | 276 | ++ | ++ |
| t810609 | Mycobacterium colombiense | 2 | V103W | 277 | + | + |
| t815552 | Mycobacterium colombiense | 2 | V103Y | 278 | + | + |
| t809096 | Mycobacterium colombiense | 2 | M105W | 279 | + | + |
| t809565 | Mycobacterium colombiense | 2 | Y106F | 280 | + | + |
| t810731 | Mycobacterium colombiense | 2 | Y106H | 281 | + | + |
| t809528 | Mycobacterium colombiense | 2 | Y106W | 282 | ++ | + |
| t810115 | Mycobacterium colombiense | 2 | F107M | 283 | ++ | + |
| t810619 | Mycobacterium colombiense | 2 | F107W | 284 | + | + |
| t810402 | Mycobacterium colombiense | 2 | F107Y | 285 | + | + |
| t809359 | Mycobacterium colombiense | 2 | H108R | 286 | − | − |
| t809705 | Mycobacterium colombiense | 2 | Y109F | 287 | + | + |
| t809119 | Mycobacterium colombiense | 2 | Y109W | 288 | + | + |
| t810504 | Mycobacterium colombiense | 2 | L110M | 289 | + | + |
| t810092 | Mycobacterium colombiense | 2 | L110W | 290 | + | + |
| t815607 | Mycobacterium colombiense | 2 | E115D | 291 | + | + |
| t809487 | Mycobacterium colombiense | 2 | G118S | 292 | ++ | + |
| t809020 | Mycobacterium colombiense | 2 | G118T | 293 | + | + |
| t810560 | Mycobacterium colombiense | 2 | E120D | 294 | + | + |
| t809109 | Mycobacterium colombiense | 2 | I121E | 295 | ++ | + |
| t809724 | Mycobacterium colombiense | 2 | I121Q | 296 | + | + |
| t810537 | Mycobacterium colombiense | 2 | M1221 | 297 | ++ | + |
| t809445 | Mycobacterium colombiense | 2 | M122L | 298 | + | + |
| t809636 | Mycobacterium colombiense | 2 | T123A | 299 | + | + |
| t810648 | Mycobacterium colombiense | 2 | D124E | 300 | + | + |
| t809426 | Mycobacterium colombiense | 2 | V127I | 301 | ++ | + |
| t810205 | Mycobacterium colombiense | 2 | R128H | 302 | + | + |
| t809567 | Mycobacterium colombiense | 2 | R128N | 303 | + | + |
| t809957 | Mycobacterium colombiense | 2 | Q132D | 304 | + | + |
| t810371 | Mycobacterium colombiense | 2 | Q132E | 305 | + | + |
| 1810654 | Mycobacterium colombiense | 2 | I134V | 306 | + | + |
| t815569 | Mycobacterium colombiense | 2 | A135E | 307 | + | + |
| t810390 | Mycobacterium colombiense | 2 | A135G | 308 | + | + |
| t808407 | Mycobacterium colombiense | 2 | D136G | 309 | + | + |
| t809954 | Mycobacterium colombiense | 2 | I139V | 310 | + | + |
| t810203 | Mycobacterium colombiense | 2 | K140H | 311 | + | + |
| t810360 | Mycobacterium colombiense | 2 | K140R | 312 | + | + |
| t808441 | Mycobacterium colombiense | 2 | T150H | 313 | + | + |
| t808451 | Mycobacterium colombiense | 2 | T150S | 314 | + | + |
| t810375 | Mycobacterium colombiense | 2 | T150Y | 315 | + | + |
| t810192 | Mycobacterium colombiense | 2 | P151D | 316 | ++ | + |
| t810556 | Mycobacterium colombiense | 2 | P151H | 317 | ++ | + |
| t810268 | Mycobacterium colombiense | 2 | P151N | 318 | ++ | + |
| t809996 | Mycobacterium colombiense | 2 | P151W | 319 | ++ | ++ |
| t810130 | Mycobacterium colombiense | 2 | V153I | 320 | + | + |
| t810627 | Mycobacterium colombiense | 2 | V153W | 321 | + | − |
| t809615 | Mycobacterium colombiense | 2 | P155D | 322 | ++ | + |
| t815679 | Mycobacterium colombiense | 2 | P155E | 323 | + | + |
| t809022 | Mycobacterium colombiense | 2 | G156W | 324 | + | + |
| t809324 | Mycobacterium colombiense | 2 | V157E | 325 | − | − |
| t815692 | Mycobacterium colombiense | 2 | E158H | 326 | + | + |
| t810509 | Mycobacterium colombiense | 2 | A165C | 327 | + | + |
| t809928 | Mycobacterium colombiense | 2 | Q166R | 328 | + | + |
| t810105 | Mycobacterium colombiense | 2 | H168Q | 329 | + | + |
| t810709 | Mycobacterium colombiense | 2 | G182D | 330 | + | + |
| t810283 | Mycobacterium colombiense | 2 | L183T | 331 | + | + |
| 1810733 | Mycobacterium colombiense | 2 | L187F | 332 | + | + |
| t815698 | Mycobacterium colombiense | 2 | L187H | 333 | + | + |
| t810346 | Mycobacterium colombiense | 2 | L187M | 334 | + | + |
| t810071 | Mycobacterium colombiense | 2 | E188C | 335 | + | − |
| t810285 | Mycobacterium colombiense | 2 | E188W | 336 | + | + |
| t810604 | Mycobacterium colombiense | 2 | Q190I | 337 | + | + |
| t810122 | Mycobacterium colombiense | 2 | Q190L | 338 | + | + |
| 1809719 | Mycobacterium colombiense | 2 | K191R | 339 | ++ | + |
| t810408 | Mycobacterium colombiense | 2 | F193L | 340 | + | + |
| t809568 | Mycobacterium colombiense | 2 | E194D | 341 | ++ | + |
| t809841 | Mycobacterium colombiense | 2 | E195D | 342 | + | + |
| t809743 | Mycobacterium colombiense | 2 | L200P | 343 | + | + |
| t810553 | Mycobacterium colombiense | 2 | S201N | 344 | + | + |
| t810024 | Mycobacterium colombiense | 2 | R202H | 345 | + | + |
| t809738 | Mycobacterium colombiense | 2 | R202K | 346 | + | + |
| t809989 | Mycobacterium colombiense | 2 | V203C | 347 | + | + |
| t810215 | Mycobacterium colombiense | 2 | V203I | 348 | + | + |
| t810040 | Mycobacterium colombiense | 2 | I214H | 349 | − | − |
| t809111 | Mycobacterium colombiense | 2 | I214M | 350 | + | + |
| t810428 | Mycobacterium colombiense | 2 | G215D | 351 | + | + |
| t809736 | Mycobacterium colombiense | 2 | G215E | 352 | + | + |
| t808951 | Mycobacterium colombiense | 2 | E219D | 353 | + | + |
| t810502 | Mycobacterium colombiense | 2 | L220I | 354 | + | + |
| t809006 | Mycobacterium colombiense | 2 | L220M | 355 | + | + |
| t810322 | Mycobacterium colombiense | 2 | L220V | 356 | + | + |
| t810252 | Mycobacterium colombiense | 2 | I221A | 357 | + | + |
| t815642 | Mycobacterium colombiense | 2 | I221C | 358 | + | + |
| t809353 | Mycobacterium colombiense | 2 | I221M | 359 | + | + |
| t815620 | Mycobacterium colombiense | 2 | A222H | 360 | + | + |
| t810501 | Mycobacterium colombiense | 2 | A223R | 361 | + | + |
| t809760 | Mycobacterium colombiense | 2 | S225C | 362 | + | + |
| t809122 | Mycobacterium colombiense | 2 | Y226W | 363 | + | + |
| t815720 | Mycobacterium colombiense | 2 | L227I | 364 | + | + |
| t810661 | Mycobacterium colombiense | 2 | L227V | 365 | + | + |
| t810359 | Mycobacterium colombiense | 2 | D235C | 366 | + | + |
| t810476 | Mycobacterium colombiense | 2 | A236H | 367 | + | + |
| t808406 | Mycobacterium colombiense | 2 | A236K | 368 | ++ | ++ |
| t815619 | Mycobacterium colombiense | 2 | A236W | 369 | + | + |
| t809037 | Mycobacterium colombiense | 2 | L238H | 370 | ++ | + |
| t809347 | Mycobacterium colombiense | 2 | L238M | 371 | + | + |
| t810569 | Mycobacterium colombiense | 2 | L238Y | 372 | + | − |
| t810596 | Mycobacterium colombiense | 2 | P239S | 373 | + | + |
| t810054 | Mycobacterium colombiense | 2 | F240D | 374 | + | + |
| t810184 | Mycobacterium colombiense | 2 | F240W | 375 | + | + |
| t810468 | Mycobacterium colombiense | 2 | F240Y | 376 | + | + |
| t815662 | Mycobacterium colombiense | 2 | E241D | 377 | + | + |
| t810009 | Mycobacterium colombiense | 2 | D242E | 378 | + | + |
| t810630 | Mycobacterium colombiense | 2 | V244C | 379 | + | + |
| t808993 | Mycobacterium colombiense | 2 | N245D | 380 | + | + |
| t809648 | Mycobacterium colombiense | 2 | N245E | 381 | + | + |
| t809522 | Mycobacterium colombiense | 2 | N245R | 382 | ++ | + |
| t810405 | Mycobacterium colombiense | 2 | T246L | 383 | ++ | + |
| t810591 | Mycobacterium colombiense | 2 | T246M | 384 | + | + |
| t810166 | Mycobacterium colombiense | 2 | V247I | 385 | + | + |
| t809775 | Mycobacterium colombiense | 2 | V247L | 386 | + | + |
| t810552 | Mycobacterium colombiense | 2 | Q249E | 387 | + | + |
| t815718 | Mycobacterium colombiense | 2 | Q249H | 388 | + | + |
| t815583 | Mycobacterium colombiense | 2 | Q249R | 389 | + | + |
| t809669 | Mycobacterium colombiense | 2 | Q249W | 390 | + | + |
| t809108 | Mycobacterium colombiense | 2 | M250L | 391 | ++ | + |
| t810325 | Mycobacterium colombiense | 2 | C251I | 392 | + | + |
| t809737 | Mycobacterium colombiense | 2 | C251V | 393 | ++ | + |
| t808978 | Mycobacterium colombiense | 2 | E252H | 394 | + | + |
| t815649 | Mycobacterium colombiense | 2 | R253N | 395 | + | + |
| t810196 | Mycobacterium colombiense | 2 | H255W | 396 | − | − |
| t810121 | Mycobacterium colombiense | 2 | H255Y | 397 | + | + |
| t809762 | Mycobacterium colombiense | 2 | K258H | 398 | + | + |
| t810453 | Mycobacterium colombiense | 2 | K258R | 399 | + | + |
| t809220 | Mycobacterium colombiense | 2 | M259I | 400 | ++ | + |
| t809822 | Mycobacterium colombiense | 2 | A271G | 401 | + | + |
| t810666 | Mycobacterium colombiense | 2 | L272C | 402 | ++ | + |
| t808967 | Mycobacterium colombiense | 2 | L272H | 403 | + | + |
| t809895 | Mycobacterium colombiense | 2 | L272M | 404 | ++ | + |
| t809936 | Mycobacterium colombiense | 2 | L272W | 405 | ++ | ++ |
| 1809363 | Mycobacterium colombiense | 2 | D274G | 406 | + | + |
| 1810233 | Mycobacterium colombiense | 2 | D274W | 407 | + | + |
| t808999 | Mycobacterium colombiense | 2 | E275K | 408 | + | + |
| t809573 | Mycobacterium colombiense | 2 | L276W | 409 | + | − |
| t810644 | Mycobacterium colombiense | 2 | V277W | 410 | + | + |
| t809667 | Mycobacterium colombiense | 2 | S278R | 411 | + | + |
| t809644 | Mycobacterium colombiense | 2 | Q279H | 412 | + | + |
| t809628 | Mycobacterium colombiense | 2 | Q279K | 413 | + | + |
| t809569 | Mycobacterium colombiense | 2 | Q279R | 414 | + | + |
| t809124 | Mycobacterium colombiense | 2 | M281F | 415 | ++ | + |
| t815554 | Mycobacterium colombiense | 2 | M281Y | 416 | ++ | + |
| t810582 | Mycobacterium colombiense | 2 | P282G | 417 | + | + |
| t809535 | Mycobacterium colombiense | 2 | N283D | 418 | ++ | + |
| t810388 | Mycobacterium colombiense | 2 | N283G | 419 | + | ++ |
| t809729 | Mycobacterium colombiense | 2 | H285G | 420 | + | ++ |
| t810688 | Mycobacterium colombiense | 2 | L287T | 421 | + | + |
| t810610 | Mycobacterium colombiense | 2 | H288F | 422 | ++ | + |
| t810555 | Mycobacterium colombiense | 2 | H288Y | 423 | ++ | + |
| t809920 | Mycobacterium colombiense | 2 | I289L | 424 | + | + |
| t810351 | Mycobacterium colombiense | 2 | I289V | 425 | + | − |
| t809831 | Mycobacterium colombiense | 2 | H290F | 426 | + | + |
| t809098 | Mycobacterium colombiense | 2 | H290L | 427 | ++ | + |
| t808363 | Mycobacterium colombiense | 2 | N291D | 428 | + | + |
| t809192 | Mycobacterium colombiense | 2 | N291E | 429 | ++ | + |
| t810084 | Mycobacterium colombiense | 2 | N291R | 430 | + | + |
| 809403 | Mycobacterium colombiense | 2 | N291T | 431 | + | + |
| t809301 | Mycobacterium colombiense | 2 | D292N | 432 | + | + |
| t810188 | Mycobacterium colombiense | 2 | D292R | 433 | ++ | − |
| t810412 | Mycobacterium colombiense | 2 | V293F | 434 | − | − |
| t810640 | Mycobacterium colombiense | 2 | V293I | 435 | + | + |
| t810572 | Mycobacterium colombiense | 2 | I294L | 436 | + | + |
| t810566 | Mycobacterium colombiense | 2 | I294V | 437 | ++ | + |
| t810535 | Mycobacterium colombiense | 2 | A296M | 438 | + | + |
| t810242 | Mycobacterium colombiense | 2 | A296R | 439 | + | − |
| t810076 | Mycobacterium colombiense | 2 | K298M | 440 | + | + |
| t808452 | Mycobacterium colombiense | 2 | K298R | 441 | + | + |
| t809334 | Mycobacterium colombiense | 2 | E299K | 442 | + | + |
| t809963 | Mycobacterium colombiense | 2 | E299Q | 443 | + | + |
| t810263 | Mycobacterium colombiense | 2 | E299R | 444 | + | + |
| t809626 | Mycobacterium colombiense | 2 | R300A | 445 | + | + |
| t809472 | Mycobacterium colombiense | 2 | T303D | 446 | ++ | + |
| t808453 | Mycobacterium colombiense | 2 | T303S | 447 | + | + |
| t815591 | Mycobacterium colombiense | 2 | D304E | 448 | + | + |
| t808982 | Mycobacterium colombiense | 2 | D304Q | 449 | ++ | + |
| t809283 | Mycobacterium colombiense | 2 | E305A | 450 | + | + |
| t809792 | Mycobacterium colombiense | 2 | E305D | 451 | + | + |
| t810336 | Mycobacterium colombiense | 2 | Q306D | 452 | + | + |
| t810177 | Mycobacterium colombiense | 2 | Q306E | 453 | + | + |
| t815606 | Mycobacterium colombiense | 2 | L307I | 454 | + | + |
| t810218 | Mycobacterium colombiense | 2 | L307V | 455 | + | + |
| t810383 | Mycobacterium colombiense | 2 | H308E | 456 | + | + |
| t809807 | Mycobacterium colombiense | 2 | H308N | 457 | ++ | + |
| t810020 | Mycobacterium colombiense | 2 | H308R | 458 | + | + |
| t809653 | Mycobacterium colombiense | 2 | T309K | 459 | + | + |
| t809400 | Mycobacterium colombiense | 2 | T309Q | 460 | + | + |
| t808409 | Mycobacterium colombiense | 2 | T309R | 461 | ++ | + |
| t809672 | Mycobacterium colombiense | 2 | L311F | 462 | + | + |
| t808408 | Mycobacterium colombiense | 2 | L311M | 463 | + | + |
| t809950 | Mycobacterium colombiense | 2 | L311T | 464 | ++ | + |
| t815555 | Mycobacterium colombiense | 2 | V312I | 465 | + | + |
| t810007 | Mycobacterium colombiense | 2 | D313E | 466 | + | + |
| t809827 | Mycobacterium colombiense | 2 | R316A | 467 | + | + |
| t810282 | Mycobacterium colombiense | 2 | R316K | 468 | + | + |
| t810403 | Mycobacterium colombiense | 2 | R316Q | 469 | + | + |
| t810296 | Mycobacterium colombiense | 2 | R317K | 470 | + | + |
| 1809973 | Mycobacterium colombiense | 2 | R317N | 471 | ++ | + |
| t810223 | Mycobacterium colombiense | 2 | I318F | 472 | + | + |
| t809512 | Mycobacterium colombiense | 2 | I318L | 473 | ++ | + |
| t815717 | Mycobacterium colombiense | 2 | I318M | 474 | + | + |
| t809948 | Mycobacterium colombiense | 2 | E320D | 475 | ++ | + |
| t809239 | Mycobacterium colombiense | 2 | E320Q | 476 | ++ | + |
| t808443 | Mycobacterium colombiense | 2 | E320S | 477 | + | + |
| t809662 | Mycobacterium colombiense | 2 | Q322E | 478 | + | + |
| t810066 | Mycobacterium colombiense | 2 | Q322K | 479 | + | + |
| t809068 | Mycobacterium colombiense | 2 | Q322R | 480 | + | + |
| t809658 | Mycobacterium colombiense | 2 | A324P | 481 | + | + |
| t809905 | Mycobacterium colombiense | 2 | A324S | 482 | + | + |
| t809855 | Mycobacterium colombiense | 2 | Y325F | 483 | + | + |
| 1808373 | Mycobacterium colombiense | 2 | Y325H | 484 | ++ | + |
| t809854 | Mycobacterium colombiense | 2 | Y325W | 485 | + | + |
| t809710 | Mycobacterium colombiense | 2 | E326K | 486 | + | ++ |
| t809971 | Mycobacterium colombiense | 2 | E326Q | 487 | + | + |
| t809675 | Mycobacterium colombiense | 2 | E326R | 488 | + | + |
| t810506 | Mycobacterium colombiense | 2 | (N/A) | 489 | + | + |
| t809986 | Mycobacterium asiaticum | 3 | H22N | 490 | − | − |
| t810369 | Mycobacterium asiaticum | 3 | H22S | 491 | − | − |
| t809081 | Mycobacterium asiaticum | 3 | H22T | 492 | − | − |
| t810460 | Mycobacterium asiaticum | 3 | E23D | 493 | − | − |
| t810558 | Mycobacterium asiaticum | 3 | H24N | 494 | − | − |
| t810392 | Mycobacterium asiaticum | 3 | V25A | 495 | + | + |
| t810055 | Mycobacterium asiaticum | 3 | V25C | 496 | + | − |
| t810653 | Mycobacterium asiaticum | 3 | V25I | 497 | + | + |
| t810267 | Mycobacterium asiaticum | 3 | V25T | 498 | + | + |
| t809538 | Mycobacterium asiaticum | 3 | I27C | 499 | + | − |
| t810590 | Mycobacterium asiaticum | 3 | I27F | 500 | + | + |
| t809903 | Mycobacterium asiaticum | 3 | I27L | 501 | + | ++ |
| t815557 | Mycobacterium asiaticum | 3 | I27M | 502 | ++ | ++ |
| t809642 | Mycobacterium asiaticum | 3 | I27T | 503 | + | + |
| t810342 | Mycobacterium asiaticum | 3 | I27V | 504 | + | − |
| t809499 | Mycobacterium asiaticum | 3 | I27W | 505 | + | − |
| t809683 | Mycobacterium asiaticum | 3 | I27Y | 506 | + | − |
| t810368 | Mycobacterium asiaticum | 3 | L68A | 507 | + | − |
| t810270 | Mycobacterium asiaticum | 3 | L68M | 508 | + | + |
| t809194 | Mycobacterium asiaticum | 3 | L68N | 509 | + | + |
| t810679 | Mycobacterium asiaticum | 3 | L68P | 510 | + | + |
| t810635 | Mycobacterium asiaticum | 3 | L68Q | 511 | + | + |
| t810701 | Mycobacterium asiaticum | 3 | T69S | 512 | + | + |
| t810539 | Mycobacterium asiaticum | 3 | V70C | 513 | + | ++ |
| t810087 | Mycobacterium asiaticum | 3 | V70P | 514 | ++ | ++ |
| t810178 | Mycobacterium asiaticum | 3 | L73C | 515 | − | − |
| t815626 | Mycobacterium asiaticum | 3 | L73H | 516 | − | − |
| t809700 | Mycobacterium asiaticum | 3 | L73M | 517 | − | − |
| t815719 | Mycobacterium asiaticum | 3 | L73V | 518 | − | − |
| t810198 | Mycobacterium asiaticum | 3 | Y98C | 519 | − | − |
| t815685 | Mycobacterium asiaticum | 3 | Y98F | 520 | − | − |
| t809317 | Mycobacterium asiaticum | 3 | Y98H | 521 | − | − |
| t810398 | Mycobacterium asiaticum | 3 | Y100D | 522 | ++ | ++ |
| t810327 | Mycobacterium asiaticum | 3 | Y100E | 523 | + | + |
| t809918 | Mycobacterium asiaticum | 3 | Y100H | 524 | + | + |
| t809465 | Mycobacterium asiaticum | 3 | Y100Q | 525 | ++ | ++ |
| t809720 | Mycobacterium asiaticum | 3 | L144I | 526 | + | + |
| t810155 | Mycobacterium asiaticum | 3 | K145C | 527 | − | − |
| t810430 | Mycobacterium asiaticum | 3 | K145E | 528 | − | − |
| t810597 | Mycobacterium asiaticum | 3 | K145Q | 529 | − | − |
| t810568 | Mycobacterium asiaticum | 3 | K145S | 530 | − | − |
| t809953 | Mycobacterium asiaticum | 3 | K145T | 531 | − | − |
| t815705 | Mycobacterium asiaticum | 3 | C146I | 532 | + | + |
| t815582 | Mycobacterium asiaticum | 3 | C146L | 533 | − | − |
| t810427 | Mycobacterium asiaticum | 3 | C146V | 534 | + | + |
| t809988 | Mycobacterium asiaticum | 3 | C146Y | 535 | − | − |
| t810633 | Mycobacterium asiaticum | 3 | A147C | 536 | + | + |
| t809852 | Mycobacterium asiaticum | 3 | A147H | 537 | − | − |
| t809596 | Mycobacterium asiaticum | 3 | A147W | 538 | − | − |
| t809826 | Mycobacterium asiaticum | 3 | T148A | 539 | + | + |
| t809095 | Mycobacterium asiaticum | 3 | T148I | 540 | + | + |
| t809706 | Mycobacterium asiaticum | 3 | T148V | 541 | ++ | + |
| t809131 | Mycobacterium asiaticum | 3 | T148W | 542 | − | − |
| t809588 | Mycobacterium asiaticum | 3 | T148Y | 543 | − | − |
| t808992 | Mycobacterium asiaticum | 3 | V164I | 544 | + | + |
| t809237 | Mycobacterium asiaticum | 3 | S176C | 545 | + | + |
| t810262 | Mycobacterium asiaticum | 3 | S176F | 546 | + | + |
| t809085 | Mycobacterium asiaticum | 3 | S176H | 547 | + | + |
| t809811 | Mycobacterium asiaticum | 3 | S176I | 548 | + | + |
| t809066 | Mycobacterium asiaticum | 3 | S176L | 549 | + | + |
| 815573 | Mycobacterium asiaticum | 3 | S176V | 550 | + | + |
| t809949 | Mycobacterium asiaticum | 3 | S176W | 551 | − | − |
| t815648 | Mycobacterium asiaticum | 3 | S176Y | 552 | + | + |
| t810423 | Mycobacterium asiaticum | 3 | T177C | 553 | + | + |
| t810400 | Mycobacterium asiaticum | 3 | H178A | 554 | − | − |
| t808973 | Mycobacterium asiaticum | 3 | H178D | 555 | + | + |
| t810230 | Mycobacterium asiaticum | 3 | H178R | 556 | − | − |
| t815709 | Mycobacterium asiaticum | 3 | H178S | 557 | − | − |
| t810019 | Mycobacterium asiaticum | 3 | H178T | 558 | − | − |
| t809294 | Mycobacterium asiaticum | 3 | T179C | 559 | + | + |
| t808437 | Mycobacterium asiaticum | 3 | T179F | 560 | − | − |
| t810362 | Mycobacterium asiaticum | 3 | T179M | 561 | − | − |
| t815581 | Mycobacterium asiaticum | 3 | A181C | 562 | − | − |
| t810415 | Mycobacterium asiaticum | 3 | A181R | 563 | − | − |
| t808403 | Mycobacterium asiaticum | 3 | A181S | 564 | + | + |
| t809331 | Mycobacterium asiaticum | 3 | Q189A | 565 | + | + |
| t810623 | Mycobacterium asiaticum | 3 | Q189C | 566 | + | + |
| t810006 | Mycobacterium asiaticum | 3 | Q189E | 567 | − | − |
| t808438 | Mycobacterium asiaticum | 3 | Q189I | 568 | + | + |
| t810678 | Mycobacterium asiaticum | 3 | Q189L | 569 | − | − |
| t808435 | Mycobacterium asiaticum | 3 | Q189M | 570 | + | + |
| t809620 | Mycobacterium asiaticum | 3 | Q189V | 571 | + | + |
| t809243 | Mycobacterium asiaticum | 3 | G206N | 572 | − | − |
| t810681 | Mycobacterium asiaticum | 3 | G206S | 573 | + | + |
| t809876 | Mycobacterium asiaticum | 3 | H207M | 574 | − | − |
| t810663 | Mycobacterium asiaticum | 3 | H207N | 575 | − | − |
| t809794 | Mycobacterium asiaticum | 3 | S208A | 576 | + | + |
| t810271 | Mycobacterium asiaticum | 3 | S208C | 577 | + | + |
| t810426 | Mycobacterium asiaticum | 3 | S208T | 578 | + | + |
| t809698 | Mycobacterium asiaticum | 3 | G209N | 579 | + | + |
| t810495 | Mycobacterium asiaticum | 3 | D210C | 580 | − | − |
| t810016 | Mycobacterium asiaticum | 3 | D210E | 581 | − | − |
| t815694 | Mycobacterium sp. 852014- | 3 | D210H | 582 | − | − |
| | 52450_SCH5900713 | | | | | |
| t815688 | Mycobacterium asiaticum | 3 | D210P | 583 | − | − |
| t810682 | Mycobacterium asiaticum | 3 | D210W | 584 | − | − |
| t810118 | Mycobacterium asiaticum | 3 | G228C | 585 | − | − |
| t809417 | Mycobacterium asiaticum | 3 | G228D | 586 | − | − |
| t809229 | Mycobacterium asiaticum | 3 | G228Q | 587 | − | − |
| t809739 | Mycobacterium asiaticum | 3 | G228S | 588 | + | + |
| t810616 | Mycobacterium asiaticum | 3 | D230N | 589 | − | − |
| t808364 | Mycobacterium asiaticum | 3 | R231H | 590 | − | − |
| t809005 | Mycobacterium asiaticum | 3 | R231M | 591 | − | − |
| t810724 | Mycobacterium asiaticum | 3 | R231N | 592 | − | − |
| t809127 | Mycobacterium asiaticum | 3 | R231Q | 593 | − | − |
| t810298 | Mycobacterium asiaticum | 3 | R231T | 594 | − | − |
| t809772 | Mycobacterium asiaticum | 3 | R231W | 595 | − | − |
| t810142 | Mycobacterium asiaticum | 3 | V260I | 596 | ++ | + |
| t809694 | Mycobacterium asiaticum | 3 | S262G | 597 | + | + |
| t810727 | Mycobacterium asiaticum | 3 | H263F | 598 | − | + |
| t810012 | Mycobacterium asiaticum | 3 | H263G | 599 | ++ | + |
| t809849 | Mycobacterium asiaticum | 3 | H263M | 600 | ++ | ++ |
| t810669 | Mycobacterium asiaticum | 3 | H263Q | 601 | ++ | + |
| t809434 | Mycobacterium asiaticum | 3 | H263T | 602 | ++ | + |
| t810629 | Mycobacterium asiaticum | 3 | D264P | 603 | − | − |
| 1810631 | Mycobacterium asiaticum | 3 | D264S | 604 | − | − |
| t810606 | Mycobacterium asiaticum | 3 | D264T | 605 | − | − |
| t809425 | Mycobacterium asiaticum | 3 | A265I | 606 | − | − |
| t809634 | Mycobacterium asiaticum | 3 | A265M | 607 | + | ++ |
| t810637 | Mycobacterium asiaticum | 3 | A265S | 608 | + | + |
| t815633 | Mycobacterium asiaticum | 3 | A265T | 609 | + | + |
| t810507 | Mycobacterium asiaticum | 3 | A265W | 610 | + | ++ |
| t809656 | Mycobacterium asiaticum | 3 | A265Y | 611 | + | ++ |
| t808426 | Mycobacterium asiaticum | 3 | N266A | 612 | ++ | ++ |
| t810033 | Mycobacterium asiaticum | 3 | N266C | 613 | ++ | ++ |
| t809848 | Mycobacterium asiaticum | 3 | N266G | 614 | ++ | ++ |
| t809789 | Mycobacterium asiaticum | 3 | N266H | 615 | + | − |
| t810692 | Mycobacterium asiaticum | 3 | N266I | 616 | ++ | ++ |
| t809349 | Mycobacterium asiaticum | 3 | N266L | 617 | ++ | ++ |
| t809582 | Mycobacterium asiaticum | 3 | N266Q | 618 | ++ | + |
| t810384 | Mycobacterium asiaticum | 3 | N266T | 619 | ++ | ++ |
| t810448 | Mycobacterium asiaticum | 3 | N266W | 620 | + | − |
| t809790 | Mycobacterium asiaticum | 3 | C267T | 621 | + | + |
| t810565 | Mycobacterium asiaticum | 3 | C267W | 622 | ++ | + |
| t810251 | Mycobacterium asiaticum | 3 | W284E | 623 | + | + |
| t815643 | Mycobacterium asiaticum | 3 | W284H | 624 | + | ++ |
| t810599 | Mycobacterium asiaticum | 3 | W284I | 625 | − | − |
| t810638 | Mycobacterium asiaticum | 3 | W284K | 626 | − | − |
| t810409 | Mycobacterium asiaticum | 3 | W284P | 627 | − | − |
| t815625 | Mycobacterium asiaticum | 3 | W284R | 628 | − | − |
| t815657 | Mycobacterium asiaticum | 3 | W284T | 629 | + | ++ |
| t815650 | Mycobacterium gordonae | 4 | L20M | 630 | + | + |
| t815629 | Mycobacterium gordonae | 4 | E23D | 631 | − | − |
| t809083 | Mycobacterium gordonae | 4 | V25L | 632 | + | + |
| t809233 | Mycobacterium gordonae | 4 | V25M | 633 | + | + |
| t809269 | Mycobacterium gordonae | 4 | F26H | 634 | + | + |
| t809184 | Mycobacterium gordonae | 4 | F26L | 635 | − | − |
| t808378 | Mycobacterium gordonae | 4 | F26M | 636 | − | − |
| t809704 | Mycobacterium gordonae | 4 | F26W | 637 | + | + |
| t809210 | Mycobacterium gordonae | 4 | I27C | 638 | + | − |
| t809955 | Mycobacterium gordonae | 4 | I27F | 639 | + | + |
| t810022 | Mycobacterium gordonae | 4 | I27M | 640 | + | + |
| t809254 | Mycobacterium gordonae | 4 | I27T | 641 | + | + |
| t809570 | Mycobacterium gordonae | 4 | I27V | 642 | + | + |
| t810549 | Mycobacterium gordonae | 4 | I27W | 643 | − | − |
| t809011 | Mycobacterium gordonae | 4 | I27Y | 644 | − | − |
| t810424 | Mycobacterium gordonae | 4 | V66I | 645 | + | + |
| t810032 | Mycobacterium gordonae | 4 | L68A | 646 | − | − |
| t815616 | Mycobacterium gordonae | 4 | L68C | 647 | + | + |
| t809296 | Mycobacterium gordonae | 4 | L68M | 648 | + | + |
| t815658 | Mycobacterium gordonae | 4 | L68N | 649 | + | + |
| t815715 | Mycobacterium gordonae | 4 | T69Q | 650 | − | − |
| t809864 | Mycobacterium gordonae | 4 | T69S | 651 | + | − |
| t809212 | Mycobacterium gordonae | 4 | L73C | 652 | − | − |
| t809008 | Mycobacterium gordonae | 4 | L73M | 653 | − | − |
| t809024 | Mycobacterium gordonae | 4 | Y98C | 654 | − | − |
| t809972 | Mycobacterium gordonae | 4 | Y98W | 655 | + | + |
| t809670 | Mycobacterium gordonae | 4 | F126C | 656 | + | − |
| t809886 | Mycobacterium gordonae | 4 | F126L | 657 | − | − |
| t815558 | Mycobacterium gordonae | 4 | F126M | 658 | + | + |
| t810508 | Mycobacterium gordonae | 4 | L144I | 659 | + | + |
| t809142 | Mycobacterium gordonae | 4 | K145C | 660 | − | − |
| t815659 | Mycobacterium gordonae | 4 | K145E | 661 | − | − |
| t810528 | Mycobacterium gordonae | 4 | K145Q | 662 | − | − |
| t810091 | Mycobacterium gordonae | 4 | K145R | 663 | − | − |
| t810548 | Mycobacterium gordonae | 4 | K145S | 664 | − | − |
| t809047 | Mycobacterium gordonae | 4 | K145T | 665 | − | − |
| t810579 | Mycobacterium gordonae | 4 | C146F | 666 | − | − |
| t809291 | Mycobacterium gordonae | 4 | C146I | 667 | − | − |
| t810065 | Mycobacterium gordonae | 4 | C146V | 668 | + | + |
| t809075 | Mycobacterium gordonae | 4 | A147G | 669 | + | + |
| t810250 | Mycobacterium gordonae | 4 | A147I | 670 | + | + |
| t809787 | Mycobacterium gordonae | 4 | A147M | 671 | ++ | + |
| t809606 | Mycobacterium gordonae | 4 | A147S | 672 | + | + |
| t809518 | Mycobacterium gordonae | 4 | T148C | 673 | + | + |
| t810419 | Mycobacterium gordonae | 4 | T148I | 674 | + | + |
| t809001 | Mycobacterium gordonae | 4 | T148M | 675 | + | + |
| t809682 | Mycobacterium gordonae | 4 | T148V | 676 | ++ | + |
| t810259 | Mycobacterium gordonae | 4 | T148W | 677 | − | − |
| t810435 | Mycobacterium gordonae | 4 | T148Y | 678 | − | − |
| t809249 | Mycobacterium gordonae | 4 | V164I | 679 | + | + |
| t810010 | Mycobacterium gordonae | 4 | V164T | 680 | + | + |
| t810137 | Mycobacterium gordonae | 4 | H168S | 681 | + | + |
| t810655 | Mycobacterium gordonae | 4 | V173C | 682 | + | + |
| t810676 | Mycobacterium gordonae | 4 | S176F | 683 | + | − |
| t809797 | Mycobacterium gordonae | 4 | S176H | 684 | + | + |
| t809699 | Mycobacterium gordonae | 4 | S176I | 685 | + | − |
| t809073 | Mycobacterium gordonae | 4 | S176L | 686 | − | − |
| t808366 | Mycobacterium gordonae | 4 | S176M | 687 | + | + |
| t815612 | Mycobacterium gordonae | 4 | S176T | 688 | + | + |
| t809162 | Mycobacterium gordonae | 4 | S176W | 689 | − | − |
| t808971 | Mycobacterium gordonae | 4 | S176Y | 690 | + | − |
| t809870 | Mycobacterium gordonae | 4 | T177C | 691 | + | − |
| t827635 | Mycobacterium gordonae | 4 | T177I | 692 | − | − |
| t810710 | Mycobacterium gordonae | 4 | T177V | 693 | − | − |
| t815704 | Mycobacterium gordonae | 4 | H178D | 694 | + | + |
| t809943 | Mycobacterium gordonae | 4 | H178Q | 695 | − | − |
| t809685 | Mycobacterium gordonae | 4 | H178R | 696 | − | − |
| t809924 | Mycobacterium gordonae | 4 | H178S | 697 | − | − |
| t809048 | Mycobacterium gordonae | 4 | H178T | 698 | − | − |
| t815632 | Mycobacterium gordonae | 4 | H178V | 699 | − | − |
| t810345 | Mycobacterium gordonae | 4 | H178Y | 700 | + | − |
| t815682 | Mycobacterium gordonae | 4 | T179D | 701 | − | − |
| t809922 | Mycobacterium gordonae | 4 | T179F | 702 | − | − |
| t809270 | Mycobacterium gordonae | 4 | T179I | 703 | − | − |
| t809017 | Mycobacterium gordonae | 4 | T179M | 704 | − | − |
| t809530 | Mycobacterium gordonae | 4 | T179N | 705 | − | − |
| t810376 | Mycobacterium gordonae | 4 | T179W | 706 | − | − |
| t808436 | Mycobacterium gordonae | 4 | A181C | 707 | − | − |
| t815693 | Mycobacterium gordonae | 4 | A181G | 708 | + | + |
| t810395 | Mycobacterium gordonae | 4 | A181H | 709 | − | − |
| t809730 | Mycobacterium gordonae | 4 | A181M | 710 | − | − |
| t809010 | Mycobacterium gordonae | 4 | A181S | 711 | + | + |
| t815670 | Mycobacterium gordonae | 4 | A181W | 712 | − | − |
| t827634 | Mycobacterium gordonae | 4 | Q189A | 713 | + | − |
| t809752 | Mycobacterium gordonae | 4 | Q189C | 714 | + | + |
| t809679 | Mycobacterium gordonae | 4 | Q189E | 715 | − | − |
| t810015 | Mycobacterium gordonae | 4 | Q189I | 716 | + | − |
| t809817 | Mycobacterium gordonae | 4 | Q189L | 717 | + | − |
| t809502 | Mycobacterium gordonae | 4 | Q189M | 718 | + | + |
| t809862 | Mycobacterium gordonae | 4 | Q189V | 719 | + | + |
| t815703 | Mycobacterium gordonae | 4 | I204V | 720 | + | + |
| t808402 | Mycobacterium gordonae | 4 | G206N | 721 | − | − |
| t808395 | Mycobacterium gordonae | 4 | G206S | 722 | + | + |
| t810272 | Mycobacterium gordonae | 4 | H207N | 723 | − | − |
| t810685 | Mycobacterium gordonae | 4 | S208A | 724 | + | − |
| t809196 | Mycobacterium gordonae | 4 | S208C | 725 | + | + |
| t809527 | Mycobacterium gordonae | 4 | S208M | 726 | − | − |
| t810116 | Mycobacterium gordonae | 4 | S208T | 727 | + | + |
| t810114 | Mycobacterium gordonae | 4 | G209C | 728 | − | − |
| t809082 | Mycobacterium gordonae | 4 | G209N | 729 | + | + |
| t815621 | Mycobacterium gordonae | 4 | D210C | 730 | − | − |
| t809812 | Mycobacterium gordonae | 4 | D210E | 731 | − | − |
| t809393 | Mycobacterium gordonae | 4 | D210M | 732 | − | − |
| t815661 | Mycobacterium gordonae | 4 | D210W | 733 | − | − |
| t810607 | Mycobacterium gordonae | 4 | G228A | 734 | − | − |
| t810354 | Mycobacterium gordonae | 4 | G228C | 735 | − | − |
| t815671 | Mycobacterium gordonae | 4 | G228Q | 736 | − | − |
| t810113 | Mycobacterium gordonae | 4 | G228S | 737 | − | − |
| t827638 | Mycobacterium gordonae | 4 | D230S | 738 | − | − |
| t809608 | Mycobacterium gordonae | 4 | R231C | 739 | − | − |
| t809147 | Mycobacterium gordonae | 4 | R231F | 740 | − | − |
| t809838 | Mycobacterium gordonae | 4 | R231G | 741 | − | − |
| t810439 | Mycobacterium gordonae | 4 | R231H | 742 | − | − |
| t815562 | Mycobacterium gordonae | 4 | R231M | 743 | − | − |
| t809842 | Mycobacterium gordonae | 4 | R231N | 744 | − | − |
| t808417 | Mycobacterium gordonae | 4 | R231Q | 745 | − | − |
| t815667 | Mycobacterium gordonae | 4 | R231T | 746 | − | − |
| t810574 | Mycobacterium gordonae | 4 | R231W | 747 | − | − |
| t810411 | Mycobacterium gordonae | 4 | V260I | 748 | + | + |
| t810647 | Mycobacterium gordonae | 4 | S262C | 749 | − | − |
| t809970 | Mycobacterium gordonae | 4 | S262G | 750 | + | + |
| t809419 | Mycobacterium gordonae | 4 | H263C | 751 | + | + |
| t810170 | Mycobacterium gordonae | 4 | H263F | 752 | − | − |
| t809727 | Mycobacterium gordonae | 4 | H263N | 753 | + | + |
| t808413 | Mycobacterium gordonae | 4 | H263S | 754 | + | + |
| t808445 | Mycobacterium gordonae | 4 | H263T | 755 | + | + |
| t810690 | Mycobacterium gordonae | 4 | H263Y | 756 | − | − |
| t809501 | Mycobacterium gordonae | 4 | D264F | 757 | − | − |
| t810378 | Mycobacterium gordonae | 4 | D264N | 758 | − | − |
| t810696 | Mycobacterium gordonae | 4 | A265C | 759 | − | − |
| t815707 | Mycobacterium gordonae | 4 | A265I | 760 | − | − |
| t815595 | Mycobacterium gordonae | 4 | A265L | 761 | + | + |
| t810567 | Mycobacterium gordonae | 4 | A265M | 762 | − | − |
| t809709 | Mycobacterium gordonae | 4 | A265Q | 763 | + | + |
| t809995 | Mycobacterium gordonae | 4 | A265S | 764 | + | + |
| t810307 | Mycobacterium gordonae | 4 | A265T | 765 | + | + |
| t809043 | Mycobacterium gordonae | 4 | A265V | 766 | − | + |
| t809266 | Mycobacterium gordonae | 4 | A265W | 767 | + | ++ |
| t809674 | Mycobacterium gordonae | 4 | A265Y | 768 | + | ++ |
| t809112 | Mycobacterium gordonae | 4 | N266A | 769 | + | ++ |
| t810503 | Mycobacterium gordonae | 4 | N266C | 770 | + | + |
| t809373 | Mycobacterium gordonae | 4 | N266G | 771 | + | + |
| t810200 | Mycobacterium gordonae | 4 | N266H | 772 | − | − |
| t808399 | Mycobacterium gordonae | 4 | N266R | 773 | − | − |
| t815608 | Mycobacterium gordonae | 4 | N266S | 774 | + | + |
| t809774 | Mycobacterium gordonae | 4 | N266T | 775 | ++ | ++ |
| t809882 | Mycobacterium gordonae | 4 | C267A | 776 | + | + |
| t809641 | Mycobacterium gordonae | 4 | C267G | 777 | + | + |
| t809557 | Mycobacterium gordonae | 4 | C267T | 778 | + | + |
| t810670 | Mycobacterium gordonae | 4 | C267W | 779 | + | + |
| t808401 | Mycobacterium gordonae | 4 | W284C | 780 | − | − |
| t809050 | Mycobacterium gordonae | 4 | W284D | 781 | − | − |
| t809956 | Mycobacterium gordonae | 4 | W284E | 782 | − | − |
| t808383 | Mycobacterium gordonae | 4 | W284F | 783 | + | ++ |
| t815551 | Mycobacterium gordonae | 4 | W284H | 784 | + | ++ |
| t809661 | Mycobacterium gordonae | 4 | W284K | 785 | − | − |
| t809632 | Mycobacterium gordonae | 4 | W284M | 786 | + | ++ |
| t809801 | Mycobacterium gordonae | 4 | W284N | 787 | + | ++ |
| t809939 | Mycobacterium gordonae | 4 | W284P | 788 | − | − |
| t809946 | Mycobacterium gordonae | 4 | W284Q | 789 | − | + |
| t809181 | Mycobacterium gordonae | 4 | W284R | 790 | − | + |
| t809614 | Mycobacterium gordonae | 4 | W284T | 791 | − | − |
| t815636 | Mycobacterium gordonae | 4 | W284Y | 792 | + | ++ |
| t809888 | Mycobacterium gordonae | 4 | Y286F | 793 | + | + |
| t808424 | Mycobacterium gordonae | 4 | Y286M | 794 | + | + |
| t809689 | Mycobacterium gordonae | 4 | Y286W | 795 | + | + |
| t815673 | Prostheco-microbium | 6 | L29I | 796 | − | − |
| | hirschii | | | | | |
| t810162 | Prostheco-microbium | 6 | H31R | 797 | − | − |
| | hirschii | | | | | |
| t815700 | Prostheco-microbium | 6 | H31S | 798 | − | − |
| | hirschii | | | | | |
| t810124 | Prostheco-microbium | 6 | L34C | 799 | − | − |
| | hirschii | | | | | |
| t810314 | Prostheco-microbium | 6 | L34I | 800 | − | − |
| | hirschii | | | | | |
| t810389 | Prostheco-microbium | 6 | L34M | 801 | − | − |
| | hirschii | | | | | |
| t815660 | Prostheco-microbium | 6 | L34V | 802 | − | − |
| | hirschii | | | | | |
| t810221 | Prostheco-microbium | 6 | L35I | 803 | − | − |
| | hirschii | | | | | |
| t815683 | Prostheco-microbium | 6 | L35M | 804 | − | − |
| | hirschii | | | | | |
| t808966 | Prostheco-microbium | 6 | H72C | 805 | − | − |
| | hirschii | | | | | |
| t810367 | Prostheco-microbium | 6 | H72D | 806 | − | − |
| | hirschii | | | | | |
| t810563 | Prostheco-microbium | 6 | H72E | 807 | − | − |
| | hirschii | | | | | |
| t815668 | Prostheco-microbium | 6 | H72F | 808 | − | − |
| | hirschii | | | | | |
| t810301 | Prostheco-microbium | 6 | H72M | 809 | − | − |
| | hirschii | | | | | |
| t810226 | Prostheco-microbium | 6 | H72N | 810 | − | − |
| | hirschii | | | | | |
| t810057 | Prostheco-microbium | 6 | H72Q | 811 | − | − |
| | hirschii | | | | | |
| t809556 | Prostheco-microbium | 6 | H72R | 812 | − | − |
| | hirschii | | | | | |
| t810416 | Prostheco-microbium | 6 | H72W | 813 | − | − |
| | hirschii | | | | | |
| t810315 | Prostheco-microbium | 6 | R73C | 814 | − | − |
| | hirschii | | | | | |
| t815635 | Prostheco-microbium | 6 | R73H | 815 | − | − |
| | hirschii | | | | | |
| t810141 | Prostheco-microbium | 6 | R73I | 816 | − | − |
| | hirschii | | | | | |
| t810081 | Prostheco-microbium | 6 | R73K | 817 | − | − |
| | hirschii | | | | | |
| t809475 | Prostheco-microbium | 6 | R73M | 818 | − | − |
| | hirschii | | | | | |
| t810147 | Prostheco-microbium | 6 | R73N | 819 | − | − |
| | hirschii | | | | | |
| t809202 | Prostheco-microbium | 6 | R73T | 820 | − | − |
| | hirschii | | | | | |
| t808953 | Prostheco-microbium | 6 | L74F | 821 | − | − |
| | hirschii | | | | | |
| t810190 | Prostheco-microbium | 6 | L74H | 822 | − | − |
| | hirschii | | | | | |
| t809541 | Prostheco-microbium | 6 | L74I | 823 | − | − |
| | hirschii | | | | | |
| t810291 | Prostheco-microbium | 6 | L74M | 824 | − | − |
| | hirschii | | | | | |
| t810343 | Prostheco-microbium | 6 | L74W | 825 | − | − |
| | hirschii | | | | | |
| t810598 | Prostheco-microbium | 6 | Q75C | 826 | − | − |
| | hirschii | | | | | |
| t809170 | Prostheco-microbium | 6 | Q75E | 827 | − | − |
| | hirschii | | | | | |
| t810253 | Prostheco-microbium | 6 | Q75F | 828 | − | − |
| | hirschii | | | | | |
| t808371 | Prostheco-microbium | 6 | Q75H | 829 | − | − |
| | hirschii | | | | | |
| t809345 | Prostheco-microbium | 6 | Q75K | 830 | − | − |
| | hirschii | | | | | |
| t810399 | Prostheco-microbium | 6 | Q75R | 831 | − | − |
| | hirschii | | | | | |
| t810600 | Prostheco-microbium | 6 | Q75T | 832 | − | − |
| | hirschii | | | | | |
| t809761 | Prostheco-microbium | 6 | I95L | 833 | − | − |
| | hirschii | | | | | |
| t810139 | Prostheco-microbium | 6 | 195M | 834 | − | − |
| | hirschii | | | | | |
| t809046 | Prostheco-microbium | 6 | 195V | 835 | − | − |
| | hirschii | | | | | |
| t810264 | Prostheco-microbium | 6 | V96C | 836 | − | − |
| | hirschii | | | | | |
| t810472 | Prostheco-microbium | 6 | V96I | 837 | − | − |
| | hirschii | | | | | |
| t809639 | Prostheco-microbium | 6 | V96T | 838 | − | − |
| | hirschii | | | | | |
| t810306 | Prostheco-microbium | 6 | L98A | 839 | − | − |
| | hirschii | | | | | |
| t809454 | Prostheco-microbium | 6 | L98C | 840 | − | − |
| | hirschii | | | | | |
| t808985 | Prostheco-microbium | 6 | L98I | 841 | − | − |
| | hirschii | | | | | |
| t809463 | Prostheco-microbium | 6 | L98M | 842 | − | − |
| | hirschii | | | | | |
| t809272 | Prostheco-microbium | 6 | L98T | 843 | − | − |
| | hirschii | | | | | |
| t809259 | Prostheco-microbium | 6 | L98V | 844 | − | − |
| | hirschii | | | | | |
| t810112 | Prostheco-microbium | 6 | T99N | 845 | − | − |
| | hirschii | | | | | |
| t810026 | Prostheco-microbium | 6 | T99S | 846 | − | − |
| | hirschii | | | | | |
| t809171 | Prostheco-microbium | 6 | T100C | 847 | − | − |
| | hirschii | | | | | |
| t810260 | Prostheco-microbium | 6 | T100N | 848 | − | − |
| | hirschii | | | | | |
| t815634 | Prostheco-microbium | 6 | T100S | 849 | − | − |
| | hirschii | | | | | |
| t809203 | Prostheco-microbium | 6 | T100V | 850 | − | − |
| | hirschii | | | | | |
| t809273 | Prostheco-microbium | 6 | I103C | 851 | − | − |
| | hirschii | | | | | |
| t809094 | Prostheco-microbium | 6 | I103M | 852 | − | − |
| | hirschii | | | | | |
| t810239 | Prostheco-microbium | 6 | V104G | 853 | − | − |
| | hirschii | | | | | |
| t810404 | Prostheco-microbium | 6 | V104R | 854 | − | − |
| | hirschii | | | | | |
| t810316 | Prostheco-microbium | 6 | D106N | 855 | − | − |
| | hirschii | | | | | |
| t809461 | Prostheco-microbium | 6 | L110I | 856 | − | − |
| | hirschii | | | | | |
| t810219 | Prostheco-microbium | 6 | F127H | 857 | − | − |
| | hirschii | | | | | |
| t809668 | Prostheco-microbium | 6 | F127L | 858 | − | − |
| | hirschii | | | | | |
| t810279 | Prostheco-microbium | 6 | F127Y | 859 | − | − |
| | hirschii | | | | | |
| t810099 | Prostheco-microbium | 6 | Y128H | 860 | + | − |
| | hirschii | | | | | |
| t815580 | Prostheco-microbium | 6 | T129G | 861 | − | − |
| | hirschii | | | | | |
| t810713 | Prostheco-microbium | 6 | T129K | 862 | − | − |
| | hirschii | | | | | |
| t815663 | Prostheco-microbium | 6 | T129M | 863 | − | − |
| | hirschii | | | | | |
| t809278 | Prostheco-microbium | 6 | T129V | 864 | − | − |
| | hirschii | | | | | |
| t810108 | Prostheco-microbium | 6 | T129Y | 865 | − | − |
| | hirschii | | | | | |
| t810451 | Prostheco-microbium | 6 | E169A | 866 | − | − |
| | hirschii | | | | | |
| t815599 | Prostheco-microbium | 6 | E169C | 867 | − | − |
| | hirschii | | | | | |
| t810308 | Prostheco-microbium | 6 | E169K | 868 | − | − |
| | hirschii | | | | | |
| t809398 | Prostheco-microbium | 6 | E169T | 869 | − | − |
| | hirschii | | | | | |
| t815681 | Prostheco-microbium | 6 | T199A | 870 | − | − |
| | hirschii | | | | | |
| t809601 | Prostheco-microbium | 6 | T199H | 871 | − | − |
| | hirschii | | | | | |
| t809303 | Prostheco-microbium | 6 | T199I | 872 | − | − |
| | hirschii | | | | | |
| t810247 | Prostheco-microbium | 6 | T199M | 873 | − | − |
| | hirschii | | | | | |
| t810366 | Prostheco-microbium | 6 | T199N | 874 | − | − |
| | hirschii | | | | | |
| t815696 | Prostheco-microbium | 6 | T199Q | 875 | − | − |
| | hirschii | | | | | |
| t809619 | Prostheco-microbium | 6 | T199S | 876 | − | − |
| | hirschii | | | | | |
| t809344 | Prostheco-microbium | 6 | T199V | 877 | − | − |
| | hirschii | | | | | |
| t809664 | Prostheco-microbium | 6 | T199W | 878 | − | − |
| | hirschii | | | | | |
| t810107 | Prostheco-microbium | 6 | T199Y | 879 | − | − |
| | hirschii | | | | | |
| t815669 | Prostheco-microbium | 6 | I200C | 880 | − | − |
| | hirschii | | | | | |
| t815641 | Prostheco-microbium | 6 | I200V | 881 | − | − |
| | hirschii | | | | | |
| t810229 | Prostheco-microbium | 6 | H201C | 882 | − | − |
| | hirschii | | | | | |
| t808381 | Prostheco-microbium | 6 | H201R | 883 | − | − |
| | hirschii | | | | | |
| t815695 | Prostheco-microbium | 6 | R204A | 884 | − | − |
| | hirschii | | | | | |
| t810028 | Prostheco-microbium | 6 | R204C | 885 | − | − |
| | hirschii | | | | | |
| t810318 | Prostheco-microbium | 6 | R204E | 886 | − | − |
| | hirschii | | | | | |
| t809480 | Prostheco-microbium | 6 | R204G | 887 | − | − |
| | hirschii | | | | | |
| t810429 | Prostheco-microbium | 6 | R204H | 888 | − | − |
| | hirschii | | | | | |
| t809103 | Prostheco-microbium | 6 | R204M | 889 | − | + |
| | hirschii | | | | | |
| t809493 | Prostheco-microbium | 6 | R204N | 890 | − | − |
| | hirschii | | | | | |
| t810695 | Prostheco-microbium | 6 | R204P | 891 | − | − |
| | hirschii | | | | | |
| t809258 | Prostheco-microbium | 6 | R204Y | 892 | − | − |
| | hirschii | | | | | |
| t809062 | Prostheco-microbium | 6 | I228L | 893 | − | − |
| | hirschii | | | | | |
| t809059 | Prostheco-microbium | 6 | I228M | 894 | − | − |
| | hirschii | | | | | |
| t810687 | Prostheco-microbium | 6 | I228V | 895 | − | − |
| | hirschii | | | | | |
| t809227 | Prostheco-microbium | 6 | G229C | 896 | − | − |
| | hirschii | | | | | |
| t809079 | Prostheco-microbium | 6 | G229N | 897 | − | + |
| | hirschii | | | | | |
| t810664 | Prostheco-microbium | 6 | G229S | 898 | − | − |
| | hirschii | | | | | |
| t809134 | Prostheco-microbium | 6 | G229T | 899 | − | − |
| | hirschii | | | | | |
| t810393 | Prostheco-microbium | 6 | M231C | 900 | + | − |
| | hirschii | | | | | |
| t809158 | Prostheco-microbium | 6 | M231I | 901 | − | − |
| | hirschii | | | | | |
| t809117 | Prostheco-microbium | 6 | M231L | 902 | − | − |
| | hirschii | | | | | |
| t809309 | Prostheco-microbium | 6 | M231V | 903 | − | − |
| | hirschii | | | | | |
| t809578 | Prostheco-microbium | 6 | D232E | 904 | − | − |
| | hirschii | | | | | |
| t815645 | Prostheco-microbium | 6 | D232G | 905 | − | − |
| | hirschii | | | | | |
| t808400 | Prostheco-microbium | 6 | D232N | 906 | − | − |
| | hirschii | | | | | |
| t809053 | Prostheco-microbium | 6 | L236F | 907 | − | − |
| | hirschii | | | | | |
| t809178 | Prostheco-microbium | 6 | L236I | 908 | − | − |
| | hirschii | | | | | |
| t809503 | Prostheco-microbium | 6 | L236M | 909 | − | − |
| | hirschii | | | | | |
| t810292 | Prostheco-microbium | 6 | L236W | 910 | − | − |
| | hirschii | | | | | |
| t809513 | Prostheco-microbium | 6 | V251L | 911 | − | − |
| | hirschii | | | | | |
| t809469 | Prostheco-microbium | 6 | V251M | 912 | − | − |
| | hirschii | | | | | |
| t809156 | Prostheco-microbium | 6 | E252A | 913 | − | − |
| | hirschii | | | | | |
| t810206 | Prostheco-microbium | 6 | E252G | 914 | − | − |
| | hirschii | | | | | |
| t810437 | Prostheco-microbium | 6 | E252N | 915 | − | − |
| | hirschii | | | | | |
| t810149 | Prostheco-microbium | 6 | E252Q | 916 | − | − |
| | hirschii | | | | | |
| t810256 | Prostheco-microbium | 6 | E252S | 917 | − | − |
| | hirschii | | | | | |
| t815691 | Prostheco-microbium | 6 | D254N | 918 | − | − |
| | hirschii | | | | | |
| t815712 | Prostheco-microbium | 6 | F255M | 919 | − | − |
| | hirschii | | | | | |
| t809805 | Prostheco-microbium | 6 | F255W | 920 | − | − |
| | hirschii | | | | | |
| t808977 | Prostheco-microbium | 6 | F255Y | 921 | − | − |
| | hirschii | | | | | |
| t809207 | Prostheco-microbium | 6 | I258F | 922 | − | − |
| | hirschii | | | | | |
| t810145 | Prostheco-microbium | 6 | I258H | 923 | − | − |
| | hirschii | | | | | |
| t810289 | Prostheco-microbium | 6 | I258K | 924 | − | − |
| | hirschii | | | | | |
| t809337 | Prostheco-microbium | 6 | I258L | 925 | − | − |
| | hirschii | | | | | |
| t809476 | Prostheco-microbium | 6 | I258N | 926 | − | − |
| | hirschii | | | | | |
| t810175 | Prostheco-microbium | 6 | I258P | 927 | − | − |
| | hirschii | | | | | |
| t809793 | Prostheco-microbium | 6 | I258V | 928 | + | + |
| | hirschii | | | | | |
| t810483 | Prostheco-microbium | 6 | I258W | 929 | − | − |
| | hirschii | | | | | |
| t809097 | Prostheco-microbium | 6 | I270C | 930 | − | − |
| | hirschii | | | | | |
| t809458 | Prostheco-microbium | 6 | I270H | 931 | − | − |
| | hirschii | | | | | |
| t809379 | Prostheco-microbium | 6 | I270L | 932 | − | − |
| | hirschii | | | | | |
| t810338 | Prostheco-microbium | 6 | I270P | 933 | − | − |
| | hirschii | | | | | |
| t809265 | Prostheco-microbium | 6 | I270V | 934 | − | − |
| | hirschii | | | | | |
| t809962 | Prostheco-microbium | 6 | S295A | 935 | − | − |
| | hirschii | | | | | |
| t809279 | Prostheco-microbium | 6 | H296C | 936 | − | − |
| | hirschii | | | | | |
| t810517 | Prostheco-microbium | 6 | H296M | 937 | − | − |
| | hirschii | | | | | |
| t809280 | Prostheco-microbium | 6 | H296N | 938 | − | − |
| | hirschii | | | | | |
| t809564 | Prostheco-microbium | 6 | H296Q | 939 | − | − |
| | hirschii | | | | | |
| t809246 | Prostheco-microbium | 6 | H296T | 940 | − | − |
| | hirschii | | | | | |
| t810391 | Prostheco-microbium | 6 | D297G | 941 | − | − |
| | hirschii | | | | | |
| t809318 | Prostheco-microbium | 6 | D297N | 942 | − | − |
| | hirschii | | | | | |
| t809598 | Prostheco-microbium | 6 | D297S | 943 | − | − |
| | hirschii | | | | | |
| t810143 | Prostheco-microbium | 6 | I298M | 944 | − | − |
| | hirschii | | | | | |
| t810093 | Prostheco-microbium | 6 | I298V | 945 | − | − |
| | hirschii | | | | | |
| t815706 | Prostheco-microbium | 6 | C299A | 946 | − | − |
| | hirschii | | | | | |
| t809586 | Prostheco-microbium | 6 | C299F | 947 | − | − |
| | hirschii | | | | | |
| t809902 | Prostheco-microbium | 6 | C299G | 948 | − | − |
| | hirschii | | | | | |
| t809351 | Prostheco-microbium | 6 | C299H | 949 | − | − |
| | hirschii | | | | | |
| t809172 | Prostheco-microbium | 6 | C299M | 950 | − | − |
| | hirschii | | | | | |
| t810364 | Prostheco-microbium | 6 | C299S | 951 | − | − |
| | hirschii | | | | | |
| t810034 | Prostheco-microbium | 6 | C299T | 952 | − | − |
| | hirschii | | | | | |
| t810182 | Prostheco-microbium | 6 | C299W | 953 | − | − |
| | hirschii | | | | | |
| t815592 | Prostheco-microbium | 6 | R303Q | 954 | − | + |
| | hirschii | | | | | |
| t809208 | Prostheco-microbium | 6 | Y312F | 955 | − | − |
| | hirschii | | | | | |
| t810278 | Prostheco-microbium | 6 | (N/A) | 956 | − | − |
| | hirschii | | | | | |

The results indicated that PTEs comprising the following amino acid substitutions relative to SEQ ID NO: 1 were found to be active against VX and VR: V25M, I27F, I27M, I27T, V66I, L68N, L68M, L68C, 169S, V70P, V70C, V70T, L144I, A147G, A147F, T148V, T148C, T148M, V164I, S176T, S176M, S176V, S176C, S176Y, S176I, T177C, T179C, T179S, A181P, A181S, A181C, S208T, S208A, G228S, H263M, H263S, H263C, H263T, H263N, A265C, A265T, A265F, A265W, A265M, N266S, N266M, N266T, N266G, N266A, C267A, C267T, C267W, W284D, W284H, W284C, W284N, W284Y, Y286M, and Y286W.

PTEs comprising the following amino acid substitutions relative to SEQ ID NO: 2 were found to be active against VX and VR: S2K, S2T, S2A, E3K, E3T, E3Q, L4I L4V, N5R, N5Q, N5M, R8L, R8T, R8C, S10P, D12E, D12P, T13P, T13A, A14S, A14E, A14D, A15D, A15Q, A15E, L16M, V18M, V18I, M28D, T29S, T30S, 130W, T30P, E31G, I32V, 132M, I32W, I32F, A33W, E34Q, N35D, Y36F, Y36W, Y36H, E38D, A39P, W40F, D42N, E43D, D44E, D44N, V47I, V47M, A48E, A48W, D49H, D49W, D49C, D49M, V51I, V51L, K52R, R53Q, R53E, N55K, E56D, E56R, E56Q, L57F, A59E, A59Q, R60A, R60H, D63N, T64S, G72D, Y76N, Y76D, I77V, P78D, P78E, I80L, I80M, I80V, A81R, R82K, R82E, V83L, V83I, A84S, A85E, A85R, E86R, T87S, E88G, L89V, L89M, N90H, I91V, V92I, V93C, T99C, V103W, V103Y, V103H, M105W, Y106F, Y106H, Y106W, F107Y, F107W, F107M, Y109W, Y109F, L110M, L110W, E115D, G118T, G118S, E120D, I121Q, I121E, M122L, M122I, T123A, D124E, V127I, R128H, R128N, Q132D, Q132E, I134V, A135E, A135G, D136G, I139V, K140H, K140R, T150Y, T150S, T150H, P151W, P151H, P151N, P151D, V153I, P155E, P155D, G156W, E158H, A165C, Q166R, H168Q, G182D, L183T, L187H, L187M, L187F, E188W, Q190I, Q190L, K191R, F193L, E194D, E195D, L200P, S201N, R202H, R202K, V203C, V2031, 1214M, G215E, G215D, E219D, L220I, L220M, L220V, I221M, I221C, I221A, A222H, A223R, S225C, Y226W, L227V, L227I, D235C, A236H, A236W, A236K, L238M, L238H, P239S, F240W, F240D, F240Y, E241D, D242E, V244C, N245D, N245E, N245R, T246M, T246L, V247I, V247L, Q249E, Q249W, Q249R, Q249H, M250L, C251I, C251V, E252H, R253N, H255Y, H255W, K258H, K258R, M259I, A271G, L272H, L272W, L272M, D274G, D274W, E275K, V277W, S278R, Q279K, Q279R, Q279H, M281Y, M281F, P282G, N283D, N283G, H285G, L287T, H288F, H288Y, 1289L, I289V, H290F, H290L, N291R, N291D, N291T, N291E, D292N, D292R, V293I, I294L, I294V, A296M, K298M, K298R, E299Q, E299K, E299R, R300A, T303S, T303D, D304E, D304Q, E305D, E305A, Q306D, Q306E, L307I, L307V, H308R, H308E, H308N, T309Q, T309K, T309R, L311M, L311F, L311T, V312I, D313E, R316A, R316K. R316Q, R317K, R317N, I318M, I318F, I318L, E320S, E320Q, E320D, Q322R, Q322E, Q322K, A324P, A324S, Y325W, Y325F, Y325H, E326Q, E326R, and E326K.

PTEs comprising the following amino acid substitutions relative to SEQ ID NO: 3 were found to be active against VX and VR: V25A, V25T, V25I, I27F, I27T, I27M, I27L, L68N, L68P, L68Q, L68M, T69S, V70P, V70C, Y100E, Y100H, Y100D, Y100Q, L144I, C146V, C146I, A147C, T148I, T148A, T148V, V164I, S176L, S176H, S176V, S176C, S176I, S176Y, S176F, T177C, H178D, T179C, A181S, Q189M, Q189V, Q189A, Q189I, Q189C, G206S, S208A, S208T, S208C, G209N, G228S, V260I, S262G, H263M, H263G, H263T, H263Q, A265T, A265S, A265M, A265W, N266L, N266I, N266G, N266T, N266A, N266C, N266Q, C267T, C267W, W284E, and W284T.

PTEs comprising the following amino acid substitutions relative to SEQ ID NO: 4 were found to be active against VX and VR: L20M, V25L, V25M, F26W, F26H, I27M, I27F, I27V, I27T, V66I, L68N, L68M, L68C, Y98W, F126M, L144I, C146V, A147I, A147G, A147S, A147M, T148M, T148C, T148I, T148V, V164T, V164I, H168S, V173C, S176T, S176M, S176H, H178D, A181S, A181G, Q189M, Q189V, Q189C, I204V, G206S, S208C, S208T, G209N, V260I, S262G, H263S, H263C, H263T, H263N, A265L, A265Q, A265T, A265S, A265Y, A265W, N266C, N266G, N266S, N266T, N266A, C267W, C267A, C267T, C267G, W284H, W284Y, W284M, W284F, W284N, Y286F, Y286M, and Y286W.

PTERs comprising the following amino acid substitution relative to SEQ ID NO: 6 were found to be active against VX and VR: I258V.

PTERs comprising the following amino acid substitutions relative to SEQ ID NO: 6 were found to be active against VX: Y128H and M231C.

PTERs comprising the following amino acid substitutions relative to SEQ ID NO: 6 were found to be active against VR: R204M, G229N, and R303Q.

### Example 3: OPNA-Active Human PTER

Human PTER (corresponding to UniProt Accession No. Q96BW5 and SEQ ID NO: 9) is not known to exhibit activity against VX, VR, other V-agents, or any OPNAs or phosphono esters or phospho esters.

In an attempt to identify human PTERs with improved activity against one or more OPNAs, the following table provides human PTERs that are created using: (1) Brevundimonas diminuta PTE, specifically variant “C23” (Goldsmith, 2016; PDB entry 6G3M), which exhibits very high activity against both VX and VR; (2) Proteus mirabilis hi4320 PTER (corresponding to UniProt Accession No. B4EXV8; PDB entry 3RHG), which exhibits very high activity against certain methylphosphono esters (Xiang et al. (2015) Biochemistry 54:2919-2930. doi: 10.1021/acs.biochem.5b00199); and (3) a PTER corresponding to SEQ ID NO: 928, described herein as expressed in strain t809793, which is a 1258V substitution mutant of Prosthecomicrobium hirschii PTER (corresponding to UniProt Accession No. AOAOP6VJM8 and SEQ ID NO: 6).

TABLE 8
Human PTER mutations to be screened for improved
activity against one or more V-agents.

| Human PTER | | | |
|---|---|---|---|
| Position | | Corresponding Residue | |
| (relative to | | | P. mirabilis |
| SEQ ID NO: 9) | Mutation(s) | C23 PTE | PTER |
| 31 | M31G | G60 | N28 |
| 98 | N98V | V101 | A95 |
| 31-75 | M31-Q75 replaced by | G60-L79 | N28-P73 |
| | C23 G60-L79 | | |
| 128 | Y128W | W131 | Y126 |
| 129 | V129E | E132 | I127 |
| 168, 169 | G168del*, E169K | K169 | G165, E166 |
| 200 | I200H | H201 | H199 |
| 201 | H201T | T202 | M200 |
| 229 | S229G | G229 | A228 |
| 231 | L231S | S231 | S230 |
| 233 | R233D | D233 | P232 |
| 255 | L255G | G254 | M254 |
| 171, 173 | G171A, S173N | A171, N173 | G168, S170 |
| 174, 175 | W174, P175 | G174, K175 | P171, F172 |
| 228, 244 | M228I, F244L | I228, L243 | L227, M243 |
| 299-311 | I299W-H311 replaced by | W302-D323 | V295-N307 |
| | C23 W302-D323 | | |

*“del” indicates that this human PTER residue is deleted.

The human PTER amino acid residue ranges provided in this example are approximate; that is, they are to be read and understood as the given residue range plus or minus 0, 1, 2, or 3 residues at either (or both) end(s) of the given residue range.

OPNA-active human PTER variants are created by mutating human PTER with any one or more the specific mutations enumerated in Table 8.

OPNA-active human PTER variants are also created by replacing, in whole or in part, a human PTER core beta-strand region (defined approximately as residue ranges R22-H28, G93-T100, V119-V129, 1166-S173, C196-P202, K225-D232, C249-F256, and R292-H300) by the corresponding core beta-strand region sequence of C23 PTE, P. mirabilis PTER, or a mixture thereof. One or more of the eight core beta-strand regions are so replaced, i.e. any one beta-strand, any combination of two beta-strands, etc., up to and including replacement of all eight beta-strands.

These core beta-strand region replacements may also be combined with one or more of the specific mutations enumerated in Table 8.

OPNA-active human PTER variants are also created by replacing, in whole or in part, a human PTER core alpha-helix region (defined approximately as residue ranges Q75-G91, A106-G118, S141-G156, T177-T187, R207-G220, D237-G248, D273-G288, and S314-G328) by the corresponding core alpha-helix region sequence of C23 PTE and/or P. mirabilis PTER, or mixture thereof. Any combination of these eight core alpha-helix regions are so replaced, i.e. any one alpha-helix, any combination of two alpha-helices, etc., up to and including replacement of all eight alpha-helices.

These core alpha-helix region replacements may also be combined with one or more of the specific mutations enumerated in Table 8.

OPNA-active human PTER variants are also created by replacing, in whole or in part, a human PTER sequence-contiguous other region, for example a beta-strand, alpha-helix, or loop region not specifically enumerated above, by the corresponding region sequence of C23 PTE, P. mirabilis PTER, or mixture thereof. Any combination of these regions are so replaced, i.e. any one region, any combination of two regions, etc., up to and including replacement of all such regions. These region replacements may also be combined with one or more of the specific mutations enumerated in Table 8.

OPNA-active human PTER variants are also created by combining one or more of the specific mutations enumerated in Table 8 with one or more of the three types (core beta-strand; core alpha-helix; or other) of regions enumerated above.

OPNA-active human PTER variants are also created as described above, wherein C23 PTE and/or P. mirabilis PTER are instead replaced by any other PTE and/or PTER that exhibits measurable activity in the hydrolysis of V-agents, G-agents, A-agents, other OPNAs, and/or other (thio)phosphino esters, (thio)phosphono esters, and/or (thio)phosphoro esters.

### Example 4: Engineering Human and Non-Human Cells that Express OPNA Hydrolyzing Enzymes

The OPNA hydrolyzing enzymes associated with the disclosure are expressed in or on human cells, genetically engineered human cells, or human-derived cell lines using methods known in the art. Nucleotide sequences are codon optimized for expression in human cells. In some embodiments, RNAs are constructed using non-natural nucleosides such as N-methyl-pseudouridine. Optimized nucleotide sequences are incorporated into a suitable expression vector. Expression vectors are known in the art and can include, for example, one or more gene promoter sequence(s), one or more gene enhancer sequence(s), and/or one or more internal ribosomal entry site(s). Expression vectors with optimized PTE or PTER nucleotide sequences are introduced into a human cell, engineered human cell or a human-derived cell line using methods known to those of skill in the art to generate transformed cells expressing a PTE or PTER. Such methods include, for example, cationic lipofection, calcium phosphate transfection, electroporation, use of lentivirus or adenovirus or adenovirus-associated virus or vesicular stomatitis virus or other such well-known viral vectors, or similar well-known nucleic acid transfection methods. In some cases, the PTE or PTER nucleotide sequence or expression vector comprising the PTE or PTER nucleotide sequence used is responsive to physiological or artificially applied signals such that expression of the OPNA hydrolyzing enzyme is controlled, for example, by application of well-known molecules such as tetracycline or rapamycin, or by altering the temperature of the cells.

### Example 5: Identification of Engineered OPNA Hydrolyzing Enzymes Described in Example 2 that have High Activity Against VX and/or VR Nerve Agents

A subset of the PTE and PTER mutants described in Example 2 were screened to identify the ones most active on VX and/or VR nerve agents. This Example describes the experimental design, general protocol, and results of this screen.

### Experimental Design

A colorimetric assay was used to measure the hydrolysis of V-type nerve agents. Hydrolysis of V-type nerve agents creates a free thiol that rapidly reacts with 5,5,-dithio-bi-(2-nitrobenzoic acid) (DTNB) in a 1:1 molar ratio, generating 2-nitro-5-thiobenzoate (TNB). TNB absorbs at 412 nm, so the hydrolysis of V-type nerve agents can be indirectly measured in real-time in a spectrophotometer.

### General Protocol

Racemic nerve agents VX and VR were issued in saline at the following concentrations: 0.716 μg/μL (VX) and 0.799 μg/μL (VR).

Enzymes were removed from storage at −80° C. and thawed slowly on ice. Two test plates were created by transferring 25 μL of each enzyme variant (Table 9) to two new 96-well microplates (VX Test Plate and VR Test Plate). Samples were run in duplicate. The test plates were briefly stored at 4° C. until used in the assay.

Aliquots of 20 mM DTNB (Sigma; St. Louis, MO) in 0.1 M KPO₄ buffer were removed from storage at −20° C. and thawed on ice. DTNB was diluted to 8 mM in 50 mM HEPES pH 7.6, 137 mM NaCl, 2.7 mM KCl, 1 mM CoCl₂. VX Test Plate was removed from storage at 4° C. and allowed to warm to room temperature for 10 minutes. During the 10 minute warming period, 45 μL of 8 mM DTNB was added to each well. Once the plate equilibrated to room temperature, 50 μL of VX was added to each well. Immediately following the addition of VX, the plate was read at 412 nm in 10 second intervals for a total duration of 5 minutes in a SpectraMax Plus 384 microplate reader (Molecular Devices; San Jose, CA). Raw values (mOD412/min) were generated using the plate reader software (SoftMax v 5.4) to calculate the slope of the linear portion of the curve. The same procedure was repeated with VR Test Plate using 50 μL of VR instead of VX.

V-Activity Values were calculated for each enzyme variant tested, and enzymes with activity values greater than 20 were re-run with varying concentrations of V-agent (Michaelis-Menten kinetics). For this assay, the above protocol was repeated with the exception that each enzyme was tested against an 8 step 2-fold serial dilution of V-agent. Enzymes were tested in singlet due to the large volume of enzyme required for the assay.

Raw absorbance values in mOD412/min were divided by 1000 and converted to OD₄₁₂/min. These absorbance values were used to calculate the concentration in mol/min using Beer's law (A=εlc). The molar extinction coefficient (6) for TNB is 13,600 M⁻¹ cm⁻¹ and the pathlength (1) is 0.3572 cm. The reaction volume in the well is 0.00012 L. The concentration in mol/min was then converted to nmol/min.

Each plate contained 8 background control wells. These wells were used to determine background levels for each plate assay and were subtracted from the Raw Values to generate the Background Removed Values.

To normalize the enzymatic activities, the activity values were calculated per μg of protein in each reaction. Activity values that were negative are reported as 0.00. The V-Activity Values (mOD₄₁₂/min/μg) were calculated using the following equation: V-Activity Value=(mOD412/min value/μg of Enzyme Variant) The following table provides the VR and VX activities of a subset of engineered enzymes initially screened in Example 2, the point mutation in each engineered enzyme relative to the enzyme template, and the source organism of each enzyme template.

TABLE 9
V-agent hydrolyzing activities of top PTEs and PTERs

| | | | | | | VR | VX |
|---|---|---|---|---|---|---|---|
| | | | Mutation | PTE or | | Activity | Activity |
| | Source organism of | Template | (relative | PTER | | Value | Value |
| | template PTE or | SEQ ID | to | SEQ ID | # of | (mOD412/ | (mOD412/ |
| Strain ID | PTER | NO: | Template) | NO: | Replicates | min/μg) | min/μg) |
| t339870 | N/A | N/A | N/A | N/A | 6 | 0.01 | 0.00 |
| (negative | | | | | | | |
| control) | | | | | | | |
| t402006 | B. diminuta variant | N/A | N/A | 5 | 6 | 3.61 | 1.65 |
| | G1-C74 | | | | | | |
| t808375 | Mycobacterium | 2 | T29S | 199 | 2 | 0.99 | 6.03 |
| | colombiense | | | | | | |
| t808406 | Mycobacterium | 2 | A236K | 368 | 2 | 1.99 | 6.47 |
| | colombiense | | | | | | |
| t809079 | Prostheco- | 6 | G229N | 897 | 2 | 0.11 | 0.01 |
| | microbium hirschii | | | | | | |
| t809345 | Prostheco- | 6 | Q75K | 830 | 2 | 0.26 | 0.00 |
| | microbium hirschii | | | | | | |
| t809349 | Mycobacterium | 3 | N266L | 617 | 2 | 42.93 | 37.21 |
| | asiaticum | | | | | | |
| t809656 | Mycobacterium | 3 | A265Y | 611 | 2 | 48.34 | 8.56 |
| | asiaticum | | | | | | |
| t809674 | Mycobacterium | 4 | A265Y | 768 | 2 | 22.25 | 3.25 |
| | gordonae | | | | | | |
| t809682 | Mycobacterium | 4 | T148V | 676 | 2 | 1.83 | 2.50 |
| | gordonae | | | | | | |
| t809729 | Mycobacterium | 2 | H285G | 420 | 2 | 7.51 | 3.01 |
| | colombiense | | | | | | |
| t809774 | Mycobacterium | 4 | N266T | 775 | 2 | 9.91 | 7.38 |
| | gordonae | | | | | | |
| t809936 | Mycobacterium | 2 | L272W | 405 | 2 | 2.46 | 6.72 |
| | colombiense | | | | | | |
| t809996 | Mycobacterium | 2 | P151W | 319 | 2 | 5.24 | 17.87 |
| | colombiense | | | | | | |
| t810027 | Mycobacterium sp. | 1 | A265Y | 147 | 2 | 46.98 | 5.52 |
| | 852014- | | | | | | |
| | 52450_SCH5900713 | | | | | | |
| t810083 | Mycobacterium sp. | 1 | W284H | 160 | 2 | 34.30 | 2.51 |
| | 852014- | | | | | | |
| | 52450_SCH5900713 | | | | | | |
| t810099 | Prostheco- | 6 | Y128H | 860 | 2 | 0.34 | 0.00 |
| | microbium hirschii | | | | | | |
| t810152 | Mycobacterium sp. | 1 | N266M | 150 | 2 | 29.18 | 15.38 |
| | 852014- | | | | | | |
| | 52450_SCH5900713 | | | | | | |
| t810304 | Mycobacterium sp. | 1 | N266G | 149 | 2 | 11.10 | 8.31 |
| | 852014- | | | | | | |
| | 52450_SCH5900713 | | | | | | |
| t810393 | Prostheco- | 6 | M231C | 900 | 2 | 0.32 | 0.00 |
| | microbium hirschii | | | | | | |
| t810398 | Mycobacterium | 3 | Y100D | 522 | 2 | 15.48 | 49.64 |
| | asiaticum | | | | | | |
| t810450 | Mycobacterium | 2 | T99C | 275 | 2 | 3.74 | 10.81 |
| | colombiense | | | | | | |
| t810455 | Mycobacterium sp. | 1 | C267W | 156 | 2 | 3.86 | 3.22 |
| | 852014- | | | | | | |
| | 52450_SCH5900713 | | | | | | |
| t810567 | Mycobacterium | 4 | A265M | 762 | 2 | 5.56 | 0.80 |
| | gordonae | | | | | | |
| t810588 | Mycobacterium | 2 | V103H | 276 | 2 | 3.75 | 9.26 |
| | colombiense | | | | | | |
| t810666 | Mycobacterium | 2 | L272C | 402 | 2 | 2.19 | 20.63 |
| | colombiense | | | | | | |
| t810670 | Mycobacterium | 4 | C267W | 779 | 2 | 0.45 | 0.58 |
| | gordonae | | | | | | |
| t810692 | Mycobacterium | 3 | N266I | 616 | 2 | 32.84 | 21.95 |
| | asiaticum | | | | | | |
| t815551 | Mycobacterium | 4 | W284H | 784 | 2 | 16.57 | 1.52 |
| | gordonae | | | | | | |
| t815554 | Mycobacterium | 2 | M281Y | 416 | 2 | 2.07 | 8.12 |
| | colombiense | | | | | | |
| t815592 | Prostheco- | 6 | R303Q | 954 | 2 | 0.58 | 0.00 |
| | microbium hirschii | | | | | | |
| t815643 | Mycobacterium | 3 | W284H | 624 | 2 | 11.59 | 0.99 |
| | asiaticum | | | | | | |

The results confirmed that PTEs comprising the following amino acid substitutions relative to SEQ ID NO: 1 were found to be active against VX and/or VR: A265Y, N266G, N266M, C267W, and W284H.

The results confirmed that PTEs comprising the following amino acid substitutions relative to SEQ ID NO: 2 were found to be active against VX and/or VR: T29S. T99C, V103H, P151W, A236K, L272C, L272W, M281Y, and H285G.

The results confirmed that PTEs comprising the following amino acid substitutions relative to SEQ ID NO: 3 were found to be active against VX and/or VR: Y100D, A265Y, N266I, N266L, and W284H.

The results confirmed that PTEs comprising the following amino acid substitutions relative to SEQ ID NO: 4 were found to be active against VX and/or VR: T148V, A265M, A265Y, N266T, C267W, and W284H.

The results confirmed that PTEs comprising the following amino acid substitution relative to SEQ ID NO: 6 was found to be active against VX and/or VR: G229N.

### Example 6: Identification of OPNA Hydrolyzing Enzymes that Hydrolyze GB and/or GD Nerve Agents

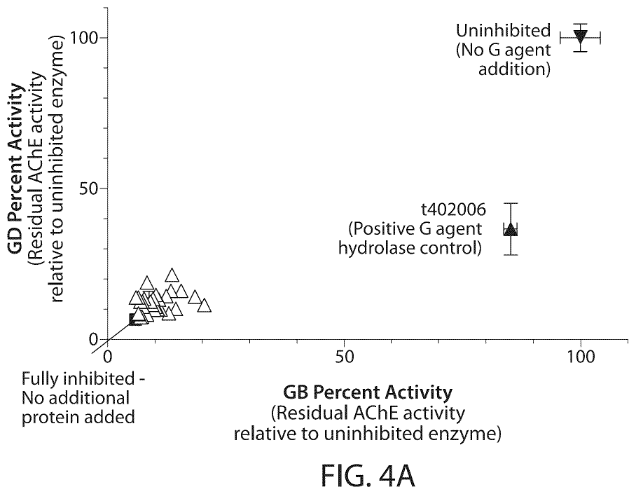

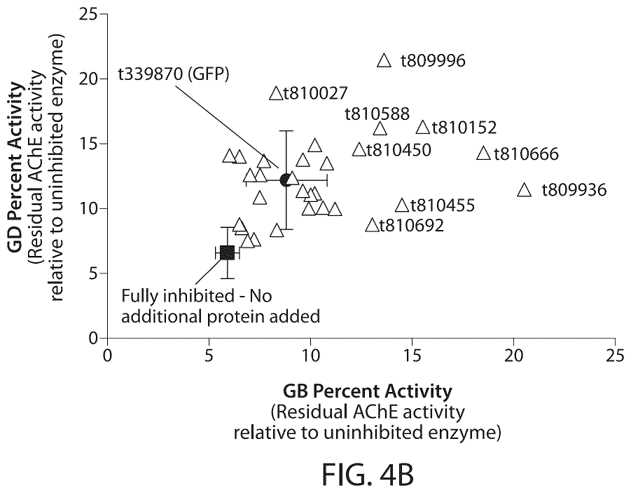

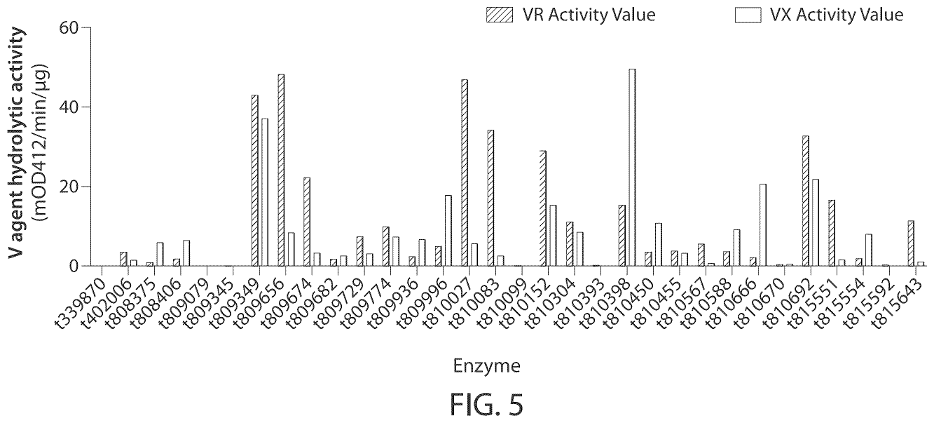

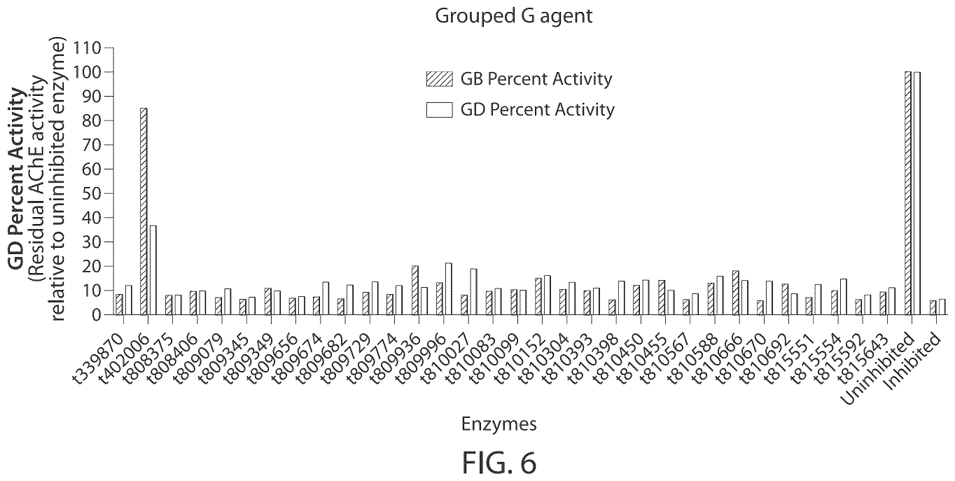

A subset of the PTE and PTER mutants with V-agent hydrolyzing activity as described in in Example 2 were screened to investigate whether they could also hydrolyze GB and/or GD nerve agents (FIGS. 4A-4B and FIG. 6). This Example describes the experimental design, general protocol, and results of the screen.

### Experimental Design

A colorimetric assay was used to measure the hydrolysis of G-type nerve agents. For G-type nerve agents, hydrolysis of acetylthiocholine by acetylcholinesterase (AChE) produces the free thiol that rapidly reacts with DTNB and generates TNB. For this assay, enzyme samples were incubated with the G-type agent. This incubation allows the enzyme to noncompetitively hydrolyze the compound before the addition of AChE. Any remaining compound will bind to and inhibit AChE, resulting in less acetylthiocholine hydrolysis and thereby, less production of TNB. Like the V-agent assay (Example 5), production of TNB was monitored at 412 nm using a spectrophotometer.

### General Protocol

Enzymes were removed from storage at −80° C. and thawed slowly on ice. Two test plates were created by transferring 50 μL of each enzyme variant to two 96-well microplates (GB Test Plate and GD Test Plate). Samples were run in singlet. Several empty wells on the dilution plate were designated to serve as internal “Inhibited AChE” and “Uninhibited AChE” controls. Either 50 μL (Inhibited AChE wells) or 60 μL (Uninhibited AChE wells) of buffer (50 mM HEPES pH 7.6, 137 mM NaCl, 2.7 mM KCl, 1 mM CoCl₂) was added to the internal control wells. The test plates were briefly stored at 4° C. until used in the assay.

Racemic G-agents were issued in saline and diluted in 50 mM HEPES pH 7.6, 137 mM NaCl, 2.7 mM KCl, 1 mM CoCl₂. GD was diluted to a concentration of 20.2 μg/L, and GB was diluted to a concentration of 33.8 μg/L. Purified human AChE (Allotropic Tech; Halethorpe, MD) was diluted to 8 μM in 0.1 M potassium phosphate (KPO₄) buffer, pH 7.4, +0.1% bovine serum albumin. Before the addition of the agent, a test plate was removed from 4° C. and allowed to warm to room temperature for 5 minutes. Once the test plate had equilibrated to room temperature, 10 μL of the diluted nerve agent was added to all wells except the “Uninhibited AChE” control wells. The plate was covered and incubated for 10 minutes at room temperature. After the incubation, 40 μL of dilute AChE was added to each well of the plate. The plate was covered and incubated for an additional 10 minutes at room temperature. Following the second incubation, 20 μL of each 100 μL reaction volume was transferred to a new plate and 180 μL of substrate [0.5 mM acetylthiocholine (Sigma; St. Louis, MO) and 1 mM DTNB (Sigma) in 0.1 M KPO₄ buffer, pH 7.4] was added to each well of the new plate. The plate was read at 412 nm in 10-second intervals for a total duration of 5 minutes in a SpectraMax Plus 384 microplate reader (Molecular Devices; San Jose, CA). Raw data (mOD412/min) were generated using the plate reader software (SoftMax v 5.4) to calculate the slope of the linear portion of the curve.

The % Activity Remaining was calculated for each sample using the following equation:

% Activity Remaining=Raw Data/(Average of “Uninhibited AChE Control” Raw Data)

100% Activity Remaining was set to equal the “Uninhibited AChE Control” average for each plate. For this assay, the “Inhibited AChE Control” has about 5-10% Activity Remaining, allowing for a large range between maximum and minimum % Activity Remaining values. The PIE expressed by positive control strain t402006 had the greatest % Activity Remaining against both GB and GD.

The following table provides the GB and GD activities of a subset of engineered enzymes described Example 2, the point mutation in each engineered enzyme relative to the enzyme template, and the source organism of each enzyme template.

TABLE 10
G-agent hydrolyzing activities of top PTEs and PTERs

| | | | Mutation | PTE or | | | |
|---|---|---|---|---|---|---|---|
| | Source organism of | Template | (relative | PTER | | GB | GD |
| | template PTE or | SEQ ID | to | SEQ ID | # of | Percent | Percent |
| Strain ID | PTER | NO: | Template) | NO: | Replicates | Activity | Activity |
| Uninhibited | N/A | N/A | N/A | N/A | 8 | 100.00% | 100.00% |
| Inhibited | N/A | N/A | N/A | N/A | 4 | 5.90% | 6.60% |
| t339870 | N/A | N/A | N/A | 20 | 3 | 8.80% | 12.20% |
| t402006 | B. diminuta variant | N/A | N/A | 5 | 3 | 85.20% | 36.70% |
| | G1-C74 | | | | | | |
| t808375 | Mycobacterium | 2 | T29S | 199 | 1 | 8.30% | 8.40% |
| | colombiense | | | | | | |
| t808406 | Mycobacterium | 2 | A236K | 368 | 1 | 9.90% | 10.00% |
| | colombiense | | | | | | |
| t809079 | Prostheco- | 6 | G229N | 897 | 1 | 7.50% | 10.90% |
| | microbium hirschii | | | | | | |
| t809345 | Prostheco- | 6 | Q75K | 830 | 1 | 6.90% | 7.50% |
| | microbium hirschii | | | | | | |
| t809349 | Mycobacterium | 3 | N266L | 617 | 1 | 11.20% | 10.00% |
| | asiaticum | | | | | | |
| t809656 | Mycobacterium | 3 | A265Y | 611 | 1 | 7.20% | 7.70% |
| | asiaticum | | | | | | |
| t809674 | Mycobacterium | 4 | A265Y | 768 | 1 | 7.70% | 13.70% |
| | gordonae | | | | | | |
| t809682 | Mycobacterium | 4 | T148V | 676 | 1 | 7.00% | 12.60% |
| | gordonae | | | | | | |
| t809729 | Mycobacterium | 2 | H285G | 420 | 1 | 9.60% | 13.80% |
| | colombiense | | | | | | |
| t809774 | Mycobacterium | 4 | N266T | 775 | 1 | 9.10% | 12.40% |
| | gordonae | | | | | | |
| t809936 | Mycobacterium | 2 | L272W | 405 | 1 | 20.50% | 11.50% |
| | colombiense | | | | | | |
| t809996 | Mycobacterium | 2 | P151W | 319 | 1 | 13.60% | 21.50% |
| | colombiense | | | | | | |
| t810027 | Mycobacterium sp. | 1 | A265Y | 147 | 1 | 8.30% | 18.90% |
| | 852014- | | | | | | |
| | 52450_SCH5900713 | | | | | | |
| t810083 | Mycobacterium sp. | 1 | W284H | 160 | 1 | 10.00% | 11.10% |
| | 852014- | | | | | | |
| | 52450_SCH5900713 | | | | | | |
| t810099 | Prostheco- | 6 | Y128H | 860 | 1 | 10.60% | 10.10% |
| | microbium hirschii | | | | | | |
| t810152 | Mycobacterium sp. | 1 | N266M | 150 | 1 | 15.50% | 16.30% |
| | 852014- | | | | | | |
| | 52450_SCH5900713 | | | | | | |
| t810304 | Mycobacterium sp. | 1 | N266G | 149 | 1 | 10.80% | 13.50% |
| | 852014- | | | | | | |
| | 52450_SCH5900713 | | | | | | |
| t810393 | Prostheco- | 6 | M231C | 900 | 1 | 10.20% | 11.20% |
| | microbium hirschii | | | | | | |
| t810398 | Mycobacterium | 3 | Y100D | 522 | 1 | 6.50% | 14.00% |
| | asiaticum | | | | | | |
| t810450 | Mycobacterium | 2 | T99C | 275 | 1 | 12.40% | 14.60% |
| | colombiense | | | | | | |
| t810455 | Mycobacterium sp. | 1 | C267W | 156 | 1 | 14.50% | 10.30% |
| | 852014- | | | | | | |
| | 52450_SCH5900713 | | | | | | |
| t810567 | Mycobacterium | 4 | A265M | 762 | 1 | 6.50% | 8.80% |
| | gordonae | | | | | | |
| t810588 | Mycobacterium | 2 | V103H | 276 | 1 | 13.40% | 16.20% |
| | colombiense | | | | | | |
| t810666 | Mycobacterium | 2 | L272C | 402 | 1 | 18.50% | 14.30% |
| | colombiense | | | | | | |
| t810670 | Mycobacterium | 4 | C267W | 779 | 1 | 6.00% | 14.10% |
| | gordonae | | | | | | |
| t810692 | Mycobacterium | 3 | N266I | 616 | 1 | 13.00% | 8.80% |
| | asiaticum | | | | | | |
| t815551 | Mycobacterium | 4 | W284H | 784 | 1 | 7.50% | 12.60% |
| | gordonae | | | | | | |
| t815554 | Mycobacterium | 2 | M281Y | 416 | 1 | 10.20% | 14.90% |
| | colombiense | | | | | | |
| t815592 | Prostheco- | 6 | R303Q | 954 | 1 | 6.60% | 8.50% |
| | microbium hirschii | | | | | | |
| t815643 | Mycobacterium | 3 | W284H | 624 | 1 | 9.60% | 11.40% |
| | asiaticum | | | | | | |

Results indicate that PTEs comprising the following amino acid substitutions relative to SEQ ID NO: 1 were found to be active against GB and/or GD: A265Y, N266G, N266M, C267W, and W284H.

Results indicate that PTEs comprising the following amino acid substitutions relative to SEQ ID NO: 2 were found to be active against GB and/or GD: T29S, T99C, V1031H, P151W, A236K, L272C, L272W, M281Y, and H285G.

Results indicate that PTEs comprising the following amino acid substitutions relative to SEQ ID NO: 3 were found to be active against GB and/or GD: Y1001D, A265Y, N266I, N266L, and W284H.

Results indicate that PTEs comprising the following amino acid substitutions relative to SEQ ID NO: 4 were found to be active against GB and/or GD: T148V, A265M, A265Y, N266T, C267W, and W284H.

Results indicate that PTEs comprising the following amino acid substitutions relative to SEQ ID NO: 6 were found to be active against GB and/or GD: Q75K, Y128H, G229N, M231C, and R303Q.

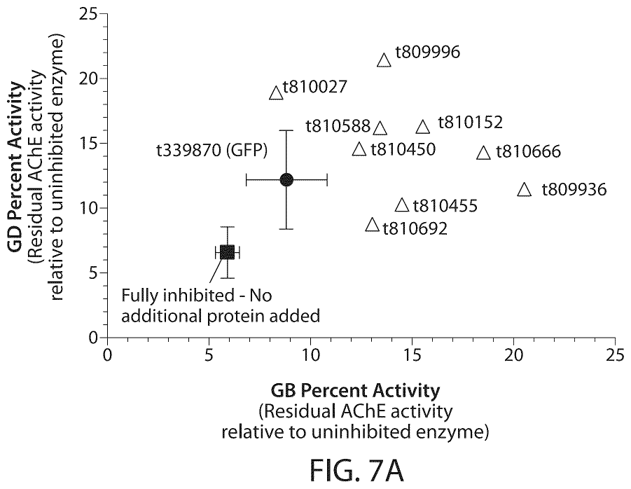

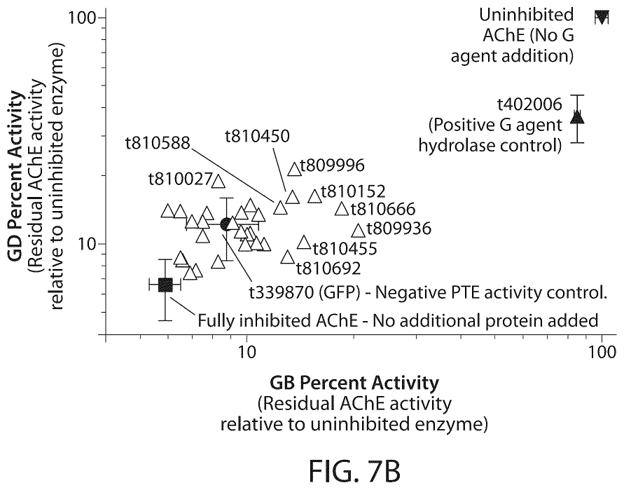

FIGS. 7A-7B depict strains expressing PTEs that are capable of hydrolyzing both V-agents and G-agents. Results indicate that PTEs comprising the following amino acid substitutions relative to SEQ ID NO: 1 were found to be active against VX, VR, GB, and GD: A265Y, N266M, and C267W. PTEs comprising the following amino acid substitutions relative to SEQ ID NO: 2 were found to be active against VX, VR, GB, and GD: T99C, V103H, P151W, L272C, and L272W. PTEs comprising the following amino acid substitutions relative to SEQ ID NO: 3 were found to be active against VX, VR, GB, and GD: A265Y, N266I, and N266L.

PTEs comprising the following amino acid substitutions relative to SEQ ID NO: 1 were found to be active against VX, VR, and GB: C267W and N266G. PTEs comprising the following amino acid substitutions relative to SEQ ID NO: 2 were found to be active against VX, VR, and GB: T99C, L272C, and L272W. PTEs comprising the following amino acid substitutions relative to SEQ ID NO: 3 were found to be active against VX, VR, and GB: N266I and N266L. PTEs comprising an A265Y substitution relative to SEQ ID NO: 1 were found to be active against VX, VR, and GD.

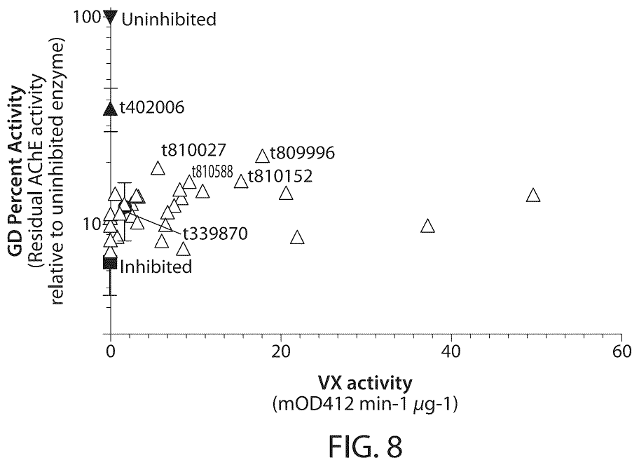

FIG. 8 depicts strains expressing PTEs that are capable of hydrolyzing VX and GD.

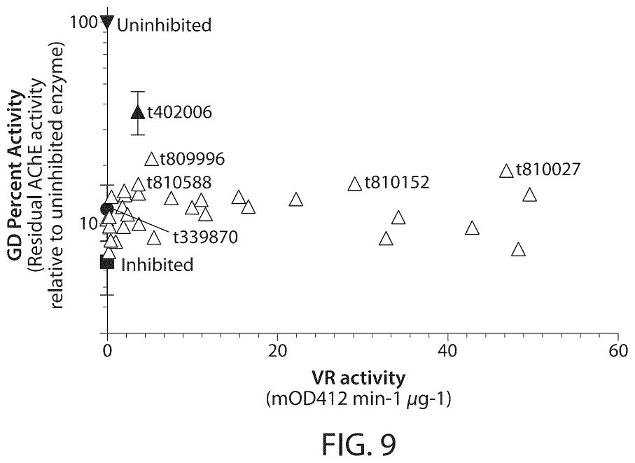

FIG. 9 depicts strains expressing PTEs that are capable of hydrolyzing VR and GD.

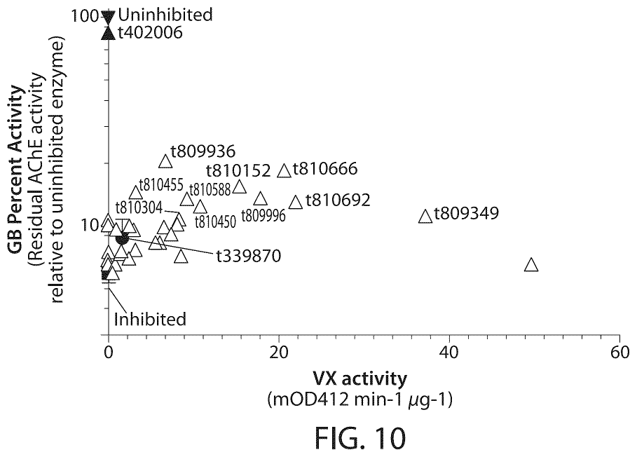

FIG. 10 depicts strains expressing PTEs that are capable of hydrolyzing VX and GB.

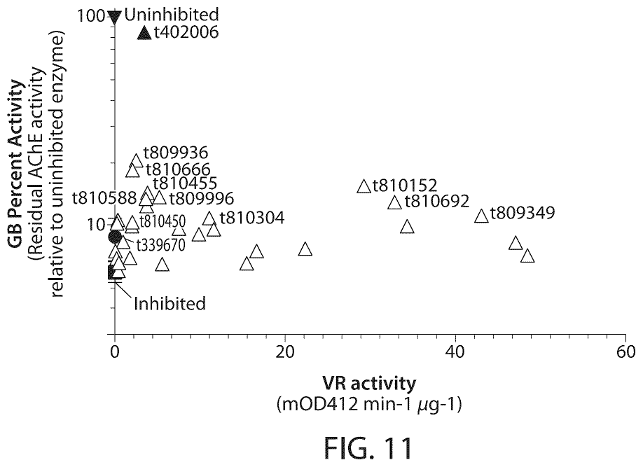

FIG. 11 depicts strains expressing PTEs that are capable of hydrolyzing VR and GB.

Several engineered PTEs identified in Example 2 showed both V-agent and G-agent hydrolysis activity (FIGS. 7A-7B). The ability of an engineered PTE to hydrolyze both V-agents and G-agents may be due to substitution mutations at important amino acid residue positions. For example, of five V-agent hydrolyzing PTEs identified in Example 2 that comprise an amino acid substitution at residue position N266, and that were tested for G-agent hydrolyzing activity, two of them were found to also exhibit G-agent hydrolyzing activity (the PTEs expressed by strain t810692 and strain t810152; FIGS. 7A-7B). Of two V-agent hydrolyzing PTEs identified in Example 2 that comprise an amino acid substitution at residue position L272, and that were tested for G-agent hydrolyzing activity, both of them also exhibited G-agent hydrolyzing activity (the PTEs expressed by strain t810666 and strain t809936; FIGS. 7A-7B). These results suggest that amino acid substitutions at residue positions N266 and L272 may be important for general OPNA hydrolyzing activity.

None of the PTERs identified in Example 2 that were tested for G-agent hydrolyzing activity were found to exhibit G-agent hydrolyzing activity.

### EQUIVALENTS

Those skilled in the art will recognize or be able to ascertain using no more than routine experimentation, many equivalents to the specific embodiments of the invention described in the present application. Such equivalents are intended to be encompassed by the following claims.

All references, including patent documents, are incorporated by reference in their entirety.

1. A host cell that comprises a heterologous polynucleotide encoding an OPNA hydrolyzing enzyme, wherein the OPNA hydrolyzing enzyme is a phosphotriesterase (PTE), and wherein the PTE comprises a sequence that is at least 90% identical to any one of SEQ ID NOs: 1-4.

2. The host cell of claim 1, wherein the PTE comprises the sequence of any one of SEQ ID NOs: 1-4.

3. The host cell of claim 1 or 2, wherein the OPNA is a V-agent (such as VX or VR), a G-agent, a VG-agent, an A-agent, an organophosphorus pesticide, or any combination thereof.

4. The host cell of any one of claims 1-3, wherein the host cell is a bacterial cell, an archaebacterial cell, a fungal cell, a yeast cell, an animal cell, a mammalian cell, or a human cell.

5. The host cell of claim 4, wherein the host cell is a bacterial cell.

6. The host cell of claim 5, wherein the bacterial cell is an Escherichia coli (E. coli) cell.

7. The host cell of claim 5, wherein the bacterial cell is a Bacillus cell.

8. The host cell of any one of claims 1-4, wherein the host cell is a filamentous fungi cell or a yeast cell.

9. The host cell of claim 6, wherein the E. coli cell is an E. coli BL21(DE3) cell.

10. The host cell of any one of claims 1-9, wherein the PTE comprises one or more amino acid substitutions relative to the sequence of any one of SEQ ID NOs: 1-4.

11. The host cell of claim 10, wherein one or more of the amino acid substitutions relative to the sequence of any one of SEQ ID NOs: 1-4 is within the active site of the PTE.

12. The host cell of claim 10 or 11, wherein the PTE comprises a sequence that is at least 90% identical to any one of SEQ ID NOs: 36-795.

13. The host cell of claim 12, wherein the PTE comprises the sequence of any one of SEQ ID NOs: 36-795.

14. The host cell of any one of claims 1-13, wherein the PTE has a K_{cat}/K_{M} value greater than 10⁷ M⁻¹ min⁻¹.

15. The host cell of any one of claims 1-14, wherein the PTE has activity against VX and VR and wherein the PTE comprises one or more of the following amino acid substitutions relative to SEQ ID NO: 1: V25M, I27F, I27M, I27T, V66I, L68N, L68M, L68C, T69S, V70P, V70C, V70T, L144I, A147G, A147F, T148V, T148C, T148M, V164I, S176T, S176M, S176V, S176C, S176Y, S176I, T177C, T179C, T179S, A181P, A181S, A181C, S208T, S208A, G228S, H263M, H263S, H263C, H263T, H263N, A265C, A265T, A265F, A265W, A265M, N266S, N266M, N266T, N266G, N266A, C267A, C267T, C267W, W284D, W284H, W284C, W284N, W284Y, Y286M, and Y286W.

16. The host cell of any one of claims 1-14 wherein the PTE has activity against VX and VR and wherein the PTE comprises one or more of the following amino acid substitutions relative to SEQ ID NO: 2: S2K, S2T, S2A, E3K, E3T, E3Q, L4I, L4V, N5R, N5Q, N5M, R8L, R8T, R8C, S10P, D12E, D12P, T13P, T13A, A14S, A14E, A14D, A15D, A15Q, A15E, L16M, V18M, V18I, M28D, T29S, T30S, T30W, T30P, E31G, I32V, I32M, I32W, 132F, A33W, E34Q, N35D, Y36F, Y36W, Y36H, E38D, A39P, W40F, D42N, E43D, D44E, D44N, V47I, V47M, A48E, A48W, D49H, D49W, D49C, D49M, V51I, V51L, K52R, R53Q, R53E, N55K, E56D, E56R, E56Q, L57F, A59E, A59Q, R60A, R60H, D63N, T64S, G72D, Y76N, Y76D, I77V, P78D, P78E, I80L, I80M, I80V, A81R, R82K, R82E, V83L, V83I, A84S, A85E, A85R, E86R, T87S, E88G, L89V, L89M, N90H, I91V, V92I, V93C, T99C, V103W, V103Y, V103H, M105W, Y106F, Y106H, Y106W, F107Y, F107W, F107M, Y109W, Y109F, L110M, L110W, E115D, G118T, G118S, E120D, I121Q, I121E, M122L, M122I, T123A, D124E, V127I, R128H, R128N, Q132D, Q132E, I134V, A135E, A135G, D136G, I139V, K140H, K140R, T150Y, T150S, T150H, P151W, P151H, P151N, P151D, V153I, P155E, P155D, G156W, E158H, A165C, Q166R, H168Q, G182D, L183T, L187H, L187M, L187F, E188W, Q190I, Q190L, K191R, F193L, E194D, E195D, L200P, S201N, R202H, R202K, V203C, V2031, 1214M, G215E, G215D, E219D, L220I, L220M, L220V, I221M, I221C, I221A, A222H, A223R, S225C, Y226W, L227V, L227I, D235C, A236H, A236W, A236K, L238M, L238H, P239S, F240W, F240D, F240Y, E241D, D242E, V244C, N245D, N245E, N245R, T246M, T246L, V247I, V247L, Q249E, Q249W, Q249R, Q249H, M250L, C251I, C251V, E252H, R253N, H255Y, H255W, K258H, K258R, M259I, A271G, L272H, L272W, L272M, D274G, D274W, E275K, V277W, S278R, Q279K, Q279R, Q279H, M281Y, M281F, P282G, N283D, N283G, H285G, L287T, H288F, H288Y, I289L, 1289V, H290F, H290L, N291R, N291D, N291T, N291E, D292N, D292R, V293I, I294L, I294V, A296M, K298M, K298R, E299Q, E299K, E299R, R300A, T303S, T303D, D304E, D304Q, E305D, E305A, Q306D, Q306E, L307I, L307V, H308R, H308E, H308N, T309Q, T309K, T309R, L311M, L311F, L311T, V312I, D313E, R316A, R316K, R316Q, R317K, R317N, I318M, I318F, I318L, E320S, E320Q, E320D, Q322R, Q322E, Q322K, A324P, A324S, Y325W, Y325F, Y325H, E326Q, E326R, and E326K.

17. The host cell of any one of claims 1-14, wherein the PTE has activity against VX and VR and wherein the PTE comprises one or more of the following amino acid substitutions relative to SEQ ID NO: 3: V25A, V25T, V25I, I27F, I27T, I27M, I27L, L68N, L68P, L68Q, L68M, T69S, V70P, V70C, Y100E, Y100H, Y100D, Y100Q, L144I, C146V, C146I, A147C, T148I, T148A, T148V, V164I, S176L, S176H, S176V, S176C, S176I, S176Y, S176F, T177C, H178D, T179C, A181S, Q189M, Q189V, Q189A, Q189I, Q189C, G206S, S208A, S208T, S208C, G209N, G228S, V260I, S262G, H263M, H263G, H263T, H263Q, A265T, A265S, A265M, A265W, N266L, N266I, N266G, N266T, N266A, N266C, N266Q, C267T, C267W, W284E, and W284T.

18. The host cell of any one of claims 1-14, wherein the PTE has activity against VX and VR and wherein the PTE comprises one or more of the following amino acid substitutions relative to SEQ ID NO: 4: L20M, V25L, V25M, F26W, F26H, I27M, I27F, I27V, I27T, V66I, L68N, L68M, L68C, Y98W, F126M, L144I, C146V, A147I, A147G, A147S, A147M, T148M, T148C, T148I, T148V, V164T, V164I, H168S, V173C, S176T, S176M, S176H, H178D, A181S, A181G, Q189M, Q189V, Q189C, I204V, G206S, S208C, S208T, G209N, V260I, S262G, H263S, H263C, H263T, H263N, A265L, A265Q, A265T, A265S, A265Y, A265W, N266C, N266G, N266S, N266T, N266A, C267W, C267A, C267T, C267G, W284H, W284Y, W284M, W284F, W284N, Y286F, Y286M, and Y286W.

19. The host cell of any one of claims 1-14 wherein the PTE has activity against VX and wherein the PTE comprises one or more of the following amino acid substitutions relative to SEQ ID NO: 1: 127C, I27Q, I27Y, L68A, S176F, T177L, T179E, G209N, and N266W.

20. The host cell of any one of claims 1-14, wherein the PTE has activity against VX and wherein the PTE comprises one or more of the following amino acid substitutions relative to SEQ ID NO: 2: E38H, I77P, V153W, E188C, L238Y, L276W, and A296R.

21. The host cell of any one of claims 1-14, wherein the PTE has activity against VX and wherein the PTE comprises one or more of the following amino acid substitutions relative to SEQ ID NO: 3: V25C, I27Y, I27C, I27W, I27V, L68A, L73V, N266W, and N266H.

22. The host cell of any one of claims 1-14, wherein the PTE has activity against VX and wherein the PTE comprises one or more of the following amino acid substitutions relative to SEQ ID NO: 4: 127C, T69S, F126C, S176Y, S176I, S176F, T177C, H178Y, Q189L, Q189I, Q189A, and S208A.

23. The host cell of any one of claims 1-14, wherein the PTE has activity against VR and wherein the PTE comprises one or more of the following amino acid substitutions relative to SEQ ID NO: 1: V25I, I27W, K145R, S176H, S208C, G228A, H263F, A265L, W284Q, W284K, W284I, and W284R.

24. The host cell of any one of claims 1-14, wherein the PTE has activity against VR and wherein the PTE comprises one or more of the following amino acid substitutions relative to SEQ ID NO: 3: Y98F, Q189L, G206N, H263F, W284K, and W284R.

25. The host cell of any one of claims 1-14, wherein the PTE has activity against VR and wherein the PTE comprises one or more of the following amino acid substitutions relative to SEQ ID NO: 4: E23D, F26M, H178V, D210C, G228S, A265M, A265C, A265I, A265V, W284C, W284Q, and W284R.

26. A method of treating or protecting against OPNA toxicity, comprising administering to a subject in need thereof a therapeutically effective amount of an OPNA hydrolyzing enzyme, or a polynucleotide encoding an OPNA hydrolyzing enzyme, wherein the OPNA hydrolyzing enzyme is a phosphotriesterase (PTE), and wherein the PTE comprises a sequence that is at least 90% identical to any one of SEQ ID NOs: 1-4.

27. A method of treating or protecting against OPNA toxicity, comprising administering to a subject in need thereof a cell comprising a heterologous polynucleotide encoding a therapeutically effective amount of an OPNA hydrolyzing enzyme, wherein the OPNA hydrolyzing enzyme is a phosphotriesterase (PTE), and wherein the PTE comprises a sequence that is at least 90% identical to any one of SEQ ID NOs: 1-4.

28. The method of claim 27, wherein the cell is a human cell, an animal cell, a yeast cell, or a bacterial cell.

29. A method of hydrolyzing or degrading an OPNA, comprising contacting an OPNA with an OPNA hydrolyzing enzyme, wherein the OPNA hydrolyzing enzyme is a phosphotriesterase (PTE), and wherein the PTE comprises a sequence that is at least 90% identical to any one of SEQ ID NOs: 1-4.

30. A method of hydrolyzing or degrading an OPNA, comprising contacting an OPNA with a cell comprising a heterologous polynucleotide encoding an OPNA hydrolyzing enzyme, wherein the OPNA hydrolyzing enzyme is a phosphotriesterase (PTE), wherein the PTE comprises a sequence that is at least 90% identical to any one of SEQ ID NOs: 1-4, and wherein the cell is in a solution, in a sprayable form, in dried form, or in immobilized form.

31. The method of claim 30, wherein the cell is an archaebacterium cell or a soil bacterium cell, such as a Bacillus cell.

32. The method of any one of claims 26-31, wherein the PTE comprises the sequence of any one of SEQ ID NOs: 1-4.

33. The method of any one of claims 26-32, wherein the OPNA is a V-agent (such as VX or VR), a G-agent, a VG-agent, an A-agent, an organophosphorus pesticide, or any combination thereof.

34. The method of any one of claims 26-33, wherein the PTE is recombinantly produced.

35. The method of claim 34, wherein the PTE is recombinantly produced in a bacterial cell or archaebacterial cell.

36. The method of claim 35, wherein the bacterial cell is an E. coli cell.

37. The method of claim 35, wherein the bacterial cell is a Bacillus cell.

38. The method of claim 34, wherein the PTE is recombinantly produced in a filamentous fungi cell or a yeast cell.

39. The method of claim 36, wherein the E. cell is an E. coli BL21(DE3) cell.

40. The method of any one of claims 26-39, wherein the PTE comprises one or more amino acid substitutions relative to the sequence of any one of SEQ ID NOs: 1-4.

41. The method of claim 40, wherein one or more of the amino acid substitutions relative to the sequence of any one of SEQ ID NOs: 1-4 is within the active site of the PTE.

42. The method of claim 40 or 41, wherein the PTE comprises a sequence that is at least 90% identical to any one of SEQ ID NOs: 36-795.

43. The method of claim 42, wherein the PTE comprises the sequence of any one of SEQ ID NOs: 36-795.

44. The method of any one of claims 26-43, wherein the PTE has a K_{cat}/K_{M} value greater than 10⁷ M⁻¹ min⁻¹.

45. The method of any one of claims 26-44, wherein the PTE is applied to an article of clothing.

46. The method of any one of claims 26-45, wherein the method is a method of protecting a subject against exposure to an OPNA.

47. The method of any one of claims 26-46, wherein the method is a method of treating a subject that has been exposed to an OPNA.

48. An OPNA hydrolyzing enzyme, wherein the OPNA hydrolyzing enzyme is a PTE, wherein the PTE comprises a sequence that is at least 90% identical to any one of SEQ ID NOs: 1-4, and wherein the sequence comprises one or more amino acid substitutions relative to the sequence of any one of SEQ ID NOs: 1-4.

49. The OPNA hydrolyzing enzyme of claim 48, wherein one or more of the amino acid substitutions relative to the sequence of any one of SEQ ID NOs: 1-4 is within the active site of the PTE.

50. The OPNA hydrolyzing enzyme of claim 48 or 49, wherein the OPNA is a V-agent (such as VX or VR), a G-agent, a VG-agent, an A-agent, an organophosphorus pesticide, or any combination thereof.

51. The OPNA hydrolyzing enzyme of any one of claims 48-50, wherein the PTE is recombinantly produced.

52. The OPNA hydrolyzing enzyme of any one of claims 48-51, wherein the PTE is recombinantly produced in a bacterial cell or an archaebacterial cell.

53. The OPNA hydrolyzing enzyme of claim 52, wherein the bacterial cell is an E. coli cell.

54. The OPNA hydrolyzing enzyme of claim 52, wherein the bacterial cell is a Bacillus cell.

55. The OPNA hydrolyzing enzyme of claim 51, wherein the PTE is recombinantly produced in a filamentous fungi cell or a yeast cell.

56. The OPNA hydrolyzing enzyme of claim 53, wherein the E. cell is an E. coli BL21(DE3) cell.

57. The OPNA hydrolyzing enzyme of any one of claims 48-56, wherein the PTE comprises a sequence that is at least 90% identical to any one of SEQ ID NOs: 36-795.

58. The OPNA hydrolyzing enzyme of claim 57, wherein the PTE comprises the sequence of any one of SEQ ID NOs: 36-795.

59. The OPNA hydrolyzing enzyme of any one of claims 48-58, wherein the PTE has a k_{cat}/K_{M} value greater than 10⁷ M⁻¹ min⁻¹.

60. The OPNA hydrolyzing enzyme of any one of claims 48-59, wherein the OPNA hydrolyzing enzyme has activity against VX and VR and wherein the OPNA hydrolyzing enzyme comprises one or more of the following amino acid substitutions relative to SEQ ID NO: 1: V25M, I27F, I27M, I27T, V66I, L68N, L68M, L68C, T69S, V70P, V70C, V70T, L144I, A147G, A147F, T148V, T148C, T148M, V164I, S176T, S176M, S176V, S176C, S176Y, S176I, T177C, T179C, T179S, A181P, A181S, A181C, S208T, S208A, G228S, H263M, H263S, H263C, H263T, H263N, A265C, A265T, A265F, A265W, A265M, N266S, N266M, N266T, N266G, N266A, C267A, C267T, C267W, W284D, W284H, W284C, W284N, W284Y, Y286M, and Y286W.

61. The OPNA hydrolyzing enzyme of any one of claims 48-59, wherein the OPNA hydrolyzing enzyme has activity against VX and VR and wherein the OPNA hydrolyzing enzyme comprises one or more of the following amino acid substitutions relative to SEQ ID NO: 2: S2K, S2T, S2A, E3K, E3T, E3Q, L4I, L4V, N5R, N5Q, N5M, R8L, R8T, R8C, S10P, D12E, D12P, T13P, T13A, A14S, A14E, A14D, A15D, A15Q, A15E, L16M, V18M, V18I, M28D, T29S, T30S, T30W, T30P, E31G, I32V, I32M, I32W, I32F, A33W, E34Q, N35D, Y36F, Y36W, Y36H, E38D, A39P, W40F, D42N, E43D, D44E, D44N, V47I, V47M, A48E, A48W, D49H, D49W, D49C, D49M, V51I, V51L, K52R, R53Q, R53E, N55K, E56D, E56R, E56Q, L57F, A59E, A59Q, R60A, R60H, D63N, T64S, G72D, Y76N, Y76D, I77V, P78D, P78E, I80L, I80M, I80V, A81R, R82K, R82E, V83L, V83I, A84S, A85E, A85R, E86R, T87S, E88G, L89V, L89M, N90H, I91V, V92I, V93C, T99C, V103W, V103Y, V103H, M105W, Y106F, Y106H, Y106W, F107Y, F107W, F107M, Y109W, Y109F, L110M, L110W, E115D, G118T, G118S, E120D, I121Q, I121E, M122L, M122I, T123A, D124E, V127I, R128H, R128N, Q132D, Q132E, I134V, A135E, A135G, D136G, I139V, K140H, K140R, T150Y, T150S, T150H, P151W, P151H, P151N, P151D, V153I, P155E, P155D, G156W, E158H, A165C, Q166R, H168Q, G182D, L183T, L187H, L187M, L187F, E188W, Q190I, Q190L, K191R, F193L, E194D, E195D, L200P, S201N, R202H, R202K, V203C, V2031, 1214M, G215E, G215D, E219D, L220I, L220M, L220V, I221M, I221C, I221A, A222H, A223R, S225C, Y226W, L227V, L227I, D235C, A236H, A236W, A236K, L238M, L238H, P239S, F240W, F240D, F240Y, E241D, D242E, V244C, N245D, N245E, N245R, T246M, T246L, V247I, V247L, Q249E, Q249W, Q249R, Q249H, M250L, C251I, C251V, E252H, R253N, H255Y, H255W, K258H, K258R, M259I, A271G, L272H, L272W, L272M, D274G, D274W, E275K, V277W, S278R, Q279K, Q279R, Q279H, M281Y, M281F, P282G, N283D, N283G, H285G, L287T, H288F, H288Y, I289L, I289V, H290F, H290L, N291R, N291D, N291T, N291E, D292N, D292R, V293I, I294L, I294V, A296M, K298M, K298R, E299Q, E299K, E299R, R300A, T303S, T303D, D304E, D304Q, E305D, E305A, Q306D, Q306E, L307I, L307V, H308R, H308E, H308N, T309Q, T309K, T309R, L311M, L311F, L311T, V312I, D313E, R316A, R316K, R316Q, R317K, R317N, I318M, I318F, I318L, E320S, E320Q, E320D, Q322R, Q322E, Q322K, A324P, A324S, Y325W, Y325F, Y325H, E326Q, E326R, and E326K.

62. The OPNA hydrolyzing enzyme of any one of claims 48-59, wherein the OPNA hydrolyzing enzyme has activity against VX and VR and wherein the OPNA hydrolyzing enzyme comprises one or more of the following amino acid substitutions relative to SEQ ID NO: 3: V25A, V25T, V25I, I27F, I27T, I27M, I27L, L68N, L68P, L68Q, L68M, T69S, V70P, V70C, Y100E, Y100H, Y100D, Y100Q, L144I, C146V, C146I, A147C, T148I, T148A, T148V, V164I, S176L, S176H, S176V, S176C, S176I, S176Y, S176F, T177C, H178D, T179C, A181S, Q189M, Q189V, Q189A, Q189I, Q189C, G206S, S208A, S208T, S208C, G209N, G228S, V260I, S262G, H263M, H263G, H263T, H263Q, A265T, A265S, A265M, A265W, N266L, N266I, N266G, N266T, N266A, N266C, N266Q, C267T, C267W, W284E, and W284T.

63. The OPNA hydrolyzing enzyme of any one of claims 48-59, wherein the OPNA hydrolyzing enzyme has activity against VX and VR and wherein the OPNA hydrolyzing enzyme comprises one or more of the following amino acid substitutions relative to SEQ ID NO: 4: L20M, V25L, V25M, F26W, F26H, I27M, I27F, I27V, I27T, V66I, L68N, L68M, L68C, Y98W, F126M, L144I, C146V, A147I, A147G, A147S, A147M, T148M, T148C, T148I, T148V, V164T, V164I, H168S, V173C, S176T, S176M, S176H, H178D, A181S, A181G, Q189M, Q189V, Q189C, I204V, G206S, S208C, S208T, G209N, V260I, S262G, H263S, H263C, H263T, H263N, A265L, A265Q, A265T, A265S, A265Y, A265W, N266C, N266G, N266S, N266T, N266A, C267W, C267A, C267T, C267G, W284H, W284Y, W284M, W284F, W284N, Y286F, Y286M, and Y286W.

64. The OPNA hydrolyzing enzyme of any one of claims 48-59, wherein the OPNA hydrolyzing enzyme has activity against VX and wherein the OPNA hydrolyzing enzyme comprises one or more of the following amino acid substitutions relative to SEQ ID NO: 1: I27C, I27Q, I27Y, L68A, S176F, T177L, T179E, G209N, and N266W.

65. The OPNA hydrolyzing enzyme of any one of claims 48-59, wherein the OPNA hydrolyzing enzyme has activity against VX and wherein the OPNA hydrolyzing enzyme comprises one or more of the following amino acid substitutions relative to SEQ ID NO: 2: E38H, I77P, V153W, E188C, L238Y, L276W, and A296R.

66. The OPNA hydrolyzing enzyme of any one of claims 48-59, wherein the OPNA hydrolyzing enzyme has activity against VX and wherein the OPNA hydrolyzing enzyme comprises one or more of the following amino acid substitutions relative to SEQ ID NO: 3: V25C, I27Y, I27C, I27W, I27V, L68A, L73V, N266W, and N266H.

67. The OPNA hydrolyzing enzyme of any one of claims 48-59, wherein the OPNA hydrolyzing enzyme has activity against VX and wherein the OPNA hydrolyzing enzyme comprises one or more of the following amino acid substitutions relative to SEQ ID NO: 4: I27C, T69S, F126C, S176Y, S176I, S176F, T177C, H178Y, Q189L, Q189I, Q189A, and S208A.

68. The OPNA hydrolyzing enzyme of any one of claims 48-59, wherein the OPNA hydrolyzing enzyme has activity against VR and wherein the OPNA hydrolyzing enzyme comprises one or more of the following amino acid substitutions relative to SEQ ID NO: 1: V25I, I27W, K145R, S176H, S208C, G228A, H263F, A265L, W284Q, W284K, W284I, and W284R.

69. The OPNA hydrolyzing enzyme of any one of claims 48-59, wherein the OPNA hydrolyzing enzyme has activity against VR and wherein the OPNA hydrolyzing enzyme comprises one or more of the following amino acid substitutions relative to SEQ ID NO: 3: Y98F, Q189L, G206N, H263F, W284K, and W284R.

70. The OPNA hydrolyzing enzyme of any one of claims 48-59, wherein the OPNA hydrolyzing enzyme has activity against VR and wherein the OPNA hydrolyzing enzyme comprises one or more of the following amino acid substitutions relative to SEQ ID NO: 4: E23D, F26M, H178V, D210C, G228S, A265M, A265C, A265I, A265V, W284C, W284Q, and W284R.

71. A host cell that comprises a heterologous polynucleotide encoding an OPNA hydrolyzing enzyme, wherein the OPNA hydrolyzing enzyme comprises a sequence that is at least 90% identical to any one of SEQ ID NOs: 6 and 796-956.

72. The host cell of claim 71, wherein the OPNA hydrolyzing enzyme comprises the sequence of any one of SEQ ID NOs: 6 and 796-956.

73. The host cell of claim 71 or 72, wherein the OPNA is a V-agent (such as VX or VR), a G-agent, a VG-agent, an A-agent, an organophosphorus pesticide, or any combination thereof.

74. The host cell of any one of claims 71-73, wherein the host cell is a bacterial cell, an archaebacterial cell, a fungal cell, a yeast cell, an animal cell, a mammalian cell, or a human cell.

75. The host cell of claim 74, wherein the host cell is a bacterial cell.

76. The host cell of claim 75, wherein the bacterial cell is an Escherichia coli (E. coli) cell.

77. The host cell of claim 75, wherein the bacterial cell is a Bacillus cell.

78. The host cell of any one of claims 71-74, wherein the host cell is a filamentous fungi cell or a yeast cell.

79. The host cell of claim 76, wherein the E. coli cell is an E. coli BL21(DE3) cell.

80. The host cell of any one of claims 71-79, wherein the PTE has a K_{cat}/K_{M} value greater than 10⁷ M⁻¹ min⁻¹.

81. A method of treating or protecting against OPNA toxicity, comprising administering to a subject in need thereof a therapeutically effective amount of an OPNA hydrolyzing enzyme, or a polynucleotide encoding an OPNA hydrolyzing enzyme, wherein the OPNA hydrolyzing enzyme comprises a sequence that is at least 90% identical to any one of SEQ ID NOs: 6 and 796-956.

82. A method of hydrolyzing or degrading an OPNA, comprising administering to a subject in need thereof a cell comprising a heterologous polynucleotide encoding a therapeutically effective amount of an OPNA hydrolyzing enzyme, wherein the OPNA hydrolyzing enzyme comprises a sequence that is at least 90% identical to any one of SEQ ID NOs: 6 and 796-956.

83. The method of claim 82, wherein the cell is a human cell, an animal cell, a yeast cell, or a bacterial cell.

84. A method of hydrolyzing or degrading an OPNA, comprising contacting an OPNA with an OPNA hydrolyzing enzyme, wherein the OPNA hydrolyzing enzyme comprises a sequence that is at least 90% identical to any one of SEQ ID NOs: 6 and 796-956.

85. A method of hydrolyzing or degrading an OPNA, comprising contacting an OPNA with a cell comprising a heterologous polynucleotide encoding an OPNA hydrolyzing enzyme, wherein the OPNA hydrolyzing enzyme comprises a sequence that is at least 90% identical to any one of SEQ ID NOs: 6 and 796-956 and wherein the cell is in a solution, in a sprayable form, in dried form, or in immobilized form.

86. The method of claim 85, wherein the cell is an archaebacterium cell or a soil bacterium cell, such as a Bacillus cell.

87. An OPNA hydrolyzing enzyme, wherein the OPNA hydrolyzing enzyme comprises a sequence that is at least 90% identical to any one of SEQ ID NOs: 6 and 796-956.

88. The OPNA hydrolyzing enzyme of claim 87, wherein the OPNA hydrolyzing enzyme comprises one or more amino acid substitutions relative to the sequence of SEQ ID NO: 6.

89. The OPNA hydrolyzing enzyme of claim 87 or 88, wherein the OPNA hydrolyzing enzyme has activity against VX and VR and wherein the OPNA hydrolyzing enzyme comprises the following amino acid substitution relative to SEQ ID NO: 6:1258V.

90. The OPNA hydrolyzing enzyme of claim 87 or 88, wherein the OPNA hydrolyzing enzyme has activity against VR and wherein the OPNA hydrolyzing enzyme comprises one or more of the following amino acid substitutions relative to SEQ ID NO: 6: R204M or G229N.

91. The host cell of any one of claims 1-14, wherein the PTE has activity against GB and GD and wherein the PTE comprises one or more of the following amino acid substitutions relative to SEQ ID NO: 1: A265Y, N266G, N266M, C267W, and W284H.

92. The host cell of any one of claims 1-14, wherein the PTE has activity against GB and GD and wherein the PTE comprises one or more of the following amino acid substitutions relative to SEQ ID NO: 2: T29S, T99C, V103H, P151W, A236K, L272C, L272W, M281Y, and H285G.

93. The host cell of any one of claims 1-14, wherein the PTE has activity against GB and GD and wherein the PTE comprises one or more of the following amino acid substitutions relative to SEQ ID NO: 3: Y100D, A265Y, N266I, N266L, and W284H.

94. The host cell of any one of claims 1-14, wherein the PTE has activity against GB and GD and wherein the PTE comprises one or more of the following amino acid substitutions relative to SEQ ID NO: 4: T148V, A265M, A265Y, N266T, C267W, and W284H.

95. The host cell of any one of claims 1-14, wherein the PTE has activity against VX, VR, GB, and GD and wherein the PTE comprises one or more of the following amino acid substitutions relative to SEQ ID NO: 1: A265Y, N266M, and C267W.

96. The host cell of any one of claims 1-14, wherein the PTE has activity against VX, VR, GB, and GD and wherein the PTE comprises one or more of the following amino acid substitutions relative to SEQ ID NO: 2: T99C, V103H, P151W, L272C, and L272W.

97. The host cell of any one of claims 1-14, wherein the PTE has activity against VX, VR, GB, and GD and wherein the PTE comprises one or more of the following amino acid substitutions relative to SEQ ID NO: 3: A265Y, N266I, and N266L.

98. The host cell of any one of claims 1-14, wherein the PTE has activity against VR, GB, and GD and wherein the PTE comprises one or more of the following amino acid substitutions relative to SEQ ID NO: 1: W284H.

99. The host cell of any one of claims 1-14, wherein the PTE has activity against VR and GD and wherein the PTE comprises one or more of the following amino acid substitutions relative to SEQ ID NO: 1: A265Y.

100. The host cell of any one of claims 1-14, wherein the PTE has activity against VX, VR and GD and wherein the PTE comprises the following amino acid substitution relative to SEQ ID NO: 1: A265Y.

101. The host cell of any one of claims 1-14, wherein the PTE has activity against VX, VR and GB and wherein the PTE comprises one or more of the following amino acid substitutions relative to SEQ ID NO: 1: C267W and N266G.

102. The host cell of any one of claims 1-14, wherein the PTE has activity against VX, VR and GB and wherein the PTE comprises one or more of the following amino acid substitutions relative to SEQ ID NO: 2: T99C, L272C, and L272W.

103. The host cell of any one of claims 1-14, wherein the PTE has activity against VX, VR and GB and wherein the PTE comprises one of the following amino acid substitutions relative to SEQ ID NO: 3: N266I or N266L.

104. The method of any one of claims 26-32, wherein the PTE has activity against VX and/or VR.

105. The method of any one of claims 26-32, wherein the PTE has activity against GB and/or GD.

106. The method of any one of claims 26-32, wherein the PTE has activity against VX, VR, GB, and GD.

107. The OPNA hydrolyzing enzyme of any one of claims 48-59, wherein the OPNA hydrolyzing enzyme has activity against GB and GD and wherein the OPNA hydrolyzing enzyme comprises one or more of the following amino acid substitutions relative to SEQ ID NO: 1: A265Y, N266G, N266M, C267W, and W284H.

108. The OPNA hydrolyzing enzyme of any one of claims 48-59, wherein the OPNA hydrolyzing enzyme has activity against GB and GD and wherein the OPNA hydrolyzing enzyme comprises one or more of the following amino acid substitutions relative to SEQ ID NO: 2: T29S, T99C, V103H, P151W, A236K, L272C, L272W, M281Y, and H285G.

109. The OPNA hydrolyzing enzyme of any one of claims 48-59, wherein the OPNA hydrolyzing enzyme has activity against GB and GD and wherein the OPNA hydrolyzing enzyme comprises one or more of the following amino acid substitutions relative to SEQ ID NO: 3: Y100D, A265Y, N266I, N266L, and W284H.

110. The OPNA hydrolyzing enzyme of any one of claims 48-59, wherein the OPNA hydrolyzing enzyme has activity against GB and GD and wherein the OPNA hydrolyzing enzyme comprises one or more of the following amino acid substitutions relative to SEQ ID NO: 4: T148V, A265M, A265Y, N266T, C267W, and W284H.

111. The OPNA hydrolyzing enzyme of any one of claims 48-59, wherein the OPNA hydrolyzing enzyme has activity against VX, VR, GB, and GD and wherein the OPNA hydrolyzing enzyme comprises one or more of the following amino acid substitutions relative to SEQ ID NO: 1: A265Y, N266M, and C267W.

112. The OPNA hydrolyzing enzyme of any one of claims 48-59, wherein the OPNA hydrolyzing enzyme has activity against VX, VR, GB, and GD and wherein the OPNA hydrolyzing enzyme comprises one or more of the following amino acid substitutions relative to SEQ ID NO: 2: T99C, V103H, P151W, L272C, and L272W.

113. The OPNA hydrolyzing enzyme of any one of claims 48-59, wherein the OPNA hydrolyzing enzyme has activity against VX, VR, GB, and GD and wherein the OPNA hydrolyzing enzyme comprises one or more of the following amino acid substitutions relative to SEQ ID NO: 3: A265Y, N266I, and N266L.

114. The OPNA hydrolyzing enzyme of any one of claims 48-59, wherein the OPNA hydrolyzing enzyme has activity against VR, GB, and GD and wherein the OPNA hydrolyzing enzyme comprises the following amino acid substitutions relative to SEQ ID NO: 1: W284H.

115. The OPNA hydrolyzing enzyme of any one of claims 48-59, wherein the OPNA hydrolyzing enzyme has activity against VR and GD and wherein the OPNA hydrolyzing enzyme comprises the following amino acid substitution relative to SEQ ID NO: 1: A265Y.

116. The OPNA hydrolyzing enzyme of any one of claims 48-59, wherein the OPNA hydrolyzing enzyme has activity against VX, VR and GD and wherein the OPNA hydrolyzing enzyme comprises the following amino acid substitution relative to SEQ ID NO: 1: A265Y.

117. The OPNA hydrolyzing enzyme of any one of claims 48-59, wherein the OPNA hydrolyzing enzyme has activity against VX, VR and GB and wherein the OPNA hydrolyzing enzyme comprises one or more of the following amino acid substitutions relative to SEQ ID NO: 1: C267W and N266G.

118. The OPNA hydrolyzing enzyme of any one of claims 48-59, wherein the OPNA hydrolyzing enzyme has activity against VX, VR and GB and wherein the OPNA hydrolyzing enzyme comprises one or more of the following amino acid substitutions relative to SEQ ID NO: 2: T99C, L272C, and L272W.

119. The OPNA hydrolyzing enzyme of any one of claims 48-59, wherein the OPNA hydrolyzing enzyme has activity against VX, VR and GB and wherein the OPNA hydrolyzing enzyme comprises one of the following amino acid substitutions relative to SEQ ID NO: 3: N266I or N266L.
